(19) United States
(12) Patent Application Publication
(10) Pub. No.: US 2020/0113852 A1
(43) Pub. Date: Apr. 16, 2020

(54) METHODS OF PATIENT SELECTION AND TREATING TRXR- OR PRDX-OVEREXPRESSED CANCERS

(71) Applicant: TRIACT THERAPEUTICS, INC., San Francico, CA (US)

(72) Inventor: Thomas F. WHITE, San Francisco, CA (US)

(21) Appl. No.: 16/626,288

(22) PCT Filed: Jun. 22, 2018
(86) PCT No.: PCT/US2018/039126

§ 371 Date: Dec. 23, 2019

Related U.S. Application Data
(60) Provisional application No. 62/523,683, filed on Jun. 22, 2017.

(51) Int. Cl. A61K 31/166; A61K 45/06; C12Q 1/6886; G01N 33/574

(52) U.S. Cl. CPC A61K 31/166; A61K 45/06; C12Q 1/6886; G01N 2333/908; C12Q 2600/158; C12Q 2600/106; G01N 2333/90212; G01N 33/57484

(57) ABSTRACT

Disclosed herein are methods and compounds for treating a cancer is characterized with an elevated expression of thioredoxin reductase (TrxR) or an elevated expression of peroxiredoxin (PRDX). In some embodiments, also disclosed herein are methods of selecting subjects for treatment or monitoring the treatment progress based on a biomarker panel.

### CROSS-REFERENCE

This application claims the benefit of U.S. Provisional Application No. 62/523,683, filed on Jun. 22, 2017, which is incorporated herein by reference in its entirety.

### BACKGROUND OF THE DISCLOSURE

Cancer can develop in any tissue or organ at any age. The etiology of cancer may not be clearly defined at times; however, mechanisms such as genetic susceptibility, chromosome breakage disorders, viruses, environmental factors and immunologic disorders have all been linked to malignant cell growth and transformation.

Worldwide, more than 10 million people are diagnosed with cancer every year and it is estimated that this number will grow to about 15 million new cases every year by 2020. Cancer causes six million deaths every year or about 12% of the deaths worldwide.

### SUMMARY OF THE DISCLOSURE

Disclosed herein, in certain embodiments, are methods of treating a subject having cancer. In some instances, the cancer is characterized with an elevated expression of thioredoxin reductase (TrxR). In other instances, the cancer is characterized with an elevated expression of peroxiredoxin (PRDX). In some embodiments, also disclosed herein are methods of selecting subjects for treatment based on a biomarker panel described herein. In additional embodiments, described herein are methods of monitoring the treatment progress based on the expression level of biomarkers from the biomarker panel described herein.

Disclosed herein, in certain embodiments, is a method of treating a subject having a cancer, comprising: (a) determining whether the subject has an elevated expression of thioredoxin reductase (TrxR) by i) measuring an expression level of TrxR from a cancer sample obtained from the subject, and ii) determining whether the expression level of TrxR from the cancer sample is elevated relative to a control sample; and (b) administering to the subject a therapeutically effective amount of 4-iodo-3-nitrobenzamide or a salt, metabolite or prodrug thereof, thereby treating the subject having the cancer characterized with the elevated expression of TrxR. In some embodiments, disclosed herein is a method of treating a subject having a cancer characterized with an elevated expression of thioredoxin reductase (TrxR), comprising: administering to the subject a therapeutically effective amount of 4-iodo-3-nitrobenzamide or a salt, metabolite or prodrug thereof, thereby treating the subject having the cancer characterized with the elevated expression of TrxR; wherein the subject is determined to have the elevated TrxR by i) measuring an expression level of TrxR from a cancer sample obtained from the subject, and ii) determining whether the expression level of TrxR from the cancer sample is elevated relative to a control sample. In some embodiments, disclosed herein is a method for treating a subject with 4-iodo-3-nitrobenzamide or a salt, metabolite or prodrug thereof, wherein the subject has a cancer, the method comprising: determining whether the subject has an elevated expression of thioredoxin reductase (TrxR) by: i) measuring an expression level of TrxR from a cancer sample obtained from the subject, and ii) determining whether the expression level of TrxR from the cancer sample is elevated relative to a control sample; if the subject has an elevated expression of TrxR, then administering 4-iodo-3-nitrobenzamide or a salt, metabolite or prodrug thereof to the subject, and if the subject does not have an elevated expression of TrxR, then administering a first-line treatment to the subject, wherein a length of disease free interval (DFI) for the subject having an elevated expression of TrxR is extended following administration of the treatment regimen comprising 4-iodo-3-nitrobenzamide or a salt, metabolite or prodrug thereof than it would be if the first-line treatment were administered. In some embodiments, the measuring comprises i) contacting a portion of the TrxR gene with a set of primers to produce amplified nucleic acids, and ii) determining the level of the amplified nucleic acids in the tumor sample. In some embodiments, the measuring comprises i) contacting the sample with an anti-TrxR antibody and ii) detecting binding between TrxR protein and the anti-TrxR antibody. In some embodiments, TrxR is thioredoxin reductase 1 (TrxR-1). In some embodiments, TrxR is thioredoxin reductase 2 (TrxR-2). In some embodiments, the elevated expression level of TrxR is about 10%, 20%, 30, 40%, 50%, 60%, 70%, 80%, 90%, 95%, or 99% higher relative to the expression level of TrxR in a cell from the control sample. In some embodiments, the elevated expression level of TrxR is about 1-fold, 2-fold, 3-fold, 4-fold, 5-fold, 6-fold, 7-fold, 8-fold, 9-fold, 10-fold, 15-fold, 20-fold or higher relative to the expression level of TrxR in a cell from the control sample. In some embodiments, the cancer is a solid tumor. In some embodiments, the solid tumor comprises brain cancer, bladder cancer, breast cancer, colorectal cancer, lung cancer, or prostate cancer. In some embodiments, the brain cancer comprises glioblastoma. In some embodiments, the glioblastoma is primary glioblastoma. In some embodiments, the glioblastoma is a secondary tumor. In some embodiments, the subject has a grade III or grade IV glioblastoma. In some embodiments, the cancer is breast cancer. In some embodiments, the breast cancer is triple negative breast cancer. In some embodiments, the cancer is a hematologic malignancy. In some embodiments, the hematologic malignancy comprises T-cell leukemia. In some embodiments, the T-cell leukemia comprises large granular lymphocytic leukemia, T-cell acute lymphoblastic leukemia (T-ALL) or T-cell prolymphocytic leukemia (T-PLL). In some embodiments, the cancer is a melanoma. In some embodiments, the cancer is a metastatic cancer. In some embodiments, the cancer is a relapsed cancer. In some embodiments, the cancer is a refractory cancer. In some embodiments, 4-iodo-3-nitrobenzamide or a salt, metabolite or prodrug thereof is administered to the subject at a range of about 5 mg/kg to about 40 mg/kg. In some embodiments, 4-iodo-3-nitrobenzamide or a salt, metabolite or prodrug thereof is administered to the subject at a range of about 6 mg/kg to about 40 mg/kg, about 6 mg/kg to about 30 mg/kg, about 6 mg/kg to about 20 mg/kg, about 6 mg/kg to about 10 mg/kg, about 6 mg/kg to about 9 mg/kg, about 7 mg/kg to about 30 mg/kg, about 7 mg/kg to about 20 mg/kg, about 7 mg/kg to about 9 mg/kg, about 7 mg/kg to about 8 mg/kg, about 8 mg/kg to about 20 mg/kg, about 8 mg/kg to about 9 mg/kg, or about 8 mg/kg to about 8.6 mg/kg. In some embodiments, 4-iodo-3-nitrobenzamide or a salt, metabolite or prodrug thereof is administered to the subject at about 5 mg/kg, about 6 mg/kg, about 7 mg/kg, about 8 mg/kg, about 8.5 mg/kg, about 8.6 mg/kg, about 9 mg/kg, about 10 mg/kg, about 15 mg/kg, about 20 mg/kg, about 25 mg/kg, about 30 mg/kg, or about 40 mg/kg. In some embodiments, the 4-iodo-3-nitrobenzamide or a salt, metabolite or prodrug thereof is administered to the subject once per day. In some embodiments, the 4-iodo-3-nitrobenzamide or a salt, metabolite or prodrug thereof is administered to the subject for about twice a week. In some embodiments, the 4-iodo-3-nitrobenzamide or a salt, metabolite or prodrug thereof is administered to the subject for about four, five or six weeks. In some embodiments, the 4-iodo-3-nitrobenzamide or a salt, metabolite or prodrug thereof is administered to the subject continuously for about 1, 2, 3, 4 or more treatment cycles. In some embodiments, the 4-iodo-3-nitrobenzamide or a salt, metabolite or prodrug thereof is administered to the subject intermittently for about 1, 2, 3, 4 or more treatment cycles. In some embodiments, a treatment cycle is about 28 days. In some embodiments, the method further comprises administering to the subject an additional therapeutic agent. In some embodiments, the additional therapeutic agent is an inhibitor of TrxR. In some embodiments, the inhibitor of TrxR is epigallocatechin-3-O-gallate (EGCG), n-butyl 2-imidazolyl disulfide, 1-methylpropyl 2-imidazolyl disulfide, n-decyl 2-imidazolyl disulfide, an alkyl 2-imidazolyl disulfide analogue, auranofin, or a dinitrohalobenzene. In some embodiments, the inhibitor of TrxR is phosphine gold(I), a gold(I) carbene complex, a gold(III)-dithiocarbamato complex, an arsenic derivative, or azelaic acid. In some embodiments, the additional therapeutic agent is an inhibitor of PRDX. In some embodiments, the inhibitor of PRDX is a pan-PRDX inhibitor. In some embodiments, the inhibitor of PRDX is Conoidin A. In some embodiments, the additional therapeutic agent is an inhibitor of glutathione (GSH). In some embodiments, the inhibitor of GSH is L-buthionine sulfoximine (BSO). In some embodiments, the additional therapeutic agent is temozolomide. In some embodiments, the additional therapeutic agent is radiation. In some embodiments, the additional therapeutic agent is a standard-of-care chemotherapy. In some embodiments, 4-iodo-3-nitrobenzamide or a salt, metabolite or prodrug thereof and the additional therapeutic agent are administered sequentially. In some embodiments, 4-iodo-3-nitrobenzamide or a salt, metabolite or prodrug thereof and the additional therapeutic agent are administered concurrently. In some embodiments, treatment of 4-iodo-3-nitrobenzamide or a salt, metabolite or prodrug thereof increases the length of disease free interval (DFI) relative to a subject not treated with 4-iodo-3-nitrobenzamide or a salt, metabolite or prodrug thereof. In some embodiments, the control sample is a non-cancerous sample. In some embodiments, the tumor sample is a tissue sample. In some embodiments, the tumor sample is a liquid sample. In some embodiments, the tumor sample is a cell-free sample.

Disclosed herein, in certain embodiments, is a method of diagnosing and treating cancer in a subject, the method comprising: (a) obtaining a cancer sample from a human subject; (b) detecting whether an expression level of thioredoxin reductase (TrxR) is elevated in the cancer sample relative to an expression level of TrxR in a control sample; (c) diagnosing the subject as having a cancer characterized with the elevated expression of TrxR; and (d) administering an effective amount of 4-iodo-3-nitrobenzamide or a salt, metabolite or prodrug thereof to the diagnosed subject. In some embodiments, the detecting comprises i) contacting a portion of the TrxR gene with a set of primers to produce amplified nucleic acids, and ii) determining the level of the amplified nucleic acids in the tumor sample. In some embodiments, the detecting comprises i) contacting the sample with an anti-TrxR antibody and ii) detecting binding between TrxR protein and the anti-TrxR antibody. In some embodiments, TrxR is thioredoxin reductase 1 (TrxR-1). In some embodiments, TrxR is thioredoxin reductase 2 (TrxR-2). In some embodiments, the elevated expression level of TrxR is about 10%, 20%, 30, 40%, 50%, 60%, 70%, 80%, 90%, 95%, or 99% higher relative to the expression level of TrxR in a cell from the control sample. In some embodiments, the elevated expression level of TrxR is about 1-fold, 2-fold, 3-fold, 4-fold, 5-fold, 6-fold, 7-fold, 8-fold, 9-fold, 10-fold, 15-fold, 20-fold or higher relative to the expression level of TrxR in a cell from the control sample. In some embodiments, the cancer is a solid tumor. In some embodiments, the solid tumor comprises brain cancer, bladder cancer, breast cancer, colorectal cancer, lung cancer, or prostate cancer. In some embodiments, the brain cancer comprises glioblastoma. In some embodiments, the glioblastoma is primary glioblastoma. In some embodiments, the glioblastoma is a secondary tumor. In some embodiments, the subject has a grade III or grade IV glioblastoma. In some embodiments, the cancer is breast cancer. In some embodiments, the breast cancer is triple negative breast cancer. In some embodiments, the cancer is a hematologic malignancy. In some embodiments, the hematologic malignancy comprises T-cell leukemia. In some embodiments, the T-cell leukemia comprises large granular lymphocytic leukemia, T-cell acute lymphoblastic leukemia (T-ALL) or T-cell prolymphocytic leukemia (T-PLL). In some embodiments, the cancer is a melanoma. In some embodiments, the cancer is a metastatic cancer. In some embodiments, the cancer is a relapsed cancer. In some embodiments, the cancer is a refractory cancer. In some embodiments, 4-iodo-3-nitrobenzamide or a salt, metabolite or prodrug thereof is administered to the subject at a range of about 5 mg/kg to about 40 mg/kg. In some embodiments, 4-iodo-3-nitrobenzamide or a salt, metabolite or prodrug thereof is administered to the subject at a range of about 6 mg/kg to about 40 mg/kg, about 6 mg/kg to about 30 mg/kg, about 6 mg/kg to about 20 mg/kg, about 6 mg/kg to about 10 mg/kg, about 6 mg/kg to about 9 mg/kg, about 7 mg/kg to about 30 mg/kg, about 7 mg/kg to about 20 mg/kg, about 7 mg/kg to about 9 mg/kg, about 7 mg/kg to about 8 mg/kg, about 8 mg/kg to about 20 mg/kg, about 8 mg/kg to about 9 mg/kg, or about 8 mg/kg to about 8.6 mg/kg. In some embodiments, 4-iodo-3-nitrobenzamide or a salt, metabolite or prodrug thereof is administered to the subject at about 5 mg/kg, about 6 mg/kg, about 7 mg/kg, about 8 mg/kg, about 8.5 mg/kg, about 8.6 mg/kg, about 9 mg/kg, about 10 mg/kg, about 15 mg/kg, about 20 mg/kg, about 25 mg/kg, about 30 mg/kg, or about 40 mg/kg. In some embodiments, the 4-iodo-3-nitrobenzamide or a salt, metabolite or prodrug thereof is administered to the subject once per day. In some embodiments, the 4-iodo-3-nitrobenzamide or a salt, metabolite or prodrug thereof is administered to the subject for about twice a week. In some embodiments, the 4-iodo-3-nitrobenzamide or a salt, metabolite or prodrug thereof is administered to the subject for about four, five or six weeks. In some embodiments, the 4-iodo-3-nitrobenzamide or a salt, metabolite or prodrug thereof is administered to the subject continuously for about 1, 2, 3, 4 or more treatment cycles. In some embodiments, the 4-iodo-3-nitrobenzamide or a salt, metabolite or prodrug thereof is administered to the subject intermittently for about 1, 2, 3, 4 or more treatment cycles. In some embodiments, a treatment cycle is about 28 days. In some embodiments, the method further comprises administering to the subject an additional therapeutic agent. In some embodiments, the additional therapeutic agent is an inhibitor of TrxR. In some embodiments, the inhibitor of TrxR is epigallocatechin-3-O-gallate (EGCG), n-butyl 2-imidazolyl disulfide, 1-methylpropyl 2-imidazolyl disulfide, n-decyl 2-imidazolyl disulfide, an alkyl 2-imidazolyl disulfide analogue, auranofin, or a dinitrohalobenzene. In some embodiments, the inhibitor of TrxR is phosphine gold(I), a gold(I) carbene complex, a gold(III)-dithiocarbamato complex, an arsenic derivative, or azelaic acid. In some embodiments, the additional therapeutic agent is an inhibitor of PRDX. In some embodiments, the inhibitor of PRDX is a pan-PRDX inhibitor. In some embodiments, the inhibitor of PRDX is Conoidin A. In some embodiments, the additional therapeutic agent is an inhibitor of glutathione (GSH). In some embodiments, the inhibitor of GSH is L-buthionine sulfoximine (BSO). In some embodiments, the additional therapeutic agent is temozolomide. In some embodiments, the additional therapeutic agent is radiation. In some embodiments, the additional therapeutic agent is a standard-of-care chemotherapy. In some embodiments, 4-iodo-3-nitrobenzamide or a salt, metabolite or prodrug thereof and the additional therapeutic agent are administered sequentially. In some embodiments, 4-iodo-3-nitrobenzamide or a salt, metabolite or prodrug thereof and the additional therapeutic agent are administered concurrently. In some embodiments, treatment of 4-iodo-3-nitrobenzamide or a salt, metabolite or prodrug thereof increases the length of disease free interval (DFI) relative to a subject not treated with 4-iodo-3-nitrobenzamide or a salt, metabolite or prodrug thereof. In some embodiments, the control sample is a non-cancerous sample. In some embodiments, the tumor sample is a tissue sample. In some embodiments, the tumor sample is a liquid sample. In some embodiments, the tumor sample is a cell-free sample.

Disclosed herein, in certain embodiments, is a method of treating a subject having a cancer, comprising: (a) determining whether the subject has an elevated expression of peroxiredoxin (PRDX) by i) measuring an expression level of PRDX from a cancer sample obtained from the subject, and ii) determining whether the expression level of PRDX from the cancer sample is elevated relative to a control sample; and (b) administering to the subject a therapeutically effective amount of 4-iodo-3-nitrobenzamide or a salt, metabolite or prodrug thereof, thereby treating the subject having the cancer characterized with the elevated expression of PRDX. In some embodiments, disclosed herein is a method of treating a subject having a cancer characterized with an elevated expression of peroxiredoxin (PRDX), comprising: administering to the subject a therapeutically effective amount of 4-iodo-3-nitrobenzamide or a salt, metabolite or prodrug thereof, thereby treating the subject having the cancer characterized with the elevated expression of PRDX; wherein the subject is determined to have the elevated PRDX by i) measuring an expression level of PRDX from a cancer sample obtained from the subject, and ii) determining whether the expression level of PRDX from the cancer sample is elevated relative to a control sample. In some embodiments, disclosed herein is a method for treating a subject with 4-iodo-3-nitrobenzamide or a salt, metabolite or prodrug thereof, wherein the subject has a cancer, the method comprising: determining whether the subject has an elevated expression of peroxiredoxin (PRDX) by: i) measuring an expression level of PRDX from a cancer sample obtained from the subject, and ii) determining whether the expression level of PRDX from the cancer sample is elevated relative to a control sample; if the subject has an elevated expression of PRDX, then administering 4-iodo-3-nitrobenzamide or a salt, metabolite or prodrug thereof to the subject, and if the subject does not have an elevated expression of PRDX, then administering a first-line treatment to the subject, wherein a length of disease free interval (DFI) for the subject having an elevated expression of PRDX is extended following administration of the treatment regimen comprising 4-iodo-3-nitrobenzamide or a salt, metabolite or prodrug thereof than it would be if the first-line treatment were administered. In some embodiments, the measuring comprises i) contacting a portion of the PRDX gene with a set of primers to produce amplified nucleic acids, and ii) determining the level of the amplified nucleic acids in the cancer sample. In some embodiments, the measuring comprises i) contacting the sample with an anti-PRDX antibody and ii) detecting binding between PRDX protein and the anti-PRDX antibody. In some embodiments, peroxiredoxin is peroxiredoxin-1 (PRDX-1). In some embodiments, the elevated expression of peroxiredoxin-1 is determined by i) measuring an expression level of PRDX-1 from a cancer sample obtained from the subject, and ii) determining whether the expression level of PRDX-1 from the tumor sample is elevated relative to a control sample. In some embodiments, the measuring comprises i) contacting a portion of the PRDX-1 gene with a set of primers to produce amplified nucleic acids, and ii) determining the level of the amplified nucleic acids in the tumor sample. In some embodiments, the measuring comprises i) contacting the sample with an anti-PRDX-1 antibody and ii) detecting binding between PRDX-1 protein and the anti-PRDX-1 antibody. In some embodiments, the elevated expression level of PRDX is about 10%, 20%, 30, 40%, 50%, 60%, 70%, 80%, 90%, 95%, or 99% higher relative to the expression level of PRDX in a cell from the control sample. In some embodiments, the elevated expression level of PRDX is about 1-fold, 2-fold, 3-fold, 4-fold, 5-fold, 6-fold, 7-fold, 8-fold, 9-fold, 10-fold, 15-fold, 20-fold or higher relative to the expression level of PRDX in a cell from the control sample. In some embodiments, the cancer is a solid tumor. In some embodiments, the solid tumor comprises brain cancer, bladder cancer, breast cancer, colorectal cancer, lung cancer, or prostate cancer. In some embodiments, the brain cancer comprises glioblastoma. In some embodiments, the glioblastoma is primary glioblastoma. In some embodiments, the glioblastoma is a secondary tumor. In some embodiments, the subject has a grade III or grade IV glioblastoma. In some embodiments, the cancer is breast cancer. In some embodiments, the breast cancer is triple negative breast cancer. In some embodiments, the cancer is a hematologic malignancy. In some embodiments, the hematologic malignancy comprises T-cell leukemia. In some embodiments, the T-cell leukemia comprises large granular lymphocytic leukemia, T-cell acute lymphoblastic leukemia (T-ALL) or T-cell prolymphocytic leukemia (T-PLL). In some embodiments, the cancer is a melanoma. In some embodiments, the cancer is a metastatic cancer. In some embodiments, the cancer is a relapsed cancer. In some embodiments, the cancer is a refractory cancer. In some embodiments, 4-iodo-3-nitrobenzamide or a salt, metabolite or prodrug thereof is administered to the subject at a range of about 5 mg/kg to about 40 mg/kg. In some embodiments, 4-iodo-3-nitrobenzamide or a salt, metabolite or prodrug thereof is administered to the subject at a range of about 6 mg/kg to about 40 mg/kg, about 6 mg/kg to about 30 mg/kg, about 6 mg/kg to about 20 mg/kg, about 6 mg/kg to about 10 mg/kg, about 6 mg/kg to about 9 mg/kg, about 7 mg/kg to about 30 mg/kg, about 7 mg/kg to about 20 mg/kg, about 7 mg/kg to about 9 mg/kg, about 7 mg/kg to about 8 mg/kg, about 8 mg/kg to about 20 mg/kg, about 8 mg/kg to about 9 mg/kg, or about 8 mg/kg to about 8.6 mg/kg. In some embodiments, 4-iodo-3-nitrobenzamide or a salt, metabolite or prodrug thereof is administered to the subject at about 5 mg/kg, about 6 mg/kg, about 7 mg/kg, about 8 mg/kg, about 8.5 mg/kg, about 8.6 mg/kg, about 9 mg/kg, about 10 mg/kg, about 15 mg/kg, about 20 mg/kg, about 25 mg/kg, about 30 mg/kg, or about 40 mg/kg. In some embodiments, the 4-iodo-3-nitrobenzamide or a salt, metabolite or prodrug thereof is administered to the subject once per day. In some embodiments, the 4-iodo-3-nitrobenzamide or a salt, metabolite or prodrug thereof is administered to the subject for about twice a week. In some embodiments, the 4-iodo-3-nitrobenzamide or a salt, metabolite or prodrug thereof is administered to the subject for about four, five or six weeks. In some embodiments, the 4-iodo-3-nitrobenzamide or a salt, metabolite or prodrug thereof is administered to the subject continuously for about 1, 2, 3, 4 or more treatment cycles. In some embodiments, the 4-iodo-3-nitrobenzamide or a salt, metabolite or prodrug thereof is administered to the subject intermittently for about 1, 2, 3, 4 or more treatment cycles. In some embodiments, a treatment cycle is about 28 days. In some embodiments, the method further comprises administering to the subject an additional therapeutic agent. In some embodiments, the additional therapeutic agent is an inhibitor of TrxR. In some embodiments, the inhibitor of TrxR is epigallocatechin-3-O-gallate (EGCG), n-butyl 2-imidazolyl disulfide, 1-methylpropyl 2-imidazolyl disulfide, n-decyl 2-imidazolyl disulfide, an alkyl 2-imidazolyl disulfide analogue, auranofin, or a dinitrohalobenzene. In some embodiments, the inhibitor of TrxR is phosphine gold(I), a gold(I) carbene complex, a gold(III)-dithiocarbamato complex, an arsenic derivative, or azelaic acid. In some embodiments, the additional therapeutic agent is an inhibitor of PRDX. In some embodiments, the inhibitor of PRDX is a pan-PRDX inhibitor. In some embodiments, the inhibitor of PRDX is Conoidin A. In some embodiments, the additional therapeutic agent is an inhibitor of glutathione (GSH). In some embodiments, the inhibitor of GSH is L-buthionine sulfoximine (BSO). In some embodiments, the additional therapeutic agent is temozolomide. In some embodiments, the additional therapeutic agent is radiation. In some embodiments, the additional therapeutic agent is a standard-of-care chemotherapy. In some embodiments, 4-iodo-3-nitrobenzamide or a salt, metabolite or prodrug thereof and the additional therapeutic agent are administered sequentially. In some embodiments, 4-iodo-3-nitrobenzamide or a salt, metabolite or prodrug thereof and the additional therapeutic agent are administered concurrently. In some embodiments, treatment of 4-iodo-3-nitrobenzamide or a salt, metabolite or prodrug thereof increases the length of disease free interval (DFI) relative to a subject not treated with 4-iodo-3-nitrobenzamide or a salt, metabolite or prodrug thereof. In some embodiments, the control sample is a non-cancerous sample. In some embodiments, the tumor sample is a tissue sample. In some embodiments, the tumor sample is a liquid sample. In some embodiments, the tumor sample is a cell-free sample.

Disclosed herein, in certain embodiments, is a method of diagnosing and treating cancer in a subject, the method comprising: (a) obtaining a cancer sample from a human subject; (b) detecting whether an expression level of peroxiredoxin (PRDX) is elevated in the cancer sample relative to an expression level of PRDX in a control sample; (c) diagnosing the subject as having a cancer characterized with the elevated expression of PRDX; and (d) administering an effective amount of 4-iodo-3-nitrobenzamide or a salt, metabolite or prodrug thereof to the diagnosed subject. In some embodiments, the detecting comprises i) contacting a portion of the PRDX gene with a set of primers to produce amplified nucleic acids, and ii) determining the level of the amplified nucleic acids in the tumor sample. In some embodiments, the detecting comprises i) contacting the sample with an anti-PRDX antibody and ii) detecting binding between PRDX protein and the anti-PRDX antibody. In some embodiments, peroxiredoxin is peroxiredoxin-1 (PRDX-1). In some embodiments, the elevated expression of peroxiredoxin-1 is determined by i) measuring an expression level of PRDX-1 from a tumor sample obtained from the subject, and ii) determining whether the expression level of PRDX-1 from the tumor sample is elevated relative to a control sample. In some embodiments, the measuring comprises i) contacting a portion of the PRDX-1 gene with a set of primers to produce amplified nucleic acids, and ii) determining the level of the amplified nucleic acids in the tumor sample. In some embodiments, the measuring comprises i) contacting the sample with an anti-PRDX-1 antibody and ii) detecting binding between PRDX-1 protein and the anti-PRDX-1 antibody. In some embodiments, the elevated expression level of PRDX is about 10%, 20%, 30, 40%, 50%, 60%, 70%, 80%, 90%, 95%, or 99% higher relative to the expression level of PRDX in a cell from the control sample. In some embodiments, the elevated expression level of PRDX is about 1-fold, 2-fold, 3-fold, 4-fold, 5-fold, 6-fold, 7-fold, 8-fold, 9-fold, 10-fold, 15-fold, 20-fold or higher relative to the expression level of PRDX in a cell from the control sample. In some embodiments, the cancer is a solid tumor. In some embodiments, the solid tumor comprises brain cancer, bladder cancer, breast cancer, colorectal cancer, lung cancer, or prostate cancer. In some embodiments, the brain cancer comprises glioblastoma. In some embodiments, the glioblastoma is primary glioblastoma. In some embodiments, the glioblastoma is a secondary tumor. In some embodiments, the subject has a grade III or grade IV glioblastoma. In some embodiments, the cancer is breast cancer. In some embodiments, the breast cancer is triple negative breast cancer. In some embodiments, the cancer is a hematologic malignancy. In some embodiments, the hematologic malignancy comprises T-cell leukemia. In some embodiments, the T-cell leukemia comprises large granular lymphocytic leukemia, T-cell acute lymphoblastic leukemia (T-ALL) or T-cell prolymphocytic leukemia (T-PLL). In some embodiments, the cancer is a melanoma. In some embodiments, the cancer is a metastatic cancer. In some embodiments, the cancer is a relapsed cancer. In some embodiments, the cancer is a refractory cancer. In some embodiments, 4-iodo-3-nitrobenzamide or a salt, metabolite or prodrug thereof is administered to the subject at a range of about 5 mg/kg to about 40 mg/kg. In some embodiments, 4-iodo-3-nitrobenzamide or a salt, metabolite or prodrug thereof is administered to the subject at a range of about 6 mg/kg to about 40 mg/kg, about 6 mg/kg to about 30 mg/kg, about 6 mg/kg to about 20 mg/kg, about 6 mg/kg to about 10 mg/kg, about 6 mg/kg to about 9 mg/kg, about 7 mg/kg to about 30 mg/kg, about 7 mg/kg to about 20 mg/kg, about 7 mg/kg to about 9 mg/kg, about 7 mg/kg to about 8 mg/kg, about 8 mg/kg to about 20 mg/kg, about 8 mg/kg to about 9 mg/kg, or about 8 mg/kg to about 8.6 mg/kg. In some embodiments, 4-iodo-3-nitrobenzamide or a salt, metabolite or prodrug thereof is administered to the subject at about 5 mg/kg, about 6 mg/kg, about 7 mg/kg, about 8 mg/kg, about 8.5 mg/kg, about 8.6 mg/kg, about 9 mg/kg, about 10 mg/kg, about 15 mg/kg, about 20 mg/kg, about 25 mg/kg, about 30 mg/kg, or about 40 mg/kg. In some embodiments, the 4-iodo-3-nitrobenzamide or a salt, metabolite or prodrug thereof is administered to the subject once per day. In some embodiments, the 4-iodo-3-nitrobenzamide or a salt, metabolite or prodrug thereof is administered to the subject for about twice a week. In some embodiments, the 4-iodo-3-nitrobenzamide or a salt, metabolite or prodrug thereof is administered to the subject for about four, five or six weeks. In some embodiments, the 4-iodo-3-nitrobenzamide or a salt, metabolite or prodrug thereof is administered to the subject continuously for about 1, 2, 3, 4 or more treatment cycles. In some embodiments, the 4-iodo-3-nitrobenzamide or a salt, metabolite or prodrug thereof is administered to the subject intermittently for about 1, 2, 3, 4 or more treatment cycles. In some embodiments, a treatment cycle is about 28 days. In some embodiments, the method further comprises administering to the subject an additional therapeutic agent. In some embodiments, the additional therapeutic agent is an inhibitor of TrxR. In some embodiments, the inhibitor of TrxR is epigallocatechin-3-O-gallate (EGCG), n-butyl 2-imidazolyl disulfide, 1-methylpropyl 2-imidazolyl disulfide, n-decyl 2-imidazolyl disulfide, an alkyl 2-imidazolyl disulfide analogue, auranofin, or a dinitrohalobenzene. In some embodiments, the inhibitor of TrxR is phosphine gold(I), a gold(I) carbene complex, a gold(III)-dithiocarbamato complex, an arsenic derivative, or azelaic acid. In some embodiments, the additional therapeutic agent is an inhibitor of PRDX. In some embodiments, the inhibitor of PRDX is a pan-PRDX inhibitor. In some embodiments, the inhibitor of PRDX is Conoidin A. In some embodiments, the additional therapeutic agent is an inhibitor of glutathione (GSH). In some embodiments, the inhibitor of GSH is L-buthionine sulfoximine (BSO). In some embodiments, the additional therapeutic agent is temozolomide. In some embodiments, the additional therapeutic agent is radiation. In some embodiments, the additional therapeutic agent is a standard-of-care chemotherapy. In some embodiments, 4-iodo-3-nitrobenzamide or a salt, metabolite or prodrug thereof and the additional therapeutic agent are administered sequentially. In some embodiments, 4-iodo-3-nitrobenzamide or a salt, metabolite or prodrug thereof and the additional therapeutic agent are administered concurrently. In some embodiments, treatment of 4-iodo-3-nitrobenzamide or a salt, metabolite or prodrug thereof increases the length of disease free interval (DFI) relative to a subject not treated with 4-iodo-3-nitrobenzamide or a salt, metabolite or prodrug thereof. In some embodiments, the control sample is a non-cancerous sample. In some embodiments, the tumor sample is a tissue sample. In some embodiments, the tumor sample is a liquid sample. In some embodiments, the tumor sample is a cell-free sample.

Disclosed herein, in certain embodiments, is a method of selecting a subject for treatment with 4-iodo-3-nitrobenzamide or a salt, metabolite or prodrug thereof, comprising: (a) contacting at least one gene selected from thioredoxin reductase 2 (TXNRD2), thioredoxin 2 (TXN2), methionine sulfoxide reductase B3 (MSRB3), methionine sulfoxide reductase A (MSRA), and glutathione transferase zeta 1 (GSTZ1) with a set of primers to produce amplified nucleic acids, wherein the at least one gene is isolated from a tumor sample obtained from the subject; (b) determining the level of the amplified nucleic acids in the tumor sample relative to a control; and (c) administering a therapeutically effective amount of 4-iodo-3-nitrobenzamide or a salt, metabolite or prodrug thereof to the subject if the level of the amplified nucleic acids is greater than the level in the control. In some embodiments, the level of at least one gene selected from TXNRD2, TXN2, MSRB3 and MSRA is determined. In some embodiments, the level of TXNRD2, TXN2, MSRB3 and MSRA are determined. In some embodiments, the level of the amplified nucleic acids from at least one gene selected from TXNRD2, TXN2, MSRB3, MSRA and GSTZ1 correlates to a decreased risk of disease progression. In some embodiments, the method further comprises determining the level of amplified nucleic acids from at least one gene selected from NAD(P)H dehydrogenase quinone 2 (NQO2), glutathione S-transferase theta 2 (GSTT2), glutathione S-transferase M3 (GSTM3), glutaredoxin (GLRX), selenoprotein O (SELO), paraoxonase 1 (PON1), glutathione S-transferase omega 1 (GSTO1), glutaredoxin 3 (GLRX3), selenoprotein X 1 (SEPX1), and thioredoxin reductase 1 (TXNRD1) and comparing the level with a control. In some embodiments, the treatment with 4-iodo-3-nitrobenzamide or a salt, metabolite or prodrug thereof is discontinued if the level of amplified nucleic acids is greater than the level in the control. In some embodiments, 4-iodo-3-nitrobenzamide or a salt, metabolite or prodrug thereof is administered to the subject at a range of about 5 mg/kg to about 40 mg/kg. In some embodiments, 4-iodo-3-nitrobenzamide or a salt, metabolite or prodrug thereof is administered to the subject at a range of about 6 mg/kg to about 40 mg/kg, about 6 mg/kg to about 30 mg/kg, about 6 mg/kg to about 20 mg/kg, about 6 mg/kg to about 10 mg/kg, about 6 mg/kg to about 9 mg/kg, about 7 mg/kg to about 30 mg/kg, about 7 mg/kg to about 20 mg/kg, about 7 mg/kg to about 9 mg/kg, about 7 mg/kg to about 8 mg/kg, about 8 mg/kg to about 20 mg/kg, about 8 mg/kg to about 9 mg/kg, or about 8 mg/kg to about 8.6 mg/kg. In some embodiments, 4-iodo-3-nitrobenzamide or a salt, metabolite or prodrug thereof is administered to the subject at about 5 mg/kg, about 6 mg/kg, about 7 mg/kg, about 8 mg/kg, about 8.5 mg/kg, about 8.6 mg/kg, about 9 mg/kg, about 10 mg/kg, about 15 mg/kg, about 20 mg/kg, about 25 mg/kg, about 30 mg/kg, or about 40 mg/kg. In some embodiments, the 4-iodo-3-nitrobenzamide or a salt, metabolite or prodrug thereof is administered to the subject once per day. In some embodiments, the 4-iodo-3-nitrobenzamide or a salt, metabolite or prodrug thereof is administered to the subject for about twice a week. In some embodiments, the 4-iodo-3-nitrobenzamide or a salt, metabolite or prodrug thereof is administered to the subject for about four, five or six weeks. In some embodiments, the 4-iodo-3-nitrobenzamide or a salt, metabolite or prodrug thereof is administered to the subject continuously for about 1, 2, 3, 4 or more treatment cycles. In some embodiments, the 4-iodo-3-nitrobenzamide or a salt, metabolite or prodrug thereof is administered to the subject intermittently for about 1, 2, 3, 4 or more treatment cycles. In some embodiments, a treatment cycle is about 28 days. In some embodiments, the cancer is a TrxR-overexpressed cancer. In some embodiments, the cancer is a PRDX-overexpressed cancer. In some embodiments, the cancer is a solid tumor. In some embodiments, the solid tumor comprises brain cancer, bladder cancer, breast cancer, colorectal cancer, lung cancer, or prostate cancer. In some embodiments, the brain cancer comprises glioblastoma. In some embodiments, the glioblastoma is primary glioblastoma. In some embodiments, the glioblastoma is a secondary tumor. In some embodiments, the subject has a grade III or grade IV glioblastoma. In some embodiments, the cancer is breast cancer. In some embodiments, the breast cancer is triple negative breast cancer. In some embodiments, the cancer is a hematologic malignancy. In some embodiments, the hematologic malignancy comprises T-cell leukemia. In some embodiments, the T-cell leukemia comprises large granular lymphocytic leukemia, T-cell acute lymphoblastic leukemia (T-ALL) or T-cell prolymphocytic leukemia (T-PLL). In some embodiments, the cancer is a melanoma. In some embodiments, the cancer is a metastatic cancer. In some embodiments, the cancer is a relapsed cancer. In some embodiments, the cancer is a refractory cancer. In some embodiments, the method further comprises administering to the subject an additional therapeutic agent. In some embodiments, the additional therapeutic agent is an inhibitor of TrxR. In some embodiments, the inhibitor of TrxR is epigallocatechin-3-O-gallate (EGCG), n-butyl 2-imidazolyl disulfide, 1-methylpropyl 2-imidazolyl disulfide, n-decyl 2-imidazolyl disulfide, an alkyl 2-imidazolyl disulfide analogue, auranofin, or a dinitrohalobenzene. In some embodiments, the inhibitor of TrxR is phosphine gold(I), a gold(I) carbene complex, a gold(III)-dithiocarbamato complex, an arsenic derivative, or azelaic acid. In some embodiments, the additional therapeutic agent is an inhibitor of PRDX. In some embodiments, the inhibitor of PRDX is a pan-PRDX inhibitor. In some embodiments, the inhibitor of PRDX is Conoidin A. In some embodiments, the additional therapeutic agent is an inhibitor of glutathione (GSH). In some embodiments, the inhibitor of GSH is L-buthionine sulfoximine (BSO). In some embodiments, the additional therapeutic agent is temozolomide. In some embodiments, the additional therapeutic agent is radiation. In some embodiments, the additional therapeutic agent is a standard-of-care chemotherapy. In some embodiments, 4-iodo-3-nitrobenzamide or a salt, metabolite or prodrug thereof and the additional therapeutic agent are administered sequentially. In some embodiments, 4-iodo-3-nitrobenzamide or a salt, metabolite or prodrug thereof and the additional therapeutic agent are administered concurrently. In some embodiments, the control is a non-cancerous sample. In some embodiments, the tumor sample is a tissue sample. In some embodiments, the tumor sample is a liquid sample. In some embodiments, the tumor sample is a cell-free sample.

Disclosed herein, in certain embodiments, is a method of monitoring a treatment regimen in a subject having a cancer, comprising: (a) administering to the subject a therapeutically effective amount of 4-iodo-3-nitrobenzamide or a salt, metabolite or prodrug thereof; (b) contacting at least one gene selected from NAD(P)H dehydrogenase quinone 2 (NQO2), glutathione S-transferase theta 2 (GSTT2), glutathione S-transferase M3 (GSTM3), glutaredoxin (GLRX), selenoprotein O (SELO), paraoxonase 1 (PON1), glutathione S-transferase omega 1 (GSTO1), glutaredoxin 3 (GLRX3), selenoprotein X 1 (SEPX1), and thioredoxin reductase 1 (TXNRD1) with a set of primers to produce amplified nucleic acids, wherein the at least one gene is isolated from a tumor sample obtained from the subject after treatment initiation; (c) determining the level of the amplified nucleic acids in the tumor sample relative to a control; and (d) continuing treatment with 4-iodo-3-nitrobenzamide or a metabolite thereof if the level of the amplified nucleic acids is lower than or is the same as the level of the control, or discontinuing treatment with 4-iodo-3-nitrobenzamide or a metabolite thereof if the level of the amplified nucleic acids is greater than the level of the control. In some embodiments, the level of at least one gene selected from NQO2, GSTT2, GSTM3, GLRX, GSTO1, GLRX3 and TXNRD1 is determined. In some embodiments, the level of at least one gene selected from NQO2, GSTT2, GSTM3, GLRX, GSTO1 and GLRX3 is determined. In some embodiments, the level of at least one gene selected from GSTT2, GSTM3, GLRX, GSTO1 and GLRX3 is determined. In some embodiments, the level of at least one gene selected from GSTT2, GSTM3, and GSTO1 is determined. In some embodiments, the level of at least one gene selected from NQO2, SELO, PON1, SEPX1 and TXNRD1 is determined. In some embodiments, the level of at least one gene selected from SELO, PON1, SEPX1 and TXNRD1 is determined. In some embodiments, the level of at least one gene selected from SELO, PON1 and SEPX1 is determined. In some embodiments, the level of amplified nucleic acids greater than the level in the control correlates to an increased risk of disease progression. In some embodiments, the method further comprises determining the level of amplified nucleic acids from at least one gene selected from thioredoxin reductase 2 (TXNRD2), thioredoxin 2 (TXN2), methionine sulfoxide reductase B3 (MSRB3), methionine sulfoxide reductase A (MSRA), and glutathione transferase zeta 1 (GSTZ1) and comparing the level with a control. In some embodiments, the level of amplified nucleic acids greater than the level in the control correlates to a decreased risk of disease progression. In some embodiments, 4-iodo-3-nitrobenzamide or a salt, metabolite or prodrug thereof is administered to the subject at a range of about 5 mg/kg to about 40 mg/kg. In some embodiments, 4-iodo-3-nitrobenzamide or a salt, metabolite or prodrug thereof is administered to the subject at a range of about 6 mg/kg to about 40 mg/kg, about 6 mg/kg to about 30 mg/kg, about 6 mg/kg to about 20 mg/kg, about 6 mg/kg to about 10 mg/kg, about 6 mg/kg to about 9 mg/kg, about 7 mg/kg to about 30 mg/kg, about 7 mg/kg to about 20 mg/kg, about 7 mg/kg to about 9 mg/kg, about 7 mg/kg to about 8 mg/kg, about 8 mg/kg to about 20 mg/kg, about 8 mg/kg to about 9 mg/kg, or about 8 mg/kg to about 8.6 mg/kg. In some embodiments, 4-iodo-3-nitrobenzamide or a salt, metabolite or prodrug thereof is administered to the subject at about 5 mg/kg, about 6 mg/kg, about 7 mg/kg, about 8 mg/kg, about 8.5 mg/kg, about 8.6 mg/kg, about 9 mg/kg, about 10 mg/kg, about 15 mg/kg, about 20 mg/kg, about 25 mg/kg, about 30 mg/kg, or about 40 mg/kg. In some embodiments, the 4-iodo-3-nitrobenzamide or a salt, metabolite or prodrug thereof is administered to the subject once per day. In some embodiments, the 4-iodo-3-nitrobenzamide or a salt, metabolite or prodrug thereof is administered to the subject for about twice a week. In some embodiments, the 4-iodo-3-nitrobenzamide or a salt, metabolite or prodrug thereof is administered to the subject for about four, five or six weeks. In some embodiments, the 4-iodo-3-nitrobenzamide or a salt, metabolite or prodrug thereof is administered to the subject continuously for about 1, 2, 3, 4 or more treatment cycles. In some embodiments, the 4-iodo-3-nitrobenzamide or a salt, metabolite or prodrug thereof is administered to the subject intermittently for about 1, 2, 3, 4 or more treatment cycles. In some embodiments, a treatment cycle is about 28 days. In some embodiments, the cancer is a TrxR-overexpressed cancer. In some embodiments, the cancer is a PRDX-overexpressed cancer. In some embodiments, the cancer is a solid tumor. In some embodiments, the solid tumor comprises brain cancer, bladder cancer, breast cancer, colorectal cancer, lung cancer, or prostate cancer. In some embodiments, the brain cancer comprises glioblastoma. In some embodiments, the glioblastoma is primary glioblastoma. In some embodiments, the glioblastoma is a secondary tumor. In some embodiments, the subject has a grade III or grade IV glioblastoma. In some embodiments, the cancer is breast cancer. In some embodiments, the breast cancer is triple negative breast cancer. In some embodiments, the cancer is a hematologic malignancy. In some embodiments, the hematologic malignancy comprises T-cell leukemia. In some embodiments, the T-cell leukemia comprises large granular lymphocytic leukemia, T-cell acute lymphoblastic leukemia (T-ALL) or T-cell prolymphocytic leukemia (T-PLL). In some embodiments, the cancer is a melanoma. In some embodiments, the cancer is a metastatic cancer. In some embodiments, the cancer is a relapsed cancer. In some embodiments, the cancer is a refractory cancer. In some embodiments, the method further comprises administering to the subject an additional therapeutic agent. In some embodiments, the additional therapeutic agent is an inhibitor of TrxR. In some embodiments, the inhibitor of TrxR is epigallocatechin-3-O-gallate (EGCG), n-butyl 2-imidazolyl disulfide, 1-methylpropyl 2-imidazolyl disulfide, n-decyl 2-imidazolyl disulfide, an alkyl 2-imidazolyl disulfide analogue, auranofin, or a dinitrohalobenzene. In some embodiments, the inhibitor of TrxR is phosphine gold(I), a gold(I) carbene complex, a gold(III)-dithiocarbamato complex, an arsenic derivative, or azelaic acid. In some embodiments, the additional therapeutic agent is an inhibitor of PRDX. In some embodiments, the inhibitor of PRDX is a pan-PRDX inhibitor. In some embodiments, the inhibitor of PRDX is Conoidin A. In some embodiments, the additional therapeutic agent is an inhibitor of glutathione (GSH). In some embodiments, the inhibitor of GSH is L-buthionine sulfoximine (BSO). In some embodiments, the additional therapeutic agent is temozolomide. In some embodiments, the additional therapeutic agent is radiation. In some embodiments, the additional therapeutic agent is a standard-of-care chemotherapy. In some embodiments, 4-iodo-3-nitrobenzamide or a salt, metabolite or prodrug thereof and the additional therapeutic agent are administered sequentially. In some embodiments, 4-iodo-3-nitrobenzamide or a salt, metabolite or prodrug thereof and the additional therapeutic agent are administered concurrently. In some embodiments, the control is a non-cancerous sample. In some embodiments, the tumor sample is a tissue sample. In some embodiments, the tumor sample is a liquid sample. In some embodiments, the tumor sample is a cell-free sample.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A-FIG. 1E illustrate structures and biochemical data for iniparib, its metabolites and biotin-derivatized iniparib. FIG. 1A shows the skeletal formula of Iniparib, its metabolites and its biotin-derivative tool compound. FIG. 1B shows the effect of Iniparib, I-NOBA and Iniparib-biotin on MDA-MB-453 and HCT116 cells viability. Cells were preincubated for 5 h in the presence (black) or absence (clear) of 1 mM BSO. Afterwards cells were treated with the indicated concentrations of Iniparib (circle), I-NOBA (triangle) or Iniparib-biotin (square), or their vehicle. After 48 h of treatment, cell viability was measured using WST-1 assay. FIG. 1C shows Iniparib metabolites release by MDA-MB-453 and HCT116 cells. Cells were preincubated for 48 h in the presence (black) or absence (clear) of 1 mM BSO. Afterwards cells were exposed to 100 μM Iniparib and aliquots of the incubation media were taken 1 h and 5 h after addition of Iniparib. Subsequently, media aliquots were analyzed by XLC-MS/MS for quantification of Iniparib and metabolites, as described in the experimental procedures section. (D) GSTP1 protein expression level in MDA-MB-453 and HCT116 cells. Cells were preincubated for 48 h in the presence or absence of 1 mM BSO. Cell lysates were analyzed by anti-GSTP1 blot as described in the Examples. FIG. 1E shows in vitro Iniparib-Glutathione conjugation by GSTP1. After incubation of Iniparib with reduced glutathione in the presence (black) or the absence (clear) of recombinant GSTP1, Iniparib and I-GS were quantified by XLC-MS/MS as described in the experimental procedures section. All values in B, C and E are expressed as means±SEM of at least three independent experiments.

FIG. 2A-FIG. 2E illustrate in vitro modification of GAPDH by Iniparib and I-NOBA. FIG. 2A shows biochemical approach. Reduced form of GAPDH was first incubated with 100 μM Iniparib or I-NOBA. The resulting samples were then reduced or not with DTT as indicated, and free cysteine sulfhydryl residues of GAPDH were biotinylated as indicated in the experimental procedures section. Modified GAPDH was finally analyzed by SDS-PAGE under non-reducing conditions followed by streptavidin-HRP blot and anti-GAPDH blot. (FIG. 2B to FIG. 2E) Nano LC-MS analysis. GAPDH was incubated with 30 μM Iniparib or I-NOBA and then reduced or not with DTT. Intact protein molecular weights were recorded as described in the Experimental Procedures section. Are shown the deconvoluted mass spectrum of recombinant GAPDH (FIG. 2B), of GAPDH after reaction with Iniparib (FIG. 2C), of GAPDH after reaction with I-NOBA (FIG. 2D) and of GAPDH after reaction with I-NOBA and reduction with DTT (FIG. 2E).

FIG. 3A-FIG. 3C show protein modification by Iniparib-biotin in HCT116 cells. HCT116 cells were pre-treated or not for 48 h with 1 mM BSO. Thereafter Iniparib-biotin was added for 4 h at various concentrations up to 100 μM (FIG. 3A), at 100 μM for increasing times from 10 to 240 min (FIG. 3B), or at 100 μM for 4 h (FIG. 3C). Cell lysates prepared in 1.5%-octylglucoside containing buffer were analyzed by SDS-PAGE under reducing conditions (FIG. 3A and FIG. 3B) or under either reducing or non-reducing conditions as indicated (FIG. 3C) followed by streptavidin-HRP blot and anti-GAPDH blot.

FIG. 4A-FIG. 4E show modification of Prx1 by Iniparib-Biotin and Iniparib. GSH-depleted HCT116 cells were incubated for 4 h either with 100 μM Iniparib-biotin (FIG. 4A) or with 100 μM Iniparib (FIG. 4C). Monomeric-avidin (FIG. 4A) and Prdx1 (FIG. 4C) precipitated complexes were separated by SDS-PAGE under reducing conditions and gels were stained with PageBlue Reagent®. In the case of monomeric-avidin pull down, aliquots were submitted in parallel to streptavidin-HRP blot (FIG. 4A). FIG. 4E is represented the sequence of T20 (169-190) peptide modified by Iniparib and Iniparib-Biotin adduct. Marker ions from b series allowed the positioning of the modification on Cys 173. FIG. 4B shows LTQ-MS/MS spectra of T20 peptide. Above: with CAM modified Cys 173 in control sample. Below: with Iniparib-biotin modified Cys 173 in Iniparib-Biotin treated sample. Inset, close-up of the region with the b₅ and y₅ ions allowing the positioning of the modification. FIG. 4D shows HCD-MS/MS spectra of T20 peptide. Above: with CAM modified Cys 173 in control sample. Below: with Iniparib modified Cys 173 in Iniparib treated sample. Inset, close-up of the region with the b₅ and y₅ ions allowing the positioning of the modification.

FIG. 5A-FIG. 5B show subcellular localization of Iniparib-Biotin targets. GSH-depleted HCT116 cells were incubated with Iniparib-Biotin at 100 μM for 30 min. FIG. 5A shows confocal planes of a stack of images were projected into a single plane at maximum intensity. Image merged fluorescence signals for Alexa 488-streptavindin and Mitotracker red CMX. FIG. 5B shows a zoomed-in region of a selected region on the image (FIG. 5A). Co-localization analysis was performed on a single central plane of the stack.

FIG. 6A-FIG. 6C show ROS product and apoptosis. FIG. 6A shows that ROS production in HCT116 cells. 2′,7′-dichlorodihydrofluorescein diacetate was used to monitor ROS production. Cells pretreated (black) or not (clear) for 20 h with 1 mM BSO were incubated with DCFH-DA for 15 min, washed three times with HBSS and incubated in HBBS with 1 μM DCFH-DA to minimize the leaking out of the product. Afterwards, Iniparib (100 circle), menadione (50 square) or their vehicle (1% DMSO, triangle) were added. Images were captured for each compound loaded on the cells incubated in a thermostated dark chamber. FIG. 6B shows Iniparib-induced cell death. Dot plots of cell- and DNA-associated fluorescence bicolor DiOC2(3)/DAPI signals. Necrotic (green), apoptotic (blue) and viable (red) cells are depicted. Left panel: Vehicle (9 h, 1% DMSO); Right panel: Iniparib (9h, 100 μM). FIG. 6C shows the time course of cell death induction. Iniparib (100 μM, black) or its vehicle (1% DMSO, clear) were added on BSO-pretreated HCT116 cells for the indicated time. Results were expressed as a percentage of necrotic (circle), apoptotic (square) and viable (triangle) cells.

FIG. 7 illustrates exemplary skeletal formula of Iniparib metabolites described herein.

FIG. 8 shows GSH depletion with BSO in HCT116 and MDA-MB-453 cell lines. Cells were seeded (96 well plates) at increasing densities, the highest density corresponding to that used for viability studies. 24 h after, cells were incubated or not in the presence of 0.5 mM BSO for 24 h. GSH amount was measured thereafter using GSH-Glo™ glutathione assay.

FIG. 9 shows comparison of the pattern of proteins modified by Iniparib-biotin in HCT116 and MDA-MB-453 cells. HCT116 and MDA-MB-453 cells were pre-treated or not for 48 h in the presence of 1 mM BSO. Then cells were incubated with 100 M Iniparib-biotin or its vehicle for 4 h. Cell lysates prepared in 1.5%-octylglucoside containing buffer were analysed by SDS-PAGE under reducing conditions followed by streptavidin-HRP blot.

FIG. 10 shows HCT116 cells labeling with Iniparib-Biotin. Cells were preincubated for 18 h in the presence or absence of 1 mM BSO. Afterwards cells were treated with the indicated concentrations of Iniparib-biotin, or its vehicle, for the indicated times. Biotin labeling was detected with Alexa 488-streptavidin as described in the experimental procedure section. Contrast and intensities are the same for the conditions displayed.

FIG. 11A-FIG. 11D show Iniparib-biotin activation/protein modification in cytosols and mitochondria of HCT116 cells. FIG. 11A and FIG. 11B show HCT116 cells were pre-treated or not for 48 h with 1 mM BSO. Thereafter Iniparib-biotin at 100 M was added for 4 h. Cell homogenates were then processed for cytosol preparation (FIG. 11A) or mitochondria immuno-purification (FIG. 11B). FIG. 11C and FIG. 11D show cells were pretreated or not for 48 h in the presence of 1 mM BSO.

FIG. 12A-FIG. 12B show MDA-MB-453 cell analysis following incubation with Iniparib. FIG. 12A shows dot plots of cell- and DNA-associated fluorescence bicolor DiOC2(3)/DAPI signals. Necrotic (green), apoptotic (blue) and viable (red) cells are depicted.

FIG. 12B shows kinetics of cell apoptosis induction by Iniparib expressed as a percentage of necrotic (circles), apoptotic (squares) and viable (triangles) cells.

FIG. 13A-FIG. 13E show TrxR modification and inhibition by Iniparib in HCT 116 cells. FIG. 13A shows the effect of transitory exposure of Iniparib and Iniparib-biotin on cell viability. Cells were preincubated for 5 h in the presence or absence of 1 mM BSO. Afterwards cells were treated with the indicated concentrations of Iniparib (circle), Iniparib-biotin (triangle) or their vehicle, either continuously for 24 h (left panel) or only for the first 1, 2 or 4 h, the compounds being removed during the following 23, 22 or 21 h, respectively. Cell viability was measured using WST-1 assay. FIG. 13B shows the effect of Iniparib and Iniparib-biotin on TrxR endogenous activity. Cells were preincubated for 48 h in the presence of 0.5 mM BSO. Afterwards cells were treated with 100 μM of Iniparib, Iniparib-biotin or their vehicle, for the indicated times (left panel), or for 4 h with the indicated concentrations of Iniparib, Iniparib-biotin or Auranofin, or their vehicle (right panel). After treatment, TrxR activities were determined on clarified cell lysates (50 μg protein) using the DTNB reduction assay. All values are expressed as means±SEM of at least three independent experiments. FIG. 13C, FIG. 13D and FIG. 13E show time-course of TrxR1 and TrxR2 modification by Iniparib-biotin. Cells were preincubated for 48 h in the presence of 1 mM BSO. Then cells were treated for various times up to 2 h in the presence of 100 μM Iniparib-biotin or its vehicle. Cell lysates were prepared in 1.5%-octylglucoside containing buffer and processed for TrxR1 or TrxR2 immunoprecipitation as described in the experimental procedures section. Cells extracts (FIG. 13C), TrxR1 and TrxR2 immunoprecipitates (FIG. 13D) were analyzed by SDS-PAGE under reducing conditions followed by streptavidin-HRP blot. (FIG. 13E) Quantification of bands of (FIG. 13D) where square and left axis represent TrxR1, and triangle and right axis TrxR2. Results in FIG. 13C, FIG. 13D and FIG. 13E are representative of at least three independent experiments.

FIG. 14A-FIG. 14D show mass spectrometry analysis of TrxR1 and TrxR2 modification by Iniparib in HCT116 cells. FIG. 14A shows HCT116 cells were pre-treated for 48 h with 1 mM BSO. Then cells were incubated for 1 h our 4 h in the presence of 100 μM Iniparib or its vehicle. TrxR1 and TrxR2 were immunoprecipitated from octylglucoside cell extracts (12 mg proteins) and precipitated complexes were separated by SDS-PAGE under reducing conditions, gels being stained with PageBlue Reagent®. FIG. 14B shows relative abundance by XIC analysis of Sec peptides identified from nano LC-MS/MS analyses of tryptic digest gel bands (residues [488-499] for TrxR1 and residues [513-524] for TrxR2) in Vehicle, 1-h and 4-h Iniparib treated samples. Percentage of peptides modified by 2 carbamidomethyl (CAM) adducts, 1 CAM and 1 Iniparib adducts or 2 Iniparib adducts are given for each condition. ND, not detected. FIG. 14C shows LTQ-MS/MS spectra of the doubly charged peptide ions at m/z [668.23]²⁺ and m/z [746.28]²⁺ for SGASILQAGCUG peptide of TrxR1. Above: with CAM modified Cys and Sec in Vehicle sample. Below: with CAM modified Cys and Iniparib modified Sec in 4 h Iniparib treated sample. All y-ions series, including y₂ are modified allowing to position the Iniparib modification on Sec residue. FIG. 14D shows LTQ-MS/MS spectra of the doubly charged peptide ions at m/z [689.73]²⁺ and m/z [767.78]²⁺ for SGLDPTVTGCUG peptide of TrxR2. Above: with CAM modified Cys and Sec in Vehicle sample. Below: with CAM modified Cys and Iniparib modified Sec in 4 h Iniparib treated sample. All y-ions series, including y₂ are modified allowing to position the Iniparib modification on Sec residue.

FIG. 15A-FIG. 15C show in vitro modification and inhibition of TrxR by Iniparib-biotin. TrxR1 and TrxR2 immunoprecipitates from HCT116 cells (pretreated for 48 h with 1 mM BSO) (FIG. 15A) and 36 pmol of rat liver TrxR (FIG. 15B) were incubated at 37° C. either for 30 min (FIG. 15A) or for various time up to 120 min as indicated (FIG. 15B) with 30 μM Iniparib-biotin in the presence or the absence of 200 μM NADPH and 5 μM FAD. TrxR Samples from FIG. 15A and FIG. 15B were analyzed by SDS-PAGE under reducing conditions followed by streptavidin-HRP blot. FIG. 15C shows 36 pmol of rat liver TrxR were pre-incubated with 100 μM Iniparib in the presence of 200 μM NADPH for the indicated time up to 120 min. Then, TrxR activity was determined using the DTNB reduction assay. Are represented on the graph: TrxR DTNB reductase activity (left axis) and TrxR-Iniparib-biotin bands intensity from FIG. 15B (right axis). All values are expressed as means±SEM of at least three independent experiments. Results in FIG. 15A and FIG. 15B are representative of three independent experiments.

FIG. 16A-FIG. 16E show radical generation by rat liver TrxR with Iniparib, Iniparib-modified rat liver TrxR and ΔSec human TrxR1. FIG. 16A shows examples of ESR spectra for DEPMPO adducts generated by rat liver TrxR. (1) TrxR (0.5 μM) was incubated at 21° C. for 1.5 h with 0.5 mM NADPH, 100 μM Iniparib and 50 mM DEPMPO under room air. (2) Same as 1 but Iniparib was replaced by its vehicle. Spectra 4 and 5 are the same as 1 and 2 with the addition of SOD (1000 U/mL). Rat liver TrxR alone was incubated with DEPMPO under room air (6). Spectrum 7 is the same as 1 but under anaerobic conditions. The EPR instrument settings are given in the experimental section. FIG. 16B shows relative amount of DEPMPO adducts formed by rat liver TrxR in the presence or the absence of Iniparib. Relative amounts of DEPMPO/HOO^{∘} and DEPMPO/HO^{∘} adducts derived from simulation of spectra 1 and 2. FIG. 16C shows relative amount of DEPMPO adducts generated by Iniparib-modified rat liver TrxR. Relative amounts of DEPMPO/HOO^{∘} and DEPMPO/HO^{∘} adducts derived from simulation of spectra obtained under conditions similar to 1 but where rat liver TrxR was replaced by Iniparib-modified rat liver TrxR or vehicle-treated rat liver TrxR. FIG. 16D shows relative amount of DEPMPO adducts for ΔSec human TrxR1 the presence or the absence of Iniparib. Relative amounts of DEPMPO/HOO^{∘} and DEPMPO/HO^{∘} adducts derived from simulation of spectra obtained under conditions similar to 1 and 2 but where TrxR1 was replaced by ΔSec-TrxR1. FIG. 16B, FIG. 16C, and FIG. 16D are expressed in arbitrary units. Results are representative of three independent experiments. FIG. 16E shows spin trapping scheme: structure of EPR-silent DEPMPO and of the EPR-visible adducts formed upon reaction with superoxide and hydroxyl radicals. Extinction of all EPR signals in experiments in the presence of SOD shows that only spin trapping of superoxide occurs and that DEPMPO/HO^{•} results from the reduction of DEPMPO/HOO^{•} by TrxR peroxidase activity.

FIG. 17A-FIG. 17B show Influence of Auranofin on Iniparib-biotin modification of TrxR1 and other protein targets in HCT116 cells. FIG. 17A shows HCT116 cells were preincubated for 48 h in the presence of 1 mM BSO. Then cells were treated for 90 min in the presence of 100 μM Iniparib, 10 μM Auranofin or their vehicle. Incubation with 30 μM Iniparib-biotin for 60 min at 37° C. was performed either directly on cells (after removal of Auranofin or Iniparib, and prior to TrxR1 immunoprecipitation), or on TrxR1 pull-down in the presence or the absence of 200 μM NADPH and 5 μM FAD as indicated. FIG. 17B shows BSO-preincubated HCT 116 cells were treated with increasing concentrations of Auranofin up to 3 M as indicated. After removal of Auranofin, cells were further incubated with 10 or 30 μM Iniparib-biotin for 60 min. TrxR1 immunoprecipitates (FIG. 17A) and cells extracts (FIG. 17B) were analysed by SDS-PAGE under reducing conditions followed by streptavidin-HRP blot and TrxR1 blot. Results are representative of at least three independent experiments.

FIG. 18A-FIG. 18B show Cell death signaling pathways induced by Iniparib in relation with Trx oxidation. FIG. 18A shows GSH-depleted HCT116 cells were treated for 7 h or 24 h in the presence of 100 μM Iniparib, 1 μM Auranofin, 100 nM Staurosporin, or their vehicle. FIG. 18B shows BSO-pretreated HCT116 cells and MDA-MB-453 cells were incubated for various times up to 24 h as indicated with 100 μM Iniparib or its vehicle. Cells extracts were analyzed by SDS-PAGE either under reducing conditions or under non-reducing conditions in the case of Trx blot followed by blots anti-phospho-JNK, anti-phospho-p38MAPK, anti-cleaved-PARP and anti-Trx. Results are representative of three independent experiments.

FIG. 19A-FIG. 19B show TrxR inhibition by Iniparib in MDA-MB-453 cells. FIG. 19A shows the effect of Iniparib on TrxR endogenous activity in HCT116 and MDA-MB-453 cells. HCT116 cells (pretreated with 1 mM BSO for 48 h) and MDA-MB-453 cells were incubated for 4 h with the indicated concentrations of Iniparib (black square: HCT-116 cells, clear square: MDA-MB-453 cells). After treatment, TrxR activities were determined on clarified cell lysates using the DTNB reduction assay. All values are expressed as means±SEM of at least three independent experiments. FIG. 19B shows TrxR1 modification by Iniparib-biotin in MDA-MB-453 cells. Cells were pretreated or not for 48 h in the presence of 1 mM BSO. Then cells were incubated with 100 μM BSI-biotin or its vehicle for 4 h. Cell lysates prepared in 1.5%-octylglucoside containing buffer were then processed for TrxR1 pull-down. Cells extracts and TrxR1 immunoprecipitates were analyzed by SDS-PAGE under reducing conditions followed by streptavidin-HRP blot and TrxR1 blot.

FIG. 20A-FIG. 20B shows mass spectrometry analysis of TrxR1 and TrxR2 modification by Iniparib in HCT116 cells. FIG. 20A shows protein sequence coverage: TrxR1 and TrxR2 were immunoprecipitated and analyzed by nanoLC-MS/MS as described in the legend of FIG. 2. Parts of the TrxR1 and TrxR2 sequences covered by MS/MS peptide identification are shown in grey shade. Cys and Sec residues are enlightened in yellow and pink, respectively. FIG. 20B shows TrxR1 cys residues modified by Iniparib in HCT116 cells: Relative abundance by XIC analysis of peptides identified from nano LC-MS/MS experiments of tryptic digest gel bands for TrxR1 in 1-h and 4-h Iniparib treated samples. Percentage of peptides modified by one Iniparib adduct are given for each condition. ND, non detected.

FIG. 21A-FIG. 21C show Mass spectrometry analysis of TrxR modification by Iniparib-biotin. FIG. 21A shows HCT116 cells were pretreated for 48 h in the presence of 1 mM BSO. Then cells were incubated with 100 μM Iniparib-biotin for 5 h. Cell lysates prepared in 1.5%-octylglucoside containing buffer were processed for TrxR1 pull-down. For in vitro modification by Iniparib-biotin, TrxR1 pull-down prepared from BSO-treated HCT116 cells and 36 pmol of rat liver TrxR were incubated with 30 μM BSI-biotin in the presence of 200 μM NADPH and 5 μM FAD for 1 h at 37° C. TrxR samples were separated by SDS-PAGE under reducing conditions, gels being stained with PageBlue Reagent®. Aliquots were submitted in parallel to streptavidin-HRP blot. FIG. 21B shows HCD-MS/MS spectra of the doubly charged peptide ion at m/z [802.79]²⁺ for SGASILQAGCUG peptide of humanTrxR1 with CAM modified Cys and Iniparib-biotin modified Sec. y-ions series, including y₂ are modified allowing to position the Iniparib-biotin modification on Sec residue. FIG. 21C shows HCD-MS/MS spectra of the doubly charged peptide ion at m/z [817.78]²⁺ for SGGDILQSGCUG peptide of rat TrxR1 with CAM modified Cys and Iniparib-biotin modified Sec. y-ions series, including y₂ are modified allowing to position the Iniparib modification on Sec residue.

FIG. 22 shows the influence of Auranofin and DNCB in vitro on Iniparib-biotin modification of TrxR. 36 pmol of rat liver TrxR1 were preincubated for 30 min at 37° C. with 100 μM Auranofin, 100 μM DNCB, 100 μM Iniparib or their vehicle in the presence of 200 μM NADPH and 5 μM FAD. Thereafter 30 μM Iniparib-biotin was added for further 60 min. TrxR Samples were analyzed by SDS-PAGE under reducing conditions followed by streptavidin-HRP blot (2 exposures are shown).

FIG. 23A-FIG. 23E show Iniparib mechanism of action and cell-redox regulation in MDA-MB-231 breast cancer cell line. FIG. 23A shows an exemplary schematic representation of Iniparib-induced inhibition of cell redox regulation. FIG. 23B shows subcellular localization of iniparib-biotin targets. Confocal images of GSH-depleted MDA-MB-231 cells after incubation with iniparib-biotin (30 min, 100 μM) are displayed. Fluorescence signals for alexa488-streptavidin and Mitotracker red CMX are merged. The right panels show the separated fluorescence signals on the selected region in left panel (white rectangle). FIG. 23C shows the effect of iniparib on endogenous TrxR reductase activity. GSH-depleted MDA-MB-231 cells were treated with iniparib (100 μM, black circle) or Auranofin (1 μM, black triangle) for various times up to 4 h. After treatment, TrxR activity was determined on clarified cell lysates (50 μg) using the DTNB reduction assay. All values are expressed as means±SEM of at least 3 independent experiments. FIG. 23D shows the effect of iniparib on Trx1/2 oxidation status and on related stress-signaling. GSH-depleted MDA-MB-231 cells were treated with iniparib (100 μM) for 6 or 18 h. Thereafter, cells were lysed in 50 mM Tris-HCl pH 7.5, 2% SDS, 1 mM EDTA containing 10 mM AMS and alkylation of free cys was carried out for 90 min at 37° C. Resulting cell extracts were submitted to western blot analysis as described in Methods. The experiment shown is representative of three independent experiments. FIG. 23E shows the effect of iniparib on ROS production. GSHdepleted MDA-MB-231 cells were treated with iniparib (100 μM) or its vehicle for 4 h. ROS and nuclei were respectively detected using CellROXOrange® (5 μM) and DRAQ5® (5 μM). Data corresponding to integrated intensities per nuclei are expressed as means±SEM of 3 independent experiments.

FIG. 24A-FIG. 24H show a predictive signature of response to iniparib-containing treatment based on gene expression segregates triple-negative breast cancer patients into iniparib-sensitive and iniparib-resistant groups. FIG. 24A shows a plot of observed progression free survival (PFS, y-axis) versus predicted differential log-hazard A (x-axis) for the overall sample of 210 patients, with actual treatment arm assignments color-coded. The values of A were established by 5-fold cross-validation, so that each prediction is based on a model trained on data independent from the sample for which it is made. Patients with Δξ<0 are predicted to have longer survival in the iniparib treatment arm, and this is qualitatively verified, with blue dots (iniparib arm patients) generally higher than red dots (control arm patients) on the left-hand side of the diagram, while the heights of the dots are intermingled on the right-hand-side. A threshold Δξc=−1 is chosen to segregate patients into “sensitive” (left) and “resistant” groups (right). The threshold is selected to keep reasonable number of patients in the sensitive group while insuring a small hazard ratio between treatment arms for that group. FIG. 24B shows 1000 independently and randomly sampled 5-fold cross-validations were performed to establish the robustness of the signature to segregate patients in responders versus non-responders. For each cross-validation, the hazard ratios between Iniparib-containing and control treatment arms for “sensitive” and “resistant” patients groups separately (hR(S) and hR(R), respectively) were computed, for fixed threshold Δξc=−1. The resulting distributions of hazard ratios as shown are robustly separated, with 95% confidence intervals indicated in the inset. The fraction f(S) of patients selected as sensitive has a median value of about 25%. FIG. 24C-FIG. 24E show survival curves versus PFS for the patient groups defined in A by the threshold Δξc=−1 (all inserts [ . . . ] denote 95% confidence intervals): FIG. 24C shows the survival curves for the two treatment arms are compared for all patients (n=210), showing a moderate but significant hazard ratio (hR=0.673 [0.49, 0.92]95%) between the treatment arms. FIG. 24D shows comparison for the “sensitive” patients only (n=53), showing a considerably smaller hazard ratio (hR=0.352[0.18, 0.68]95%) between treatment arms. FIG. 24E shows comparison for the “resistant” patients, showing a statistically nonsignificant hazard ratio (hR=0.858[0.60, 1.22]95%). FIG. 24F-FIG. 24H shows survival curves versus OS for the patient groups defined in FIG. 24A by the threshold Δξc=−1: FIG. 24F shows the OS curves for the two treatment arms are compared for all patients (n=210) with hazard ratio close to 1 (hR=0.776[0.56, 1.08]95%). FIG. 24G shows comparison on OS for the “sensitive” patients only (n=53), showing a smaller hazard ratio (hR=0.584[0.30, 1.15]95%) between treatment arms, statistically non-significant but with the same trend as observed for PFS. FIG. 24H shows comparison on OS for the “resistant” patients, showing a hazard ratio closer to 1(hR=0.817[0.56, 1.2]95%).

FIG. 25 illustrates an exemplary Phase I treatment schema.

FIG. 26 illustrates an exemplary Phase II treatment schema.

FIG. 27 shows an overall survival analysis from the Phase II trial described herein.

FIG. 28 illustrates an overall survival by MGMT status from the Phase II trial described herein.

FIG. 29 illustrates an increase in the percentage of patients with 2 year survival and 3 year survival.

FIG. 30 shows overall survival (OS) benefit achieved in 2^{nd}/3^{rd} line metastatic triple negative breast cancer (TNBC) at primary analysis.

FIG. 31 shows OA benefit achieved in 2^{nd}/3^{rd} line metastatic triple negative breast cancer (TNBC) at updated analysis.

FIG. 32 illustrates overall survival (OS) benefit in all metastatic triple negative breast cancer (mTNBC) patients.

FIG. 33 illustrates overall survival (OS) benefit in 2^{nd}/3^{rd} line mTNBC patients.

FIG. 34 illustrates Phase 3 clinical trial missed OS end point.

FIG. 35 illustrates impact of relapsed patient with short DFI on phase 3 OS.

FIG. 36 illustrates 2^{nd}/3^{rd} line benefit across Phase 2 and Phase 3 trials and at endpoints.

### DETAILED DESCRIPTION OF THE DISCLOSURE

Tumor cells have increased rates of glucose uptake as compared to nonmalignant cells. Glucose, in addition to its role in energy production, plays a role in the metabolism of reactive oxygen species (ROS) through the pentose phosphate pathway with the generation of NADPH. In some instances, NADPH is the major electron donor for thioredoxin reductase (TrxR) and glutathione reductase (GR), two enzymes for maintaining glutathione (GSH) and thioredoxin (Trx) in their reduced state. In addition, GSH and Trx are two cellular thiol redox components responsible for decomposition of ROS, maintaining the cell redox potential and preventing or repairing oxidative damage.

Mammalian TrxR belongs to a small family of proteins that contains selenocysteine (Sec) residues in their sequence. Mammalian cells have a homodimeric TrxR1 in the cytosol and nucleus, and a homodimeric TrxR2 in the mitochondria. There is also a third member of the family named thioredoxin-glutathione reductase (TGR) which is expressed mainly in the testis. TrxRs contain NADPH- and FAD-binding domains, a redox-active disulfide site in the N-terminal region, and another redox-active site, based on a selenylsulfide sequence in the C-terminal region. In a catalytic cycle, electrons are transferred from NADPH to FAD, then to the first redox site which is used to reduce the selenylsulfide site. The C-terminal site is responsible for the reduction of the disulfide in the active site of Trx which is the main substrate of TrxR, as well as several other protein disulfide substrates, and low molecular weight natural and synthetic substrates. Trx, the protein target of TrxR, is involved in maintaining the reducing environment in the cell by interacting and reducing a number of proteins.

Peroxiredoxin (Prx) is located downstream of Trx and constitutes a family of peroxidases. Prx receives electrons from Trx and participates in the removal of hydrogen peroxide from the ROS system.

In some instances, cancer cells have been characterized with an elevated expression of thioredoxin reductase (TrxR) or an elevated expression of peroxiredoxin (PRDX). In some embodiments, disclosed herein is a method for treating a cancer characterized with an elevated expression of TrxR with a therapeutically effective amount of a nitrobenzamide compound (e.g., 4-iodo-3-nitrobenzamide or a salt, metabolite or prodrug thereof). In other embodiments, disclosed herein is a method for treating a cancer characterized with an elevated expression of PRDX with a therapeutically effective amount of a nitrobenzamide compound (e.g., 4-iodo-3-nitrobenzamide or a salt, metabolite or prodrug thereof).

In additional embodiments, also disclosed herein are methods of selecting subjects for treatment based on a biomarker panel. In some instances, the biomarker panel comprises thioredoxin reductase 2 (TXNRD2), thioredoxin 2 (TXN2), methionine sulfoxide reductase B3 (MSRB3), methionine sulfoxide reductase A (MSRA), and glutathione transferase zeta 1 (GSTZ1).

In further embodiments, described herein are methods of monitoring the treatment progress based on the expression level of biomarkers from a biomarker panel. In some instances, the biomarker panel comprises NAD(P)H dehydrogenase quinone 2 (NQO2), glutathione S-transferase theta 2 (GSTT2), glutathione S-transferase M3 (GSTM3), glutaredoxin (GLRX), selenoprotein O (SELO), paraoxonase 1 (PON1), glutathione S-transferase omega 1 (GSTO1), glutaredoxin 3 (GLRX3), selenoprotein X 1 (SEPX1), and thioredoxin reductase 1 (TXNRD1).

### Nitrobenzamide Compounds

In some embodiments, disclosed herein are compounds of Formula (I):

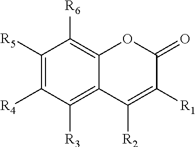

wherein R₁, R₂, R₃, R₄, and R₅ are, independently selected from the group consisting of hydrogen, hydroxy, amino, nitro, iodo, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen, at least one of the five substituents are always nitro, and at least one substituent positioned adjacent to a nitro is always iodo, and pharmaceutically acceptable salts, solvates, isomers, tautomers, metabolites, analogs, or prodrugs thereof. R₁, R₂, R₃, R₄, and R₅ can also be a halide such as chloro, fluoro, or bromo.

In some embodiments, a compound disclosed herein is 4-iodo-3-nitrobenzamide (also known as iniparib and BSI201). In some instances, 4-iodo-3-nitrobenzamide has the structure

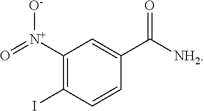 [SMILES: NC(=O)c1ccc(I)c([N+](=O)[O-])c1]

In some embodiments, disclosed herein is a compound described in U.S. Pat. No. 5,464,871.

### Methods of Use

### Methods of Treating TrxR-Overexpressed or PRDX-Overexpressed Cancers

In certain embodiments, disclosed herein are methods of treating a TrxR-overexpressed cancer or a PRDX-overexpressed cancer with a nitrobenzamide compound described supra. In some instances, the nitrobenzamide compound is a compound encompassed by Formula (I). In some instances, the nitrobenzamide compound is 4-iodo-3-nitrobenzamide or a salt, metabolite or prodrug thereof.

In some embodiments, the method comprises treating a TrxR-overexpressed cancer or a PRDX-overexpressed cancer with 4-iodo-3-nitrobenzamide or a salt, metabolite or prodrug thereof. In some embodiments, the method comprises treating a TrxR-overexpressed cancer with 4-iodo-3-nitrobenzamide or a salt, metabolite or prodrug thereof. In some embodiments, the method of treating a TrxR-overexpressed cancer comprises treating a subject having a cancer characterized with an elevated expression of thioredoxin reductase (TrxR), comprising: (a) determining whether the subject has an elevated expression of TrxR by i) measuring an expression level of TrxR from a cancer sample obtained from the subject, and ii) determining whether the expression level of TrxR from the cancer sample is elevated relative to a control sample; and (b) administering to the subject a therapeutically effective amount of 4-iodo-3-nitrobenzamide or a salt, metabolite or prodrug thereof, thereby treating the subject having the cancer characterized with the elevated expression of TrxR. In some embodiments, the measuring comprises either evaluating the TrxR gene expression or the TrxR protein expression. In some instances, the measuring comprises i) contacting a portion of the TrxR gene with a set of primers to produce amplified nucleic acids, and ii) determining the level of the amplified nucleic acids in the tumor sample. In other instances, the measuring comprises i) contacting the sample with an anti-TrxR antibody and ii) detecting binding between TrxR protein and the anti-TrxR antibody.

In some embodiments, the method of diagnosing and treating cancer in a subject comprises (a) obtaining a cancer sample from a human subject; (b) detecting whether an expression level of thioredoxin reductase (TrxR) is elevated in the cancer sample relative to an expression level of TrxR in a control sample; (c) diagnosing the subject as having a cancer characterized with the elevated expression of TrxR; and (d) administering an effective amount of 4-iodo-3-nitrobenzamide or a salt, metabolite or prodrug thereof to the diagnosed subject. In some embodiments, the detecting comprises either evaluating the TrxR gene expression or the TrxR protein expression. In some instances, the detecting comprises i) contacting a portion of the TrxR gene with a set of primers to produce amplified nucleic acids, and ii) determining the level of the amplified nucleic acids in the tumor sample. In other instances, the detecting comprises i) contacting the sample with an anti-TrxR antibody and ii) detecting binding between TrxR protein and the anti-TrxR antibody.

In some embodiments, disclosed herein is a method of treating a subject having a cancer characterized with an elevated expression of thioredoxin reductase (TrxR), comprising: administering to the subject a therapeutically effective amount of 4-iodo-3-nitrobenzamide or a salt, metabolite or prodrug thereof, thereby treating the subject having the cancer characterized with the elevated expression of TrxR; wherein the subject is determined to have the elevated TrxR by i) measuring an expression level of TrxR from a cancer sample obtained from the subject, and ii) determining whether the expression level of TrxR from the cancer sample is elevated relative to a control sample.

In some embodiments, disclosed herein is a method for treating a subject with 4-iodo-3-nitrobenzamide or a salt, metabolite or prodrug thereof, wherein the subject has a cancer, the method comprising: determining whether the subject has an elevated expression of thioredoxin reductase (TrxR) by: i) measuring an expression level of TrxR from a cancer sample obtained from the subject, and ii) determining whether the expression level of TrxR from the cancer sample is elevated relative to a control sample; if the subject has an elevated expression of TrxR, then administering 4-iodo-3-nitrobenzamide or a salt, metabolite or prodrug thereof to the subject, and if the subject does not have an elevated expression of TrxR, then administering a first-line treatment to the subject, wherein a length of disease free interval (DFI) for the subject having an elevated expression of TrxR is extended following administration of the treatment regimen comprising 4-iodo-3-nitrobenzamide or a salt, metabolite or prodrug thereof than it would be if the first-line treatment were administered.

In some cases, TrxR is thioredoxin reductase 1 (TrxR-1). In other cases, TrxR is thioredoxin reductase 2 (TrxR-2).

In some embodiments, the elevated expression level of TrxR is about 10%, 20%, 30, 40%, 50%, 60%, 70%, 80%, 90%, 95%, or 99% higher relative to the expression level of TrxR in a cell from a control sample. In some instances, the cell from the control sample is a non-cancerous cell. In some instances, the cell from the control sample is obtained from a healthy subject. In some cases, the elevated expression level of TrxR is about 10% higher relative to the expression level of TrxR in a cell from a control sample. In some cases, the elevated expression level of TrxR is about 20% higher relative to the expression level of TrxR in a cell from a control sample. In some cases, the elevated expression level of TrxR is about 30% higher relative to the expression level of TrxR in a cell from a control sample. In some cases, the elevated expression level of TrxR is about 40% higher relative to the expression level of TrxR in a cell from a control sample. In some cases, the elevated expression level of TrxR is about 50% higher relative to the expression level of TrxR in a cell from a control sample. In some cases, the elevated expression level of TrxR is about 60% higher relative to the expression level of TrxR in a cell from a control sample. In some cases, the elevated expression level of TrxR is about 70% higher relative to the expression level of TrxR in a cell from a control sample. In some cases, the elevated expression level of TrxR is about 80% higher relative to the expression level of TrxR in a cell from a control sample. In some cases, the elevated expression level of TrxR is about 90% higher relative to the expression level of TrxR in a cell from a control sample. In some cases, the elevated expression level of TrxR is about 95% higher relative to the expression level of TrxR in a cell from a control sample. In some cases, the elevated expression level of TrxR is about 9% higher relative to the expression level of TrxR in a cell from a control sample.

In some instances, the elevated expression level of TrxR is about 1-fold, 2-fold, 3-fold, 4-fold, 5-fold, 6-fold, 7-fold, 8-fold, 9-fold, 10-fold, 15-fold, 20-fold or higher relative to the expression level of TrxR in a cell from a control sample. In some instances, the cell from the control sample is a non-cancerous cell. In some instances, the cell from the control sample is obtained from a healthy subject. In some cases, the elevated expression level of TrxR is about 1-fold or higher relative to the expression level of TrxR in a cell from a control sample. In some cases, the elevated expression level of TrxR is about 2-fold or higher relative to the expression level of TrxR in a cell from a control sample. In some cases, the elevated expression level of TrxR is about 3-fold or higher relative to the expression level of TrxR in a cell from a control sample. In some cases, the elevated expression level of TrxR is about 4-fold or higher relative to the expression level of TrxR in a cell from a control sample. In some cases, the elevated expression level of TrxR is about 5-fold or higher relative to the expression level of TrxR in a cell from a control sample. In some cases, the elevated expression level of TrxR is about 6-fold or higher relative to the expression level of TrxR in a cell from a control sample. In some cases, the elevated expression level of TrxR is about 7-fold or higher relative to the expression level of TrxR in a cell from a control sample. In some cases, the elevated expression level of TrxR is about 8-fold or higher relative to the expression level of TrxR in a cell from a control sample. In some cases, the elevated expression level of TrxR is about 9-fold or higher relative to the expression level of TrxR in a cell from a control sample. In some cases, the elevated expression level of TrxR is about 10-fold or higher relative to the expression level of TrxR in a cell from a control sample. In some cases, the elevated expression level of TrxR is about 15-fold or higher relative to the expression level of TrxR in a cell from a control sample. In some cases, the elevated expression level of TrxR is about 20-fold or higher relative to the expression level of TrxR in a cell from a control sample.

In some embodiments, the method comprises treating a PRDX-overexpressed cancer with 4-iodo-3-nitrobenzamide or a salt, metabolite or prodrug thereof. In some embodiments, the method of treating a PRDX-overexpressed cancer comprises treating a subject having a cancer characterized with an elevated expression of peroxiredoxin (PRDX), comprising (a) determining whether the subject has an elevated expression of peroxiredoxin by i) measuring an expression level of PRDX from a cancer sample obtained from the subject, and ii) determining whether the expression level of PRDX from the cancer sample is elevated relative to a control sample; and (b) administering to the subject a therapeutically effective amount of 4-iodo-3-nitrobenzamide or a salt, metabolite or prodrug thereof, thereby treating the subject having the cancer characterized with the elevated expression of PRDX. In some embodiments, the measuring comprises either evaluating the PRDX gene expression or the PRDX protein expression. In some instances, the measuring comprises i) contacting a portion of the PRDX gene with a set of primers to produce amplified nucleic acids, and ii) determining the level of the amplified nucleic acids in the tumor sample. In other instances, the measuring comprises i) contacting the sample with an anti-PRDX antibody and ii) detecting binding between PRDX protein and the anti-PRDX antibody.

In some embodiments, the method of diagnosing and treating cancer in a subject comprises (a) obtaining a cancer sample from a human subject; (b) detecting whether an expression level of peroxiredoxin (PRDX) is elevated in the cancer sample relative to an expression level of PRDX in a control sample; (c) diagnosing the subject as having a cancer characterized with the elevated expression of PRDX; and (d) administering an effective amount of 4-iodo-3-nitrobenzamide or a salt, metabolite or prodrug thereof to the diagnosed subject. In some embodiments, the detecting comprises either evaluating the PRDX gene expression or the PRDX protein expression. In some instances, the detecting comprises i) contacting a portion of the PRDX gene with a set of primers to produce amplified nucleic acids, and ii) determining the level of the amplified nucleic acids in the tumor sample. In other instances, the detecting comprises i) contacting the sample with an anti-PRDX antibody and ii) detecting binding between PRDX protein and the anti-PRDX antibody.

In some embodiments, disclosed herein is a method of treating a subject having a cancer characterized with an elevated expression of peroxiredoxin (PRDX), comprising: administering to the subject a therapeutically effective amount of 4-iodo-3-nitrobenzamide or a salt, metabolite or prodrug thereof, thereby treating the subject having the cancer characterized with the elevated expression of PRDX; wherein the subject is determined to have the elevated PRDX by i) measuring an expression level of PRDX from a cancer sample obtained from the subject, and ii) determining whether the expression level of PRDX from the cancer sample is elevated relative to a control sample.

In some embodiments, disclosed herein is a method for treating a subject with 4-iodo-3-nitrobenzamide or a salt, metabolite or prodrug thereof, wherein the subject has a cancer, the method comprising: determining whether the subject has an elevated expression of peroxiredoxin (PRDX) by: i) measuring an expression level of PRDX from a cancer sample obtained from the subject, and ii) determining whether the expression level of PRDX from the cancer sample is elevated relative to a control sample; if the subject has an elevated expression of PRDX, then administering 4-iodo-3-nitrobenzamide or a salt, metabolite or prodrug thereof to the subject, and if the subject does not have an elevated expression of PRDX, then administering a first-line treatment to the subject, wherein a length of disease free interval (DFI) for the subject having an elevated expression of PRDX is extended following administration of the treatment regimen comprising 4-iodo-3-nitrobenzamide or a salt, metabolite or prodrug thereof than it would be if the first-line treatment were administered.

In some instances, peroxiredoxin is peroxiredoxin-1 (PRDX-1). In some instances, the elevated expression of peroxiredoxin-1 is determined by i) measuring an expression level of PRDX-1 from a cancer sample obtained from the subject, and ii) determining whether the expression level of PRDX-1 from the cancer sample is elevated relative to a control sample. In some cases, the measuring comprises i) contacting a portion of the PRDX-1 gene with a set of primers to produce amplified nucleic acids, and ii) determining the level of the amplified nucleic acids in the tumor sample. In other cases, the measuring comprises i) contacting the sample with an anti-PRDX-1 antibody and ii) detecting binding between PRDX-1 protein and the anti-PRDX-1 antibody.

In some embodiments, the elevated expression level of PRDX is about 10%, 20%, 30, 40%, 50%, 60%, 70%, 80%, 90%, 95%, or 99% higher relative to the expression level of PRDX in a cell from a control sample. In some instances, the cell from the control sample is a non-cancerous cell. In some instances, the cell from the control sample is obtained from a healthy subject. In some cases, the elevated expression level of PRDX is about 10% higher relative to the expression level of PRDX in a cell from a control sample. In some cases, the elevated expression level of PRDX is about 20% higher relative to the expression level of PRDX in a cell from a control sample. In some cases, the elevated expression level of PRDX is about 30% higher relative to the expression level of PRDX in a cell from a control sample. In some cases, the elevated expression level of PRDX is about 40% higher relative to the expression level of PRDX in a cell from a control sample. In some cases, the elevated expression level of PRDX is about 50% higher relative to the expression level of PRDX in a cell from a control sample. In some cases, the elevated expression level of PRDX is about 60% higher relative to the expression level of PRDX in a cell from a control sample. In some cases, the elevated expression level of PRDX is about 70% higher relative to the expression level of PRDX in a cell from a control sample. In some cases, the elevated expression level of PRDX is about 80% higher relative to the expression level of PRDX in a cell from a control sample. In some cases, the elevated expression level of PRDX is about 90% higher relative to the expression level of PRDX in a cell from a control sample. In some cases, the elevated expression level of PRDX is about 95% higher relative to the expression level of PRDX in a cell from a control sample. In some cases, the elevated expression level of PRDX is about 9% higher relative to the expression level of PRDX in a cell from a control sample.

In some embodiments, the elevated expression level of PRDX is about 1-fold, 2-fold, 3-fold, 4-fold, 5-fold, 6-fold, 7-fold, 8-fold, 9-fold, 10-fold, 15-fold, 20-fold or higher relative to the expression level of PRDX in a cell from a control sample. In some instances, the cell from the control sample is a non-cancerous cell. In some instances, the cell from the control sample is obtained from a healthy subject. In some cases, the elevated expression level of PRDX is about 1-fold or higher relative to the expression level of PRDX in a cell from a control sample. In some cases, the elevated expression level of PRDX is about 2-fold or higher relative to the expression level of PRDX in a cell from a control sample. In some cases, the elevated expression level of PRDX is about 3-fold or higher relative to the expression level of PRDX in a cell from a control sample. In some cases, the elevated expression level of PRDX is about 4-fold or higher relative to the expression level of PRDX in a cell from a control sample. In some cases, the elevated expression level of PRDX is about 5-fold or higher relative to the expression level of PRDX in a cell from a control sample. In some cases, the elevated expression level of PRDX is about 6-fold or higher relative to the expression level of PRDX in a cell from a control sample. In some cases, the elevated expression level of PRDX is about 7-fold or higher relative to the expression level of PRDX in a cell from a control sample. In some cases, the elevated expression level of PRDX is about 8-fold or higher relative to the expression level of PRDX in a cell from a control sample. In some cases, the elevated expression level of PRDX is about 9-fold or higher relative to the expression level of PRDX in a cell from a control sample. In some cases, the elevated expression level of PRDX is about 10-fold or higher relative to the expression level of PRDX in a cell from a control sample. In some cases, the elevated expression level of PRDX is about 15-fold or higher relative to the expression level of PRDX in a cell from a control sample. In some cases, the elevated expression level of PRDX is about 20-fold or higher relative to the expression level of PRDX in a cell from a control sample.

In some embodiments, 4-iodo-3-nitrobenzamide or a salt, metabolite or prodrug thereof is administered from about 2 mg/kg to about 200 mg/kg. In some instances, 4-iodo-3-nitrobenzamide or a salt, metabolite or prodrug thereof is administered from about 2 mg/kg to about 150 mg/kg, from about 2 mg/kg to about 100 mg/kg, or from about 2 mg/kg to about 60 mg/kg. In some instances, 4-iodo-3-nitrobenzamide or a salt, metabolite or prodrug thereof is administered from about 5 mg/kg to about 150 mg/kg, from about 5 mg/kg to about 100 mg/kg, or from about 5 mg/kg to about 60 mg/kg. In some instances, 4-iodo-3-nitrobenzamide or a salt, metabolite or prodrug thereof is administered at a range of about 5 mg/kg to about 50 mg/kg, about 5 mg/kg to about 40 mg/kg, about 5 mg/kg to about 30 mg/kg, about 5 mg/kg to about 20 mg/kg, about 5 mg/kg to about 10 mg/kg, about 6 mg/kg to about 60 mg/kg, about 6 mg/kg to about 50 mg/kg, about 6 mg/kg to about 40 mg/kg, about 6 mg/kg to about 30 mg/kg, about 6 mg/kg to about 20 mg/kg, about 6 mg/kg to about 10 mg/kg, about 6 mg/kg to about 9 mg/kg, about 7 mg/kg to about 60 mg/kg, about 7 mg/kg to about 50 mg/kg, about 7 mg/kg to about 40 mg/kg, about 7 mg/kg to about 30 mg/kg, about 7 mg/kg to about 20 mg/kg, about 7 mg/kg to about 10 mg/kg, about 7 mg/kg to about 9 mg/kg, about 7 mg/kg to about 8 mg/kg, about 8 mg/kg to about 60 mg/kg, about 8 mg/kg to about 40 mg/kg, about 8 mg/kg to about 30 mg/kg, about 8 mg/kg to about 20 mg/kg, about 8 mg/kg to about 10 mg/kg, about 8 mg/kg to about 9 mg/kg, or about 8 mg/kg to about 8.6 mg/kg. In some embodiments, 4-iodo-3-nitrobenzamide or a salt, metabolite or prodrug thereof is administered at a range of about 6 mg/kg to about 40 mg/kg, about 6 mg/kg to about 30 mg/kg, about 6 mg/kg to about 20 mg/kg, about 6 mg/kg to about 10 mg/kg, about 6 mg/kg to about 9 mg/kg, about 7 mg/kg to about 30 mg/kg, about 7 mg/kg to about 20 mg/kg, about 7 mg/kg to about 9 mg/kg, about 7 mg/kg to about 8 mg/kg, about 8 mg/kg to about 20 mg/kg, about 8 mg/kg to about 9 mg/kg, or about 8 mg/kg to about 8.6 mg/kg. In some embodiments, 4-iodo-3-nitrobenzamide or a salt, metabolite or prodrug thereof is administered at a range of about 5 mg/kg to about 40 mg/kg. In some embodiments, 4-iodo-3-nitrobenzamide or a salt, metabolite or prodrug thereof is administered at a range of about 6 mg/kg to about 9 mg/kg. In some embodiments, 4-iodo-3-nitrobenzamide or a salt, metabolite or prodrug thereof is administered at a range of about 6 mg/kg to about 8.6 mg/kg. In some embodiments, 4-iodo-3-nitrobenzamide or a salt, metabolite or prodrug thereof is administered at a range of about 6 mg/kg to about 8 mg/kg. In some embodiments, 4-iodo-3-nitrobenzamide or a salt, metabolite or prodrug thereof is administered at a range of about 7 mg/kg to about 9 mg/kg. In some embodiments, 4-iodo-3-nitrobenzamide or a salt, metabolite or prodrug thereof is administered at a range of about 7 mg/kg to about 8.6 mg/kg. In some embodiments, 4-iodo-3-nitrobenzamide or a salt, metabolite or prodrug thereof is administered at a range of about 7 mg/kg to about 8 mg/kg. In some embodiments, 4-iodo-3-nitrobenzamide or a salt, metabolite or prodrug thereof is administered at a range of about 8 mg/kg to about 9 mg/kg. In some embodiments, 4-iodo-3-nitrobenzamide or a salt, metabolite or prodrug thereof is administered at a range of about 8 mg/kg to about 8.6 mg/kg.

In some embodiments, 4-iodo-3-nitrobenzamide or a salt, metabolite or prodrug thereof is administered at about 2 mg/kg, about 3 mg/kg, about 4 mg/kg, about 5 mg/kg, about 6 mg/kg, about 7 mg/kg, about 8 mg/kg, about 8.5 mg/kg, about 8.6 mg/kg, about 9 mg/kg, about 10 mg/kg, about 15 mg/kg, about 20 mg/kg, about 25 mg/kg, about 30 mg/kg, about 40 mg/kg, about 50 mg/kg, about 60 mg/kg, about 100 mg/kg, about 150 mg/kg, or about 200 mg/kg. In some embodiments, 4-iodo-3-nitrobenzamide or a salt, metabolite or prodrug thereof is administered at about 5 mg/kg, about 6 mg/kg, about 7 mg/kg, about 8 mg/kg, about 8.5 mg/kg, about 8.6 mg/kg, about 9 mg/kg, about 10 mg/kg, about 15 mg/kg, about 20 mg/kg, about 25 mg/kg, about 30 mg/kg, or about 40 mg/kg. In some embodiments, 4-iodo-3-nitrobenzamide or a salt, metabolite or prodrug thereof is administered at about 2 mg/kg. In some embodiments, 4-iodo-3-nitrobenzamide or a salt, metabolite or prodrug thereof is administered at about 3 mg/kg. In some embodiments, 4-iodo-3-nitrobenzamide or a salt, metabolite or prodrug thereof is administered at about 4 mg/kg. In some embodiments, 4-iodo-3-nitrobenzamide or a salt, metabolite or prodrug thereof is administered at about 5 mg/kg. In some embodiments, 4-iodo-3-nitrobenzamide or a salt, metabolite or prodrug thereof is administered at about 6 mg/kg. In some embodiments, 4-iodo-3-nitrobenzamide or a salt, metabolite or prodrug thereof is administered at about 7 mg/kg. In some embodiments, 4-iodo-3-nitrobenzamide or a salt, metabolite or prodrug thereof is administered at about 8 mg/kg. In some embodiments, 4-iodo-3-nitrobenzamide or a salt, metabolite or prodrug thereof is administered at about 8.5 mg/kg. In some embodiments, 4-iodo-3-nitrobenzamide or a salt, metabolite or prodrug thereof is administered at about 8.6 mg/kg. In some embodiments, 4-iodo-3-nitrobenzamide or a salt, metabolite or prodrug thereof is administered at about 9 mg/kg. In some embodiments, 4-iodo-3-nitrobenzamide or a salt, metabolite or prodrug thereof is administered at about 10 mg/kg. In some embodiments, 4-iodo-3-nitrobenzamide or a salt, metabolite or prodrug thereof is administered at about 15 mg/kg. In some embodiments, 4-iodo-3-nitrobenzamide or a salt, metabolite or prodrug thereof is administered at about 20 mg/kg. In some embodiments, 4-iodo-3-nitrobenzamide or a salt, metabolite or prodrug thereof is administered at about 30 mg/kg. In some embodiments, 4-iodo-3-nitrobenzamide or a salt, metabolite or prodrug thereof is administered at about 40 mg/kg. In some embodiments, 4-iodo-3-nitrobenzamide or a salt, metabolite or prodrug thereof is administered at about 50 mg/kg. In some embodiments, 4-iodo-3-nitrobenzamide or a salt, metabolite or prodrug thereof is administered at about 60 mg/kg.

In some embodiments, 4-iodo-3-nitrobenzamide or a salt, metabolite or prodrug thereof is administered to a subject at one or more dosing schedules. In some embodiments, the dosing schedule comprises administering to the subject 4-iodo-3-nitrobenzamide or a salt, metabolite or prodrug thereof once per day, twice a week, three times a week, four times a week, five times a week, daily, every other day, once a month, twice a month, or every week. In some instances, the dosing schedule comprises administering to the subject 4-iodo-3-nitrobenzamide or a salt, metabolite or prodrug thereof once per day.

In some embodiments, the dosing schedule comprises administering to the subject 4-iodo-3-nitrobenzamide or a salt, metabolite or prodrug thereof continuously for about 1, 2, 3, 4, 5, 6, 7, 8, 9, 10, 11, 12 or more weeks. In some instances, the dosing schedule comprises administering to the subject 4-iodo-3-nitrobenzamide or a salt, metabolite or prodrug thereof continuously for about 1, 2, 3, 4, 5, 6, 7, 8, 9, 10 or more weeks. In some instances, the dosing schedule comprises administering to the subject 4-iodo-3-nitrobenzamide or a salt, metabolite or prodrug thereof continuously for about 1, 2, 3, 4, 5, 6 or more weeks. In some instances, the dosing schedule comprises administering to the subject 4-iodo-3-nitrobenzamide or a salt, metabolite or prodrug thereof continuously for about 5 weeks. In some instances, the dosing schedule comprises administering to the subject 4-iodo-3-nitrobenzamide or a salt, metabolite or prodrug thereof continuously for about 6 weeks. In some instances, the dosing schedule comprises administering to the subject 4-iodo-3-nitrobenzamide or a salt, metabolite or prodrug thereof continuously for about 7 weeks. In some instances, the dosing schedule comprises administering to the subject 4-iodo-3-nitrobenzamide or a salt, metabolite or prodrug thereof continuously for about 8 weeks. In some instances, the dosing schedule comprises administering to the subject 4-iodo-3-nitrobenzamide or a salt, metabolite or prodrug thereof continuously for about 9 weeks. In some instances, the dosing schedule comprises administering to the subject 4-iodo-3-nitrobenzamide or a salt, metabolite or prodrug thereof continuously for about 10 weeks. In some instances, a 5-week dosing schedule is considered as one cycle. In some instances, a 6-week dosing schedule is considered as one cycle. In some instances, a 7-week dosing schedule is considered as one cycle. In some instances, a 8-week dosing schedule is considered as one cycle. In some instances, a 9-week dosing schedule is considered as one cycle. In some instances, a 10-week dosing schedule is considered as one cycle.

In some embodiments, the dosing schedule comprises administering to the subject 4-iodo-3-nitrobenzamide or a salt, metabolite or prodrug thereof continuously for about 1, 2, 3, 4, 5, 6, 7, 8, 9, 10, 11, 12 or more months. In some instances, the dosing schedule comprises administering to the subject 4-iodo-3-nitrobenzamide or a salt, metabolite or prodrug thereof continuously for about 1, 2, 3, 4, 5, 6 or more months.

In some embodiments, the dosing schedule comprises administering to the subject 4-iodo-3-nitrobenzamide or a salt, metabolite or prodrug thereof continuously for about 1, 2, 3, 4, 5, 6, 7, 8, 9, 10 or more treatment cycles. In some instances, the dosing schedule comprises administering to the subject 4-iodo-3-nitrobenzamide or a salt, metabolite or prodrug thereof continuously for about 1, 2, 3, 4, 5, 6 or more treatment cycles. In some instances, the dosing schedule comprises administering to the subject 4-iodo-3-nitrobenzamide or a salt, metabolite or prodrug thereof continuously for about 1, 2, 3, 4 or more treatment cycles. In some instances, the dosing schedule comprises administering to the subject 4-iodo-3-nitrobenzamide or a salt, metabolite or prodrug thereof continuously for about 1 or more cycles. In some instances, the dosing schedule comprises administering to the subject 4-iodo-3-nitrobenzamide or a salt, metabolite or prodrug thereof continuously for about 2 or more cycles. In some instances, the dosing schedule comprises administering to the subject 4-iodo-3-nitrobenzamide or a salt, metabolite or prodrug thereof continuously for about 3 or more cycles. In some instances, the dosing schedule comprises administering to the subject 4-iodo-3-nitrobenzamide or a salt, metabolite or prodrug thereof continuously for about 4 or more cycles. In some instances, each treatment cycle is up to 28 days. In some cases, each treatment cycle is about 28 days. In other instances, each treatment cycle is up to 5 weeks. In other instances, each treatment cycle is about 5 weeks. In other instances, each treatment cycle is up to 6 weeks. In other instances, each treatment cycle is about 6 weeks. In other instances, each treatment cycle is up to 7 weeks. In other instances, each treatment cycle is about 7 weeks. In other instances, each treatment cycle is up to 8 weeks. In other instances, each treatment cycle is about 8 weeks. In other instances, each treatment cycle is up to 9 weeks. In other instances, each treatment cycle is about 9 weeks. In other instances, each treatment cycle is up to 10 weeks. In other instances, each treatment cycle is about 10 weeks.

In some embodiments, the dosing schedule comprises administering to the subject 4-iodo-3-nitrobenzamide or a salt, metabolite or prodrug thereof intermittently for about 1, 2, 3, 4, 5, 6, 7, 8, 9, 10, 11, 12 or more weeks. In some instances, the dosing schedule comprises administering to the subject 4-iodo-3-nitrobenzamide or a salt, metabolite or prodrug thereof intermittently for about 1, 2, 3, 4, 5, 6, 7, 8, 9, 10 or more weeks. In some instances, the dosing schedule comprises administering to the subject 4-iodo-3-nitrobenzamide or a salt, metabolite or prodrug thereof intermittently for about 1, 2, 3, 4, 5, 6 or more weeks. In some instances, the dosing schedule comprises administering to the subject 4-iodo-3-nitrobenzamide or a salt, metabolite or prodrug thereof intermittently for about 5 weeks. In some instances, the dosing schedule comprises administering to the subject 4-iodo-3-nitrobenzamide or a salt, metabolite or prodrug thereof intermittently for about 6 weeks. In some instances, the dosing schedule comprises administering to the subject 4-iodo-3-nitrobenzamide or a salt, metabolite or prodrug thereof intermittently for about 7 weeks. In some instances, the dosing schedule comprises administering to the subject 4-iodo-3-nitrobenzamide or a salt, metabolite or prodrug thereof intermittently for about 8 weeks. In some instances, the dosing schedule comprises administering to the subject 4-iodo-3-nitrobenzamide or a salt, metabolite or prodrug thereof intermittently for about 9 weeks. In some instances, the dosing schedule comprises administering to the subject 4-iodo-3-nitrobenzamide or a salt, metabolite or prodrug thereof intermittently for about 10 weeks. In some instances, a 5-week dosing schedule is considered as one cycle. In some instances, a 6-week dosing schedule is considered as one cycle. In some instances, a 7-week dosing schedule is considered as one cycle. In some instances, a 8-week dosing schedule is considered as one cycle. In some instances, a 9-week dosing schedule is considered as one cycle. In some instances, a 10-week dosing schedule is considered as one cycle.

In some embodiments, the dosing schedule comprises administering to the subject 4-iodo-3-nitrobenzamide or a salt, metabolite or prodrug thereof intermittently for about 1, 2, 3, 4, 5, 6, 7, 8, 9, 10, 11, 12 or more months. In some instances, the dosing schedule comprises administering to the subject 4-iodo-3-nitrobenzamide or a salt, metabolite or prodrug thereof intermittently for about 1, 2, 3, 4, 5, 6 or more months.

In some embodiments, the dosing schedule comprises administering to the subject 4-iodo-3-nitrobenzamide or a salt, metabolite or prodrug thereof intermittently for about 1, 2, 3, 4, 5, 6, 7, 8, 9, 10 or more treatment cycles. In some instances, the dosing schedule comprises administering to the subject 4-iodo-3-nitrobenzamide or a salt, metabolite or prodrug thereof intermittently for about 1, 2, 3, 4, 5, 6 or more treatment cycles. In some instances, the dosing schedule comprises administering to the subject 4-iodo-3-nitrobenzamide or a salt, metabolite or prodrug thereof intermittently for about 1, 2, 3, 4 or more treatment cycles. In some instances, the dosing schedule comprises administering to the subject 4-iodo-3-nitrobenzamide or a salt, metabolite or prodrug thereof intermittently for about 1 or more cycles. In some instances, the dosing schedule comprises administering to the subject 4-iodo-3-nitrobenzamide or a salt, metabolite or prodrug thereof intermittently for about 2 or more cycles. In some instances, the dosing schedule comprises administering to the subject 4-iodo-3-nitrobenzamide or a salt, metabolite or prodrug thereof intermittently for about 3 or more cycles. In some instances, the dosing schedule comprises administering to the subject 4-iodo-3-nitrobenzamide or a salt, metabolite or prodrug thereof intermittently for about 4 or more cycles. In some instances, the dosing schedule comprises administering to the subject 4-iodo-3-nitrobenzamide or a salt, metabolite or prodrug thereof intermittently for about 5 or more cycles. In some instances, the dosing schedule comprises administering to the subject 4-iodo-3-nitrobenzamide or a salt, metabolite or prodrug thereof intermittently for about 6 or more cycles. In some instances, each treatment cycle is up to 28 days. In some cases, each treatment cycle is about 28 days. In other instances, each treatment cycle is up to 5 weeks. In other instances, each treatment cycle is about 5 weeks. In other instances, each treatment cycle is up to 6 weeks. In other instances, each treatment cycle is about 6 weeks. In other instances, each treatment cycle is up to 7 weeks. In other instances, each treatment cycle is about 7 weeks. In other instances, each treatment cycle is up to 8 weeks. In other instances, each treatment cycle is about 8 weeks. In other instances, each treatment cycle is up to 9 weeks. In other instances, each treatment cycle is about 9 weeks. In other instances, each treatment cycle is up to 10 weeks. In other instances, each treatment cycle is about 10 weeks.

In some embodiments, the cancer, for example, either TrxR-overexpressed or PRDX-overexpressed, is a solid tumor, a hematologic malignancy, or a melanoma. In some cases, the cancer is a metastatic cancer. In some cases, the cancer is a relapsed cancer. In other cases, the cancer is a refractory cancer.

In some instances, the cancer, for example, either TrxR-overexpressed or PRDX-overexpressed, is a solid tumor. In some instances, the solid tumor comprises brain cancer, bladder cancer, breast cancer, colorectal cancer, lung cancer, or prostate cancer. In some cases, the solid tumor is brain cancer. In some cases, the brain cancer comprises glioblastoma (or glioblastoma multiforme, GBM). Glioblastomas are tumors that arise from astrocytes or the star-shaped cells that make up the “glue-like,” or supportive tissue of the brain. In some cases, the glioblastoma is a primary glioblastoma or a de novo glioblastoma. In other instances, the glioblastoma is a secondary tumor. In some cases, glioblastoma is further classified into grade I, grade II, grade III and grade IV glioblastoma. In some cases, a subject is diagnosed with a grade I or grade II glioblastoma. In other cases, a subject is diagnosed with a grade III or a grade IV glioblastoma. In some cases, the glioblastoma is a metastasized glioblastoma.

In some embodiments, the solid tumor is breast cancer. In some instances, the breast cancer is further classified into ductal carcinoma in situ (DCIS), invasive ductal carcinoma (IDC), invasive lobular carcinoma (ILC), inflammatory breast cancer, lobular carcinoma in situ (LCIS), male breast cancer, Paget's disease of the Nipple, phyllodes tumors of the breast, triple negative breast cancer, HER2 positive breast cancer, Luminal A, Luminal B, Liminal B-like (HER2 negative), HER2-enriched, and normal-like breast cancer. Luminal A breast cancer is characterized as a hormone-receptor positive (estrogen-receptor and/or progesterone-receptor positive), HER2 negative and low level of protein Ki-67, relative to a normal breast cell. Luminal B breast cancer is characterized as hormone-receptor positive (estrogen-receptor and/or progesterone-receptor positive) and either HER2 positive or HER2 negative with a high level of Ki-67 relative to a normal breast cell. HER2-enriched breast cancer is hormone-receptor negative (estrogen-receptor and progesterone-receptor negative) and HER2 positive. Normal-like breast cancer is similar to luminal A breast cancer in that normal-like is characterized with hormone-receptor positive (estrogen-receptor and/or progesterone-receptor positive), HER2 negative, and a low-level of protein Ki-67. In some instances, IDC further comprises tubular carcinoma of the breast, medullary carcinoma of the breast, mucinous carcinoma of the breast, papillary carcinoma of the breast and cribriform carcinoma of the breast. In some cases, the breast cancer is a metastasized breast cancer. In some cases, the breast cancer is a relapsed breast cancer. In other cases, the breast cancer is a refractory breast cancer.

In some embodiments, the solid tumor is bladder cancer. In some instances, bladder cancer further comprises transitional cell bladder cancer (or urothelial cancer), non muscle invasive bladder cancer, invasive bladder cancer, squamous cell bladder cancer, adenocarcinoma of the urinary bladder, sarcoma, and small cell cancer of the bladder. In some cases, non muscle invasive bladder cancer further comprises carcinoma in situ (CIS) and high grade Ti tumors. In some cases, the bladder cancer is a metastasized bladder cancer. In some cases, the bladder cancer is a relapsed bladder cancer. In other cases, the bladder cancer is a refractory bladder cancer.

In some embodiments, the solid tumor is colorectal cancer. In some instances, colorectal cancer further comprises colorectal adenocarcinomas, carcinoid tumors, gastrointestinal stromal tumors (GISTs), lymphomas and sarcomas. In some cases, the colorectal cancer is a metastasized colorectal cancer. In some cases, the colorectal cancer is a relapsed colorectal cancer. In other cases, the colorectal cancer is a refractory colorectal cancer.

In some embodiments, the solid tumor is lung cancer. In some cases, lung cancer comprises non-small cell lung cancer (NSCLC), small cell lung cancer (SCLC), mesothelioma and carcinoid tumors. In some cases, NSCLC further comprises adenocarcinoma of lungs, adenocarcinoma in situ (AIS), minimally invasive adenocarcinoma (MIA), squamous cell carcinoma, large cell carcinoma, and large cell neuroendocrine tumors. In some cases, the lung cancer is a metastasized lung cancer. In some cases, the lung cancer is a relapsed lung cancer. In other cases, the lung cancer is a refractory lung cancer.

In some embodiments, the solid tumor is prostate cancer. In some instances, the prostate cancer further comprises acinar adenocarcinoma, ductal adenocarcinoma, transitional cell (or urothelial) cancer, squamous cell cancer, small cell prostate cancer, carcinoid, and sarcoma. In some cases, the prostate cancer is a metastasized prostate cancer. In some cases, the prostate cancer is a relapsed prostate cancer. In other cases, the prostate cancer is a refractory prostate cancer.

In some embodiments, the cancer, for example, either TrxR-overexpressed or PRDX-overexpressed, is a hematologic malignancy. In some instances, the hematologic malignancy comprises a T-cell leukemia. In some cases, the T-cell leukemia comprises large granular lymphocytic leukemia, T-cell acute lymphoblastic leukemia (T-ALL) or T-cell prolymphocytic leukemia (T-PLL). In some cases, the T-cell leukemia is a metastasized T-cell leukemia. In some cases, the T-cell leukemia is a relapsed T-cell leukemia. In other cases, the T-cell leukemia is a refractory T-cell leukemia.

In some embodiments, the cancer, for example, either TrxR-overexpressed or PRDX-overexpressed, is a melanoma. In some cases, the melanoma is a metastasized melanoma. In some cases, the melanoma is a relapsed melanoma. In other cases, the melanoma is a refractory melanoma.

### Methods of Patient Selection

In certain embodiments, disclosed herein are methods of selecting a subject for treatment with a nitrobenzamide compound described supra. In some instances, the nitrobenzamide compound is a compound encompassed by Formula (I). In some instances, the nitrobenzamide compound is 4-iodo-3-nitrobenzamide or a salt, metabolite or prodrug thereof.

In some embodiments, the method comprises selecting a subject for treatment with 4-iodo-3-nitrobenzamide or a salt, metabolite or prodrug thereof. In some embodiments, disclosed herein is a method of selecting a subject for treatment with 4-iodo-3-nitrobenzamide or a salt, metabolite or prodrug thereof, comprising: (a) contacting at least one gene selected from thioredoxin reductase 2 (TXNRD2), thioredoxin 2 (TXN2), methionine sulfoxide reductase B3 (MSRB3), methionine sulfoxide reductase A (MSRA), and glutathione transferase zeta 1 (GSTZ1) with a set of primers to produce amplified nucleic acids, wherein the at least one gene is isolated from a tumor sample obtained from the subject; (b) determining the level of the amplified nucleic acids in the tumor sample relative to a control; and (c) administering a therapeutically effective amount of 4-iodo-3-nitrobenzamide or a salt, metabolite or prodrug thereof to the subject if the level of the amplified nucleic acids is greater than the level in the control.

In some embodiments, the level of at least one gene selected from TXNRD2, TXN2, MSRB3 and MSRA is determined. In some cases, the level of two or more genes selected from TXNRD2, TXN2, MSRB3 and MSRA are determined. In some cases, the level of TXNRD2 is determined. In some cases, the level of TXN2 is determined. In some cases, the level of MSRB3 is determined. In some cases, the level of MSRA is determined. In some cases, the level of TXNRD2, TXN2, MSRB3 and MSRA are determined.

In some embodiments, disclosed herein is a method of detecting at least one gene from thioredoxin reductase 2 (TXNRD2), thioredoxin 2 (TXN2), methionine sulfoxide reductase B3 (MSRB3), methionine sulfoxide reductase A (MSRA), and glutathione transferase zeta 1 (GSTZ1) in a subject, comprising a) obtaining a tumor sample from a subject; and b) detecting whether at least one gene from TXNRD2, TXN2, MSRB3, MSRA, and GSTZ1 is present in the tumor sample by contacting the tumor sample with a set of nucleic acid probes and detecting binding between TXNRD2, TXN2, MSRB3, MSRA, or GSTZ1 and the nucleic acid probes, wherein the set of nucleic acid probes hybridizes to five and no more than five markers, and the five markers are TXNRD2, TXN2, MSRB3, MSRA, and GSTZ1.

In some embodiments, the level of the amplified nucleic acids from at least one gene selected from TXNRD2, TXN2, MSRB3, MSRA and GSTZ1 correlates to a decreased risk of disease progression.

In some embodiments, the method of selecting a subject for treatment with 4-iodo-3-nitrobenzamide or a salt, metabolite or prodrug thereof further comprise determining the level of amplified nucleic acids from at least one gene selected from NAD(P)H dehydrogenase quinone 2 (NQO2), glutathione S-transferase theta 2 (GSTT2), glutathione S-transferase M3 (GSTM3), glutaredoxin (GLRX), selenoprotein O (SELO), paraoxonase 1 (PON1), glutathione S-transferase omega 1 (GSTO1), glutaredoxin 3 (GLRX3), selenoprotein X 1 (SEPX1), and thioredoxin reductase 1 (TXNRD1) and comparing the level with a control. In some cases, the treatment with 4-iodo-3-nitrobenzamide or a salt, metabolite or prodrug thereof is discontinued if the level of amplified nucleic acids is greater than the level in the control.

In some embodiments, 4-iodo-3-nitrobenzamide or a salt, metabolite or prodrug thereof is administered from about 2 mg/kg to about 200 mg/kg. In some instances, 4-iodo-3-nitrobenzamide or a salt, metabolite or prodrug thereof is administered from about 2 mg/kg to about 150 mg/kg, from about 2 mg/kg to about 100 mg/kg, or from about 2 mg/kg to about 60 mg/kg. In some instances, 4-iodo-3-nitrobenzamide or a salt, metabolite or prodrug thereof is administered from about 5 mg/kg to about 150 mg/kg, from about 5 mg/kg to about 100 mg/kg, or from about 5 mg/kg to about 60 mg/kg. In some instances, 4-iodo-3-nitrobenzamide or a salt, metabolite or prodrug thereof is administered at a range of about 5 mg/kg to about 50 mg/kg, about 5 mg/kg to about 40 mg/kg, about 5 mg/kg to about 30 mg/kg, about 5 mg/kg to about 20 mg/kg, about 5 mg/kg to about 10 mg/kg, about 6 mg/kg to about 60 mg/kg, about 6 mg/kg to about 50 mg/kg, about 6 mg/kg to about 40 mg/kg, about 6 mg/kg to about 30 mg/kg, about 6 mg/kg to about 20 mg/kg, about 6 mg/kg to about 10 mg/kg, about 6 mg/kg to about 9 mg/kg, about 7 mg/kg to about 60 mg/kg, about 7 mg/kg to about 50 mg/kg, about 7 mg/kg to about 40 mg/kg, about 7 mg/kg to about 30 mg/kg, about 7 mg/kg to about 20 mg/kg, about 7 mg/kg to about 10 mg/kg, about 7 mg/kg to about 9 mg/kg, about 7 mg/kg to about 8 mg/kg, about 8 mg/kg to about 60 mg/kg, about 8 mg/kg to about 40 mg/kg, about 8 mg/kg to about 30 mg/kg, about 8 mg/kg to about 20 mg/kg, about 8 mg/kg to about 10 mg/kg, about 8 mg/kg to about 9 mg/kg, or about 8 mg/kg to about 8.6 mg/kg. In some embodiments, 4-iodo-3-nitrobenzamide or a salt, metabolite or prodrug thereof is administered at a range of about 6 mg/kg to about 40 mg/kg, about 6 mg/kg to about 30 mg/kg, about 6 mg/kg to about 20 mg/kg, about 6 mg/kg to about 10 mg/kg, about 6 mg/kg to about 9 mg/kg, about 7 mg/kg to about 30 mg/kg, about 7 mg/kg to about 20 mg/kg, about 7 mg/kg to about 9 mg/kg, about 7 mg/kg to about 8 mg/kg, about 8 mg/kg to about 20 mg/kg, about 8 mg/kg to about 9 mg/kg, or about 8 mg/kg to about 8.6 mg/kg. In some embodiments, 4-iodo-3-nitrobenzamide or a salt, metabolite or prodrug thereof is administered at a range of about 5 mg/kg to about 40 mg/kg. In some embodiments, 4-iodo-3-nitrobenzamide or a salt, metabolite or prodrug thereof is administered at a range of about 6 mg/kg to about 9 mg/kg. In some embodiments, 4-iodo-3-nitrobenzamide or a salt, metabolite or prodrug thereof is administered at a range of about 6 mg/kg to about 8.6 mg/kg. In some embodiments, 4-iodo-3-nitrobenzamide or a salt, metabolite or prodrug thereof is administered at a range of about 6 mg/kg to about 8 mg/kg. In some embodiments, 4-iodo-3-nitrobenzamide or a salt, metabolite or prodrug thereof is administered at a range of about 7 mg/kg to about 9 mg/kg. In some embodiments, 4-iodo-3-nitrobenzamide or a salt, metabolite or prodrug thereof is administered at a range of about 7 mg/kg to about 8.6 mg/kg. In some embodiments, 4-iodo-3-nitrobenzamide or a salt, metabolite or prodrug thereof is administered at a range of about 7 mg/kg to about 8 mg/kg. In some embodiments, 4-iodo-3-nitrobenzamide or a salt, metabolite or prodrug thereof is administered at a range of about 8 mg/kg to about 9 mg/kg. In some embodiments, 4-iodo-3-nitrobenzamide or a salt, metabolite or prodrug thereof is administered at a range of about 8 mg/kg to about 8.6 mg/kg.

In some embodiments, 4-iodo-3-nitrobenzamide or a salt, metabolite or prodrug thereof is administered at about 2 mg/kg, about 3 mg/kg, about 4 mg/kg, about 5 mg/kg, about 6 mg/kg, about 7 mg/kg, about 8 mg/kg, about 8.5 mg/kg, about 8.6 mg/kg, about 9 mg/kg, about 10 mg/kg, about 15 mg/kg, about 20 mg/kg, about 25 mg/kg, about 30 mg/kg, about 40 mg/kg, about 50 mg/kg, about 60 mg/kg, about 100 mg/kg, about 150 mg/kg, or about 200 mg/kg. In some embodiments, 4-iodo-3-nitrobenzamide or a salt, metabolite or prodrug thereof is administered at about 5 mg/kg, about 6 mg/kg, about 7 mg/kg, about 8 mg/kg, about 8.5 mg/kg, about 8.6 mg/kg, about 9 mg/kg, about 10 mg/kg, about 15 mg/kg, about 20 mg/kg, about 25 mg/kg, about 30 mg/kg, or about 40 mg/kg. In some embodiments, 4-iodo-3-nitrobenzamide or a salt, metabolite or prodrug thereof is administered at about 2 mg/kg. In some embodiments, 4-iodo-3-nitrobenzamide or a salt, metabolite or prodrug thereof is administered at about 3 mg/kg. In some embodiments, 4-iodo-3-nitrobenzamide or a salt, metabolite or prodrug thereof is administered at about 4 mg/kg. In some embodiments, 4-iodo-3-nitrobenzamide or a salt, metabolite or prodrug thereof is administered at about 5 mg/kg. In some embodiments, 4-iodo-3-nitrobenzamide or a salt, metabolite or prodrug thereof is administered at about 6 mg/kg. In some embodiments, 4-iodo-3-nitrobenzamide or a salt, metabolite or prodrug thereof is administered at about 7 mg/kg. In some embodiments, 4-iodo-3-nitrobenzamide or a salt, metabolite or prodrug thereof is administered at about 8 mg/kg. In some embodiments, 4-iodo-3-nitrobenzamide or a salt, metabolite or prodrug thereof is administered at about 8.5 mg/kg. In some embodiments, 4-iodo-3-nitrobenzamide or a salt, metabolite or prodrug thereof is administered at about 8.6 mg/kg. In some embodiments, 4-iodo-3-nitrobenzamide or a salt, metabolite or prodrug thereof is administered at about 9 mg/kg. In some embodiments, 4-iodo-3-nitrobenzamide or a salt, metabolite or prodrug thereof is administered at about 10 mg/kg. In some embodiments, 4-iodo-3-nitrobenzamide or a salt, metabolite or prodrug thereof is administered at about 15 mg/kg. In some embodiments, 4-iodo-3-nitrobenzamide or a salt, metabolite or prodrug thereof is administered at about 20 mg/kg. In some embodiments, 4-iodo-3-nitrobenzamide or a salt, metabolite or prodrug thereof is administered at about 30 mg/kg. In some embodiments, 4-iodo-3-nitrobenzamide or a salt, metabolite or prodrug thereof is administered at about 40 mg/kg. In some embodiments, 4-iodo-3-nitrobenzamide or a salt, metabolite or prodrug thereof is administered at about 50 mg/kg. In some embodiments, 4-iodo-3-nitrobenzamide or a salt, metabolite or prodrug thereof is administered at about 60 mg/kg.

In some embodiments, 4-iodo-3-nitrobenzamide or a salt, metabolite or prodrug thereof is administered to a subject at one or more dosing schedules. In some embodiments, the dosing schedule comprises administering to the subject 4-iodo-3-nitrobenzamide or a salt, metabolite or prodrug thereof once per day, twice a week, three times a week, four times a week, five times a week, daily, every other day, once a month, twice a month, or every week. In some instances, the dosing schedule comprises administering to the subject 4-iodo-3-nitrobenzamide or a salt, metabolite or prodrug thereof once per day.

In some embodiments, the dosing schedule comprises administering to the subject 4-iodo-3-nitrobenzamide or a salt, metabolite or prodrug thereof continuously for about 1, 2, 3, 4, 5, 6, 7, 8, 9, 10, 11, 12 or more weeks. In some instances, the dosing schedule comprises administering to the subject 4-iodo-3-nitrobenzamide or a salt, metabolite or prodrug thereof continuously for about 1, 2, 3, 4, 5, 6, 7, 8, 9, 10 or more weeks. In some instances, the dosing schedule comprises administering to the subject 4-iodo-3-nitrobenzamide or a salt, metabolite or prodrug thereof continuously for about 1, 2, 3, 4, 5, 6 or more weeks. In some instances, the dosing schedule comprises administering to the subject 4-iodo-3-nitrobenzamide or a salt, metabolite or prodrug thereof continuously for about 5 weeks. In some instances, the dosing schedule comprises administering to the subject 4-iodo-3-nitrobenzamide or a salt, metabolite or prodrug thereof continuously for about 6 weeks. In some instances, the dosing schedule comprises administering to the subject 4-iodo-3-nitrobenzamide or a salt, metabolite or prodrug thereof continuously for about 7 weeks. In some instances, the dosing schedule comprises administering to the subject 4-iodo-3-nitrobenzamide or a salt, metabolite or prodrug thereof continuously for about 8 weeks. In some instances, the dosing schedule comprises administering to the subject 4-iodo-3-nitrobenzamide or a salt, metabolite or prodrug thereof continuously for about 9 weeks. In some instances, the dosing schedule comprises administering to the subject 4-iodo-3-nitrobenzamide or a salt, metabolite or prodrug thereof continuously for about 10 weeks. In some instances, a 5-week dosing schedule is considered as one cycle. In some instances, a 6-week dosing schedule is considered as one cycle. In some instances, a 7-week dosing schedule is considered as one cycle. In some instances, a 8-week dosing schedule is considered as one cycle. In some instances, a 9-week dosing schedule is considered as one cycle. In some instances, a 10-week dosing schedule is considered as one cycle.

In some embodiments, the dosing schedule comprises administering to the subject 4-iodo-3-nitrobenzamide or a salt, metabolite or prodrug thereof continuously for about 1, 2, 3, 4, 5, 6, 7, 8, 9, 10, 11, 12 or more months. In some instances, the dosing schedule comprises administering to the subject 4-iodo-3-nitrobenzamide or a salt, metabolite or prodrug thereof continuously for about 1, 2, 3, 4, 5, 6 or more months.

In some embodiments, the dosing schedule comprises administering to the subject 4-iodo-3-nitrobenzamide or a salt, metabolite or prodrug thereof continuously for about 1, 2, 3, 4, 5, 6, 7, 8, 9, 10 or more treatment cycles. In some instances, the dosing schedule comprises administering to the subject 4-iodo-3-nitrobenzamide or a salt, metabolite or prodrug thereof continuously for about 1, 2, 3, 4, 5, 6 or more treatment cycles. In some instances, the dosing schedule comprises administering to the subject 4-iodo-3-nitrobenzamide or a salt, metabolite or prodrug thereof continuously for about 1, 2, 3, 4 or more treatment cycles. In some instances, the dosing schedule comprises administering to the subject 4-iodo-3-nitrobenzamide or a salt, metabolite or prodrug thereof continuously for about 1 or more cycles. In some instances, the dosing schedule comprises administering to the subject 4-iodo-3-nitrobenzamide or a salt, metabolite or prodrug thereof continuously for about 2 or more cycles. In some instances, the dosing schedule comprises administering to the subject 4-iodo-3-nitrobenzamide or a salt, metabolite or prodrug thereof continuously for about 3 or more cycles. In some instances, the dosing schedule comprises administering to the subject 4-iodo-3-nitrobenzamide or a salt, metabolite or prodrug thereof continuously for about 4 or more cycles. In some instances, each treatment cycle is up to 28 days. In some cases, each treatment cycle is about 28 days. In other instances, each treatment cycle is up to 5 weeks. In other instances, each treatment cycle is about 5 weeks. In other instances, each treatment cycle is up to 6 weeks. In other instances, each treatment cycle is about 6 weeks. In other instances, each treatment cycle is up to 7 weeks. In other instances, each treatment cycle is about 7 weeks. In other instances, each treatment cycle is up to 8 weeks. In other instances, each treatment cycle is about 8 weeks. In other instances, each treatment cycle is up to 9 weeks. In other instances, each treatment cycle is about 9 weeks. In other instances, each treatment cycle is up to 10 weeks. In other instances, each treatment cycle is about 10 weeks.

In some embodiments, the dosing schedule comprises administering to the subject 4-iodo-3-nitrobenzamide or a salt, metabolite or prodrug thereof intermittently for about 1, 2, 3, 4, 5, 6, 7, 8, 9, 10, 11, 12 or more weeks. In some instances, the dosing schedule comprises administering to the subject 4-iodo-3-nitrobenzamide or a salt, metabolite or prodrug thereof intermittently for about 1, 2, 3, 4, 5, 6, 7, 8, 9, 10 or more weeks. In some instances, the dosing schedule comprises administering to the subject 4-iodo-3-nitrobenzamide or a salt, metabolite or prodrug thereof intermittently for about 1, 2, 3, 4, 5, 6 or more weeks. In some instances, the dosing schedule comprises administering to the subject 4-iodo-3-nitrobenzamide or a salt, metabolite or prodrug thereof intermittently for about 5 weeks. In some instances, the dosing schedule comprises administering to the subject 4-iodo-3-nitrobenzamide or a salt, metabolite or prodrug thereof intermittently for about 6 weeks. In some instances, the dosing schedule comprises administering to the subject 4-iodo-3-nitrobenzamide or a salt, metabolite or prodrug thereof intermittently for about 7 weeks. In some instances, the dosing schedule comprises administering to the subject 4-iodo-3-nitrobenzamide or a salt, metabolite or prodrug thereof intermittently for about 8 weeks. In some instances, the dosing schedule comprises administering to the subject 4-iodo-3-nitrobenzamide or a salt, metabolite or prodrug thereof intermittently for about 9 weeks. In some instances, the dosing schedule comprises administering to the subject 4-iodo-3-nitrobenzamide or a salt, metabolite or prodrug thereof intermittently for about 10 weeks. In some instances, a 5-week dosing schedule is considered as one cycle. In some instances, a 6-week dosing schedule is considered as one cycle. In some instances, a 7-week dosing schedule is considered as one cycle. In some instances, a 8-week dosing schedule is considered as one cycle. In some instances, a 9-week dosing schedule is considered as one cycle. In some instances, a 10-week dosing schedule is considered as one cycle.

In some embodiments, the dosing schedule comprises administering to the subject 4-iodo-3-nitrobenzamide or a salt, metabolite or prodrug thereof intermittently for about 1, 2, 3, 4, 5, 6, 7, 8, 9, 10, 11, 12 or more months. In some instances, the dosing schedule comprises administering to the subject 4-iodo-3-nitrobenzamide or a salt, metabolite or prodrug thereof intermittently for about 1, 2, 3, 4, 5, 6 or more months.

In some embodiments, the dosing schedule comprises administering to the subject 4-iodo-3-nitrobenzamide or a salt, metabolite or prodrug thereof intermittently for about 1, 2, 3, 4, 5, 6, 7, 8, 9, 10 or more treatment cycles. In some instances, the dosing schedule comprises administering to the subject 4-iodo-3-nitrobenzamide or a salt, metabolite or prodrug thereof intermittently for about 1, 2, 3, 4, 5, 6 or more treatment cycles. In some instances, the dosing schedule comprises administering to the subject 4-iodo-3-nitrobenzamide or a salt, metabolite or prodrug thereof intermittently for about 1, 2, 3, 4 or more treatment cycles. In some instances, the dosing schedule comprises administering to the subject 4-iodo-3-nitrobenzamide or a salt, metabolite or prodrug thereof intermittently for about 1 or more cycles. In some instances, the dosing schedule comprises administering to the subject 4-iodo-3-nitrobenzamide or a salt, metabolite or prodrug thereof intermittently for about 2 or more cycles. In some instances, the dosing schedule comprises administering to the subject 4-iodo-3-nitrobenzamide or a salt, metabolite or prodrug thereof intermittently for about 3 or more cycles. In some instances, the dosing schedule comprises administering to the subject 4-iodo-3-nitrobenzamide or a salt, metabolite or prodrug thereof intermittently for about 4 or more cycles. In some instances, the dosing schedule comprises administering to the subject 4-iodo-3-nitrobenzamide or a salt, metabolite or prodrug thereof intermittently for about 5 or more cycles. In some instances, the dosing schedule comprises administering to the subject 4-iodo-3-nitrobenzamide or a salt, metabolite or prodrug thereof intermittently for about 6 or more cycles. In some instances, each treatment cycle is up to 28 days. In some cases, each treatment cycle is about 28 days. In other instances, each treatment cycle is up to 5 weeks. In other instances, each treatment cycle is about 5 weeks. In other instances, each treatment cycle is up to 6 weeks. In other instances, each treatment cycle is about 6 weeks. In other instances, each treatment cycle is up to 7 weeks. In other instances, each treatment cycle is about 7 weeks. In other instances, each treatment cycle is up to 8 weeks. In other instances, each treatment cycle is about 8 weeks. In other instances, each treatment cycle is up to 9 weeks. In other instances, each treatment cycle is about 9 weeks. In other instances, each treatment cycle is up to 10 weeks. In other instances, each treatment cycle is about 10 weeks.

In some embodiments, the cancer is a TrxR-overexpressed cancer or a PRDX-overexpressed cancer. In some instances, the cancer is a TrxR-overexpressed cancer. In other instances, the cancer is a PRDX-overexpressed cancer. In some cases, the TrxR-overexpressed cancer is a metastatic TrxR-overexpressed cancer. In some cases, the PRDX-overexpressed cancer is metastatic PRDX-overexpressed cancer. In some cases, the TrxR-overexpressed cancer is a relapsed TrxR-overexpressed cancer. In some cases, the PRDX-overexpressed cancer is a relapsed PRDX-overexpressed cancer. In other cases, the TrxR-overexpressed cancer is a refractory TrxR-overexpressed cancer. In other cases, the PRDX-overexpressed cancer is a refractory PRDX-overexpressed cancer.

In some embodiments, the cancer is a solid tumor, a hematologic malignancy, or a melanoma. In some cases, the cancer is a metastatic cancer. In some cases, the cancer is a relapsed cancer. In other cases, the cancer is a refractory cancer.

In some instances, the cancer is a solid tumor. In some instances, the solid tumor comprises brain cancer, bladder cancer, breast cancer, colorectal cancer, lung cancer, or prostate cancer. In some cases, the solid tumor is brain cancer. In some cases, the brain cancer comprises glioblastoma (or glioblastoma multiforme, GBM). In some cases, the glioblastoma is a primary glioblastoma or a de novo glioblastoma. In other instances, the glioblastoma is a secondary tumor. In some cases, glioblastoma is further classified into grade I, grade II, grade III and grade IV glioblastoma. In some cases, a subject is diagnosed with a grade I or grade II glioblastoma. In other cases, a subject is diagnosed with a grade III or a grade IV glioblastoma. In some cases, the glioblastoma is a metastasized glioblastoma.

In some embodiments, the solid tumor is breast cancer. In some instances, the breast cancer is further classified into ductal carcinoma in situ (DCIS), invasive ductal carcinoma (IDC), invasive lobular carcinoma (ILC), inflammatory breast cancer, lobular carcinoma in situ (LCIS), male breast cancer, Paget's disease of the Nipple, phyllodes tumors of the breast, triple negative breast cancer, HER2 positive breast cancer, Luminal A, Luminal B, Liminal B-like (HER2 negative), HER2-enriched, and normal-like breast cancer. In some instances, IDC further comprises tubular carcinoma of the breast, medullary carcinoma of the breast, mucinous carcinoma of the breast, papillary carcinoma of the breast and cribriform carcinoma of the breast. In some cases, the breast cancer is a metastasized breast cancer. In some cases, the breast cancer is a relapsed breast cancer. In other cases, the breast cancer is a refractory breast cancer.

In some embodiments, the solid tumor is bladder cancer. In some instances, bladder cancer further comprises transitional cell bladder cancer (or urothelial cancer), non muscle invasive bladder cancer, invasive bladder cancer, squamous cell bladder cancer, adenocarcinoma of the urinary bladder, sarcoma, and small cell cancer of the bladder. In some cases, non muscle invasive bladder cancer further comprises carcinoma in situ (CIS) and high grade Ti tumors. In some cases, the bladder cancer is a metastasized bladder cancer. In some cases, the bladder cancer is a relapsed bladder cancer. In other cases, the bladder cancer is a refractory bladder cancer.

In some embodiments, the solid tumor is colorectal cancer. In some instances, colorectal cancer further comprises colorectal adenocarcinomas, carcinoid tumors, gastrointestinal stromal tumors (GISTs), lymphomas and sarcomas. In some cases, the colorectal cancer is a metastasized colorectal cancer. In some cases, the colorectal cancer is a relapsed colorectal cancer. In other cases, the colorectal cancer is a refractory colorectal cancer.

In some embodiments, the solid tumor is lung cancer. In some cases, lung cancer comprises non-small cell lung cancer (NSCLC), small cell lung cancer (SCLC), mesothelioma and carcinoid tumors. In some cases, NSCLC further comprises adenocarcinoma of lungs, adenocarcinoma in situ (AIS), minimally invasive adenocarcinoma (MIA), squamous cell carcinoma, large cell carcinoma, and large cell neuroendocrine tumors. In some cases, the lung cancer is a metastasized lung cancer. In some cases, the lung cancer is a relapsed lung cancer. In other cases, the lung cancer is a refractory lung cancer.

In some embodiments, the solid tumor is prostate cancer. In some instances, the prostate cancer further comprises acinar adenocarcinoma, ductal adenocarcinoma, transitional cell (or urothelial) cancer, squamous cell cancer, small cell prostate cancer, carcinoid, and sarcoma. In some cases, the prostate cancer is a metastasized prostate cancer. In some cases, the prostate cancer is a relapsed prostate cancer. In other cases, the prostate cancer is a refractory prostate cancer.

In some embodiments, the cancer, for example, either TrxR-overexpressed or PRDX-overexpressed, is a hematologic malignancy. In some instances, the hematologic malignancy comprises a T-cell leukemia. In some cases, the T-cell leukemia comprises large granular lymphocytic leukemia, T-cell acute lymphoblastic leukemia (T-ALL) or T-cell prolymphocytic leukemia (T-PLL). In some cases, the T-cell leukemia is a metastasized T-cell leukemia. In some cases, the T-cell leukemia is a relapsed T-cell leukemia. In other cases, the T-cell leukemia is a refractory T-cell leukemia.

In some embodiments, the cancer, for example, either TrxR-overexpressed or PRDX-overexpressed, is a melanoma. In some cases, the melanoma is a metastasized melanoma. In some cases, the melanoma is a relapsed melanoma. In other cases, the melanoma is a refractory melanoma.

### Methods of Monitoring of the Treatment Progress

In certain embodiments, disclosed herein are methods of monitoring treatment progression. In some embodiments, the method of monitoring a treatment regimen in a subject having a cancer, comprising: (a) administering to the subject a therapeutically effective amount of a nitrobenzamide compound described supra; (b) contacting at least one gene selected from NAD(P)H dehydrogenase quinone 2 (NQO2), glutathione S-transferase theta 2 (GSTT2), glutathione S-transferase M3 (GSTM3), glutaredoxin (GLRX), selenoprotein O (SELO), paraoxonase 1 (PON1), glutathione S-transferase omega 1 (GSTO1), glutaredoxin 3 (GLRX3), selenoprotein X 1 (SEPX1), and thioredoxin reductase 1 (TXNRD1) with a set of primers to produce amplified nucleic acids, wherein the at least one gene is isolated from a tumor sample obtained from the subject after treatment initiation; (c) determining the level of the amplified nucleic acids in the tumor sample relative to a control; and (d) continuing treatment with nitrobenzamide compound if the level of the amplified nucleic acids is lower than or is the same as the level of the control, or discontinuing treatment with nitrobenzamide compound if the level of the amplified nucleic acids is greater than the level of the control. In some instances, the nitrobenzamide compound is a compound encompassed by Formula (I). In some instances, the nitrobenzamide compound is 4-iodo-3-nitrobenzamide or a salt, metabolite or prodrug thereof.

In some embodiments, the method of monitoring a treatment regimen in a subject having a cancer, comprising: (a) administering to the subject a therapeutically effective amount of 4-iodo-3-nitrobenzamide or a salt, metabolite or prodrug thereof, (b) contacting at least one gene selected from NAD(P)H dehydrogenase quinone 2 (NQO2), glutathione S-transferase theta 2 (GSTT2), glutathione S-transferase M3 (GSTM3), glutaredoxin (GLRX), selenoprotein O (SELO), paraoxonase 1 (PON1), glutathione S-transferase omega 1 (GSTO1), glutaredoxin 3 (GLRX3), selenoprotein X 1 (SEPX1), and thioredoxin reductase 1 (TXNRD1) with a set of primers to produce amplified nucleic acids, wherein the at least one gene is isolated from a tumor sample obtained from the subject after treatment initiation; (c) determining the level of the amplified nucleic acids in the tumor sample relative to a control; and (d) continuing treatment with 4-iodo-3-nitrobenzamide or a metabolite thereof if the level of the amplified nucleic acids is lower than or is the same as the level of the control, or discontinuing treatment with 4-iodo-3-nitrobenzamide or a metabolite thereof if the level of the amplified nucleic acids is greater than the level of the control.

In some embodiments, the level of at least one gene selected from NQO2, GSTT2, GSTM3, GLRX, GSTO1, GLRX3 and TXNRD1 is determined. In some instances, the level of at least one gene selected from NQO2, GSTT2, GSTM3, GLRX, GSTO1 and GLRX3 is determined. In some instances, the level of at least one gene selected from GSTT2, GSTM3, GLRX, GSTO1 and GLRX3 is determined. In some instances, the level of at least one gene selected from GSTT2, GSTM3, and GSTO1 is determined. In some instances, the level of at least one gene selected from NQO2, SELO, PON1, SEPX1 and TXNRD1 is determined. In some instances, the level of at least one gene selected from SELO, PON1, SEPX1 and TXNRD1 is determined. In some instances, the level of at least one gene selected from SELO, PON1 and SEPX1 is determined. In some instances, the level of NQO2 is determined. In some instances, the level of GSTT2 is determined. In some instances, the level of GSTM3 is determined. In some instances, the level of GLRX is determined. In some instances, the level of GSTO1 is determined. In some instances, the level of GLRX3 is determined. In some instances, the level of TXNRD1 is determined.

In some embodiments, the level of amplified nucleic acids greater than the level in the control correlates to an increased risk of disease progression.

In some embodiments, the method further comprises determining the level of amplified nucleic acids from at least one gene selected from thioredoxin reductase 2 (TXNRD2), thioredoxin 2 (TXN2), methionine sulfoxide reductase B3 (MSRB3), methionine sulfoxide reductase A (MSRA), and glutathione transferase zeta 1 (GSTZ1) and comparing the level with a control. In some cases, the level of amplified nucleic acids greater than the level in the control correlates to a decreased risk of disease progression.

In some embodiments, 4-iodo-3-nitrobenzamide or a salt, metabolite or prodrug thereof is administered from about 2 mg/kg to about 200 mg/kg. In some instances, 4-iodo-3-nitrobenzamide or a salt, metabolite or prodrug thereof is administered from about 2 mg/kg to about 150 mg/kg, from about 2 mg/kg to about 100 mg/kg, or from about 2 mg/kg to about 60 mg/kg. In some instances, 4-iodo-3-nitrobenzamide or a salt, metabolite or prodrug thereof is administered from about 5 mg/kg to about 150 mg/kg, from about 5 mg/kg to about 100 mg/kg, or from about 5 mg/kg to about 60 mg/kg. In some instances, 4-iodo-3-nitrobenzamide or a salt, metabolite or prodrug thereof is administered at a range of about 5 mg/kg to about 50 mg/kg, about 5 mg/kg to about 40 mg/kg, about 5 mg/kg to about 30 mg/kg, about 5 mg/kg to about 20 mg/kg, about 5 mg/kg to about 10 mg/kg, about 6 mg/kg to about 60 mg/kg, about 6 mg/kg to about 50 mg/kg, about 6 mg/kg to about 40 mg/kg, about 6 mg/kg to about 30 mg/kg, about 6 mg/kg to about 20 mg/kg, about 6 mg/kg to about 10 mg/kg, about 6 mg/kg to about 9 mg/kg, about 7 mg/kg to about 60 mg/kg, about 7 mg/kg to about 50 mg/kg, about 7 mg/kg to about 40 mg/kg, about 7 mg/kg to about 30 mg/kg, about 7 mg/kg to about 20 mg/kg, about 7 mg/kg to about 10 mg/kg, about 7 mg/kg to about 9 mg/kg, about 7 mg/kg to about 8 mg/kg, about 8 mg/kg to about 60 mg/kg, about 8 mg/kg to about 40 mg/kg, about 8 mg/kg to about 30 mg/kg, about 8 mg/kg to about 20 mg/kg, about 8 mg/kg to about 10 mg/kg, about 8 mg/kg to about 9 mg/kg, or about 8 mg/kg to about 8.6 mg/kg. In some embodiments, 4-iodo-3-nitrobenzamide or a salt, metabolite or prodrug thereof is administered at a range of about 6 mg/kg to about 40 mg/kg, about 6 mg/kg to about 30 mg/kg, about 6 mg/kg to about 20 mg/kg, about 6 mg/kg to about 10 mg/kg, about 6 mg/kg to about 9 mg/kg, about 7 mg/kg to about 30 mg/kg, about 7 mg/kg to about 20 mg/kg, about 7 mg/kg to about 9 mg/kg, about 7 mg/kg to about 8 mg/kg, about 8 mg/kg to about 20 mg/kg, about 8 mg/kg to about 9 mg/kg, or about 8 mg/kg to about 8.6 mg/kg. In some embodiments, 4-iodo-3-nitrobenzamide or a salt, metabolite or prodrug thereof is administered at a range of about 5 mg/kg to about 40 mg/kg. In some embodiments, 4-iodo-3-nitrobenzamide or a salt, metabolite or prodrug thereof is administered at a range of about 6 mg/kg to about 9 mg/kg. In some embodiments, 4-iodo-3-nitrobenzamide or a salt, metabolite or prodrug thereof is administered at a range of about 6 mg/kg to about 8.6 mg/kg. In some embodiments, 4-iodo-3-nitrobenzamide or a salt, metabolite or prodrug thereof is administered at a range of about 6 mg/kg to about 8 mg/kg. In some embodiments, 4-iodo-3-nitrobenzamide or a salt, metabolite or prodrug thereof is administered at a range of about 7 mg/kg to about 9 mg/kg. In some embodiments, 4-iodo-3-nitrobenzamide or a salt, metabolite or prodrug thereof is administered at a range of about 7 mg/kg to about 8.6 mg/kg. In some embodiments, 4-iodo-3-nitrobenzamide or a salt, metabolite or prodrug thereof is administered at a range of about 7 mg/kg to about 8 mg/kg. In some embodiments, 4-iodo-3-nitrobenzamide or a salt, metabolite or prodrug thereof is administered at a range of about 8 mg/kg to about 9 mg/kg. In some embodiments, 4-iodo-3-nitrobenzamide or a salt, metabolite or prodrug thereof is administered at a range of about 8 mg/kg to about 8.6 mg/kg.

In some embodiments, 4-iodo-3-nitrobenzamide or a salt, metabolite or prodrug thereof is administered at about 2 mg/kg, about 3 mg/kg, about 4 mg/kg, about 5 mg/kg, about 6 mg/kg, about 7 mg/kg, about 8 mg/kg, about 8.5 mg/kg, about 8.6 mg/kg, about 9 mg/kg, about 10 mg/kg, about 15 mg/kg, about 20 mg/kg, about 25 mg/kg, about 30 mg/kg, about 40 mg/kg, about 50 mg/kg, about 60 mg/kg, about 100 mg/kg, about 150 mg/kg, or about 200 mg/kg. In some embodiments, 4-iodo-3-nitrobenzamide or a salt, metabolite or prodrug thereof is administered at about 2 mg/kg. In some embodiments, 4-iodo-3-nitrobenzamide or a salt, metabolite or prodrug thereof is administered at about 3 mg/kg. In some embodiments, 4-iodo-3-nitrobenzamide or a salt, metabolite or prodrug thereof is administered at about 4 mg/kg. In some embodiments, 4-iodo-3-nitrobenzamide or a salt, metabolite or prodrug thereof is administered at about 5 mg/kg, about 6 mg/kg, about 7 mg/kg, about 8 mg/kg, about 8.5 mg/kg, about 8.6 mg/kg, about 9 mg/kg, about 10 mg/kg, about 15 mg/kg, about 20 mg/kg, about 25 mg/kg, about 30 mg/kg, or about 40 mg/kg. In some embodiments, 4-iodo-3-nitrobenzamide or a salt, metabolite or prodrug thereof is administered at about 5 mg/kg. In some embodiments, 4-iodo-3-nitrobenzamide or a salt, metabolite or prodrug thereof is administered at about 6 mg/kg. In some embodiments, 4-iodo-3-nitrobenzamide or a salt, metabolite or prodrug thereof is administered at about 7 mg/kg. In some embodiments, 4-iodo-3-nitrobenzamide or a salt, metabolite or prodrug thereof is administered at about 8 mg/kg. In some embodiments, 4-iodo-3-nitrobenzamide or a salt, metabolite or prodrug thereof is administered at about 8.5 mg/kg. In some embodiments, 4-iodo-3-nitrobenzamide or a salt, metabolite or prodrug thereof is administered at about 8.6 mg/kg. In some embodiments, 4-iodo-3-nitrobenzamide or a salt, metabolite or prodrug thereof is administered at about 9 mg/kg. In some embodiments, 4-iodo-3-nitrobenzamide or a salt, metabolite or prodrug thereof is administered at about 10 mg/kg. In some embodiments, 4-iodo-3-nitrobenzamide or a salt, metabolite or prodrug thereof is administered at about 15 mg/kg. In some embodiments, 4-iodo-3-nitrobenzamide or a salt, metabolite or prodrug thereof is administered at about 20 mg/kg. In some embodiments, 4-iodo-3-nitrobenzamide or a salt, metabolite or prodrug thereof is administered at about 30 mg/kg. In some embodiments, 4-iodo-3-nitrobenzamide or a salt, metabolite or prodrug thereof is administered at about 40 mg/kg. In some embodiments, 4-iodo-3-nitrobenzamide or a salt, metabolite or prodrug thereof is administered at about 50 mg/kg. In some embodiments, 4-iodo-3-nitrobenzamide or a salt, metabolite or prodrug thereof is administered at about 60 mg/kg.

In some embodiments, 4-iodo-3-nitrobenzamide or a salt, metabolite or prodrug thereof is administered to a subject at one or more dosing schedules. In some embodiments, the dosing schedule comprises administering to the subject 4-iodo-3-nitrobenzamide or a salt, metabolite or prodrug thereof once per day, twice a week, three times a week, four times a week, five times a week, daily, every other day, once a month, twice a month, or every week. In some instances, the dosing schedule comprises administering to the subject 4-iodo-3-nitrobenzamide or a salt, metabolite or prodrug thereof once per day.

In some embodiments, the dosing schedule comprises administering to the subject 4-iodo-3-nitrobenzamide or a salt, metabolite or prodrug thereof continuously for about 1, 2, 3, 4, 5, 6, 7, 8, 9, 10, 11, 12 or more weeks. In some instances, the dosing schedule comprises administering to the subject 4-iodo-3-nitrobenzamide or a salt, metabolite or prodrug thereof continuously for about 1, 2, 3, 4, 5, 6, 7, 8, 9, 10 or more weeks. In some instances, the dosing schedule comprises administering to the subject 4-iodo-3-nitrobenzamide or a salt, metabolite or prodrug thereof continuously for about 1, 2, 3, 4, 5, 6 or more weeks. In some instances, the dosing schedule comprises administering to the subject 4-iodo-3-nitrobenzamide or a salt, metabolite or prodrug thereof continuously for about 5 weeks. In some instances, the dosing schedule comprises administering to the subject 4-iodo-3-nitrobenzamide or a salt, metabolite or prodrug thereof continuously for about 6 weeks. In some instances, the dosing schedule comprises administering to the subject 4-iodo-3-nitrobenzamide or a salt, metabolite or prodrug thereof continuously for about 7 weeks. In some instances, the dosing schedule comprises administering to the subject 4-iodo-3-nitrobenzamide or a salt, metabolite or prodrug thereof continuously for about 8 weeks. In some instances, the dosing schedule comprises administering to the subject 4-iodo-3-nitrobenzamide or a salt, metabolite or prodrug thereof continuously for about 9 weeks. In some instances, the dosing schedule comprises administering to the subject 4-iodo-3-nitrobenzamide or a salt, metabolite or prodrug thereof continuously for about 10 weeks. In some instances, a 5-week dosing schedule is considered as one cycle. In some instances, a 6-week dosing schedule is considered as one cycle. In some instances, a 7-week dosing schedule is considered as one cycle. In some instances, a 8-week dosing schedule is considered as one cycle. In some instances, a 9-week dosing schedule is considered as one cycle. In some instances, a 10-week dosing schedule is considered as one cycle.

In some embodiments, the dosing schedule comprises administering to the subject 4-iodo-3-nitrobenzamide or a salt, metabolite or prodrug thereof continuously for about 1, 2, 3, 4, 5, 6, 7, 8, 9, 10, 11, 12 or more months. In some instances, the dosing schedule comprises administering to the subject 4-iodo-3-nitrobenzamide or a salt, metabolite or prodrug thereof continuously for about 1, 2, 3, 4, 5, 6 or more months.

In some embodiments, the dosing schedule comprises administering to the subject 4-iodo-3-nitrobenzamide or a salt, metabolite or prodrug thereof continuously for about 1, 2, 3, 4, 5, 6, 7, 8, 9, 10 or more treatment cycles. In some instances, the dosing schedule comprises administering to the subject 4-iodo-3-nitrobenzamide or a salt, metabolite or prodrug thereof continuously for about 1, 2, 3, 4, 5, 6 or more treatment cycles. In some instances, the dosing schedule comprises administering to the subject 4-iodo-3-nitrobenzamide or a salt, metabolite or prodrug thereof continuously for about 1, 2, 3, 4 or more treatment cycles. In some instances, the dosing schedule comprises administering to the subject 4-iodo-3-nitrobenzamide or a salt, metabolite or prodrug thereof continuously for about 1 or more cycles. In some instances, the dosing schedule comprises administering to the subject 4-iodo-3-nitrobenzamide or a salt, metabolite or prodrug thereof continuously for about 2 or more cycles. In some instances, the dosing schedule comprises administering to the subject 4-iodo-3-nitrobenzamide or a salt, metabolite or prodrug thereof continuously for about 3 or more cycles. In some instances, the dosing schedule comprises administering to the subject 4-iodo-3-nitrobenzamide or a salt, metabolite or prodrug thereof continuously for about 4 or more cycles. In some instances, each treatment cycle is up to 28 days. In some cases, each treatment cycle is about 28 days. In other instances, each treatment cycle is up to 5 weeks. In other instances, each treatment cycle is about 5 weeks. In other instances, each treatment cycle is up to 6 weeks. In other instances, each treatment cycle is about 6 weeks. In other instances, each treatment cycle is up to 7 weeks. In other instances, each treatment cycle is about 7 weeks. In other instances, each treatment cycle is up to 8 weeks. In other instances, each treatment cycle is about 8 weeks. In other instances, each treatment cycle is up to 9 weeks. In other instances, each treatment cycle is about 9 weeks. In other instances, each treatment cycle is up to 10 weeks. In other instances, each treatment cycle is about 10 weeks.

In some embodiments, the dosing schedule comprises administering to the subject 4-iodo-3-nitrobenzamide or a salt, metabolite or prodrug thereof intermittently for about 1, 2, 3, 4, 5, 6, 7, 8, 9, 10, 11, 12 or more weeks. In some instances, the dosing schedule comprises administering to the subject 4-iodo-3-nitrobenzamide or a salt, metabolite or prodrug thereof intermittently for about 1, 2, 3, 4, 5, 6, 7, 8, 9, 10 or more weeks. In some instances, the dosing schedule comprises administering to the subject 4-iodo-3-nitrobenzamide or a salt, metabolite or prodrug thereof intermittently for about 1, 2, 3, 4, 5, 6 or more weeks. In some instances, the dosing schedule comprises administering to the subject 4-iodo-3-nitrobenzamide or a salt, metabolite or prodrug thereof intermittently for about 5 weeks. In some instances, the dosing schedule comprises administering to the subject 4-iodo-3-nitrobenzamide or a salt, metabolite or prodrug thereof intermittently for about 6 weeks. In some instances, the dosing schedule comprises administering to the subject 4-iodo-3-nitrobenzamide or a salt, metabolite or prodrug thereof intermittently for about 7 weeks. In some instances, the dosing schedule comprises administering to the subject 4-iodo-3-nitrobenzamide or a salt, metabolite or prodrug thereof intermittently for about 8 weeks. In some instances, the dosing schedule comprises administering to the subject 4-iodo-3-nitrobenzamide or a salt, metabolite or prodrug thereof intermittently for about 9 weeks. In some instances, the dosing schedule comprises administering to the subject 4-iodo-3-nitrobenzamide or a salt, metabolite or prodrug thereof intermittently for about 10 weeks. In some instances, a 5-week dosing schedule is considered as one cycle. In some instances, a 6-week dosing schedule is considered as one cycle. In some instances, a 7-week dosing schedule is considered as one cycle. In some instances, a 8-week dosing schedule is considered as one cycle. In some instances, a 9-week dosing schedule is considered as one cycle. In some instances, a 10-week dosing schedule is considered as one cycle.

In some embodiments, the dosing schedule comprises administering to the subject 4-iodo-3-nitrobenzamide or a salt, metabolite or prodrug thereof intermittently for about 1, 2, 3, 4, 5, 6, 7, 8, 9, 10, 11, 12 or more months. In some instances, the dosing schedule comprises administering to the subject 4-iodo-3-nitrobenzamide or a salt, metabolite or prodrug thereof intermittently for about 1, 2, 3, 4, 5, 6 or more months.

In some embodiments, the dosing schedule comprises administering to the subject 4-iodo-3-nitrobenzamide or a salt, metabolite or prodrug thereof intermittently for about 1, 2, 3, 4, 5, 6, 7, 8, 9, 10 or more treatment cycles. In some instances, the dosing schedule comprises administering to the subject 4-iodo-3-nitrobenzamide or a salt, metabolite or prodrug thereof intermittently for about 1, 2, 3, 4, 5, 6 or more treatment cycles. In some instances, the dosing schedule comprises administering to the subject 4-iodo-3-nitrobenzamide or a salt, metabolite or prodrug thereof intermittently for about 1, 2, 3, 4 or more treatment cycles. In some instances, the dosing schedule comprises administering to the subject 4-iodo-3-nitrobenzamide or a salt, metabolite or prodrug thereof intermittently for about 1 or more cycles. In some instances, the dosing schedule comprises administering to the subject 4-iodo-3-nitrobenzamide or a salt, metabolite or prodrug thereof intermittently for about 2 or more cycles. In some instances, the dosing schedule comprises administering to the subject 4-iodo-3-nitrobenzamide or a salt, metabolite or prodrug thereof intermittently for about 3 or more cycles. In some instances, the dosing schedule comprises administering to the subject 4-iodo-3-nitrobenzamide or a salt, metabolite or prodrug thereof intermittently for about 4 or more cycles. In some instances, the dosing schedule comprises administering to the subject 4-iodo-3-nitrobenzamide or a salt, metabolite or prodrug thereof intermittently for about 5 or more cycles. In some instances, the dosing schedule comprises administering to the subject 4-iodo-3-nitrobenzamide or a salt, metabolite or prodrug thereof intermittently for about 6 or more cycles. In some instances, each treatment cycle is up to 28 days. In some cases, each treatment cycle is about 28 days. In other instances, each treatment cycle is up to 5 weeks. In other instances, each treatment cycle is about 5 weeks. In other instances, each treatment cycle is up to 6 weeks. In other instances, each treatment cycle is about 6 weeks. In other instances, each treatment cycle is up to 7 weeks. In other instances, each treatment cycle is about 7 weeks. In other instances, each treatment cycle is up to 8 weeks. In other instances, each treatment cycle is about 8 weeks. In other instances, each treatment cycle is up to 9 weeks. In other instances, each treatment cycle is about 9 weeks. In other instances, each treatment cycle is up to 10 weeks. In other instances, each treatment cycle is about 10 weeks.

In some embodiments, the cancer is a TrxR-overexpressed cancer or a PRDX-overexpressed cancer. In some instances, the cancer is a TrxR-overexpressed cancer. In other instances, the cancer is a PRDX-overexpressed cancer. In some cases, the TrxR-overexpressed cancer is a metastatic TrxR-overexpressed cancer. In some cases, the PRDX-overexpressed cancer is metastatic PRDX-overexpressed cancer. In some cases, the TrxR-overexpressed cancer is a relapsed TrxR-overexpressed cancer. In some cases, the PRDX-overexpressed cancer is a relapsed PRDX-overexpressed cancer. In other cases, the TrxR-overexpressed cancer is a refractory TrxR-overexpressed cancer. In other cases, the PRDX-overexpressed cancer is a refractory PRDX-overexpressed cancer.

In some embodiments, the cancer is a solid tumor, a hematologic malignancy, or a melanoma. In some cases, the cancer is a metastatic cancer. In some cases, the cancer is a relapsed cancer. In other cases, the cancer is a refractory cancer.

In some instances, the cancer is a solid tumor. In some instances, the solid tumor comprises brain cancer, bladder cancer, breast cancer, colorectal cancer, lung cancer, or prostate cancer. In some cases, the solid tumor is brain cancer. In some cases, the brain cancer comprises glioblastoma (or glioblastoma multiforme, GBM). In some cases, the glioblastoma is a primary glioblastoma or a de novo glioblastoma. In other instances, the glioblastoma is a secondary tumor. In some cases, glioblastoma is further classified into grade I, grade II, grade III and grade IV glioblastoma. In some cases, a subject is diagnosed with a grade I or grade II glioblastoma. In other cases, a subject is diagnosed with a grade III or a grade IV glioblastoma. In some cases, the glioblastoma is a metastasized glioblastoma.

In some embodiments, the solid tumor is breast cancer. In some instances, the breast cancer is further classified into ductal carcinoma in situ (DCIS), invasive ductal carcinoma (IDC), invasive lobular carcinoma (ILC), inflammatory breast cancer, lobular carcinoma in situ (LCIS), male breast cancer, Paget's disease of the Nipple, phyllodes tumors of the breast, triple negative breast cancer, HER2 positive breast cancer, Luminal A, Luminal B, Liminal B-like (HER2 negative), HER2-enriched, and normal-like breast cancer. In some instances, IDC further comprises tubular carcinoma of the breast, medullary carcinoma of the breast, mucinous carcinoma of the breast, papillary carcinoma of the breast and cribriform carcinoma of the breast. In some cases, the breast cancer is a metastasized breast cancer. In some cases, the breast cancer is a relapsed breast cancer. In other cases, the breast cancer is a refractory breast cancer.

In some embodiments, the solid tumor is bladder cancer. In some instances, bladder cancer further comprises transitional cell bladder cancer (or urothelial cancer), non muscle invasive bladder cancer, invasive bladder cancer, squamous cell bladder cancer, adenocarcinoma of the urinary bladder, sarcoma, and small cell cancer of the bladder. In some cases, non muscle invasive bladder cancer further comprises carcinoma in situ (CIS) and high grade Ti tumors. In some cases, the bladder cancer is a metastasized bladder cancer. In some cases, the bladder cancer is a relapsed bladder cancer. In other cases, the bladder cancer is a refractory bladder cancer.

In some embodiments, the solid tumor is colorectal cancer. In some instances, colorectal cancer further comprises colorectal adenocarcinomas, carcinoid tumors, gastrointestinal stromal tumors (GISTs), lymphomas and sarcomas. In some cases, the colorectal cancer is a metastasized colorectal cancer. In some cases, the colorectal cancer is a relapsed colorectal cancer. In other cases, the colorectal cancer is a refractory colorectal cancer.

In some embodiments, the solid tumor is lung cancer. In some cases, lung cancer comprises non-small cell lung cancer (NSCLC), small cell lung cancer (SCLC), mesothelioma and carcinoid tumors. In some cases, NSCLC further comprises adenocarcinoma of lungs, adenocarcinoma in situ (AIS), minimally invasive adenocarcinoma (MIA), squamous cell carcinoma, large cell carcinoma, and large cell neuroendocrine tumors. In some cases, the lung cancer is a metastasized lung cancer. In some cases, the lung cancer is a relapsed lung cancer. In other cases, the lung cancer is a refractory lung cancer.

In some embodiments, the solid tumor is prostate cancer. In some instances, the prostate cancer further comprises acinar adenocarcinoma, ductal adenocarcinoma, transitional cell (or urothelial) cancer, squamous cell cancer, small cell prostate cancer, carcinoid, and sarcoma. In some cases, the prostate cancer is a metastasized prostate cancer. In some cases, the prostate cancer is a relapsed prostate cancer. In other cases, the prostate cancer is a refractory prostate cancer.

In some embodiments, the cancer, for example, either TrxR-overexpressed or PRDX-overexpressed, is a hematologic malignancy. In some instances, the hematologic malignancy comprises a T-cell leukemia. In some cases, the T-cell leukemia comprises large granular lymphocytic leukemia, T-cell acute lymphoblastic leukemia (T-ALL) or T-cell prolymphocytic leukemia (T-PLL). In some cases, the T-cell leukemia is a metastasized T-cell leukemia. In some cases, the T-cell leukemia is a relapsed T-cell leukemia. In other cases, the T-cell leukemia is a refractory T-cell leukemia.

In some embodiments, the cancer, for example, either TrxR-overexpressed or PRDX-overexpressed, is a melanoma. In some cases, the melanoma is a metastasized melanoma. In some cases, the melanoma is a relapsed melanoma. In other cases, the melanoma is a refractory melanoma.

### Additional Therapeutic Agents

In some embodiments, disclosed herein comprises administering to a subject 4-iodo-3-nitrobenzamide or a salt, metabolite or prodrug thereof with an additional therapeutic agent. In some instances, the additional therapeutic agent is an inhibitor of TrxR. In some cases, the TrxR inhibitor is epigallocatechin-3-O-gallate (EGCG), n-butyl 2-imidazolyl disulfide, 1-methylpropyl 2-imidazolyl disulfide, n-decyl 2-imidazolyl disulfide, an alkyl 2-imidazolyl disulfide analogue, auranofin, or a dinitrohalobenzene. In some instances, the TrxR inhibitor is phosphine gold(I), a gold(I) carbene complex, a gold(III)-dithiocarbamato complex, an arsenic derivative, or azelaic acid. In some cases, the TrxR inhibitor is an inhibitor described in Saccoccia et al., “Thioredoxin reductase and its inhibitors,” Current Protein and Peptide Science 15:621-646 (2014).

In some embodiments, the additional therapeutic agent is an inhibitor of PRDX. In some cases, the PRDX inhibitor is a pan-PRDX inhibitor. In some cases, the PRDX inhibitor is Conoidin A.

In some instances, the additional therapeutic agent is an inhibitor of glutathione (GSH). In some cases, the GSH inhibitor is L-buthionine sulfoximine (BSO).

In some instances, the additional therapeutic agent is temozolomide. In some cases, temozolomide is administered to a subject at a dosing range of 70 mg/m² to about 200 mg/m², about 70 mg/m² to about 80 mg/m², or about 150 mg/m² to about 200 mg/m². In some cases, temozolomide is administered to a subject at a dosing range of about 70 mg/m² to about 80 mg/m². In some cases, temozolomide is administered to a subject at a dosing range of about 150 mg/m² to about 200 mg/m². In some cases, the dosing range of about 150 mg/m² to about 200 mg/m² is administered to the subject as a maintenance regimen.

In some cases, temozolomide is administered to a subject at a dosing range of about 60 mg/m², about 65 mg/m², about 70 mg/m², about 75 mg/m², about 80 mg/m², about 85 mg/m², about 90 mg/m², about 95 mg/m², or about 100 mg/m². In some cases, temozolomide is administered to a subject at a dose of about 60 mg/m². In some cases, temozolomide is administered to a subject at a dose of about 65 mg/m². In some cases, temozolomide is administered to a subject at a dose of about 70 mg/m². In some cases, temozolomide is administered to a subject at a dose of about 75 mg/m². In some cases, temozolomide is administered to a subject at a dose of about 80 mg/m². In some cases, temozolomide is administered to a subject at a dose of about 85 mg/m². In some cases, temozolomide is administered to a subject at a dose of about 90 mg/m². In some cases, temozolomide is administered to a subject at a dose of about 95 mg/m². In some cases, temozolomide is administered to a subject at a dose of about 100 mg/m².

In some cases, temozolomide is administered to a subject at a dosing range of 0 mg/m² to about 90 mg/m², about 0 mg/m² to about 80 mg/m², about 0 mg/m² to about 70 mg/m², about 10 mg/m² to about 80 mg/m², about 10 mg/m² to about 70 mg/m², about 10 mg/m² to about 60 mg/m², about 20 mg/m² to about 80 mg/m², about 20 mg/m² to about 70 mg/m², about 20 mg/m² to about 60 mg/m², about 30 mg/m² to about 80 mg/m², about 30 mg/m² to about 70 mg/m², or about 30 mg/m² to about 60 mg/m². In some cases, temozolomide is administered to a subject at a dosing range of 0 mg/m² to about 70 mg/m². In some cases, temozolomide is administered to a subject at a dosing range of 10 mg/m² to about 70 mg/m². In some cases, temozolomide is administered to a subject at a dosing range of 20 mg/m² to about 70 mg/m². In some cases, temozolomide is administered to a subject at a dosing range of 30 mg/m² to about 70 mg/m².

In some cases, temozolomide is administered to a subject at a dose of about 0 mg/m², 5 mg/m², 10 mg/m², 15 mg/m², 20 mg/m², 25 mg/m², 30 mg/m², 35 mg/m², 40 mg/m², 50 mg/m², 60 mg/m², 70 mg/m², 80 mg/m², or 90 mg/m². In some cases, temozolomide is administered to a subject at a dose of about 0 mg/m². In some cases, temozolomide is administered to a subject at a dose of about 5 mg/m². In some cases, temozolomide is administered to a subject at a dose of about 10 mg/m². In some cases, temozolomide is administered to a subject at a dose of about 15 mg/m². In some cases, temozolomide is administered to a subject at a dose of about 20 mg/m². In some cases, temozolomide is administered to a subject at a dose of about 25 mg/m². In some cases, temozolomide is administered to a subject at a dose of about 30 mg/m². In some cases, temozolomide is administered to a subject at a dose of about 35 mg/m². In some cases, temozolomide is administered to a subject at a dose of about 40 mg/m². In some cases, temozolomide is administered to a subject at a dose of about 50 mg/m². In some cases, temozolomide is administered to a subject at a dose of about 60 mg/m². In some cases, temozolomide is administered to a subject at a dose of about 70 mg/m². In some cases, temozolomide is administered to a subject at a dose of about 80 mg/m². In some cases, temozolomide is administered to a subject at a dose of about 90 mg/m².

In some instances, the additional therapeutic agent is radiation. In some cases, the total dose of radiation administered to a subject is up to 60 gray (Gy). In some cases, the total dose of radiation administered to a subject is up to 20 Gy, 30 Gy, 35 Gy, 40 Gy, 45 Gy, 50 Gy, 55 Gy, or 60 Gy. In some cases, the total dose of radiation administered to a subject is up to 20 Gy. In some cases, the total dose of radiation administered to a subject is up to 30 Gy. In some cases, the total dose of radiation administered to a subject is up to 35 Gy. In some cases, the total dose of radiation administered to a subject is up to 40 Gy. In some cases, the total dose of radiation administered to a subject is up to 45 Gy. In some cases, the total dose of radiation administered to a subject is up to 50 Gy. In some cases, the total dose of radiation administered to a subject is up to 55 Gy. In some cases, the total dose of radiation administered to a subject is up to 60 Gy.

In some instances, the total radiation dose is the dose a subject receives over the course of a treatment cycle. In some instances, the treatment cycle is from 5 to 10 weeks. In some instances, the treatment cycle is about 10 weeks.

In some instances, the additional therapeutic agent is a standard-of-care chemotherapy. In some cases, the standard-of-care chemotherapy comprises abraxane, bevacizumab, capecitabine, carboplatin, cisplatin, cyclophosphamide, docetaxel, doxorubicin, etoposide, gemcitabine, irinotecan, paclitaxel, pemetrexed, topotecan, vinorelbine, carboplatin/gemcitabine, carboplatin/irinotecan, bevacizumab/gemcitabine or a combination thereof.

In some instances, 4-iodo-3-nitrobenzamide or a salt, metabolite or prodrug thereof and the additional therapeutic agent are administered concurrently. In other instances, 4-iodo-3-nitrobenzamide or a salt, metabolite or prodrug thereof and the additional therapeutic agent are administered sequentially.

### Samples and Detection Methods

### Samples

In some embodiments, a sample described herein is obtained from a mammalian source. In some instances, the mammalian source comprises human and non-human primates. In other cases, the mammalian source comprises a rodent (e.g., mouse, rat), cat, rabbit, dog, and the like.

In some cases, a sample described herein is a tissue sample. In some cases, the sample is a biopsy sample. In some cases, the sample is a tumor sample, e.g., a tumor sample obtained from brain cancer, bladder cancer, breast cancer, colorectal cancer, lung cancer, or prostate cancer.

In some cases, a sample described herein is a liquid sample. In some cases, the liquid sample comprises blood and other liquid samples of biological origin (including, but not limited to, peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood). In some embodiments, the sample is blood, a blood derivative or a blood fraction, e.g., serum or plasma.

In some embodiments, the liquid sample also encompasses a sample that has been manipulated in any way after their procurement, such as by centrifugation, filtration, precipitation, dialysis, chromatography, treatment with reagents, washed, or enriched for certain cell populations.

In some embodiments, a sample described herein is a cell sample, e.g., obtained from a tumor or a cancer cell line. In some instances, the cell sample is obtained from cells of brain cancer, bladder cancer, breast cancer, colorectal cancer, lung cancer, prostate cancer, large granular lymphocytic leukemia, T-cell acute lymphoblastic leukemia (T-ALL), T-cell prolymphocytic leukemia (T-PLL) or a melanoma.

In some instances, a sample described herein is a cell-free sample.

In some embodiments, the samples are obtained from the individual by any suitable means of obtaining the sample using well-known and routine clinical methods. Procedures for obtaining fluid samples from an individual are well known. For example, procedures for drawing and processing whole blood and lymph are well-known and can be employed to obtain a sample for use in the methods provided. Typically, for collection of a blood sample, an anti-coagulation agent (e.g., EDTA, or citrate and heparin or CPD (citrate, phosphate, dextrose) or comparable substances) is added to the sample to prevent coagulation of the blood. In some examples, the blood sample is collected in a collection tube that contains an amount of EDTA to prevent coagulation of the blood sample.

In some embodiments, the collection of a sample from the subject is performed at regular intervals, such as, for example, one day, two days, three days, four days, five days, six days, one week, two weeks, weeks, four weeks, one month, two months, three months, four months, five months, six months, one year, daily, weekly, bimonthly, quarterly, biyearly or yearly.

In some embodiments, the collection of a sample is performed at a predetermined time or at regular intervals relative to treatment with 4-iodo-3-nitrobenzamide or a salt, metabolite or prodrug thereof. In some cases, the collection of a sample is performed at a predetermined time or at regular intervals relative to treatment with 4-iodo-3-nitrobenzamide or a salt, metabolite or prodrug thereof and an additional therapeutic agent described herein.

### Detection Methods

In some embodiments, methods of detecting the expression level of one or more biomarkers described herein include, but are not limited to, Western blots, Northern blots, Southern blots, enzyme-linked immunosorbent assay (ELISA), immunoprecipitation, immunofluorescence, radioimmunoassay, immunocytochemistry, nucleic acid hybridization techniques, nucleic acid reverse transcription methods, nucleic acid amplification methods, or a combination thereof. In some cases, the biomarkers described herein comprise genes: TXNRD2, TXN2, MSRB3, MSRA, GSTZ1, NQO2, GSTT2, GSTM3, GLRX, GSTO1, GLRX3, TXNRD1, SELO, PON1, and SEPX1 and the proteins encoded by the respective genes.

In some embodiments, the expression level of one or more biomarkers described herein is determined at the nucleic acid level. Nucleic acid-based techniques for assessing expression are well known in the art and include, for example, determining the level of biomarker mRNA in a biological sample. Many expression detection methods use isolated RNA. Any RNA isolation technique that does not select against the isolation of mRNA is utilized for the purification of RNA (see, e.g., Ausubel et al., ed. (1987-1999) Current Protocols in Molecular Biology (John Wiley & Sons, New York). Additionally, large numbers of tissue samples are readily processed using techniques well known to those of skill in the art, such as, for example, the single-step RNA isolation process disclosed in U.S. Pat. No. 4,843,155.

As used herein, the term “nucleic acid probe” refers to any molecule that is capable of selectively binding to a specifically intended target nucleic acid molecule, for example, a nucleotide transcript. Suitable methods for synthesizing nucleic acid probes are also described in Caruthers, Science, 230:281-285, (1985). In some instances, probes suitable for use herein include those formed from nucleic acids, such as RNA and/or DNA, nucleic acid analogs, locked nucleic acids, modified nucleic acids, and chimeric probes of a mixed class including a nucleic acid with another organic component such as peptide nucleic acids. In some cases, probes are single stranded. In other cases, probes are double stranded. Exemplary nucleotide analogs include phosphate esters of deoxyadenosine, deoxycytidine, deoxyguanosine, deoxythymnidine, adenosine, cytidine, guanosine, and uridine. Other examples of non-natural nucleotides include a xanthine or hypoxanthine; 5-bromouracil, 2-aminopurine, deoxyinosine, or methylated cytosine, such as 5-methylcytosine, and N4-methoxydeoxycytosine. Also included are bases of polynucleotide mimetics, such as methylated nucleic acids, e.g., 2′-0-methRNA, peptide nucleic acids, modified peptide nucleic acids, and any other structural moiety that can act substantially like a nucleotide or base, for example, by exhibiting base-complementarity with one or more bases that occur in DNA or RNA.

In some cases, a probe used for detection optionally includes a detectable label, such as a radiolabel, fluorescent label, or enzymatic label. See for example Lancaster et al., U.S. Pat. No. 5,869,717. In some embodiments, the probe is fluorescently labeled. Fluorescently labeled nucleotides may be produced by various techniques, such as those described in Kambara et al, Bio/Technol., 6:816-21, (1988); Smith et al., Nucl. Acid Res., 13:2399-2412, (1985); and Smith et al., Nature, 321: 674-679, (1986). The fluorescent dye may be linked to the deoxyribose by a linker arm that is easily cleaved by chemical or enzymatic means. There are numerous linkers and methods for attaching labels to nucleotides, as shown in Oligonucleotides and Analogues: A Practical Approach, IRL Press, Oxford, (1991); Zuckerman et al., Polynucleotides Res., 15: 5305-5321, (1987); Sharma et al., Polynucleotides Res., 19:3019, (1991); Giusti et al., PCR Methods and Applications, 2:223-227, (1993); Fung et al. (U.S. Pat. No. 4,757,141); Stabinsky (U.S. Pat. No. 4,739,044); Agrawal et al., Tetrahedron Letters, 31: 1543-1546, (1990); Sproat et al., Polynucleotides Res., 15:4837, (1987); and Nelson et al, Polynucleotides Res., 17:7187-7194, (1989). Extensive guidance exists in the literature for derivatizing fluorophore and quencher molecules for covalent attachment via common reactive groups that may be added to a nucleotide. Many linking moieties and methods for attaching fluorophore moieties to nucleotides also exist, as described in Oligonucleotides and Analogues, supra; Guisti et al., supra; Agrawal et al, sLupra; and Sproat et al., supra.

In some cases, the detectable label attached to the probe is either directly or indirectly detectable. In some embodiments, the exact label may be selected based, at least in part, on the particular type of detection method used. Exemplary detection methods include radioactive detection, optical absorbance detection, e.g., UV-visible absorbance detection, optical emission detection, e.g., fluorescence; phosphorescence or chemilurninescence; Raman scattering. Preferred labels include optically-detectable labels, such as fluorescent labels. Examples of fluorescent labels include, but are not limited to, 4-acetamido-4′-isothiocyanatostilbene-2,2′disulfonic acid; acridine and derivatives: acridine, acridine isothiocyanate; 5-(2′-aminoethyl)aminonaphthaiene-1-sulfonic acid (EDANS); 4-amino-N-[3-vinylsulfonyl)phenyl]naphthalimide-3,5 disulfonate; N-(4-anilino-l-naphthyl)maleimide; anthranilamide; BODIPY; alexa; fluorescien; conjugated multi-dyes; Brilliant Yellow; coumarin and derivatives; coumarin, 7-amino-4-methylcoumnarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-m ethylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR 1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodanmine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (T-AMRA); tetramethyl rhodamine; tetramethyl rhodamine isothiocyanate (TRITC); riboflavin; rosolic acid; terbium chelate derivatives; Atto dyes, Cy3; Cy5; Cy5.5; Cy7; IRD 700; IRD 800; La Jolta Blue; phthalo cyanine; and naphthalo cyanine. Labels other than fluorescent labels are contemplated by the invention, including other optically-detectable labels.

Detection of a bound probe may be measured using any of a variety of techniques dependent upon the label used, such as those known to one of skill in the art. Exemplary detection methods include radioactive detection, optical absorbance detection, e.g., UV-visible absorbance detection, optical emission detection, e.g., fluorescence or chemiluminescence. Devices capable of sensing fluorescence from a single molecule include scanning tunneling microscope (siM) and the atomic force microscope (AFM). Hybridization patterns may also be scanned using a CCD camera (e.g., Model TE/CCD512SF, Princeton Instruments, Trenton, N.J.) with suitable optics (Ploem, in Fluorescent and Luminescent Probes for Biological Activity Mason, T. G. Ed., Academic Press, Landon, pp. 1-11 (1993)), such as described in Yershov et al., Proc. Natl. Acad. Sci. 93:4913 (1996), or may be imaged by TV monitoring. For radioactive signals, a phosphorimager device can be used (Johnston et al., Electrophoresis, 13:566, 1990; Drmanac et al., Electrophoresis, 13:566, 1992; 1993). Other commercial suppliers of imaging instruments include General Scanning Inc., (Watertown, Mass. on the World Wide Web at genscancomrn), Genix Technologies (Waterloo, Ontario, Canada; on the World Wide Web at confocal.com), and Applied Precision Inc.

In certain embodiments, the target nucleic acid or nucleic acid ligand or both are quantified using methods known in the art. For example, isolated mRNA are used in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that hybridize to the mRNA encoded by the gene being detected. The nucleic acid probe comprises of, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding a biomarker, biomarker described herein above. Hybridization of an mRNA with the probe indicates that the biomarker or other target protein of interest is being expressed.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in a gene chip array. A skilled artisan readily adapts known mRNA detection methods for use in detecting the level of mRNA encoding the biomarkers or other proteins of interest.

An alternative method for determining the level of an mRNA of interest in a sample involves the process of nucleic acid amplification, e.g., by RT-PCR (see, for example, U.S. Pat. No. 4,683,202), ligase chain reaction (Barany (1991) Proc. Natl. Acad. Sci. USA 88:189 193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, biomarker expression is assessed by quantitative fluorogenic RT-PCR (i.e., the TaqMan System).

Modifications or expression levels of an RNA of interest are monitored using a membrane blot (such as used in hybridization analysis such as Northern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection of expression also comprises using nucleic acid probes in solution.

In some embodiments, microarrays are used to determine expression or presence of one or more biomarkers. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning Hybridization intensities for each probe on the array are determined and converted to a quantitative value representing relative gene expression levels. See, U.S. Pat. Nos. 6,040,138, 5,800,992, 6,020,135, 6,033,860, 6,344,316, and U.S. Pat. Application 20120208706. High-density oligonucleotide arrays are particularly useful for determining the gene expression profile for a large number of RNA's in a sample. Exemplary microarray chips include FoundationOne and FoundationOne Heme from Foundation Medicine, Inc; GeneChip® Human Genome U133 Plus 2.0 array from Affymetrix; and Human DiscoveryMAP® 250+v. 2.0 from Myraid RBM.

Techniques for the synthesis of these arrays using mechanical synthesis methods are described in, e.g., U.S. Pat. No. 5,384,261. In some embodiments, an array is fabricated on a surface of virtually any shape or even a multiplicity of surfaces. In some embodiments, an array is a planar array surface. In some embodiments, arrays include peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, each of which is hereby incorporated in its entirety for all purposes. In some embodiments, arrays are packaged in such a manner as to allow for diagnostics or other manipulation of an all-inclusive device.

In some instances, a method for quantitation is quantitative polymerase chain reaction (QPCR). As used herein, “QPCR” refers to a PCR reaction performed in such a way and under such controlled conditions that the results of the assay are quantitative, that is, the assay is capable of quantifying the amount or concentration of a nucleic acid ligand present in the test sample. QPCR is a technique based on the polymerase chain reaction, and is used to amplify and simultaneously quantify a targeted nucleic acid molecule. QPCR allows for both detection and quantification (as absolute number of copies or relative amount when normalized to DNA input or additional normalizing genes) of a specific sequence in a DNA sample. The procedure follows the general principle of PCR, with the additional feature that the amplified DNA is quantified as it accumulates in the reaction in real time after each amplification cycle. QPCR is described, for example, in Kurnit et al. (U.S. Pat. No. 6,033,854), Wang et al. (U.S. Pat. Nos. 5,567,583 and 5,348,853), Ma et al. (The Journal of American Science, 2(3), (2006)), Heid et al. (Genome Research 986-994, (1996)), Sambrook and Russell (Quantitative PCR, Cold Spring Harbor Protocols, (2006)), and Higuchi (U.S. Pat. Nos. 6,171,785 and 5,994,056).

In some embodiments, the expression level is a protein expression and the level of the protein expression of a gene described herein is detected. In some cases, the detection method comprises contacting a biological sample with an antibody that specifically recognizes or specifically binds to a protein (e.g., a protein encoded by TXNRD2, TXN2, MSRB3, MSRA, GSTZ1, NQO2, GSTT2, GSTM3, GLRX, GSTO1, GLRX3, TXNRD1, SELO, PON1, or SEPX1) and detecting the complex between the antibody and the protein. In some cases, the antibody is an anti-TXNRD2 antibody. In some cases, the antibody is an anti-TXN2 antibody. In some instances, the antibody is an anti-MSRB3 antibody. In some cases, the antibody is an anti-MSRA antibody. In some cases, the antibody is an anti-GSTZ1 antibody. In some cases, the antibody is an anti-NQO2 antibody. In some cases, the antibody is an anti-GSTT2 antibody. In some cases, the antibody is an anti-GSTM3 antibody. In some cases, the antibody is an anti-GLRX antibody. In some cases, the antibody is an anti-GSTO1 antibody. In some cases, the antibody is an anti-GLRX3 antibody. In some cases, the antibody is an anti-TXNRD1 antibody. In some cases, the antibody is an anti-SELO antibody. In some cases, the antibody is an anti-PON1 antibody. In some cases, the antibody is an anti-SEPX1 antibody. In some cases, the level of the protein expression is determined by immunoassays including, but not limited to, radioimmunoassay, Western blot assay, ELISA, immunofluorescent assay, enzyme immunoassay, immunoprecipitation, chemiluminescent assay, immunohistochemical assay, dot blot assay, and slot blot assay.

### Pharmaceutical Formulations, Dosage Forms and Treatment Regimens

Another aspect of the present invention relates to formulations and routes of administration for pharmaceutical compositions comprising a nitrobenzamide compound. Such pharmaceutical compositions can be used to treat cancer in the methods described in detail above.

The compounds of Formula I may be provided as a prodrug and/or may be allowed to interconvert to a nitrosobenzamide form in vivo after administration. That is, either the nitrobenzamide form and/or the nitrosobenzamide form, or pharmaceutically acceptable salts may be used in developing a formulation for use in the present invention. Further, in some embodiments, the compound may be used in combination with one or more other compounds or in one or more other forms. For example a formulation may comprise both the nitrobenzamide compound and acid forms in particular proportions, depending on the relative potencies of each and the intended indication. The two forms may be formulated together, in the same dosage unit e.g. in one cream, suppository, tablet, capsule, or packet of powder to be dissolved in a beverage; or each form may be formulated in a separate unit, e.g., two creams, two suppositories, two tablets, two capsules, a tablet and a liquid for dissolving the tablet, a packet of powder and a liquid for dissolving the powder, etc.

In compositions comprising combinations of a nitrobenzamide compound and another active agent can be effective. The two compounds and/or forms of a compound may be formulated together, in the same dosage unit e.g. in one cream, suppository, tablet, capsule, or packet of powder to be dissolved in a beverage; or each form may be formulated in separate units, e.g., two creams, suppositories, tablets, two capsules, a tablet and a liquid for dissolving the tablet, a packet of powder and a liquid for dissolving the powder, etc.

The term “pharmaceutically acceptable salt” means those salts which retain the biological effectiveness and properties of the compounds used in the present invention, and which are not biologically or otherwise undesirable. For example, a pharmaceutically acceptable salt does not interfere with the beneficial effect of the compound of the invention in treating a cancer.

Typical salts are those of the inorganic ions, such as, for example, sodium, potassium, calcium and magnesium ions. Such salts include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the compounds used in the present invention contain a carboxy group or other acidic group, it may be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine and triethanolamine.

For oral administration, the compounds can be formulated readily by combining the active compound(s) with pharmaceutically acceptable carriers well known in the art. Such carriers enable the compounds of the invention to be formulated as tablets, including chewable tablets, pills, dragees, capsules, lozenges, hard candy, liquids, gels, syrups, slurries, powders, suspensions, elixirs, wafers, and the like, for oral ingestion by a patient to be treated. Such formulations can comprise pharmaceutically acceptable carriers including solid diluents or fillers, sterile aqueous media and various non-toxic organic solvents. Generally, the compounds of the invention will be included at concentration levels ranging from about 0.5%, about 5%, about 10%, about 20%, or about 30% to about 50%, about 60%, about 70%, about 80% or about 90% by weight of the total composition of oral dosage forms, in an amount sufficient to provide a desired unit of dosage.

Aqueous suspensions may contain a nitrobenzamide compound with pharmaceutically acceptable excipients, such as a suspending agent (e.g., methyl cellulose), a wetting agent (e.g., lecithin, lysolecithin and/or a long-chain fatty alcohol), as well as coloring agents, preservatives, flavoring agents, and the like.

In some embodiments, oils or non-aqueous solvents may be required to bring the compounds into solution, due to, for example, the presence of large lipophilic moieties. Alternatively, emulsions, suspensions, or other preparations, for example, liposomal preparations, may be used. With respect to liposomal preparations, any known methods for preparing liposomes for treatment of a condition may be used. See, for example, Bangham et al., J. Mol. Biol, 23: 238-252 (1965) and Szoka et al., Proc. Natl Acad. Sci 75: 4194-4198 (1978), incorporated herein by reference. Ligands may also be attached to the liposomes to direct these compositions to particular sites of action. Compounds of this invention may also be integrated into foodstuffs, e.g, cream cheese, butter, salad dressing, or ice cream to facilitate solubilization, administration, and/or compliance in certain patient populations.

Pharmaceutical preparations for oral use may be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; flavoring elements, cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents may be added, such as the cross-linked polyvinyl pyrrolidone, agar, or alginic acid or a salt thereof such as sodium alginate. The compounds may also be formulated as a sustained release preparation.

Dragee cores can be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for administration.

For injection, the inhibitors of the present invention may be formulated in aqueous solutions, preferably in physiologically compatible buffers such as Hank's solution, Ringer's solution, or physiological saline buffer. Such compositions may also include one or more excipients, for example, preservatives, solubilizers, fillers, lubricants, stabilizers, albumin, and the like. Methods of formulation are known in the art, for example, as disclosed in Remington's Pharmaceutical Sciences, latest edition, Mack Publishing Co., Easton Pa. These compounds may also be formulated for transmucosal administration, buccal administration, for administration by inhalation, for parental administration, for transdermal administration, and rectal administration.

In addition to the formulations described previously, the compounds may also be formulated as a depot preparation. Such long acting formulations may be administered by implantation or transcutaneous delivery (for example subcutaneously or intramuscularly), intramuscular injection or use of a transdermal patch. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

As described elsewhere herein, in some instances 4-iodo-3-nitrobenzamide or a salt, metabolite or prodrug thereof is administered from about 5 mg/kg to about 200 mg/kg, from about 5 mg/kg to about 150 mg/kg, from about 5 mg/kg to about 100 mg/kg, or from about 5 mg/kg to about 60 mg/kg. In other instances, 4-iodo-3-nitrobenzamide or a salt, metabolite or prodrug thereof is administered at about 5 mg/kg, about 6 mg/kg, about 7 mg/kg, about 8 mg/kg, about 8.5 mg/kg, about 8.6 mg/kg, about 9 mg/kg, about 10 mg/kg, about 15 mg/kg, about 20 mg/kg, about 25 mg/kg, about 30 mg/kg, about 40 mg/kg, about 50 mg/kg, or about 60 mg/kg.

In the case wherein the patient's status does improve, upon the doctor's discretion the administration of the compounds may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, the severity of the disease, the identity (e.g., weight) of the subject or host in need of treatment, but can nevertheless be routinely determined in a manner known in the art according to the particular circumstances surrounding the case, including, e.g., the specific agent being administered, the route of administration, and the subject or host being treated. The desired dose may conveniently be presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it can be expressed as the ratio between LD50 and ED50. Compounds exhibiting high therapeutic indices are preferred. The data obtained from cell culture assays and animal studies can be used in formulating a range of dosage for use in human. The dosage of such compounds lies preferably within a range of circulating concentrations that include the ED50 with minimal toxicity. The dosage may vary within this range depending upon the dosage form employed and the route of administration utilized.

### Kits/Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include iniparib, optionally in a composition or in combination with an additional therapeutic agent as disclosed herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, the term “control sample(s)” refers to a non-cancerous sample. In some instances, cells from the control sample are obtained from a healthy subject. In other instances, cells from the control sample are obtained from a subject having cancer, but from a region of the subject that is healthy or cancer-free.

As used herein, the term “first-line treatment” refers to a primary treatment for a subject with a cancer. In some instances, the cancer is a primary cancer. In other instances, the cancer is a metastatic or recurrent cancer. In some cases, the first-line treatment comprises chemotherapy. In other cases, the first-line treatment comprises radiation therapy. A skilled artisan would readily understand that different first-line treatments may be applicable to different type of cancers.

### EXAMPLES

These examples are provided for illustrative purposes only and not to limit the scope of the claims provided herein.

### Example 1—Iniparib Cytotoxicity is Dependent of in-Cell Reductive Bioactivation and Modification of Reactive Thiol Groups in Proteins

In some embodiments, 5-iode-3 nitro-benzamide (Iniparib) is designed to be activated by a two-electron enzymatic reduction to yield a nitroso derivative (I-NOBA) capable of inhibiting the DNA repair enzyme, poly [ADP-ribose] polymerase 1, (PARP1) leading to tumor cell apoptosis. In some cases, the killing activity of Iniparib in several tumor cell lines has been demonstrated in previous studies, and differences in susceptibility are attributed to the capacity of the different cell lines to reduce Iniparib to the nitroso metabolite. However, the nitroso metabolite has not been directly detected in cellular systems, and the indirect evidence to support the presence of the nitroso metabolite has been the identification of the fully reduced 4-iodo-3 aminobenzamide which pointed to the transient existence of the reduced nitroso intermediate and hydroxylamine metabolites. Additionally, a low potency of iniparib against PARP1 is also observed in several models suggesting the presence of additional mechanisms of activity. In some embodiments, studies described below illustrate the identification of additional mechanisms of action for Iniparib.

Materials

All cell lines were purchased from the ATCC cell biology collection. Cell culture reagents were purchased from LifeTechnologies. All regular chemicals or reagents were obtained from Sigma-Aldrich Chemicals, unless otherwise specified.

Synthesis of Iniparib-Biotin Derivative

(3aS,4S,6aR)-4-(5-(1H-imidazol-1-yl)-5-oxopentyl)tetrahydro-1H-thieno[3,4-d]imidazol-2(3H)-one: N,N′-Carbonyldiimidazole 4.98 g (30.7 mmol) was added in several portions to a suspension of D-Biotin, 5 g (20.5 mmol) in 40 ml DMF under nitrogen and heated at 50° C. for 2 h before it was cooled to room temperature, diluted with 50% ether, filtered and dried to give the imidazolide, 5.71 g (19.41 mmol-94.8%) as a white solid.

N-(2-aminoethyl)-5-((3 aS,4S,6aR)-2-oxohexahydro-1H-thieno[3,4-d]imidazol-4-yl)pentanamide: The imidazolide, 5.71 g (19.41 mmol) was added portion wise during 4 h to ethane-1,2-diamine 175 gr (2.91 mol) warmed to 50° C. under nitrogen atmosphere.

After evaporation of the diamine, the crude product was precipitated as an off-white powder in methylene chloride, filtered and washed successively with methylene chloride and ether and dried in vacuo to afford the amide, 5.135 g (92.4%).

2,5-dioxopyrrolidin-1-yl 4-iodo-3-nitrobenzoate: 1-(3-Dimethylaminopropyl)-3-ethylcarbodiimide hydrochloride 9.52 g (49.66 mmol) was added in one portion to a suspension of N-hydroxysuccinimide 9.52 g (82.8 mmol) and 4-Iodo-3-nitrobenzoic acid 9.7 g (33.1 mmol) in dry CH₂Cl₂ (70 mL) under nitrogen at ambient temperature. The suspension was stirred overnight. The precipitate formed was filtered, washed twice with CH₂Cl₂ and dried to afford the NHS active ester, 12.1 g (27.94 mmol-84.4%).

4-iodo-3-nitro-N-(2-(5-((3 aS,4S,6aR)-2-oxohexahydro-1H-thieno[3,4-d]imidazol-4-yl)pentanamido)ethyl)benzamide: To a suspension of succinimide ester, 5 g (12.82 mmol) in 100 ml THF, was added the amide, 3.67 g (12.82 mmol) dissolved in 50 ml of water. After 4 h stirring at room temperature the reaction mixture became clear and after one night a new precipitate was formed. The product was collected by filtration and successively washed with water, acetone and CH₂Cl₂. After vacuum drying 6.6 g (91.7% yield) of compound was obtained as an off-white solid (LCMS purity: 97-98%).

All other chemicals were synthesized according to the publication of Mendeleyev et al. (7).

Cell Lines Culture

Human cancer cell lines HCT116 (colorectal carcinoma) and MDA-MB-453 (breast metastatic carcinoma) were cultured in DMEM medium supplemented either with 10% fetal bovine serum (MDA-MB-453) or with 10% decomplemented fetal bovine serum (HCT116), 2 mM glutamine, 1 mM sodium pyruvate and 10 μg/ml ciprofloxacine (Euromedex) in a humidified 5% CO₂ atmosphere at 37° C.

Cytotoxicity Assays

Cell viability following drug treatments was assessed using WST-1 cell proliferation assay (Roche) Briefly, cells were plated into 96-well plates with 8,000 (HCT116) or 20,000 (MDA-MB-453) cells per well and allowed to attach overnight. Cells were then preincubated for 5 h in the presence or absence of 1 mM BSO. Afterwards cells were treated with Iniparib, its metabolites or their vehicle, 1% DMSO, and cell viability was measured 48 h after by addition of WST-1 reagent. After 3 h of incubation at 37° C., the amount of formazan dye was quantified by measuring the optical density at 450 nm with a scanning multiwell spectrophotometer (PerkinElmer).

XLC-MS/MS Analysis of Iniparib and Metabolites

Online solid phase extraction (SPE) was performed using the fully automated Spark Holland Symbiosis™ (Emmen, The Netherlands) in eXtraction Liquid Chromatography (XLC) mode. The Symbiosis™ has previously been described in detail (18). HySphere™ C18 HD 7 μm SPE cartridges were used (Spark Holland). Each cartridge was initially conditioned with 2 ml methanol+0.5% formic acid (FA) and then equilibrated with 2 ml water+0.5% FA, both at flow-rates of 4 ml/min. Sample (25 μl) was aspirated and loaded onto the cartridge with 1 ml water+0.5% FA at a flow-rate of 1 ml/min. Cartridge was put in line with LC pumps for 20 min for Iniparib and metabolites elution. After elution, the right clamp was washed successively with 2 ml water+0.5% FA, 2 ml methanol+0.5% FA and 2 ml water+0.5% FA at 4 ml/min. Liquid Chromatography was performed using water+ammonium formate 5 mM for mobile phase A and methanol+ammonium formate 5 mM for mobile phase B. Iniparib and metabolites were eluted from the SPE cartridges onto a Symmetry C18 3.5 μm, 2.1 mm×50 mm column (Waters) at 0.150 ml/min. Initial conditions were 95/5 (v/v) mobile phase A:mobile phase B. The proportion of mobile phase B was gradually increased to 50% over 10 min, maintained at 50% during 4 min and then increased to 95% over 1 min and maintained during 5 min. The mobile phase composition was returned to starting conditions over 1 min and maintained during 6 min for column equilibration. The total run time was 27 min. Mass spectrometry was performed using the Thermo® TSQ Quantum Discovery Max in electrospray positive ionization mode (Thermo, US) with the following parameters: capillary voltage=4.0 kV, capillary temperature=300° C., sheath gas pressure=40, auxiliary gas pressure=5. Iniparib and metabolites were analyzed in Multiple Reaction Monitoring (MRM) mode, with the following specific mass to charge (m/z) transitions: 292.8-119.8@28, 276.8-246.8@15 and 472.0-343.0@18 for Iniparib, I-NOBA and Iniparib-glutathione conjugated (I-GS), respectively. For quantitation, 2-chloroAdenosine (2-ClAde) was used as Internal Standard and peak area response ratios of Iniparib & metabolites/2-ClAde were calculated using the Thermo LC-Quan software.

In Vitro Iniparib-Glutathione Conjugation.

Iniparib at 30 μM was incubated at 20° C. for 150 min in 50 mM Tris-HCl pH 7.5 containing 1 mM reduced glutathione in the presence or the absence of human recombinant glutathione S-transferase pi (GSTP1, Acris) at 1.5 μM.

In Vitro Modification of GAPDH by Iniparib and I-NOBA.

For biochemical analysis, recombinant human GAPDH (Acris) was first reduced by a 15,000-fold excess of DTT at 56° C. for 30 min, the reducing agent being removed thereafter by chloroform/methanol precipitation. Reduced form of GAPDH at 1.4 μM was then incubated in 50 mM Tris-HCl pH 7.5 at 37° C. for 15 min with 100 μM Iniparib, 100 μM I-NOBA, or their vehicle, 1% DMSO. Afterwards the samples were submitted or not to another reducing step with 25 mM DTT (56° C., 30 min), and after removal of DTT by chloroform/methanol precipitation, biotinylation of free cysteine sulfhydryl groups of GAPDH was performed with HPDP-biotin (N-[6-(Biotinamido)hexyl]-3′-(2′-pyridyldithio)propionamide) at 0.8 M for 2 h at 20° C.

For LC/MS analysis, GAPDH at 3 μM was incubated in 10 mM Tris-HCl pH 7.5 with 30 μM Iniparib or I-NOBA for 1 h at 20° C. Sample mixtures were then reduced or not with 25 mM DTT (56° C., 30 min). Intact molecular weights were measured by mass spectrometry. Liquid chromatography-electrospray ionization mass spectrometry (LC/MS) was carried out using LTQ-Orbitrap Elite mass spectrometer (Thermo Fisher Scientific) coupled to a Famos Autosampler and an Ultimate Pump (LC-Packing, Dionex). Reverse phase chromatography was performed with a binary buffer system consisting of 0.2% formic acid (buffer A) and 80% acetonitrile in 0.2% formic acid (buffer B). After dilution to 1 pmole/μl in 0.2% formic acid, 1 μl of samples were loaded on a Poros 1 R/H column (75 μm×15 cm, Dionex). The proteins were eluted by a linear gradient of buffer B (25% to 50% in 10 min, 50% to 90% in 2 min) for a total 35 min gradient run with a flow rate of 250 nl/min. Mass spectra (m/z 500-2,000) were acquired in the positive ITMS mode with 5 μscans accumulation, a target value of 30,000 and a maximum injection time of 100 ms. The acquired raw files were converted in MassLynx format (Waters) using an home-made program and then were deconvoluted using MaxEnt software (Waters).

Cell Exposure to Iniparib-Biotin Derivative and Cell Lysis.

MDA-MB-453 and HCT116 cells were first pre-treated or not for 48 h with 1 mM BSO. Afterwards cells were incubated in serum-containing conditioned medium with increasing concentrations of Iniparib-biotin derivative for up to 4 hours. Then cells were solubilized in ice-cold octyl-glucoside buffer (1.5% octyl-glucoside, 150 mM NaCl, 25 mM Tris-HCl, pH 7.5) supplemented with protease and phosphatase inhibitors (Pierce, Thermo scientific). After 2 h at 4° C., lysates were clarified by centrifugation and protein amounts were measured using the BCA assay (Pierce, Thermo scientific).

SDS-PAGE and Western Blot Analysis.

Equal amounts of proteins were resolved by SDS-PAGE under either non-reducing or reducing (5% 2-mercaptoethanol, 20 min at 60° C.) conditions, using 4-20% gels (Novex, Invitrogen), then subjected to semi-dry electrophoretic transfer onto nitrocellulose membranes. Membranes were blotted with either Streptavidin-HRP (GE Healthcare), monoclonal antibody against GAPDH (Santa Cruz Biotechnology), polyclonal antibody raised against GSTP1 (Santa Cruz Biotechnology) or with MitoProfile® Total OXPHOS human antibody cocktail (Abcam). Detection of reactive bands was performed by enhanced chemiluminescence (West DURA substrate, Pierce, Thermo scientific).

Monomeric Avidin Pull-Down and Prdx1 Immunoprecipitation for Identification of Prdx1 Modification by Iniparib.

BSO pretreated HCT116 cells were incubated for 4 h either with 100 μM Iniparib-biotin (for monomeric-avidin pull-down) or with 100 μM Iniparib (for Prdx1 immunoprecipitation). Then clarified octyl-glucoside lysates (50 and 12 mg proteins for monomeric-avidin and peroxiredoxin-1 (Prdx1) pull-down, respectively) were combined with either monomeric avidin-agarose (Pierce, Thermo scientific) or anti-Prdx1 monoclonal antibody coupled to protein G plus agarose (Santa Cruz Biotechnology) and mixed overnight at 4° C. Beads were recovered by centrifugation for 2 min at 1,000×g and extensively washed in octyl-glucoside lysis buffer. Precipitated complexes were eluted by incubating the beads for 20 min either at 20° C. in 0.1 M glycine, pH2.8, for monomeric-avidin pull-down, or at 60° C. in SDS sample buffer for Prdx1 immunoprecipitation.

After separation by SDS-PAGE under reducing conditions and PageBlue® protein staining (Thermo scientific), bands of interest were excised, reduced with DTT, alkylated with iodoacetamide and in-gel digested with trypsin (Promega). Peptides were extracted with acetonitrile 50% in 0.2% formic acid and analyzed by Nano LC-MS/MS after partial evaporation in a speed-vac concentrator. LC-MS/MS experiments were performed on an NanoAcquity UPLC (Waters) coupled to a hybrid LTQ Orbitrap Velos mass spectrometer (Thermo Fisher Scientific) equipped with a nanoelectrospray source. Tryptic digests were loaded onto a nanoAcquity UPLC Trap column (Symmetry C18, 5 μm, 180 μm×20 mm, Waters) and washed with 0.2% formic acid at 20 μL/min for 5 min. Peptides were then eluted on a C18 reverse-phase nanoAcquity column (BEH130 C18, 1.7 μm, 75 μm×250 mm, Waters) with a linear gradient of 7-30% solvent B (H₂O/CH₃CN/HCOOH, 10:90:0.2, by vol.) for 85 min, 30-90% solvent B for 10 min, and 90% solvent B for 5 min, at a flow rate of 250 nL/min.

The mass spectrometer was operated in the data-dependent mode to automatically switch between MS and MS/MS acquisition. Survey full scan MS spectra (m/z 300-1,600) were acquired in the Orbitrap with a resolution of 60,000 at m/z 400. The AGC was set to 1×10⁶ with a maximum injection time of 100 ms. The ions were then isolated for fragmentation either in the LTQ linear ion trap or in the Orbitrap. For LTQ linear ion trap fragmentation, most intense ions (up to 20) were fragmented with normalized collision energy of 28% at the default activation q of 0.25 with an AGC of 5×10³ and a maximum injection time of 200 ms. For HCD fragmentation, most intense ions (up to 10) were fragmented with normalized collision energy of 40%, an AGC of 5×10⁴, a maximum injection time of 150 ms and a resolution of 17,500 at m/z 400. In all cases, the dynamic exclusion time window was set to 80 s. LC-MS/MS data, acquired using the Xcalibur software (Thermo-Fisher Scientific), were processed using a homemade Visual Basic program software developed using XRawfile libraries (distributed by Thermo-Fisher Scientific) to generate a MS/MS peak list (MGF file) which will be used for database searching. This MGF file contained the exact parent mass and the retention time (RT) associated with each MS/MS spectrum. The exact parent mass is the ¹²C isotope ion mass of the most intense isotopic pattern detected on the high resolution Orbitrap MS parallel scan and included in the MS/MS selection window. The RT is issued from the MS/MS scan. Database searches were done using our internal MASCOT server (version 2.3, matrix Science; http://www.matrixscience.com/) using the SwissProt human database. The search parameters used for post-translational modifications were dynamic modifications of +57.02146 Da (carbamidomethylation), of +164.02164 (Iniparib minus I adduct) or +433.14199 Da (Iniparib-biotin minus I adduct) on cysteine residues, of +15.99491 Da on methionine residues (oxidation) and −17.026549 Da on N-terminal glutamine residues (N-PyroGlu). The precursor mass tolerance was set to 5 ppm and the fragment ion tolerance was set to 0.5 Da or 20 mmu. The number of missed cleavage sites for trypsin was set to 3. Mascot result files (“.dat” files) were imported into Scaffold software. Scaffold (version Scaffold_3.4.5, Proteome Software Inc., Portland, Oreg.) was used to validate MS/MS based peptide and protein identifications. Queries were also used for XTandem parallel Database Search. The compiled results of both database searches were exported.

Fluorescence Microscopy.

HCT116 cells were platted on polylysine D coated thin glass bottom microscope chambers (Ibidi). After 24 h of culture cells were first pre-incubated or not with BSO at 1 mM for 18 h and then treated with 100 M Iniparib-Biotin or its vehicle (DMSO 1%) for various times. For subcellular localization experiments mitochondria were stained with 100 nM Mitotracker Red CMX (Molecular probes) added for 10 min. This stain was performed before fixation (Paraformaldehyde 3.7% in PBS pH 7.4). Biotin was developed, after Triton X100 (0.3% in PBS, 15 min) permeabilization and saturation (1% BSA+1% gelatin in PBS: saturation buffer), with Alexa-488 streptavidin (Molecular Probes) conjugate (1 μg/ml in saturation buffer). Nuclei were stained with Hoechst (Molecular Probes) and samples mounted in anti-fading solution (Ibidi).

Cells were imaged with a PLAN NeoFluar 40× (NA 1.3) or 100×, (NA 1.46) oil objectives on a LSM510 (Zeiss) confocal microscope. Laser lines, filters and dichroic mirrors were selected for maximal separation of the green (Ex./Em. 488/530 nm) and the red fluorescence (Ex./Em. 543/LP 585 nm). Nuclei were observed (Ex./Em. 405/460 nm). For co-localization stacks of images separated by 400 nm along z-axis were acquired. Post-capture processing was done using LSM510 software, and Photoshop (Adobe) was used to make linear adjustments to brightness and contrast.

ROS Production and Video-Microscopy.

HCT116 cells pretreated or not for 20h with 1 mM BSO were loaded with 5 μM 2′,7′ dichloroflurescein diacetate (H₂DCF). Image acquisition was performed with an Axiovert 200 Zeiss (Carl Zeiss Jena Germany) microscope equipped with a 40× C-Apochromat objective (N.A.=0.6). H₂DCF fluorescence was excited with a LED light source (490 nm) and emitted light was collected at 520-550 nm. For video, H₂DCF-loaded cells were washed with HBSS and kept for measurement in 2 μM H₂DCF in HBSS. Iniparib (100 μM) menadione (50 μM) or their vehicule (1% DMSO) were added and images were recorded (1 image/40 s) on a CCD camera (CoolSnap HQ2), with a fixed exposition time of 50 ms.

For quantification the MetaMorph® software was used. ROIs were traced on representative cells and integrated fluorescence intensities were estimated. Values correspond to the average intensities of the ROIs and are expressed as the ratio F/FO, where F is the averaged intensity recorded at a given time and FO corresponds to an averaged intensity recorded on images captured before adding the compounds.

Apoptosis and Necrosis Assays

Cells were seeded in 6-well tissue culture plates (2.5×10⁵ cells/well). MDA-MB-453 and HCT116 cells were first pre-treated or not for 48 h with 1 mM BSO. Afterwards, cells were incubated with 100 μM Iniparib or its vehicle for various times. Cell-conditioned culture media, that may contain detached cells, were then collected and attached cells were trypsinized. Cells were combined with their corresponding conditioned media and collected by centrifugation at 1500 rpm for 10 min at 4° C. For analysis of apoptosis cells were stained with 3,3-diethyloxacarbocyanine iodide, DiOC₂(3) (50 nM DiOC₂(3) (Molecular probes, M34150), incubation at 37° C., for 30 min and counterstaining with 500 ng/ml 4′,6-diamidino-2-phenylindole, DAPI). Cell- and DNA-associated fluorescence signals were quantified using a FACS-Aria (BD Biosciences) and data were analyzed with the FACSDIVA software (BD Biosciences).

Subcellular Fractionation: Cytosol and Mitochondria Preparation.

Cells were homogenized in an ice-cold buffer containing 10 mM Tris-HCl, pH 7.5, protease and phosphatase inhibitors, and 250 mM sucrose in the case of mitochondria preparation, using a glass dounce tissue grinder (30 strokes). After centrifugation at 1,000×g for 5 min at 4° C., homogenates supernatants were collected. For cytosol preparation the 1000×g supernatants were submitted to differential centrifugation in Tris-HCl buffer: a first centrifugation was carried out at 22,000×g for 20 min and the resulting supernatants were further centrifuged at 100,000×g for 60 min. The final supernatants were referred to as the cytosolic fractions. Functional mitochondria were isolated from the 1,000×g homogenates supernatants by affinity chromatography using anti-TOM22 magnetic microbeads according to the manufacturer's protocol (Miltenyi), the final mitochondria pellet being washed in 10 mM Tris-HCl, pH 7.5, 250 mM sucrose.

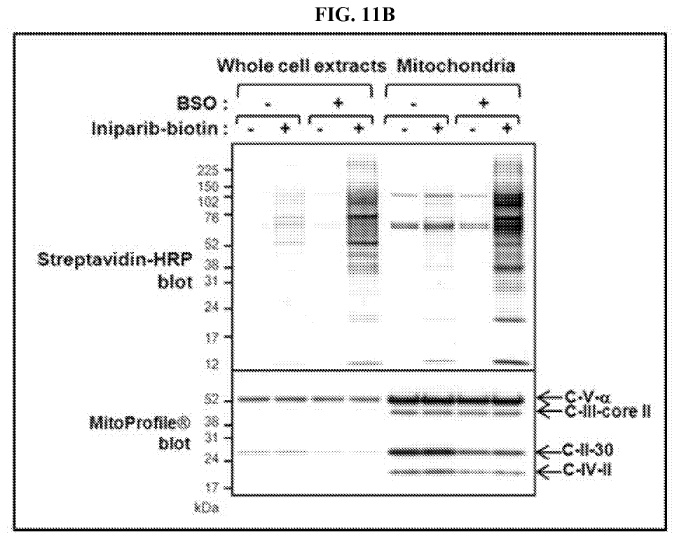

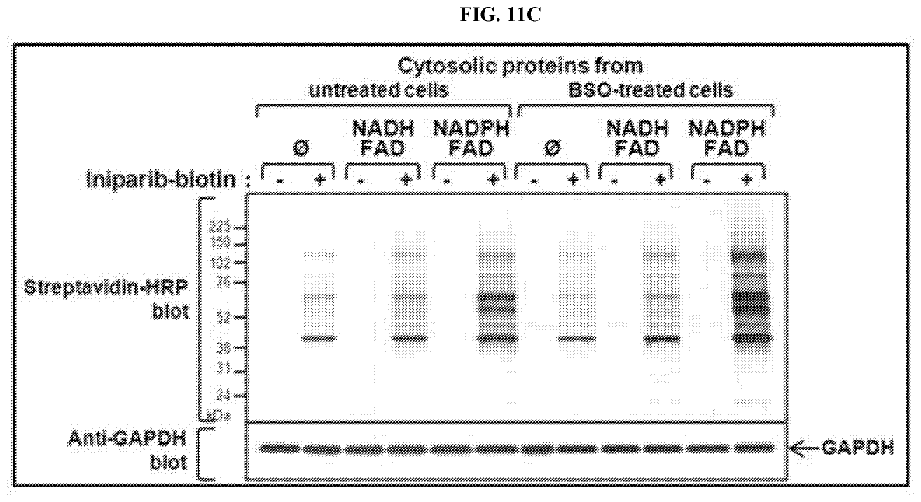

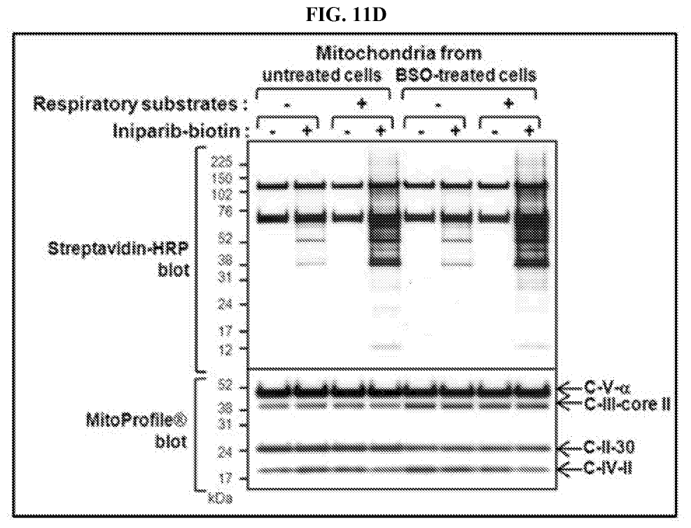

FIG. 11A and FIG. 11B show HCT116 cells were pre-treated or not for 48 h with 1 mM BSO. Thereafter Iniparib-biotin at 100 μM was added for 4 h. Cell homogenates were then processed for cytosol preparation (FIG. 11A) or mitochondria immuno-purification (FIG. 11B).

FIG. 11C and FIG. 11D show cells were pretreated or not for 48 h in the presence of 1 mM BSO. Cell homogenates were then processed for cytosol preparation or mitochondria immuno-purification. Cytosolic proteins were incubated for 3 h 30 min at 37° C. with 30 μM Iniparib-biotin or its vehicle in the presence or the absence of 200 μM NADH/5 μM FAD or 200 μM NADPH/5 μM FAD (FIG. 11C). Isolated mitochondria were incubated for 3 h 30 at 37° C. with 30 μM Iniparib-biotin or its vehicle either in buffer A (5 mM HEPES pH 7.2, 70 mM Sucrose, 210 mM Mannitol, 1 mM EGTA, 0.5% fatty acid-free BSA) or in buffer B which contained «respiratory substrates» (2 mM HEPES pH 7.2, 70 mM Sucrose, 220 mM Mannitol, 1 mM EGTA, 0.2% fatty acid-free BSA, 10 mM KH2PO4, 5 mM MgCl2+10 mM Pyruvate, 2 mM Malate, 10 mM Succinate, 4 mM ADP) (FIG. 11D).

Iniparib Metabolites and Cell Cytotoxicity

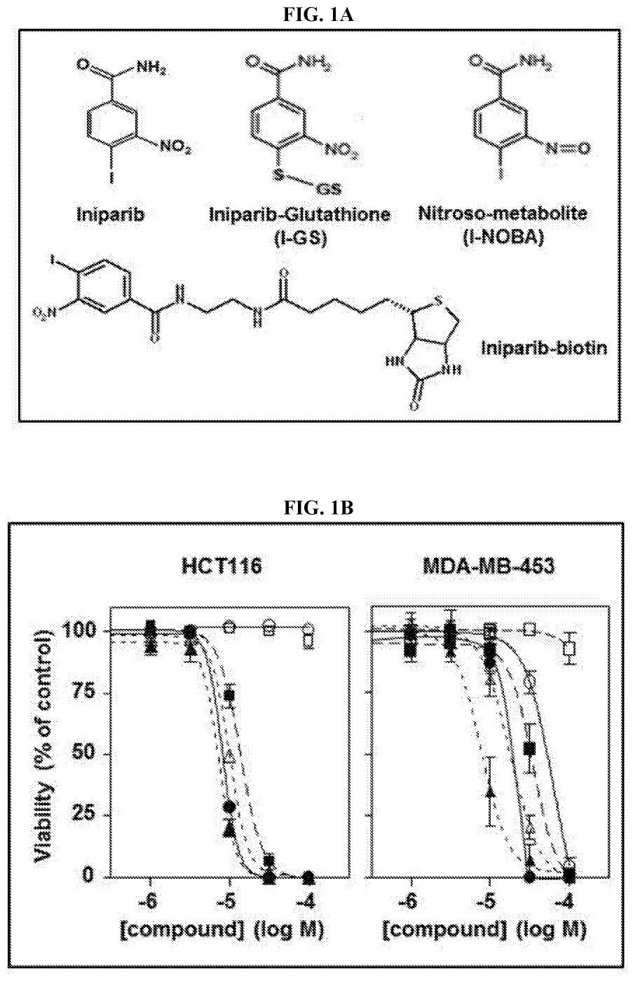

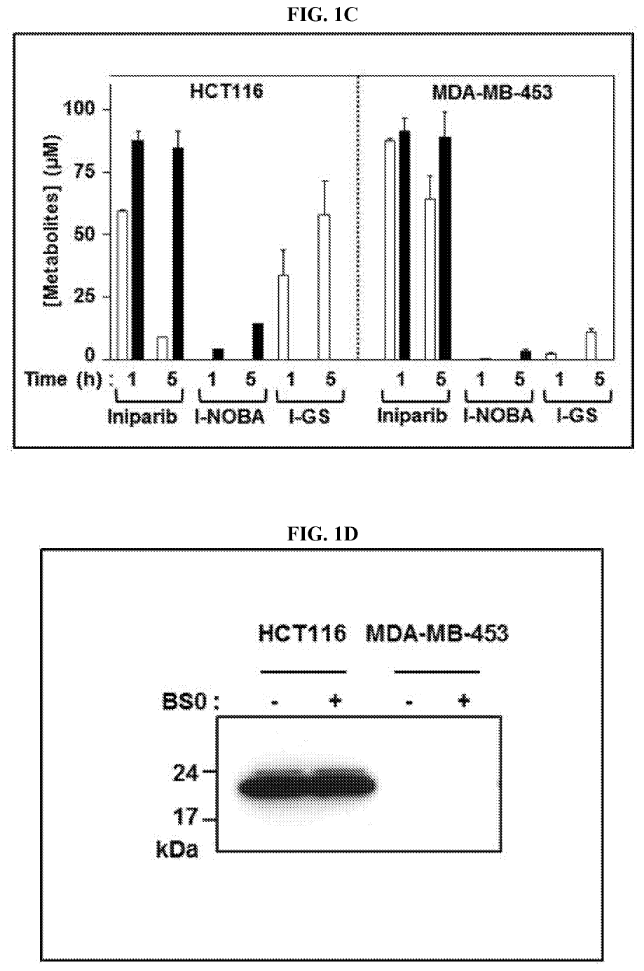

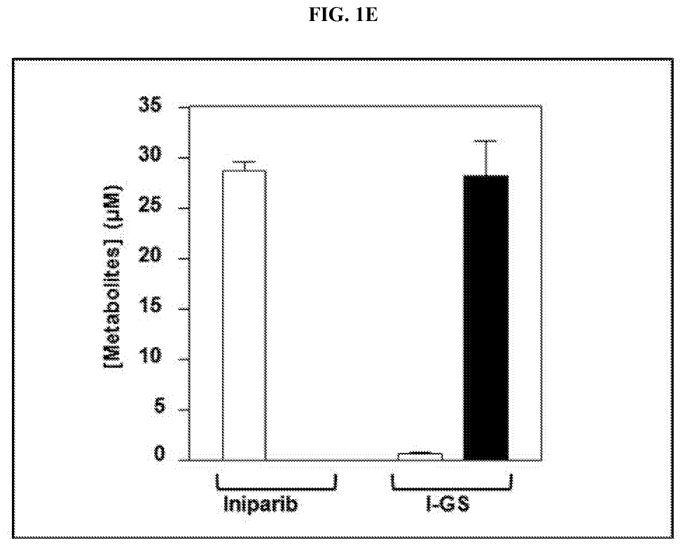

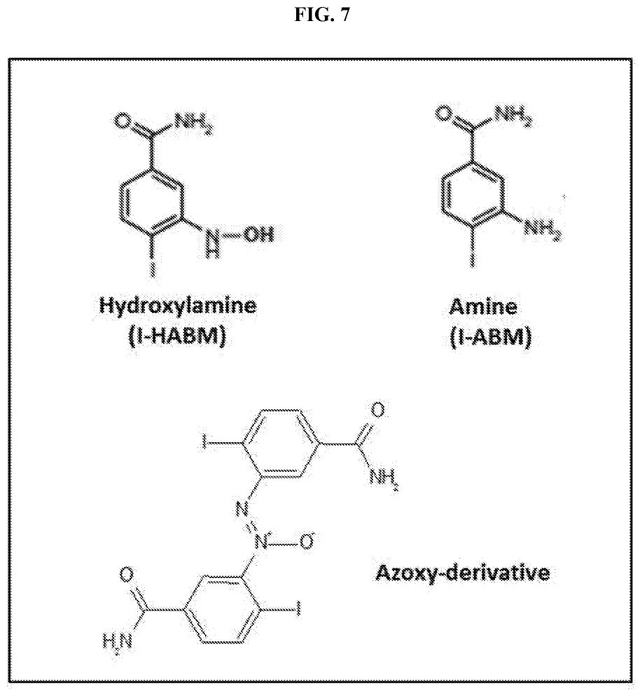

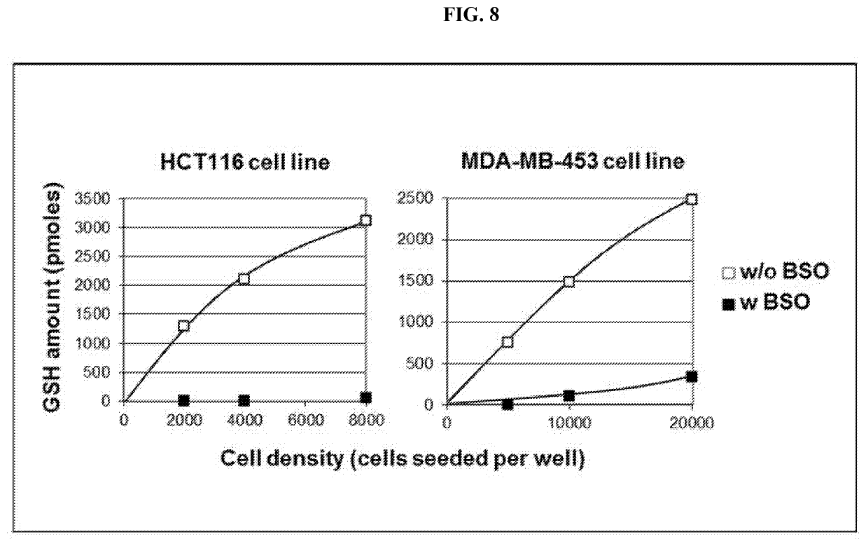

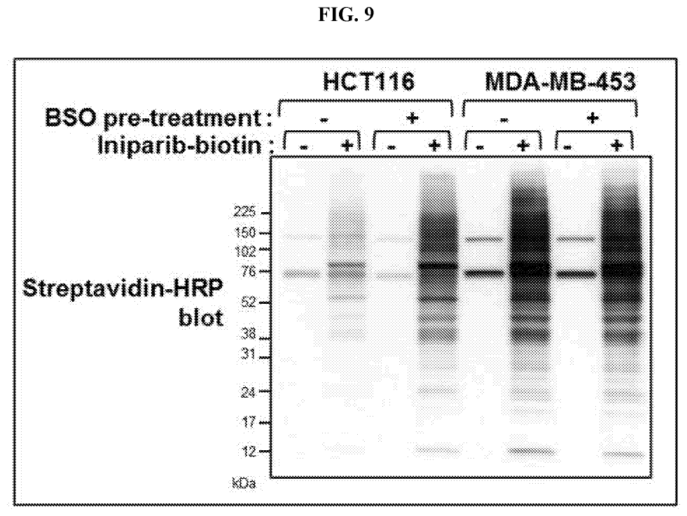

Once inside cells, Iniparib is either conjugated with glutathione and enter a detoxification pathway, or forms cytotoxic metabolites through nitroreductive pathways. To investigate the nature of the metabolites involved in the cellular toxicity of the molecule, several expected metabolites in both the detoxification and the proposed two-electron nitroreductive pathways of Iniparib were synthesized (FIG. 1A and FIG. 7), and the cytotoxic activity of the molecules on several cancer cell lines were evaluated. Since it has been shown that depletion of intracellular GSH potentiates the cytotoxic activity of Iniparib, the different cell lines were either depleted or not of GSH by a preincubation of 24 h with BSO. In all cell lines, I-NOBA was more active than Iniparib, and GSH depletion by BSO had a marked effect on the cytotoxicity of Iniparib while modifying very little the activity of I-NOBA. Based on the preliminary screening, two cell lines were chosen for further analysis of the mechanism of action of Iniparib: a breast adenocarcinoma cell line (MDA-MB-453), and a colon cancer cell line (HCT116). In MDA-MB-453 cells Iniparib was cytotoxic without BSO pre-treatment (IC50: 84±6.5 μM) and GSH depletion increased the cytotoxicity (4 fold augmentation, IC50: 17.8±2.9 M) (FIG. 1B). In HCT116 no cytotoxicity was detected up to 100 μM of Iniparib but GSH depletion rendered these cells very sensitive to the compound (IC50 8.4±0.5 μM). Other metabolites synthesized and tested were inactive on both cell lines before or after depletion of GSH (not shown). Measurement of cellular GSH amounts showed that under similar culture conditions, HCT116 cells had twice as much GSH than MDA-MB-453, and that preincubation of both cell lines with 1 mM BSO for 18 h reduced GSH content to levels below 5% of controls (FIG. 8).

Iniparib Metabolites' Production

Next Iniparib metabolites generation was investigated in HCT116 and MDA-MB-453 cells incubated with Iniparib for various times, following or not GSH depletion. Quantitative analysis first showed that production of Iniparib-GS conjugate was much efficient in HCT116 cells than in MDA-MB-453 cells (FIG. 1C and Table 1). As expected BSO has a dramatic effect in both cell lines on Iniparib-GS formation. By contrast, the production of I-NOBA was detected in cells depleted of GSH by BSO pretreatment. In some instances, higher concentrations of I-NOBA was observed in HCT116 cells as compared with MDA-MB-453 cells. The NH₂ derivative (I-ABM) was observed in both cell lines with or without GSH depletion, while the OH-derivative (I-HABM) was not detected, which suggests a rapid reduction of this metabolite into the corresponding amine. These results show that 1) the nitro group of Iniparib is submitted inside the cells to a two-electron reductive pathway producing small but significant amounts of the highly reactive I-NOBA and 2) show a slow conversion rate of Iniparib into I-NOBA as compared with the rapid conjugation to GSH.

The observation that GSH-Iniparib conjugation was much less efficient in MDA-MB-453 than in HCT116 cells pointed to possible differences in glutathione-S-transferase (GST) enzymes content/activity in the two cell lines. Thus the levels of GST isoforms transcript expression were investigated and, as illustrated in Table 2, among the 19 candidates evaluated only one, GSTP1, was abundantly expressed in HCT116 when compared with MDA-MB-453 cells. And, as shown in FIG. 1E, the difference observed at mRNA level was confirmed at the protein level: GSTP1 is highly present in HCT116 cells but virtually absent in MDA-MB-453. Based on these results whether or not GSTP1 was able in vitro to produce Iniparib-GS conjugate was investigated. As seen in FIG. 1F, Iniparib (30 μM) did not react when incubated for 2.5 h with a large excess of GSH (1 mM), but when recombinant GSTP1 was added, a complete conversion of Iniparib to Iniparib-GS occurred. Under the same conditions I-NOBA reacted with GSH in the absence of GSTP1 to yield the hydroxyl-amine derivative.

Iniparib does not Modify Proteins, I-NOBA Forms Adducts In Vitro with Free Thiol Groups, Adducts which are Sensitive to Reducing Conditions

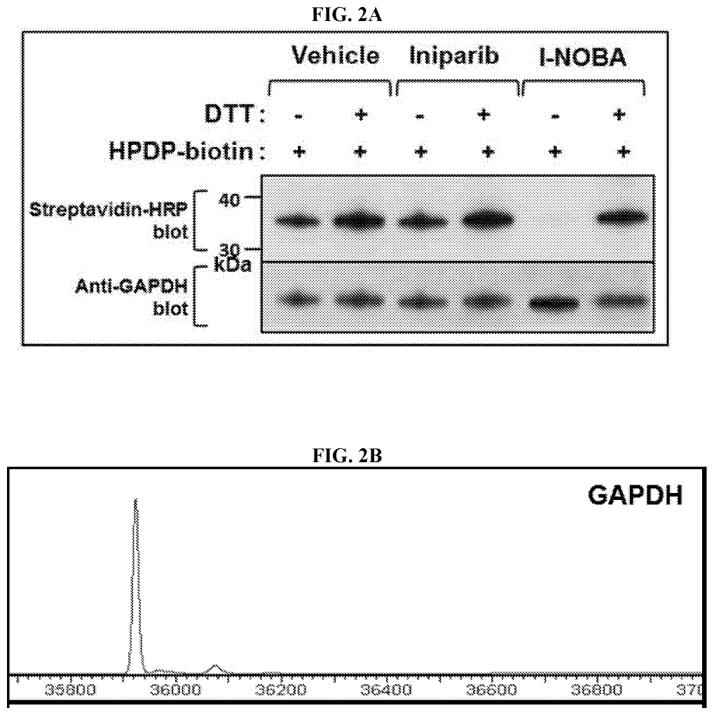

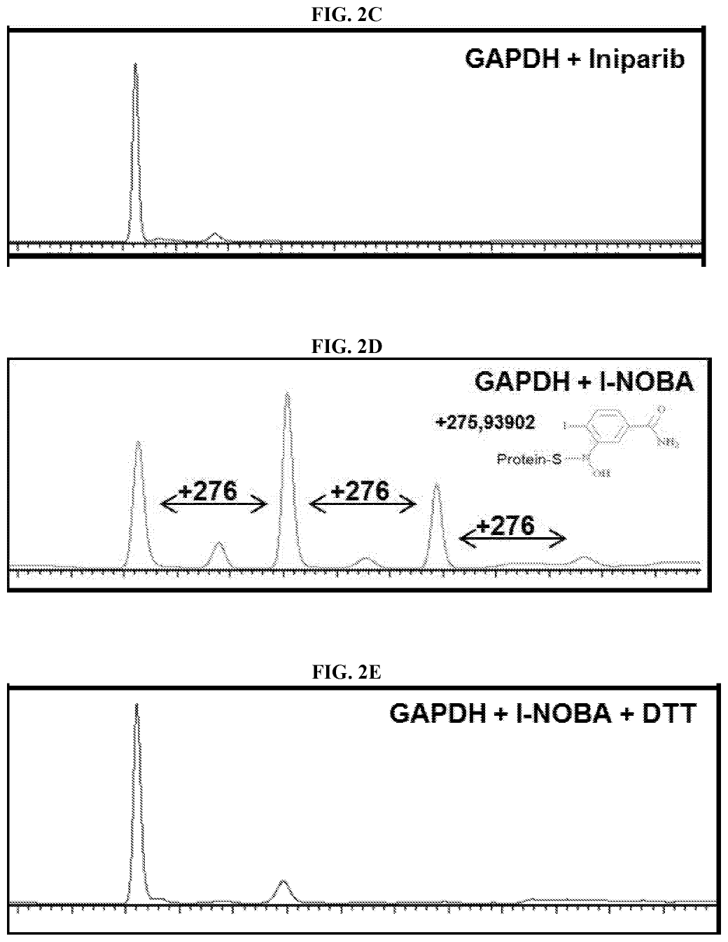

In some instances, GAPDH was suggested as a target for covalent modification by Iniparib and/or I-NOBA. This protein has three free thiol groups, two in the active site (Cys152 and Cys156) and one on the surface (Cys247). The thiol modifying capacity of Iniparib and I-NOBA was evaluated using purified GAPDH. After incubation with either Iniparib or I-NOBA, thiol groups of GAPDH left free were labeled with biotin-HPDP and revealed by streptavidin-HRP blot. As illustrated in FIG. 2A, Iniparib was ineffective to block free thiol groups in GAPDH, but treatment with I-NOBA completely blocked free thiol groups on the purified protein. In some cases, the adducts formed by I-NOBA on the thiol groups were reversed when the modified protein was reduced with DTT prior to labeling with biotin-HPDP.

Mass spectrometry was used to further investigate the nature of the I-NOBA adducts. As shown in FIG. 2C, MS analysis of purified GAPDH incubated with Iniparib confirmed that this compound was not able to covalently modify the protein since no differences were seen when compared with the untreated protein (FIG. 2B). The analysis of the protein following incubation with I-NOBA clearly showed the formation of protein adducts (FIG. 2D), which were reversed under reducing conditions (FIG. 2E). High resolution MS analysis of GAPDH-I-NOBA adducts revealed that two thirds of the modified GAPDH displayed a mass shift compatible with the modification of one cysteine by I-NOBA, the delta mass of +276 being fully compatible with the formation of an initial Cys semimercaptal adduct. The rest of the modified protein showed similar adducts on two Cys, and a very small amount of GAPDH showed a mass shift corresponding to three modified Cys (FIG. 2D). Attempts to directly identify the adducts in the modified protein, after tryptic digestion followed by HPLC peptide separation and MS analysis, were not successful in indicating that the modification was not stable under the conditions of analysis as expected regarding a Cys-semimercaptal adduct.

A Tool Compound for Understanding the Mechanism of Action of Iniparib

To further investigate the metabolism and mechanism of action of Iniparib in cell biology and proteomics experiments, a biotin-derivative of Iniparib was designed and synthesized (FIG. 1A). The tagged synthetic Iniparib-biotin was compared with Iniparib in terms of cytotoxic potency in several tumor cell lines and, as shown in particular for HCT116 and MDA-MB-453 cells (FIG. 1B), the two compounds displayed similar dose-response profiles. Furthermore, GSH, depletion enhanced to the same extent Iniparib and Iniparib-biotin potency (illustrated by similar shifts in IC₅₀ in both cell lines). Taken together these results indicate that the biotin tag did not modify the compound behavior towards the detoxification and activation pathways.

Bio Activated Iniparib Forms Stable Adducts with Cellular Proteins

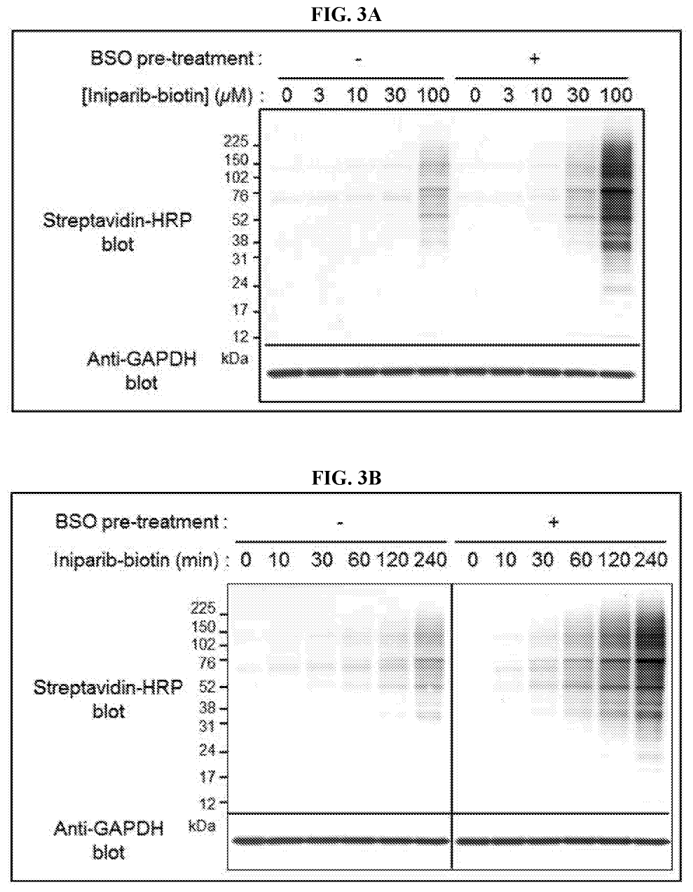

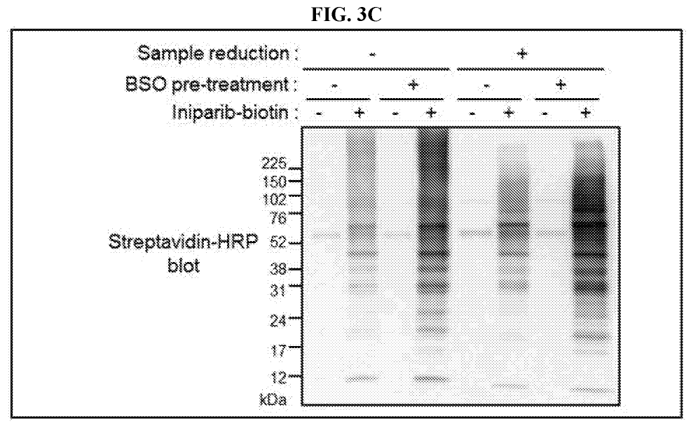

Having confirmed that Iniparib and Iniparib-biotin display similar biological activity on the tested cell lines, Iniparib-biotin, as Iniparib, did not modify free thiol groups in purified GAPDH as well as on several other proteins. Whether or not Iniparib-biotin was in-cell bio activated to metabolite(s) able to form adduct with cellular proteins was then investigated. HCT116 cells, depleted or not of GSH, were incubated either for 4 h with increasing concentrations up to 100 μM of Iniparib-biotin (FIG. 3A) or for various times up to 4 h with 100 M Iniparib-biotin (FIG. 3B), a concentration either almost ineffective in the absence of GSH depletion or inducing full cell killing when cells were depleted of GSH. In cells depleted of GSH, a strong protein labeling with Iniparib-biotin was observed, which was concentration- and time-dependent. By contrast, in HCT116 cells not depleted of GSH, protein modifications by Iniparib-biotin were barely detectable. Thus, the intensity of the total protein labeling appeared correlated with Iniparib cytotoxicity. In control experiments performed with heat-treated cellular extracts, no labeling was observed indicating that Iniparib most probably needs an enzyme for activation to be able to modify intracellular proteins. These results confirm that Iniparib, if not detoxified by conjugation to GSH, can be activated intracellulary, and thereafter covalently modifies proteins.

The protein modifications following incubation with Iniparib-biotin in HCT116 cells were also observed in MDA-MB-453 cells (FIG. 9) and in all the cell lines studied, with similar pattern of labeled protein targets. Furthermore, there was also a correlation depending on GSH depletion between the extend of total protein labeling and the relative cytotoxicity of iniparib in these tumor cell lines.

As shown in FIG. 3C, the adducts formed following in cell bio activation of Iniparib-biotin had limited sensitivity to reducing conditions; suggesting that the majority of the adducts resulting from the incubation of the cells with Iniparib was different to the one characterized in vitro with I-NOBA which was sensitive to reducing conditions as described above (FIG. 2).

The Adducts Formed by Bioactivated Iniparib with Thiol Groups Indicate a One-Electron Reductive Activation of the Molecule

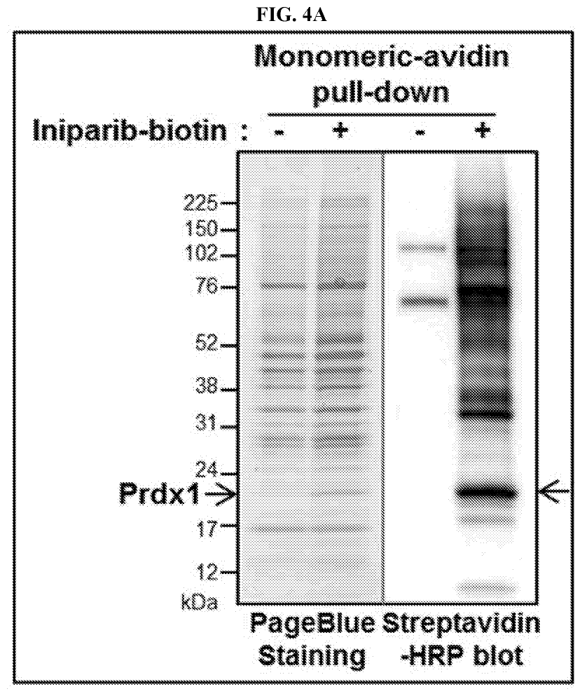

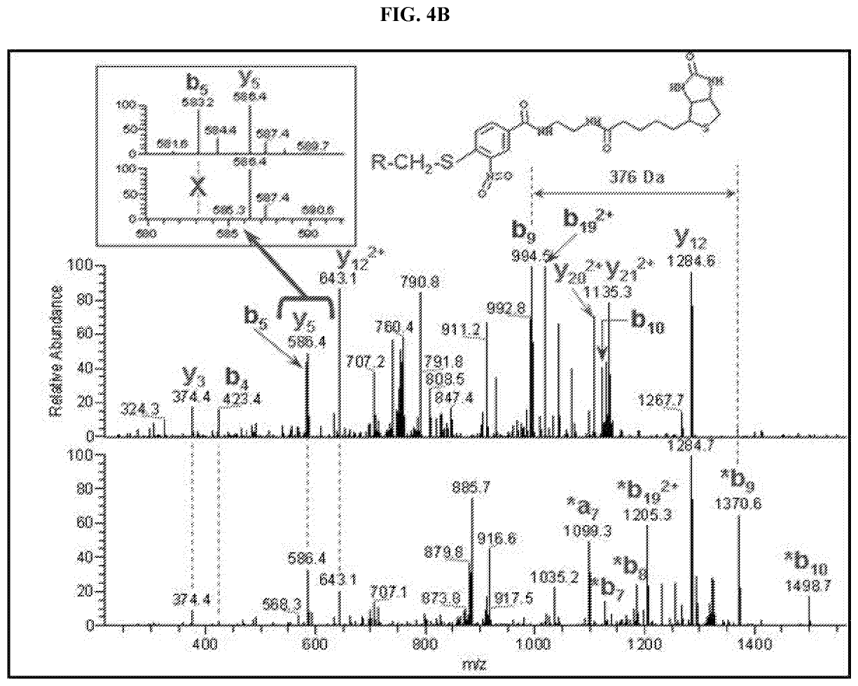

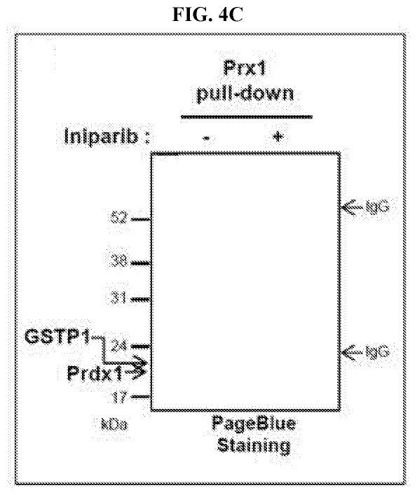

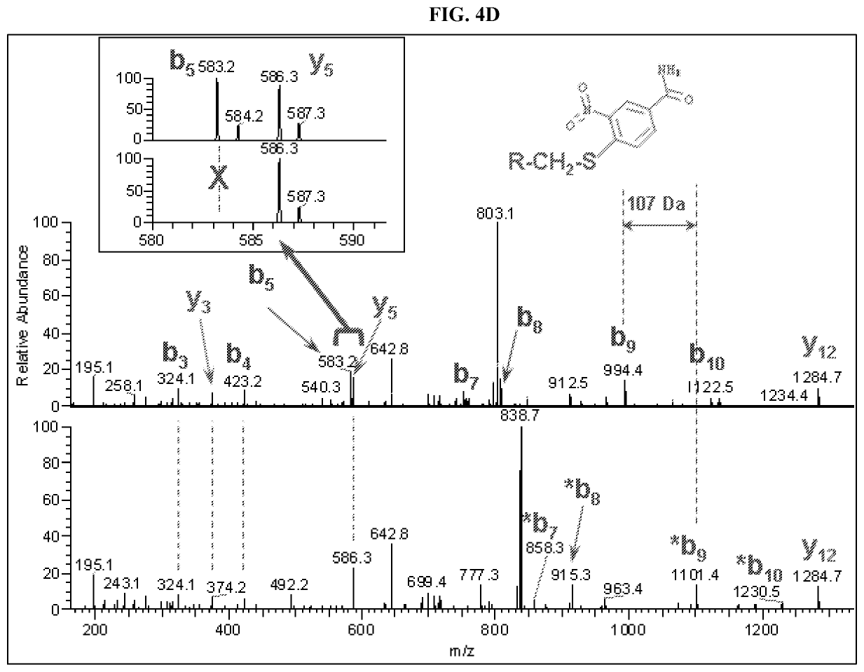

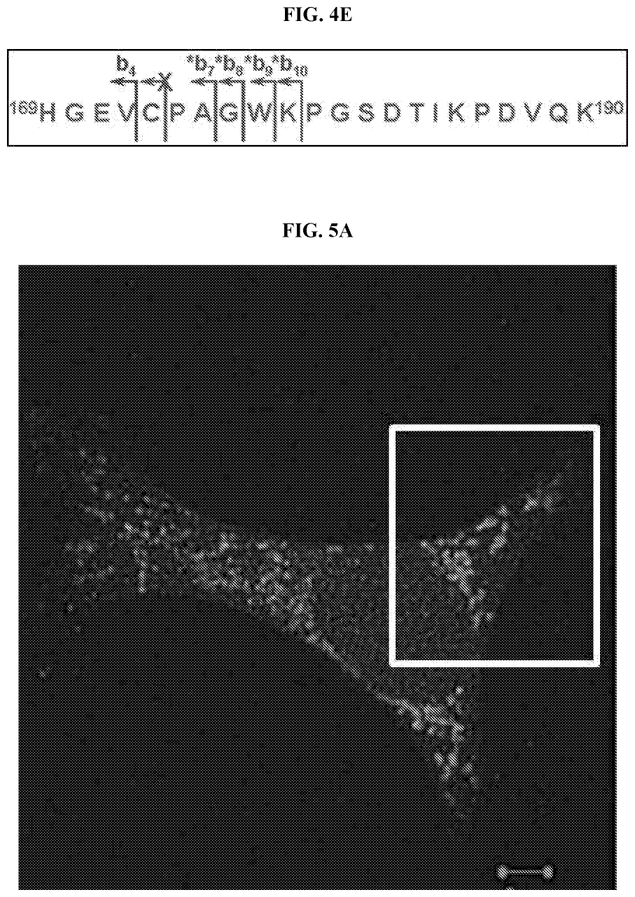

Preliminary experiments, in preparation for global proteomics analysis of Iniparib-biotin targeted proteins, resulted in a clear enrichment of a labeled 22-kDa protein following isolation on monomeric-avidin-beads (FIG. 4A). The protein band was cut and digested with trypsin and the resulting peptides were analyzed by LC/MS-MS. This allowed the unambiguous identification of the 22-kDa protein as Prdx1 with sequence coverage of about 90% (not shown). A search for modified peptides resulted in the identification of a tryptic T20 peptide corresponding to residues 169-190 (FIG. 4E) with a 433-Da delta positive mass (+376 Da delta mass compared to carbamidomethylated peptide of non-modified Prdx 1). The ms/ms spectrum of this modified peptide allowed positioning the modification on Cys173 residue (FIG. 4B). The delta mass value corresponded to an Iniparib-biotin-thiol adduct which structure is given in FIG. 4B insert. To rule out the possibility that this adduct could be an artifact due to the biotin modification of Iniparib, we treated cells with Iniparib and then immunopurified Prdx1. After SDS-PAGE, the expected 22-kDa stained band (FIG. 4C) was cut, digested and the resulting peptides subsequently analyzed by LC/MS-MS. Prdx1 was identified with sequence coverage of about 90%, and the T20 peptide containing the Cys173 with the adduct resulting from Iniparib treatment (+164 Da). As illustrated in FIG. 4D, the delta mass on Cys173 (+107 Da compared to carbamidomethylated peptide of non-modified Prdx1), corresponded to the same adduct structure as that observed with Iniparib-biotin. Thus, the in-cell bio activation of either Iniparib or Iniparib-biotin generates a metabolite which modifies Cys173 in Prxd1 (FIG. 4E) and the identified adduct supports a nucleophilic aromatic substitution where a thiol group substitutes a leaving iodine group in Iniparib. The protein co-immunoprecipitated with Prdx1 (FIG. 4C) was identified by MS/MS analysis as GSTP1 (not shown). GSTP1 is described as a functional partner of Prdx family members. Following Iniparib treatment, the interaction between GSTP1 and Prdx1 was lost.

The Iniparib-biotin labeled proteins enriched on monomeric-avidin beads were not only directly analyzed by SDS-PAGE, as illustrated in FIG. 4A, but also digested; and the resulting peptides bearing Iniparib-biotin adduct were further purified using again a monomeric-avidin column. MS/MS qualitative analysis showed that the Iniparib adducts found on Prdx1 actually occurred on more than 200 cellular proteins (Table 3). In some instances, the majority of the Iniparib-modified proteins are cytoplasmic, but mitochondrial, nuclear and a small number of membrane proteins targeted by Iniparib were also identified.

Microscopy and Cell Fractionation Studies Confirm Cytosolic and Mitochondrial Localization of Iniparib-Biotin Targets

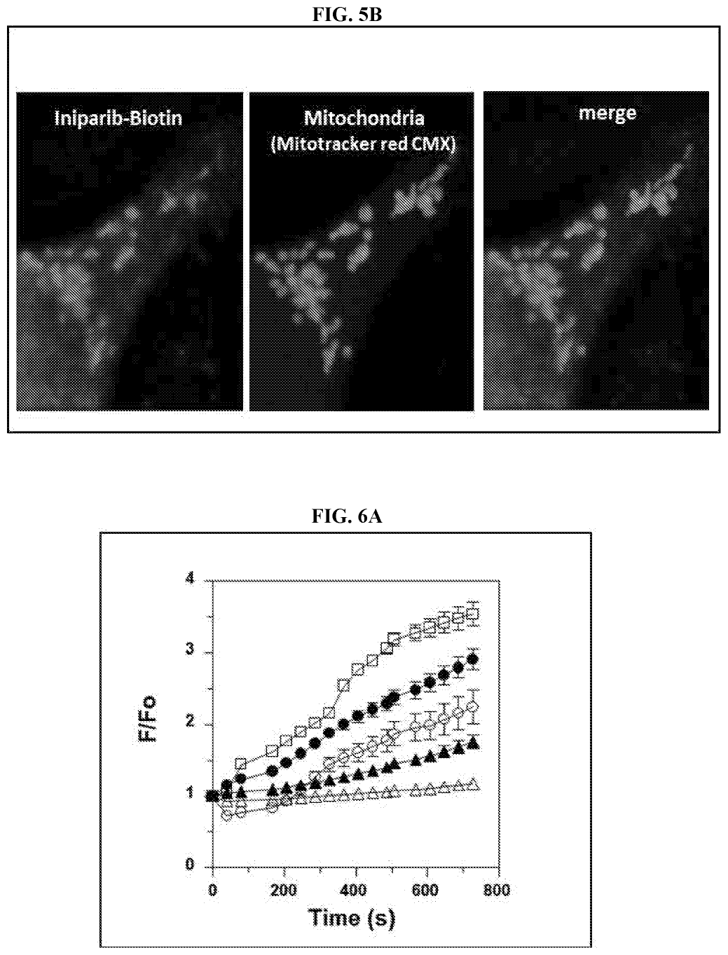

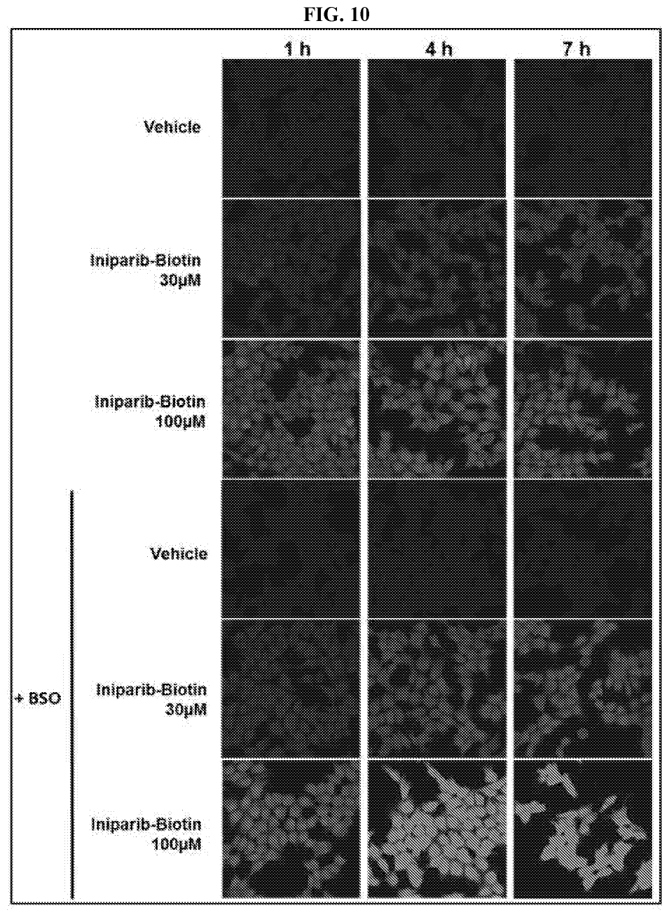

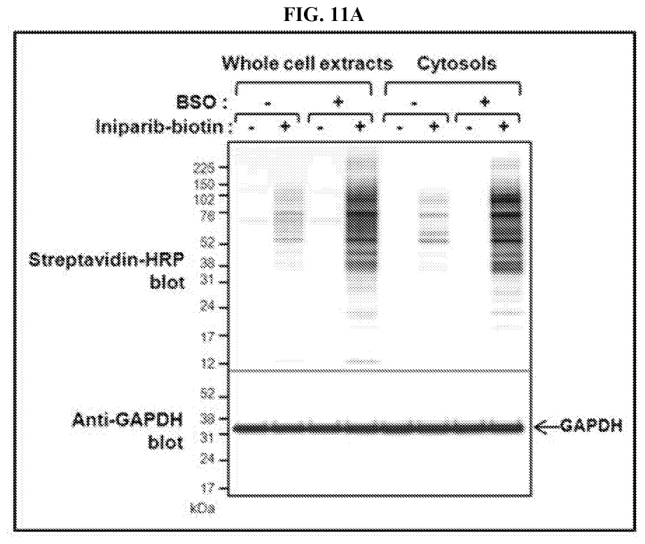

Experiments with the compound tool Iniparib-biotin to evaluate Iniparib-targets subcellular localization were performed. HCT116 cells, depleted or not of GSH, were treated with Iniparib-biotin and fixed after different incubation times. Thereafter the localization of Iniparib-biotin targets was imaged using a fluorescently tagged avidin as a developer for confocal microscopy studies. As illustrated in FIG. 5A, 30 min after adding Iniparib-biotin, cellular proteins were labeled in the cytosol as well as in the nucleus and cytoplasmic structures. As shown in FIG. 5B, adducts were formed with proteins colocalized with the mitochondrial probe, Mitotracker Red CMX, suggesting the modification of mitochondrial proteins. In cells treated with vehicle, a basal mitochondria avidin-labeling due to endogenous biotinylated-carboxylases was detected but this labeling was clearly enhanced in Iniparib-biotin treated cells. The intensity of cell labeling by Iniparib-biotin increased with incubation times and was dependent of the GSH content (FIG. 10), which suggested a correlation between protein adduct formation and cytotoxicity.

The observations above were extended in cell fractionation studies. Cytosol and mitochondria from Iniparib-biotin treated cells were isolated (FIG. 11A and FIG. 11B), protein targets of Iniparib-biotin were present in both cell compartments. The extend of the labeling depended on GSH depletion. Proteins in an isolated cytosolic fraction of HCT116 cells were labeled in vitro with Iniparib-biotin mainly when NADPH was added (FIG. 11C). Similarly, the labeling of a mitochondrial fraction with Iniparib-biotin was observed if pyruvate, malate and succinate were added in vitro to the reaction mixture, and as for the cytoplasmic fraction, the adducts formed were resistant to reduction (FIG. 11D).

Oxidative Stress Induction

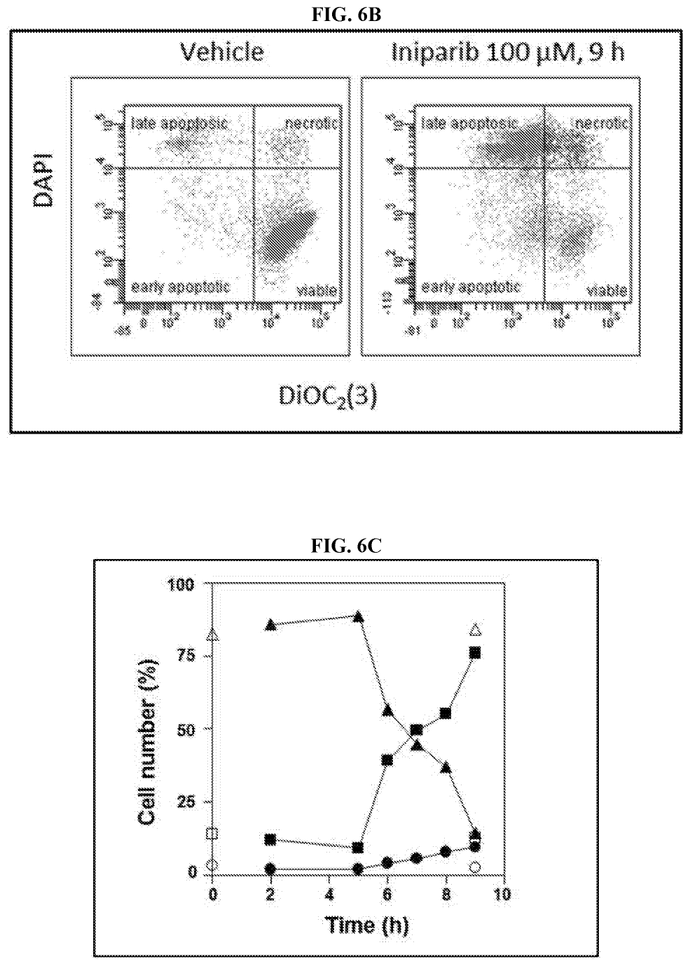

The unstable anion radical species resulting from the one-electron reduction of Iniparib can, in the presence of oxygen, enter a redox cycling process with the associated generation of reactive oxygen species. Thus, we investigated the generation of ROS in cells treated with Iniparib. Incubation of cells loaded with H₂DCF and Iniparib (100 μM) induced an increase in ROS production (FIG. 6A). The ROS production was lower than that produced by incubating the cell with 2-methyl-1,4-naphtoquinone (menadione), a molecule which is known to be reduced in a one-electron pathway and, in the presence of oxygen, to enter into a redox cycle and generation of ROS. When cells were depleted of GSH, the production of ROS started immediately after exposure of the cells to Iniparib, whereas in cells with no depletion of GSH, the production of ROS was significantly delayed most probably due, as described above, to the conjugation of Iniparib to GSH.

Iniparib Induces Both Apoptotic and Necrotic Phenotypes

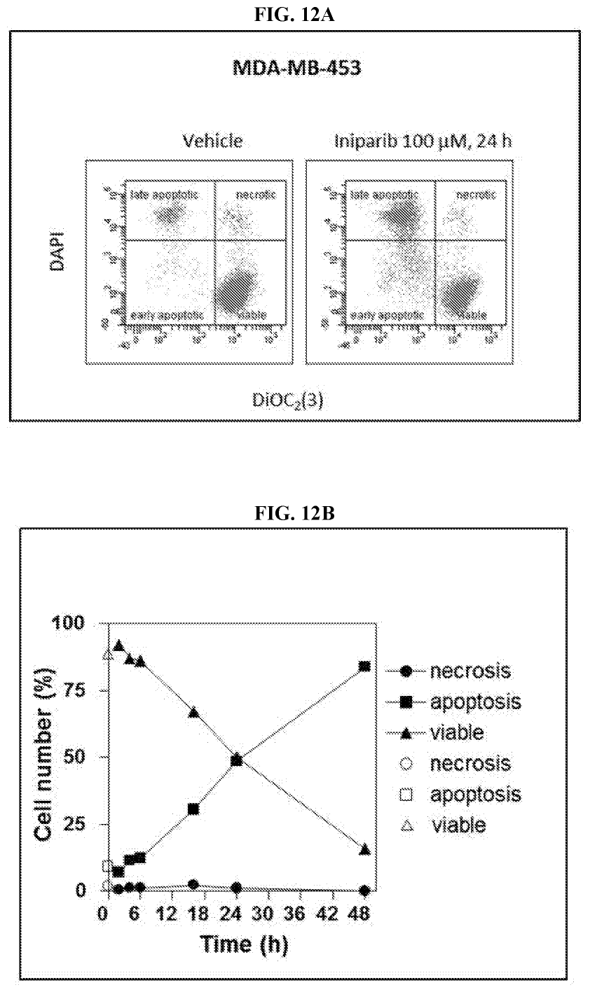

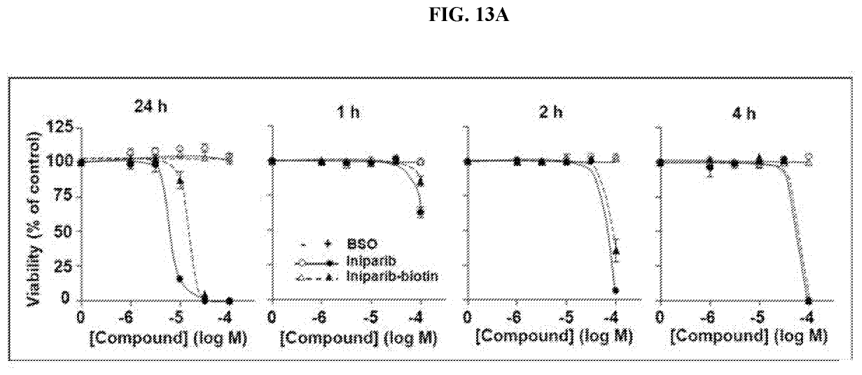

To investigate the time course of events leading to cell death, GSH-depleted HCT 116 cells were exposed to Iniparib for different periods of time and then we evaluated viable, apoptotic, and necrotic cells by flow cytometry, by DAPI, and DiOC₂(3) double staining. As illustrated in FIG. 6B, cells were incubated for 9 h either with vehicle (left panel) or with Iniparib (right panel), and the cells show that Iniparib induced both necrotic and late apoptotic related phenotypes. A change in cell viability was not observed during the first 5 h of Iniparib exposure (FIG. 6C). Apoptotic phenotype, associated with a progressive loss of mitochondrial membrane potential, was clearly detected from 6-h treatment and developed dramatically between 6 and 9 h. Concomitantly some cells entered a necrotic process evidenced by loss of plasma membrane integrity. These data suggest that Iniparib-triggered cell death might combine apoptotic and necrotic simultaneous/independent mechanisms. In MDA-MB-453 cells, without GSH depletion, Iniparib seemed to induce an apoptotic phenotype, but was much slower than in HCT116 cells (FIG. 12).

Table 1 illustrates Iniparib metabolites release by MDA-MB-453 and HCT116 cells. Cells were preincubated for 48 h in the presence or absence of 1 mM BSO. Afterwards cells were exposed to 100 μM Iniparib and aliquots of the incubation media were taken 1 h. 5 h and 24 h after addition of Iniparib Subsequently, media aliquots were analysed by XLC-MS/MS for quantification of Iniparib and metabolites. All values are expressed as means±SEM of three independent experiments. ND., non detectable.

TABLE 1

| Iniparib | | HCT116 | | MDA-MB-453 | |
|---|---|---|---|---|---|
| 100 μM | | w/o BSO | w BSO | w/o BSO | w BSO |
| 1 h | Iniparib | 59.2 ± 0.6 | 87.6 ± 3.5 | 87.1 ± 1.3 | 91.4 ± 5.1 |
| | I-NOBA | ND | 4.4 ± 0.0 | ND | 0.6 ± 0.1 |
| | I-HABM | ND | ND | ND | ND |
| | I-ABM | 0.2 ± 0.1 | 0.3 ± 0.1 | 0.2 ± 0.0 | 0.3 ± 0.1 |
| | I-GS | 33.8 ± 10.2 | 0.1 ± 0.0 | 2.3 ± 0.4 | 0.1 ± 0.0 |
| 5 h | Iniparib | 8.9 ± 0.4 | 84.5 ± 6.7 | 64.0 ± 8.8 | 88.9 ± 10.2 |
| | I-NOBA | ND | 14.6 ± 0.1 | ND | 3.5 ± 0.7 |
| | I-HABM | ND | ND | ND | ND |
| | I-ABM | 0.4 ± 0.1 | 1.1 ± 0.3 | 0.5 ± 0.2 | 1.4 ± 0.1 |
| | I-GS | 57.7 ± 13.7 | 0.1 ± 0.0 | 11.0 ± 1.5 | 0.1 ± 0.0 |
| 24 | Iniparib | ND | 72.9 ± 7.4 | 12.2 ± 4.3 | 74.2 ± 10.8 |
| h | I-NOBA | ND | 26.5 ± 2.9 | 0.2 ± 0.1 | 2.9 ± 1.0 |
| | I-HABM | ND | 0.1 ± 0.0 | ND | ND |
| | I-ABM | 0.5 ± 0.2 | 4.4 ± 1.2 | 2.6 ± 0.2 | 5.4 ± 0.2 |
| | I-GS | 48.2 ± 13.5 | 0.1 ± 0.0 | 35.6 ± 6.4 | 0.1 ± 0.0 |

Table 2 illustrates Quantitative RT-PCR analysis of GST isoforms trancript expression in HCT116 and MDA-MB-453 cells. Total RNAs were extracted from 2 10⁶ cells using total RNA purification kit from Norgen Biotek (Ref: 17200). Genomic DNA was removed by DNAse I treatment using Turbo DNAse-free kit from Ambion (Ref: AM 1907) and pure total RNAs were recovered by using RNA Clean-up kit from Norgen Biotek (Ref:23600). Quality control of RNAs was achieved on nano labchip processed by the 2100 Expert Bioanalyzer (Agilent). 1 μg of total RNAs was reverse transcribed using the SuperScript Vilo cDNA synthesis kit (Life Technologies) and 20 ng of the reaction product was used as a template for quantitative Polymerase Chain Reaction. TaqMan Probes references (Applied Biosystems) are given in Table 1 and Real-time PCR was performed with a 7900FT Fast Real-Time PCR system (Applied Biosystems) according to the following run:2 min at 50° C., denaturing step at 95° C. during 10 min followed by 40 cycles of denaturation step of 15 seconds at 95° C. an annealing/elongation step at 60° C. during 1 min. Results are expressed in threshold cycles (Ct), where Ct corresponds to the level of fluorescence marked by the intersection between the exponential amplification sigmoid curve of each experiment and a threshold fluorescent line, which is above the noise of the experiment and sufficiently low in order to characterize the exponential phase of the measure.

TABLE 2

| | | | | Ct in |
|---|---|---|---|---|
| | | | | MDA- |
| | | | Ct in | MB- |
| | | | HCT116 | 453 |
| GENE | NAME | PROBE | cells | cells |
| GSTM1 | glutathione S-transferase mu 1 | Hs 01683722- | 40.0 | 40.0 |
| | | gH | | |
| GSTM2 | glutathione S-transferase mu 2 | Hs 00265266- | 31.3 | 33.5 |
| | (muscle) | g1 | | |
| GSTM3 | glutathione S-transferase mu 3 | Hs 00356079- | 37.8 | 40.0 |
| | (brain) | m1 | | |
| GSTM4 | glutathione S-transferase mu 4 | Hs 00426432- | 26.9 | 25.9 |
| | | m1 | | |
| GSTM5 | glutathione S-transferase mu 5 | Hs 00757076- | 40.0 | 40.0 |
| | | m1 | | |
| GSTO1 | glutathione S-transferase omega 1 | Hs 02383465-s1 | 26.0 | 27.4 |
| GSTO2 | glutathione S-transferase omega 2 | Hs 01598184- | 25.0 | 25.8 |
| | | m1 | | |
| GSTP1 | glutathione S-transferase pi 1 | Hs 00168310- | 20.0 | 36.0 |
| | | m1 | | |
| GSTK1 | glutathione S-transferase kappa 1 | Hs 1114170-m1 | 24.3 | 24.6 |
| GSTA2 | glutathione S-transferase alpha 1 | Hs 00747232- | 31.9 | 35.4 |
| | (alpha2) | mH | | |
| GSTA3 | glutathione S-transferase alpha 3 | Hs 00374175- | 34.7 | 40.0 |
| | | m1 | | |
| GSTA4 | glutathione S-transferase alpha 4 | Hs 00155308- | 29.6 | 34.8 |
| | | m1 | | |
| GSTT1 | glutathione S-transferase theta 1 | Hs 00184475- | 40.0 | 21.4 |
| | | m1 | | |
| GSTT2 | glutathione S-transferase theta 2 | Hs 00168315- | 27.3 | 28.6 |
| | | m1 | | |
| GSTTP1 | glutathione S-transferase theta | Hs 00894004- | 40.0 | 40.0 |
| | pseudogene 1 | m1 | | |
| GSTCD | glutathione S-transferase. C-terminal | Hs 00226937- | 25.4 | 23.1 |
| | domain containing | m1 | | |
| MGST1 | microsomal glutathione S-transferase 1 | Hs 00220393- | 24.9 | 22.3 |
| | | m1 | | |
| MGST2 | microsomal glutathione S-transferase 2 | Hs 00182064- | 27.3 | 26.7 |
| | | m1 | | |
| MGST3 | microsomal glutathione S-transferase 3 | Hs 01058946- | 25.0 | 24.5 |
| | | m1 | | |
| GAPDH | Glyceride-3-phosphate | 4333764F | 19.8 | 19.8 |
| | dehydrogenase | | | |

Table 3 illustrates Iniparib targets' identification in HCT116 cells. Further, the Iniparib targets identified in Table 3 were identified utilizing the following protocol. BSO pretreated HCT116 cells were incubated for 4 h with 100 μM Iniparib-biotin. Then clarified octyl-glucoside lysates were combined with monomeric avidin-agarose and mixed overnight at 4° C. Beads were recovered by centrifugation for 2 min at 1,000×g and extensively washed in octyl-glucoside lysis buffer. Final wash was performed with 0.15% octyl-glucoside, 50 mM NaCl, 10 mM Tris-HCl, pH 7.5 prior to Iniparib-biotin modified proteins' elution in 0.1 M glycine, pH 2.8, 20 min at 20° C. After pH adjustment to 8.5 by Ammonium Bicarbonate addition (50 mM final), eluted proteins were reduced (10 mM DTT, 30 min at 56° C.), alkylated (30 mM iodoacetamide, 30 min at 20° C.) and digested with trypsin (10 μg, overnight at 37° C.). Tryptic Iniparib-biotin modified peptides were further purified by monomeric avidin pull-down as described above except that the buffer used was 50 mM NaCl, 10 mM Tris-HCl, pH 7.5. After elution in 0.1 M glycine, pH 2.8, 20 min at 20° C., enriched Iniparib-biotin modified peptides were analyzed by LC-MS/MS. LC-MS/MS experiments were performed on a NanoAcquity UPLC (Waters) coupled to a hybrid LTQ Orbitrap Velos mass spectrometer (Thermo Fisher Scientific) equipped with a nanoelectrospray source. Tryptic digests were loaded onto an UPLC Trap column (Symmetry C18, 5 μm, 180 μm×20 mm, Waters) and washed with 0.2% formic acid at 20 μL/min for 5 min. Peptides were then eluted on a C₁₈ reverse-phase nanoAcquity column (BEH130 C18, 1.7 μm, 75 μm×250 mm, Waters) with a linear gradient of 7-30% solvent B (H₂O/CH₃CN/HCOOH, 10:90:0.2, by vol.) for 85 min, 30-90% solvent B for 10 min, and 90% solvent B for 5 min, at a flow rate of 250 nL/min. The mass spectrometer was operated in the data-dependent mode to automatically switch between MS and MS/MS acquisition. Survey full scan MS spectra (m/z 300-1,600) were acquired in the Orbitrap with a resolution of 60,000 at m/z 400. The AGC was set to 1×10⁶ with a maximum injection time of 100 ms. For LTQ linear ion trap fragmentation, most intense ions (up to 20) were fragmented with normalized collision energy of 28% at the default activation q of 0.25 with an AGC of 5×10³ and a maximum injection time of 200 ms. The dynamic exclusion time window was set to 80 s. LC-MS/MS data, acquired using the Xcalibur software (Thermo-Fisher Scientific), were processed using a homemade Visual Basic program software developed using XRawfile libraries (distributed by Thermo-Fisher Scientific) to generate a MS/MS peak list (MGF file) which will be used for database searching. The exact parent mass is the ¹²C isotope ion mass of the most intense isotopic pattern detected on the high resolution Orbitrap MS parallel scan and included in the MS/MS selection window. Database searches were done using our internal MASCOT server (version 2.4, matrix Science) using the SwissProt human database (Uniprot database release-2013_01, 20248 Homo sapiens entries). The search parameters used for post-translational modifications were dynamic modifications of +57.02146 Da (carbamidomethylation) or +433.14199 Da (Iniparib-biotin minus I adduct) on cysteine residues, of +15.99491 Da on methionine residues (oxidation) and −17.026549 Da on N-terminal glutamine residues (N-PyroGlu). The precursor mass tolerance was set to 5 ppm and the fragment ion tolerance was set to 0.5. The number of missed cleavage sites for trypsin was set to 3. Mascot result files (“.dat” files) were imported into Scaffold software. Scaffold (version Scaffold_4.0.3, Proteome Software Inc., Portland, Oreg.) was used to validate MS/MS based peptide and protein identifications. Peptide identifications were accepted if they could be established at greater than 85.0% probability by the Scaffold Local FDR algorithm (<1% FDR). Protein identifications were accepted if they could be established at greater than 97.0% probability and contained at least 1 identified peptide (FDR<1%). Protein probabilities were assigned by the Protein Prophet algorithm (Nesvizhskii, Al et al Anal. Chem. 2003; 75(17):4646-58). Proteins that contained similar peptides and could not be differentiated based on MS/MS analysis alone were grouped to satisfy the principles of parsimony.

TABLE 3

| Protein | | | |
|---|---|---|---|
| accession | | | |
| numbers | HUGO-ID | Protein name | Location |
| L0R804 | AAK1 | Alternative protein AAK1 | Cytoplasm |
| P49588 | AARS | alanyl-tRNA synthetase | Cytoplasm |
| Q16186 | ADRM1 | adhesion regulating molecule 1 | Cytoplasm |
| Q01433 | AMPD2 | AMP deaminase 2 | Cytoplasm |
| Q52LW3 | ARHGAP29 | Rho GTPase activating protein 29 | Cytoplasm |
| O95671 | ASMTL | acetylserotonin O-methyltransferase-like | Cytoplasm |
| Q9BZE9 | ASPSCR1 | Tether containing UBX domain for GLUT4 | Cytoplasm |
| O95817 | BAG3 | BCL2-associated athanogene 3 | Cytoplasm |
| P14635 | CCNB1 | cyclin B1 | Cytoplasm |
| P78371 | CCT2 | T-complex protein 1 subunit beta | Cytoplasm |
| P48643 | CCT5 | chaperonin containing TCP1, subunit 5 (epsilon) | Cytoplasm |
| P48643 | CCT5 | chaperonin containing TCP1, subunit 5 (epsilon) | Cytoplasm |
| Q99832 | CCT7 | chaperonin containing TCP1, subunit 7 (eta) | Cytoplasm |
| Q9UHD1 | CHORDC1 | cysteine and histidine-rich domain (CHORD) | Cytoplasm |
| | | containing 1 | |
| Q6FI81 | CIAPIN1 | cytokine induced apoptosis inhibitor 1 | Cytoplasm |
| Q14008 | CKAP5 | Cytoskeleton-associated protein 5 | Cytoplasm |
| Q14008 | CKAP5 | Cytoskeleton-associated protein 5 | Cytoplasm |
| Q00610 | CLTC | clathrin, heavy chain | Cytoplasm |
| O75153 | CLUH | Clustered mitochondria protein homolog | Cytoplasm |
| O75153 | CLUH | Clustered mitochondria protein homolog | Cytoplasm |
| P62633 | CNBP | Cellular nucleic acid-binding protein | Cytoplasm |
| P62633 | CNBP | Cellular nucleic acid-binding protein | Cytoplasm |
| P62633 | CNBP | Cellular nucleic acid-binding protein | Cytoplasm |
| Q99439 | CNN2 | calponin 2 | Cytoplasm |
| Q13057 | COASY | CoA synthase | Cytoplasm |
| Q9ULV4 | CORO1C | coronin, actin binding protein, 1C | Cytoplasm |
| Q99829 | CPNE1 | copine I | Cytoplasm |
| O75131 | CPNE3 | copine III | Cytoplasm |
| P46109 | CRKL | v-crk sarcoma virus CT10 oncogene homolog (avian)- | Cytoplasm |
| | | like | |
| O75534 | CSDE1 | Cold shock domain-containing protein E1 | Cytoplasm |
| Q14247 | CTTN | cortactin | Cytoplasm |
| Q14247 | CTTN | cortactin | Cytoplasm |
| Q7L576 | CYFIP1 | cytoplasmic FMR1 interacting protein 1 | Cytoplasm |
| Q16643 | DBN1 | drebrin 1 | Cytoplasm |
| Q9BW61 | DDA1 | DET1 and DDB1 associated 1 | Cytoplasm |
| Q6XZF7 | DNMBP | Dynamin-binding protein | Cytoplasm |
| P60981 | DSTN | destrin (actin depolymerizing factor) | Cytoplasm |
| P60981 | DSTN | destrin (actin depolymerizing factor) | Cytoplasm |
| P60981 | DSTN | destrin (actin depolymerizing factor) | Cytoplasm |
| Q14204 | DYNC1H1 | dynein, cytoplasmic 1, heavy chain 1 | Cytoplasm |
| P13639 | EEF2 | eukaryotic translation elongation factor 2 | Cytoplasm |
| Q96EB1 | ELP4 | elongation protein 4 homolog | Cytoplasm |
| B1AK53 | ESPN | Espin | Cytoplasm |
| P21333 | FLNA | filamin A, alpha | Cytoplasm |
| Q7Z6J6 | FRMD5 | FERM domain containing 5 | Cytoplasm |
| P51114 | FXR1 | Fragile X mental retardation syndrome-related protein 1 | Cytoplasm |
| P11413 | G6PD | Glucose-6-phosphate 1-dehydrogenase | Cytoplasm |
| P41250 | GARS | Glycine--tRNA ligase | Cytoplasm |
| P22102 | GART | phosphoribosylglycinamide formyltransferase | Cytoplasm |
| P48506 | GCLC | glutamate-cysteine ligase, catalytic subunit | Cytoplasm |
| P48507 | GCLM | glutamate-cysteine ligase, modifier subunit | Cytoplasm |
| Q06210 | GFPT1 | glutamine--fructose-6-phosphate transaminase 1 | Cytoplasm |
| Q04760 | GLO1 | glyoxalase I | Cytoplasm |
| P36959 | GMPR | guanosine monophosphate reductase | Cytoplasm |
| Q9P2T1 | GMPR2 | guanosine monophosphate reductase 2 | Cytoplasm |
| P49915 | GMPS | guanine monophosphate synthetase | Cytoplasm |
| P49915 | GMPS | guanine monophosphate synthetase | Cytoplasm |
| P63244 | GNB2L1 | guanine nucleotide binding protein (G protein), beta | Cytoplasm |
| | | polypeptide 2-like 1 | |
| P09211 | GSTP1 | glutathione S-transferase pi 1 | Cytoplasm |
| Q8WW33 | GTSF1 | Gametocyte-specific factor 1 | Cytoplasm |
| Q8WW33 | GTSF1 | Gametocyte-specific factor 1 | Cytoplasm |
| Q8WW33 | GTSF1 | Gametocyte-specific factor 1 | Cytoplasm |
| Q9ULT8 | HECTD1 | HECT domain containing 1 | Cytoplasm |
| P30519 | HMOX2 | heme oxygenase (decycling) 2 | Cytoplasm |
| Q9UJC3 | HOOK1 | hook homolog 1 | Cytoplasm |
| P08238 | HSP90AB1 | Heat shock protein HSP 90-beta | Cytoplasm |
| O95757 | HSPA4L | heat shock 70 kDa protein 4-like | Cytoplasm |
| Q9NZL4 | HSPBP1 | Hsp70-binding protein 1 | Cytoplasm |
| Q7Z6Z7 | HUWE1 | HECT, UBA and WWE domain containing 1 | Cytoplasm |
| Q9H0C8 | ILKAP | integrin-linked kinase-associated serine/threonine | Cytoplasm |
| | | phosphatase | |
| Q9P2X3 | IMPACT | Impact homolog | Cytoplasm |
| O15357 | INPPL1 | inositol polyphosphate phosphatase-like 1 | Cytoplasm |
| P48200 | IREB2 | iron-responsive element binding protein 2 | Cytoplasm |
| P35568 | IRS1 | Insulin receptor substrate 1 | Cytoplasm |
| P35568 | IRS1 | insulin receptor substrate 1 | Cytoplasm |
| Q14145 | KEAP1 | kelch-like ECH-associated protein 1 | Cytoplasm |
| Q14145 | KEAP1 | Kelch-like ECH-associated protein 1 | Cytoplasm |
| Q92615 | LARP4B | La ribonucleoprotein domain family, member 4B | Cytoplasm |
| Q9UIC8 | LCMT1 | leucine carboxyl methyltransferase 1 | Cytoplasm |
| P00338 | LDHA | lactate dehydrogenase A | Cytoplasm |
| Q8WXG6 | MADD | MAP-kinase activating death domain | Cytoplasm |
| Q9UPT6 | MAPK8IP3 | C-Jun-amino-terminal kinase-interacting protein 3 | Cytoplasm |
| P10636 | MAPT | microtubule-associated protein tau | Cytoplasm |
| O60502 | MGEA5 | Bifunctional protein NCOAT | Cytoplasm |
| P04732 | MT1E | metallothionein 1E | Cytoplasm |
| P04732 | MT1E | metallothionein 1E | Cytoplasm |
| P02795 | MT2A | Metallothionein-2 | Cytoplasm |
| P02795 | MT2A | Metallothionein-2 | Cytoplasm |
| P11586 | MTHFD1 | methylenetetrahydrofolate dehydrogenase (NADP+ | Cytoplasm |
| | | dependent) 1, methenyltetrahydrofolate | |
| | | cyclohydrolase, formyltetrahydrofolate synthetase | |
| P53602 | MVD | Diphosphomevalonate decarboxylase | Cytoplasm |
| Q9NX02 | NLRP2 | NLR family, pyrin domain containing 2 | Cytoplasm |
| Q9Y314 | NOSIP | nitric oxide synthase interacting protein | Cytoplasm |
| P53384 | NUBP1 | nucleotide binding protein 1 | Cytoplasm |
| Q9ULE6 | PALD1 | Paladin | Cytoplasm |
| Q8WX93 | PALLD | Palladin | Cytoplasm |
| O43252 | PAPSS1 | Bifunctional 3′-phosphoadenosine 5′-phosphosulfate | Cytoplasm |
| | | synthase 1 | |
| O95340 | PAPSS2 | 3′-phosphoadenosine 5′-phosphosulfate synthase 2 | Cytoplasm |
| Q96RG2 | PASK | PAS domain-containing serine/threonine-protein | Cytoplasm |
| | | kinase | |
| Q96RG2 | PASK | PAS domain-containing serine/threonine-protein | Cytoplasm |
| | | kinase | |
| Q8WV24 | PHLDA1 | pleckstrin homology-like domain, family A, member 1 | Cytoplasm |
| P14618 | PKM | Pyruvate kinase isozymes M1/M2 | Cytoplasm |
| P19174 | PLCG1 | 1-phosphatidylinositol 4,5-bisphosphate | Cytoplasm |
| | | phosphodiesterase gamma-1 | |
| P19174 | PLCG1 | 1-phosphatidylinositol 4,5-bisphosphate | Cytoplasm |
| | | phosphodiesterase gamma-1 | |
| O60664 | PLIN3 | perilipin 3 | Cytoplasm |
| O75688 | PPM1B | protein phosphatase, Mg2+/Mn2+ dependent, 1B | Cytoplasm |
| O15355 | PPM1G | protein phosphatase, Mg2+/Mn2+ dependent, 1G | Cytoplasm |
| O15355 | PPM1G | protein phosphatase, Mg2+/Mn2+ dependent, 1G | Cytoplasm |
| Q9Y570 | PPME1 | protein phosphatase methylesterase 1 | Cytoplasm |
| P30153 | PPP2R1A | Serine/threonine-protein phosphatase 2A 65 kDa | Cytoplasm |
| | | regulatory subunit A alpha isoform | |
| Q06830 | PRDX1 | peroxiredoxin 1 | Cytoplasm |
| Q9Y520 | PRRC2C | Protein PRRC2C | Cytoplasm |
| Q9UL46 | PSME2 | proteasome (prosome, macropain) activator subunit 2 | Cytoplasm |
| Q15185 | PTGES3 | prostaglandin E synthase 3 | Cytoplasm |
| Q05397 | PTK2 | Focal adhesion kinase 1 | Cytoplasm |
| Q06124 | PTPN11 | Tyrosine-protein phosphatase non-receptor type 11 Cytoplasm | |
| P11216 | PYGB | phosphorylase, glycogen; brain | Cytoplasm |
| P62820 | RAB1A | RAB1A, member RAS oncogene family | Cytoplasm |
| P15153 | RAC2 | ras-related C3 botulinum toxin substrate 2 (rho family, | Cytoplasm |
| | | small GTP binding protein Rac2) | |
| Q6VN20 | RANBP10 | RAN binding protein 10 | Cytoplasm |
| A6NED2 | RCCD1 | RCC1 domain containing 1 | Cytoplasm |
| Q6NUM9 | RETSAT | retinol saturase (all-trans-retinol 13,14-reductase) | Cytoplasm |
| Q6NUM9 | RETSAT | retinol saturase (all-trans-retinol 13,14-reductase) | Cytoplasm |
| Q6NUM9 | RETSAT | retinol saturase (all-trans-retinol 13,14-reductase) | Cytoplasm |
| O43353 | RIPK2 | Receptor-interacting serine/threonine-protein kinase 2 | Cytoplasm |
| P27635 | RPL10 | ribosomal protein L10 | Cytoplasm |
| P62913 | RPL11 | ribosomal protein L11 | Cytoplasm |
| P49207 | RPL34 | 60S ribosomal protein L34 | Cytoplasm |
| P32969 | RPL9 | ribosomal protein L9 | Cytoplasm |
| P62244 | RPS15A | ribosomal protein S15a | Cytoplasm |
| P62249 | RPS16 | 40S ribosomal protein S16 | Cytoplasm |
| P60866 | RPS20 | 40S ribosomal protein S20 | Cytoplasm |
| P46782 | RPS5 | 40S ribosomal protein S5 | Cytoplasm |
| Q9UBS0 | RPS6KB2 | ribosomal protein S6 kinase, 70 kDa, polypeptide 2 | Cytoplasm |
| P60468 | SEC61B | Protein transport protein Sec61 subunit beta | Cytoplasm |
| Q8NC51 | SERBP1 | Plasminogen activator inhibitor 1 RNA-binding | Cytoplasm |
| | | protein | |
| P31947 | SFN | stratifin | Cytoplasm |
| A1X283 | SH3PXD2B | SH3 and PX domains 2B | Cytoplasm |
| Q9H0W8 | SMG9 | smg-9 homolog, nonsense mediated mRNA decay | Cytoplasm |
| | | factor | |
| Q9C004 | SPRY4 | sprouty homolog 4 | Cytoplasm |
| P28290 | SSFA2 | Sperm-specific antigen 2 | Cytoplasm |
| P28290 | SSFA2 | Sperm-specific antigen 2 | Cytoplasm |
| O95793 | STAU1 | staufen, RNA binding protein, homolog 1 | Cytoplasm |
| P31948 | STIP1 | stress-induced-phosphoprotein 1 | Cytoplasm |
| O94804 | STK10 | serine/threonine kinase 10 | Cytoplasm |
| Q9UMX1 | SUFU | Suppressor of fused homolog | Cytoplasm |
| P37802 | TAGLN2 | Transgelin-2 | Cytoplasm |
| Q92844 | TANK | TRAF family member-associated NFKB activator | Cytoplasm |
| O60343 | TBC1D4 | TBC1 domain family member 4 | Cytoplasm |
| O60343 | TBC1D4 | TBC1 domain family member 4 | Cytoplasm |
| O60343 | TBC1D4 | TBC1 domain family, member 4 | Cytoplasm |
| Q13009 | TIAM1 | T-cell lymphoma invasion and metastasis 1 | Cytoplasm |
| P04183 | TK1 | thymidine kinase 1, soluble | Cytoplasm |
| O14545 | TRAFD1 | TRAF-type zinc finger domain containing 1 | Cytoplasm |
| Q12931 | TRAP1 | TNF receptor-associated protein 1 | Cytoplasm |
| Q6IBS0 | TWF2 | twinfilin, actin-binding protein, homolog 2 | Cytoplasm |
| P10599 | TXN | Thioredoxin | Cytoplasm |
| A0AVT1 | UBA6 | Ubiquitin-like modifier-activating enzyme 6 | Cytoplasm |
| Q9C0C9 | UBE2O | Ubiquitin-conjugating enzyme E2O | Cytoplasm |
| Q9C0C9 | UBE2O | ubiquitin-conjugating enzyme E2O | Cytoplasm |
| Q9C0C9 | UBE2O | ubiquitin-conjugating enzyme E2O | Cytoplasm |
| O94888 | UBXN7 | UBX domain-containing protein 7 | Cytoplasm |
| P26640 | VARS | valyl-tRNA synthetase | Cytoplasm |
| Q9NNW5 | WDR6 | WD repeat-containing protein 6 | Cytoplasm |
| Q8N5D0 | WDTC1 | WD and tetratricopeptide repeats 1 | Cytoplasm |
| P61981 | YWHAG | tyrosine 3-monooxygenase/tryptophan 5- | Cytoplasm |
| | | monooxygenase activation protein, gamma | |
| | | polypeptide | |
| Q7Z2W4 | ZC3HAV1 | zinc finger CCCH-type, antiviral 1 | Cytoplasm |
| Q7L5D6 | GET4 | golgi to ER traffic protein 4 homolog | ER |
| | | | membrane |
| P48449 | LSS | lanosterol synthase (2,3-oxidosqualene-lanosterol | ER |
| | | cyclase) | membrane |
| Q15005 | SPCS2 | signal peptidase complex subunit 2 homolog | ER |
| | | | membrane |
| O15270 | SPTLC2 | serine palmitoyltransferase, long chain base subunit 2 | ER |
| | | | membrane |
| P08240 | SRPR | signal recognition particle receptor | ER |
| | | | membrane |
| Q9P246 | STIM2 | stromal interaction molecule 2 | ER |
| | | | membrane |
| P61221 | ABCE1 | ATP-binding cassette sub-family E member 1 | mitochondria |
| P24752 | ACAT1 | Acetyl-CoA acetyltransferase 1 | mitochondria |
| P24752 | ACAT1 | Acetyl-CoA acetyltransferase 1 | mitochondria |
| P54886 | ALDH18A1 | Delta-1-pyrroline-5-carboxylate synthase | mitochondria |
| Q5TC12 | ATPAF1 | ATP synthase mitochondrial F1 complex assembly | mitochondria |
| | | factor 1 | |
| Q9Y696 | CLIC4 | chloride intracellular channel 4 | mitochondria |
| Q9BQ52 | ELAC2 | elaC homolog 2 | mitochondria |
| O95571 | ETHE1 | ethylmalonic encephalopathy 1 | mitochondria |
| P49327 | FASN | Fatty acid synthase | mitochondria |
| P49327 | FASN | Fatty acid synthase | mitochondria |
| P49327 | FASN | Fatty acid synthase | mitochondria |
| P49327 | FASN | Fatty acid synthase | mitochondria |
| P49327 | FASN | Fatty acid synthase | mitochondria |
| P49327 | FASN | Fatty acid synthase | mitochondria |
| Q9HAV7 | GRPEL1 | GrpE-like 1 | mitochondria |
| Q3SXM5 | HSDL1 | Inactive hydroxysteroid dehydrogenase-like protein 1 | mitochondria |
| Q6UB35 | MTHFD1L | methylenetetrahydrofolate dehydrogenase (NADP+ | mitochondria |
| | | dependent) 1-like | |
| Q9P0J1 | PDP1 | pyruvate dehyrogenase phosphatase catalytic subunit 1 | mitochondria |
| Q9H2U2 | PPA2 | pyrophosphatase (inorganic) 2 | mitochondria |
| Q9H2U2 | PPA2 | pyrophosphatase (inorganic) 2 | mitochondria |
| P49411 | TUFM | Elongation factor Tu | mitochondria |
| Q9H9F9 | ACTR5 | ARP5 actin-related protein 5 homolog | Nucleus |
| Q9H981 | ACTR8 | Actin-related protein 8 | Nucleus |
| Q96P47 | AGAP3 | Arf-GAP with GTPase, ANK repeat and PH domain- | Nucleus |
| | | containing protein 3 | |
| O95081 | AGFG2 | ArfGAP with FG repeats 2 | Nucleus |
| O00170 | AIP | aryl hydrocarbon receptor interacting protein | Nucleus |
| Q6NXT1 | ANKRD54 | Ankyrin repeat domain-containing protein 54 | Nucleus |
| Q66PJ3 | ARL6IP4 | ADP-ribosylation-like factor 6 interacting protein 4 | Nucleus |
| O60566 | BUB1B | Mitotic checkpoint serine/threonine-protein kinase | Nucleus |
| | | BUB1 beta | |
| Q9Y224 | C14orf166 | chromosome 14 open reading frame 166 | Nucleus |
| Q9NV56 | C20orf20 | MRG/MORF4L-binding protein | Nucleus |
| P22681 | CBL | Cas-Br-M (murine) ecotropic retroviral transforming | Nucleus |
| | | sequence | |
| Q9UK39 | CCRN4L | Nocturnin | Nucleus |
| O15446 | CD3EAP | DNA-directed RNA polymerase I subunit RPA34 | Nucleus |
| P23528 | CFL1 | cofilin 1 | Nucleus |
| Q13111 | CHAF1A | chromatin assembly factor 1, subunit A (p150) | Nucleus |
| O00299 | CLIC1 | chloride intracellular channel 1 | Nucleus |
| O95833 | CLIC3 | chloride intracellular channel 3 | Nucleus |
| Q16630 | CPSF6 | cleavage and polyadenylation specific factor 6, 68 kDa | Nucleus |
| P55060 | CSE1L | Exportin-2 | Nucleus |
| Q9H0L4 | CSTF2T | cleavage stimulation factor, 3′ pre-RNA, subunit 2, | Nucleus |
| | | 64 kDa, tau variant | |
| Q9Y5B0 | CTDP1 | RNA polymerase II subunit A C-terminal domain | Nucleus |
| | | phosphatase | |
| P17812 | CTPS | CTP synthase | Nucleus |
| P27707 | DCK | deoxycytidine kinase | Nucleus |
| Q13838 | DDX39B | DEAD (Asp-Glu-Ala-Asp) box polypeptide 39B | Nucleus |
| O76075 | DFFB | DNA fragmentation factor, 40 kDa, beta polypeptide | Nucleus |
| Q96DF8 | DGCR14 | Protein DGCR14 | Nucleus |
| O75190 | DNAJB6 | DnaJ (Hsp40) homolog, subfamily B, member 6 | Nucleus |
| Q9H410 | DSN1 | DSN1, MIND kinetochore complex component, | Nucleus |
| | | homolog | |
| Q6PJG2 | ELMSAN1 | ELM2 and SANT domain-containing protein 1 | Nucleus |
| Q6NXG1 | ESRP1 | epithelial splicing regulatory protein 1 | Nucleus |
| Q5RKV6 | EXOSC6 | exosome component 6 | Nucleus |
| Q13451 | FKBP5 | FK506 binding protein 5 | Nucleus |
| P85037 | FOXK1 | Forkhead box protein K1 | Nucleus |
| Q96I24 | FUBP3 | far upstream element (FUSE) binding protein 3 | Nucleus |
| Q96I24 | FUBP3 | far upstream element (FUSE) binding protein 3 | Nucleus |
| Q12789 | GTF3C1 | General transcription factor 3C polypeptide 1 | Nucleus |
| P31943 | HNRNPH1 | heterogeneous nuclear ribonucleoprotein H1 | Nucleus |
| P61978 | HNRNPK | heterogeneous nuclear ribonucleoprotein K | Nucleus |
| P14866 | HNRNPL | heterogeneous nuclear ribonucleoprotein L | Nucleus |
| P14866 | HNRNPL | heterogeneous nuclear ribonucleoprotein L | Nucleus |
| Q00839 | HNRNPU | heterogeneous nuclear ribonucleoprotein U | Nucleus |
| Q00613 | HSF1 | heat shock transcription factor 1 | Nucleus |
| Q96ST2 | IWS1 | IWS1 homolog | Nucleus |
| Q7LBC6 | KDM3B | lysine (K)-specific demethylase 3B | Nucleus |
| Q92945 | KHSRP | KH-type splicing regulatory protein | Nucleus |
| Q15004 | KIAA0101 | PCNA-associated factor | Nucleus |
| Q15004 | KIAA0101 | PCNA-associated factor | Nucleus |
| O60870 | KIN | KIN, antigenic determinant of recA protein homolog | Nucleus |
| Q9Y2U8 | LEMD3 | LEM domain containing 3 | Nucleus |
| P02545 | LMNA | lamin A/C | Nucleus |
| P25205 | MCM3 | DNA replication licensing factor MCM3 | Nucleus |
| P25205 | MCM3 | DNA replication licensing factor MCM3 | Nucleus |
| Q9BTE3 | MCMBP | minichromosome maintenance complex binding | Nucleus |
| | | protein | |
| Q9Y2X0 | MED16 | Mediator of RNA polymerase II transcription subunit | Nucleus |
| | | 16 | |
| Q9Y2X0 | MED16 | mediator complex subunit 16 | Nucleus |
| Q9NX70 | MED29 | mediator complex subunit 29 | Nucleus |
| P41227 | NAA10 | N(alpha)-acetyltransferase 10, NatA catalytic subunit | Nucleus |
| Q9BXJ9 | NAA15 | N(alpha)-acetyltransferase 15, NatA auxiliary subunit | Nucleus |
| Q15742 | NAB2 | NGFI-A binding protein 2 (EGR 1 binding protein 2) | Nucleus |
| Q15021 | NCAPD2 | non-SMC condensin I complex, subunit D2 | Nucleus |
| Q15021 | NCAPD2 | non-SMC condensin I complex, subunit D2 | Nucleus |
| Q00653 | NFKB2 | nuclear factor of kappa light polypeptide gene | Nucleus |
| | | enhancer in B-cells 2 (p49/p100) | |
| Q96IY1 | NSL1 | NSL1, MIND kinetochore complex component, | Nucleus |
| | | homolog | |
| Q9Y5A7 | NUB1 | negative regulator of ubiquitin-like proteins 1 | Nucleus |
| Q9Y5Y2 | NUBP2 | nucleotide binding protein 2 | Nucleus |
| P49790 | NUP153 | Nuclear pore complex protein Nup153 | Nucleus |
| P49790 | NUP153 | Nuclear pore complex protein Nup153 | Nucleus |
| Q9UKX7 | NUP50 | nucleoporin 50 kDa | Nucleus |
| Q15365 | PCBP1 | Poly(rC)-binding protein 1 | Nucleus |
| Q15365 | PCBP1 | Poly(rC)-binding protein 1 | Nucleus |
| Q53EL6 | PDCD4 | programmed cell death 4 | Nucleus |
| O00541 | PES1 | pescadillo homolog 1, containing BRCT domain | Nucleus |
| Q8IXK0 | PHC2 | Polyhomeotic-like protein 2 | Nucleus |
| Q96BK5 | PINX1 | PIN2/TERF1 interacting, telomerase inhibitor 1 | Nucleus |
| Q9Y2S7 | POLDIP2 | Polymerase delta-interacting protein 2 | Nucleus |
| Q9BY77 | POLDIP3 | polymerase (DNA-directed), delta interacting protein 3 | Nucleus |
| O15355 | PPM1G | protein phosphatase, Mg2+/Mn2+ dependent, 1G | Nucleus |
| Q9HCU5 | PREB | Prolactin regulatory element-binding protein | Nucleus |
| Q5VTL8 | PRPF38B | Pre-mRNA-splicing factor 38B | Nucleus |
| P61289 | PSME3 | Proteasome activator complex subunit 3 | Nucleus |
| O60942 | RNGTT | RNA guanylyltransferase and 5′-phosphatase | Nucleus |
| O43148 | RNMT | mRNA cap guanine-N7 methyltransferase | Nucleus |
| Q9H6T3 | RPAP3 | RNA polymerase II-associated protein 3 | Nucleus |
| Q9Y3B4 | SF3B14 | splicing factor 3B, 14 kDa subunit | Nucleus |
| O75643 | SNRNP200 | small nuclear ribonucleoprotein 200 kDa | Nucleus |
| O75391 | SPAG7 | sperm associated antigen 7 | Nucleus |
| P84103 | SRSF3 | serine/arginine-rich splicing factor 3 | Nucleus |
| Q9Y6J9 | TAF6L | TAF6-like RNA polymerase II p300/CBP-associated | Nucleus |
| | | factor (PCAF)-associated factor, 65 kDa | |
| Q9BQ70 | TCF25 | transcription factor 25 | Nucleus |
| P42166 | TMPO | Lamina-associated polypeptide 2, isoform alpha | Nucleus |
| P42166 | TMPO | Lamina-associated polypeptide 2, isoform alpha | Nucleus |
| Q9UPN9 | TRIM33 | tripartite motif containing 33 | Nucleus |
| O15042 | U2SURP | U2 snRNP-associated SURP motif-containing protein | Nucleus |
| Q05086 | UBE3A | ubiquitin protein ligase E3A | Nucleus |
| Q8IWV8 | UBR2 | ubiquitin protein ligase E3 component n-recognin 2 | Nucleus |
| Q96RL1 | UIMC1 | ubiquitin interaction motif containing 1 | Nucleus |
| O75717 | WDHD1 | WD repeat and HMG-box DNA-binding protein 1 | Nucleus |
| O75152 | ZC3H11A | zinc finger CCCH-type containing 11A | Nucleus |
| Q15973 | ZNF124 | Zinc finger protein 124 | Nucleus |
| Q5T7W0 | ZNF618 | Zinc finger protein 618 | Nucleus |
| P05023 | ATP1A1 | ATPase, Na+/K+ transporting, alpha 1 polypeptide | Plasma |
| | | | Membrane |
| Q02487 | DSC2 | desmocollin 2 | Plasma |
| | | | Membrane |
| P00533 | EGFR | Epidermal growth factor receptor | Plasma |
| | | | Membrane |
| P14923 | JUP | junction plakoglobin | Plasma |
| | | | Membrane |
| Q6N075 | MFSD5 | major facilitator superfamily domain containing 5 | Plasma |
| | | | Membrane |
| Q9Y4D7 | PLXND1 | plexin D1 | Plasma |
| | | | Membrane |
| Q6AI12 | ANKRD40 | Ankyrin repeat domain-containing protein 40 | unknown |
| Q8TCD1 | C18orf32 | Putative NF-kappa-B-activating protein 200 | unknown |
| Q9BQ61 | C19orf43 | chromosome 19 open reading frame 43 | Unknown |
| Q9H5V9 | CXorf56 | UPF0428 protein CXorf56 | Unknown |
| Q9Y4C2 | FAM115A | family with sequence similarity 115, member A | unknown |
| Q7Z309 | FAM122B | family with sequence similarity 122B | unknown |
| P0CB43 | FAM203A | family with sequence similarity 203, member A | unknown |
| Q8NCA5 | FAM98A | family with sequence similarity 98, member A | unknown |
| Q9HA64 | FN3KRP | fructosamine 3 kinase related protein | unknown |
| Q8WZA9 | IRGQ | immunity-related GTPase family, Q | unknown |
| Q96FF7 | LOC113230 | Uncharacterized protein LOC113230 | unknown |
| Q9Y546 | LRRC42 | leucine rich repeat containing 42 | unknown |
| Q86W50 | METTL16 | methyltransferase like 16 | unknown |
| A6NDG6 | PGP | phosphoglycolate phosphatase | unknown |
| Q8IYS1 | PM20D2 | peptidase M20 domain containing 2 | unknown |
| Q8IXT5 | RBM12B | RNA binding motif protein 12B | unknown |
| A3KN83 | SBNO1 | Protein strawberry notch homolog 1 | unknown |
| O60232 | SSSCA1 | Sjoegren syndrome/scleroderma autoantigen 1 | unknown |
| Q66K14 | TBC1D9B | TBC1 domain family member 9B | unknown |
| Q9NXH9 | TRMT1 | TRM1 tRNA methyltransferase 1 homolog | unknown |
| Q9NUE0 | ZDHHC18 | zinc finger, DHHC-type containing 18 | unknown |

### Example 2—Thioredoxin Reductase 1 and 2 are Enzymes for the Cytotoxic Activity of Iniparib

Materials

All cell lines were purchased from the ATCC cell biology collection. Cell culture reagents were purchased from LifeTechnologies. All regular chemicals or reagents were obtained from Sigma-Aldrich Chemicals, unless otherwise specified. Iniparib and its biotin-derivative tool compound were synthesized and purified as described in Example 1. Wild type TrxR purified from rat liver was from Sigma-Aldrich Chemicals. Human recombinant TrxR1 lacking the two C-term amino acids Sec-Glu (Asec TrxR1) was from Abnova. Streptavidin-HRP was purchased from GE Healthcare. Mouse monoclonal antibodies directed against TrxR1 (clone 5A5), TrxR2 (clone 25B3) and Trx (clone A5) were from Santa Cruz Biotechnology. Rabbit polyclonal antibodies against phospho-JNK, phospho-p38MAPK and cleaved-PARP were from Cell Signaling Technology.

Cell Lines Culture

Human cancer cell lines HCT116 (colorectal carcinoma) and MDA-MB-453 (breast metastatic carcinoma) were cultured in DMEM medium supplemented either with 10% fetal bovine serum (MDA-MB-453) or with 10% decomplemented fetal bovine serum (HCT116), 2 mM glutamine, 1 mM sodium pyruvate and 10 μg/ml ciprofloxacine (Euromedex) in a humidified 5% CO₂ atmosphere at 37° C.

Cytotoxicity Assay

Cell viability following drug treatments was assessed using WST-1 cell proliferation assay (Roche). Briefly, cells were plated into 96-well plates with 8,000 (HCT116) or 20,000 (MDA-MB-453) cells per well and allowed to attach overnight. Cells were then preincubated for 5 h in the presence or absence of 1 mM BSO. Afterwards cells were treated with Iniparib, its metabolites or their vehicle, 1% DMSO, and cell viability was measured 48 h after by addition of WST-1 reagent. After 3 h of incubation at 37° C., the amount of formazan dye was quantified by measuring the optical density at 450 nm with a scanning multiwell spectrophotometer (PerkinElmer).

Cell Treatments and Lysis

Cell incubations with Iniparib, Iniparib-biotin, Auranofin, Staurosporine or their vehicle (1% DMSO) were performed in serum-containing conditioned medium. Afterwards cells were solubilized in ice-cold octyl-glucoside buffer (1.5% octyl-glucoside, 150 mM NaCl, 25 mM Tris-HCl, pH 7.5) supplemented with protease and phosphatase inhibitors (Pierce, Thermo scientific). After 2 h at 4° C., lysates were clarified by centrifugation and protein amounts were measured using the BCA assay (Pierce, Thermo scientific).

TrxR Activity

TrxR activity was measured by the reduction of 5, 5′-dithiobis-2-nitrobenzoic acid (DTNB) according to the manufacturer's instructions (TrxR assay kit, Sigma). Briefly, all incubations were performed at 37° C. in 96-well microplates in 0.1 M potassium phosphate (pH 7.4), 10 mM EDTA and 240 μM NADPH. TrxR activity was measured by recording the initial increase in A₄₁₂ during the first 10 min upon addition of 3 mM DTNB with a scanning multiwell spectrophotometer (Molecular devices). Endogenous TrxR activity was determined using clarified octyl-glucoside cell lysates (50 and 75 μg proteins for HCT116 and MDA-MB-453 cells, respectively) and in vitro studies were performed with 36 pmol of purified rat liver TrxR.

SDS-PAGE and Western Blot Analysis

Equal amounts of proteins were resolved by SDS-PAGE under either non-reducing or reducing (5% 2-mercaptoethanol, 20 min at 60° C.) conditions, using 4-20% gels (Novex, Invitrogen), then subjected to semi-dry electrophoretic transfer onto nitrocellulose membranes.

After membrane blotting, detection of reactive bands was performed by enhanced chemiluminescence (West DURA substrate, Pierce, Thermo scientific). Bands were quantified using a GS-800 calibrated densitometer (Bio-Rad), and the Quantity One software (Bio-Rad) was used to set a background region and give a quantitative value from which the background was subtracted.

TrxR1 and TrxR2 Immunoprecipitation

Clarified octyl-glucoside lysates (12 mg proteins) were combined with anti-TrxR1 or anti-TrxR2 monoclonal antibody coupled to protein G plus agarose (Santa Cruz Biotechnology) and mixed overnight at 4° C. Beads were recovered by centrifugation for 2 min at 1,000×g and extensively washed in octyl-glucoside lysis buffer. Precipitated complexes were eluted by incubating the beads for 20 min at 60° C. in SDS sample buffer. The amount of immunoprecipitated TrxR1 and TrxR2 was systematically checked by blots anti-TrxR1 and anti-TrxR2, respectively. With the antibodies selected, immunoprecipitation yield was similar (over 75%) for TrxR1 and TrxR2. For in vitro Iniparib-biotin TrxR adduct formation, immunoprecipitated TrxR1 and TrxR2 complexes were incubated at 37° C. in 50 mM Tris-HCl pH 7.5 with 30 μM Iniparib-biotin in the presence or the absence of 200 μM NADPH and 5 μM FAD prior to elution from agarose beads as described above.

Characterization of TrxR-Iniparib Adducts

After separation by SDS-PAGE under reducing conditions and PageBlue® protein staining (Thermo scientific), bands of interest were excised, reduced with DTT, alkylated with iodoacetamide and in-gel digested with trypsin (Promega), according to the method of Shevchenko et al. (24). Peptides were extracted with 50 mM ammonium bicarbonate and 50% acetonitrile in 0.2% formic acid and analyzed by Nano LC-MS/MS after partial evaporation in a speed-vac concentrator. LC-MS/MS experiments were performed on an NanoAcquity UPLC (Waters) coupled to a hybrid LTQ Orbitrap Velos mass spectrometer (Thermo Fisher Scientific) equipped with a nanoelectrospray source. Tryptic digests were loaded onto a nanoAcquity UPLC Trap column (Symmetry C18, 5 μm, 180 μm×20 mm, Waters) and washed with 0.2% FA at 20 μl/min for 5 min. Peptides were then eluted on a C18 reverse-phase nanoAcquity column (BEH130 C18, 1.7 μm, 75 μm×250 mm, Waters) with a linear gradient of 7-30% solvent B (H₂O/CH₃CN/HCOOH, 10:90:0.2, by vol.) for 85 min, 30-90% solvent B for 10 min, and 90% solvent B for 5 min, at a flow rate of 250 nL/min. The mass spectrometer was operated in the data-dependent mode to automatically switch between MS and MS/MS acquisition. Survey full scan MS spectra (m/z 300-1,600) were acquired in the Orbitrap with a resolution of 60,000 at m/z 400. The AGC was set to 1×10⁶ with a maximum injection time of 100 ms. The most intense ions (up to 20) were then isolated for fragmentation in the LTQ linear ion trap with normalized collision energy of 28% at the default activation q of 0.25 with an AGC of 5×10³ and a maximum injection time of 200 ms. The dynamic exclusion time window was set to 80 s. LC-MS/MS data, acquired using the Xcalibur software (Thermo-Fisher Scientific), were processed using a homemade Visual Basic program software developed using XRawfile libraries (distributed by Thermo-Fisher Scientific) to generate a MS/MS peak list (MGF file) which will be used for database searching. This MGF file contained the exact parent mass and the retention time (RT) associated with each MS/MS spectrum. The exact parent mass is the 12C isotope ion mass of the most intense isotopic pattern detected on the high resolution Orbitrap MS parallel scan and included in the MS/MS selection window. The RT is issued from the LTQ-MS/MS scan. Database searches were done using our internal MASCOT server (version 2.1, matrix Science) using the SwissProt human database. The search parameters used for post-translational modifications were dynamic modification of +57.02146 Da (carbamidomethylation), of +164.02164 Da (Iniparib minus I adduct) or +433.14199 Da (Iniparib-biotin minus I adduct) on cysteine residues, of +15.99491 Da on methionine residues (oxidation), of +42.010565 Da on protein N-terminal residues (N-terminal acetylation) and −17.026549 Da on N-terminal glutamine residues (N-PyroGlu). The precursor mass tolerance was set to 5 ppm and the fragment ion tolerance was set to 0.5 Da. The number of missed cleavage sites for trypsin was set to 3. Mascot result files (“.dat” files) were imported into Scaffold software. Scaffold (version 3.4.5, Proteome software Inc., Portland, Oreg.) was used to validate MS/MS based peptide and protein identifications. Queries were also used for XTandem parallel Database Search. The compiled results of both database searches were exported.

Electron Paramagnetic Resonance

EPR experiments were carried out at 21° C. with an Elexsys 540 X-band spectrometer (Bruker Biospin; Silberstreifen, Germany) controlled by the Xepr software and equipped with an ER 4103TMS resonating cavity. The instrument settings were as follows: microwave frequency, 9.81 GHz; modulation frequency, 100 kHz; microwave power, 10 mW; modulation amplitude, 0.2 mT; receiver gain, 60 dB; time constant, 40.96 ms; conversion time, 41.08 ms; data points, 1024; scan time, 42.07 s; scan width, 15 mT. Computer simulations of the spectra were performed using the program written by Rockenbauer and Korecz (25). Purified rat liver TrxR (0.5 μM) was incubated in a micro tube for 1.5 h in the presence of 50 mM 5-dietbylphosphono-5-methyl-1-pyrroline N-oxide (DEPMPO), 500 μM NADPH, and 100 μM Iniparib in 100 μL Tris buffer (50 mM, pH 7.5) containing 1 mM diethylene triamine pentaacetic acid (DTPA). Then the reaction mixture was transferred by aspiration into a gas permeable PTFE tubing (Extruded Sub-Lite-Wall®, inside diameter: 0.635 mm, wall thickness: 0.051 mm, Zeus Industrial Products Ltd., Ireland). The tubing was folded twice in a W-shape and inserted into a 4-mm EPR quartz tube for EPR analysis. For control experiments, either TrxR or NADPH was omitted or Iniparib was replaced by its vehicule (1% DMSO). To confirm the involvement of superoxide in the appearance of the EPR signal, 100 units of superoxide dismutase (SOD) were added to the incubation prior to TrxR addition. For anaerobic experiments, the reaction mixture was immediately transferred into the gas permeable tubing and nitrogen gas was flushed through a septum cap in the sealed EPR quartz tube during the whole experiment. Similar EPR experiments were repeated using either ΔSec-TrxR1 or Iniparib-modified TrxR (prepared as described below) instead of TrxR. Additional experiments were performed with the same protocol on TrxR and ΔSec-TrxR1 that had been preincubated for 1, 2 or 4 h with auranofin at 4 μM. “Iniparib-sec compromised” form of TrxR was prepared as follows: purified rat liver TrxR (0.5 μM) was incubated for 3.5 h in 50 mM Tris-HCl pH 7.5, 200 μM NADPH and 5 μM FAD in the presence of 100 μM Iniparib or its vehicle. Thereafter, Iniparib-modified TrxR was ultra-filtrated (Amicon Ultracel®-10K membrane) and extensively washed to eliminate NADPH, FAD and Iniparib.

Inhibition of Human TrxR Activity is an Early Event in Cells Treated with Iniparib

In some instances, the redox status plays a role in the cytotoxicity of Iniparib. In some cases following bioactivation, Iniparib forms adducts with reactive thiol groups in proteins. TrxR is an enzyme that contributes to cell survival in an oxidative environment of tumor cells, and contains two tandems of highly redox-active residues, a Cys210-Cys214 and a Cys496-Sec497 in the N-terminal and C-terminal domains of the protein, respectively. In some instances, TrxR is a target for several cytotoxics, and based on the structural similarity between Iniparib and some other TrxR-inhibitors such as 1-chloro-2,4-dinitro chlorobenzene (DNCB).

Two cell lines were selected to investigate the early events leading to cell death following exposure to the drug: a colon cancer cell line (HCT116) and a breast adenocarcinoma cell line (MDA-MB-453). In HCT116 cells marginal cytotoxicity is detected after 24 h with concentrations of Iniparib up to 100 M, but GSH depletion rendered these cells very sensitive to the compound (IC₅₀=8.2 μM), and in MDA-MB-453 Iniparib is cytotoxic in the absence of BSO (IC₅₀=85 μM), and GSH depletion increased the cytotoxicity (IC₅₀=16.2 μM).

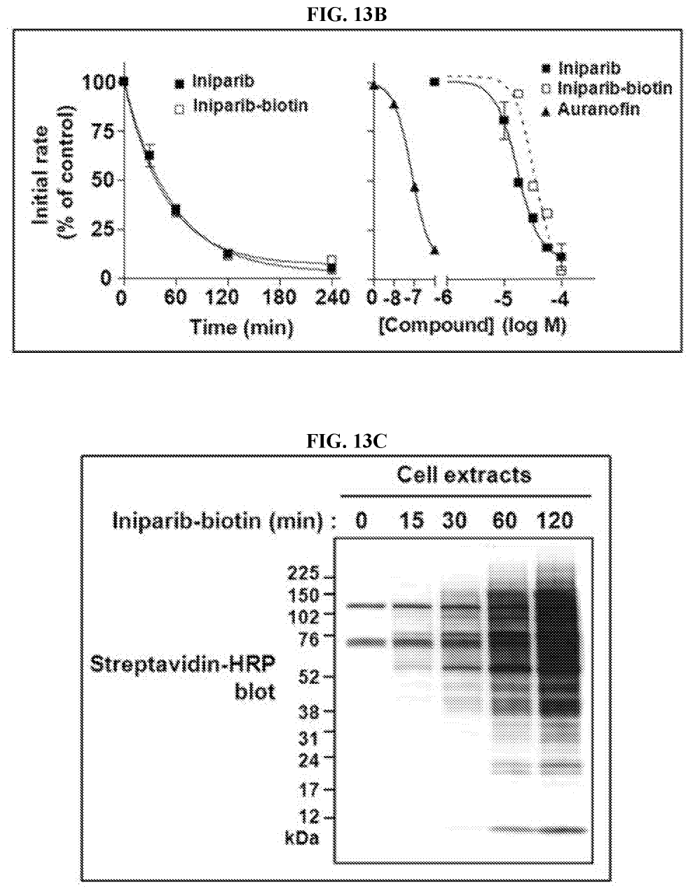

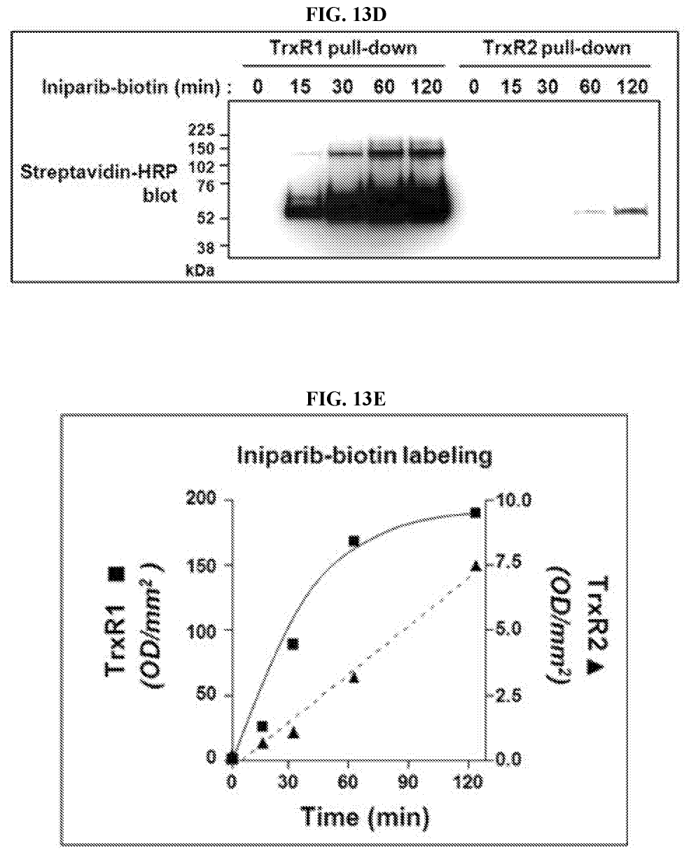

The time-dependence of fixed-dose exposure to achieve full cytotoxicity was studied. As illustrated in FIG. 13A, a transitory exposure of HCT116 cells to 100 μM Iniparib for 2 to 4 h was sufficient to have a cytotoxic effect similar to the one observed when the cells were incubated continuously with the drug for 24 h. In some cases, GSH-depleted cells incubated with Iniparib for 2 to 4 h were still adherent and did not show obvious signs of toxicity immediately after these transitory exposures. Also, the activity was dependent on the redox status of the cell since cells not depleted of GSH were much less sensitive to the drug (IC₅₀>>100 μM). Iniparib-biotin showed similar cytotoxic profile as Iniparib in the cell-based test.

TrxR activity in extracts from HCT116 cells treated with Iniparib were measured using an assay based on the reduction of DTNB. As shown in FIG. 13B (left panel), 100 μM of Iniparib, as well as Iniparib-biotin, inhibited in a time-dependent manner more than 90% of the reductase activity in the first 2 h of incubation and near 100% at 4h. Auranofin, a known inhibitor of TrxR1, at a concentration of 1 μM and under the same assay conditions, inhibited almost completely TrxR activity immediately after adding the compound to the cells (results not shown) as previously described (21). For the three compounds the inhibition was dose-dependent and the IC₅₀-values were estimated to 0.07, 17 and 33 μM for auranofin, Iniparib and Iniparib-biotin, respectively (FIG. 13B, right panel).

FIG. 13C shows the time dependence of proteins' modification by bioactivated Iniparib-biotin in HCT116 cells depleted of GSH and exposed to 100 μM of Iniparib-biotin for up to 2 h. The adducts formed were irreversible under reducing conditions. Besides, in HCT116 cells not depleted of GSH, protein labeling by Iniparib-biotin appeared to a much lesser extent, which correlated with the low cytotoxicity of the compound in these cells not pre-treated with BSO.

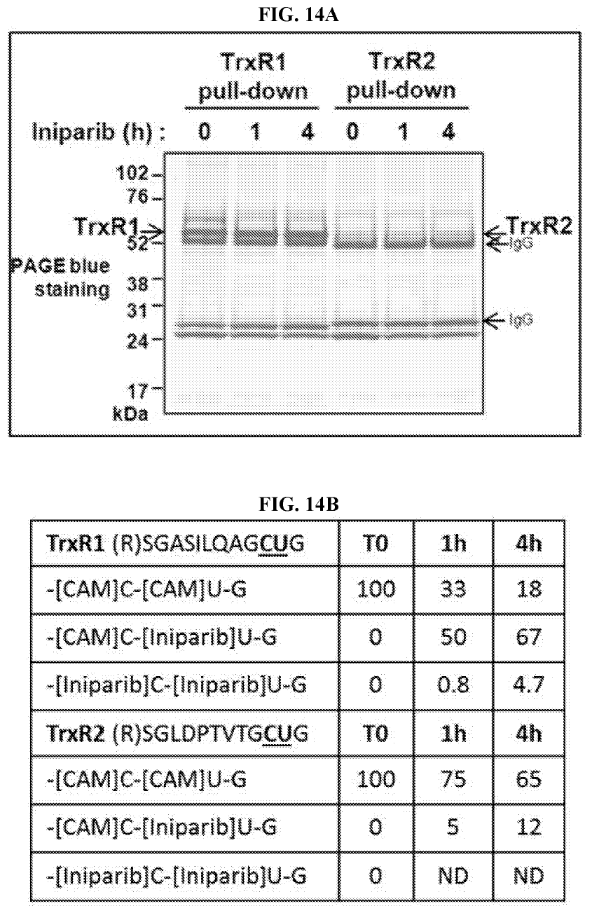

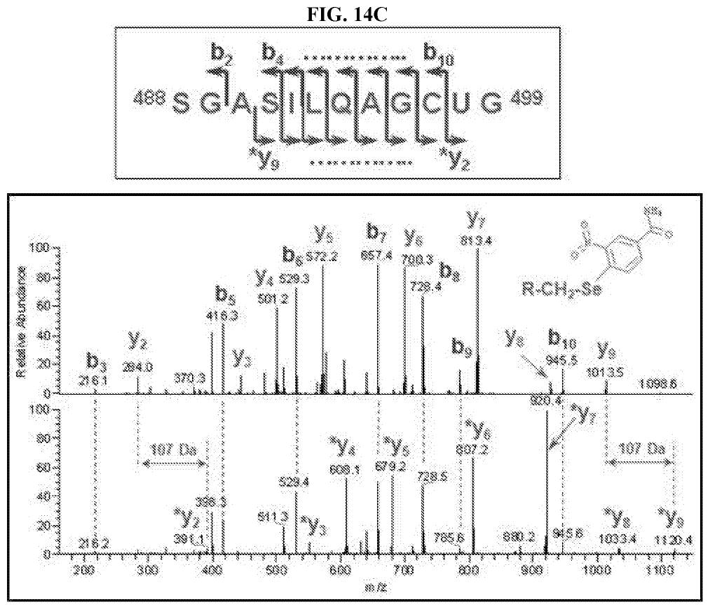

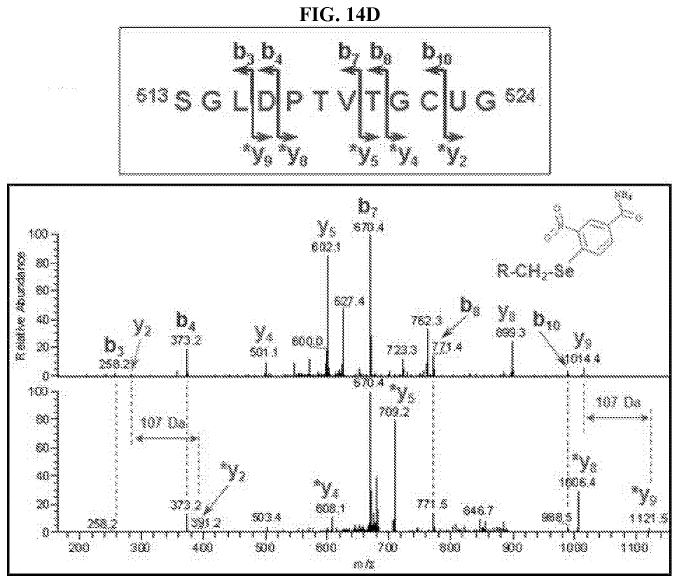

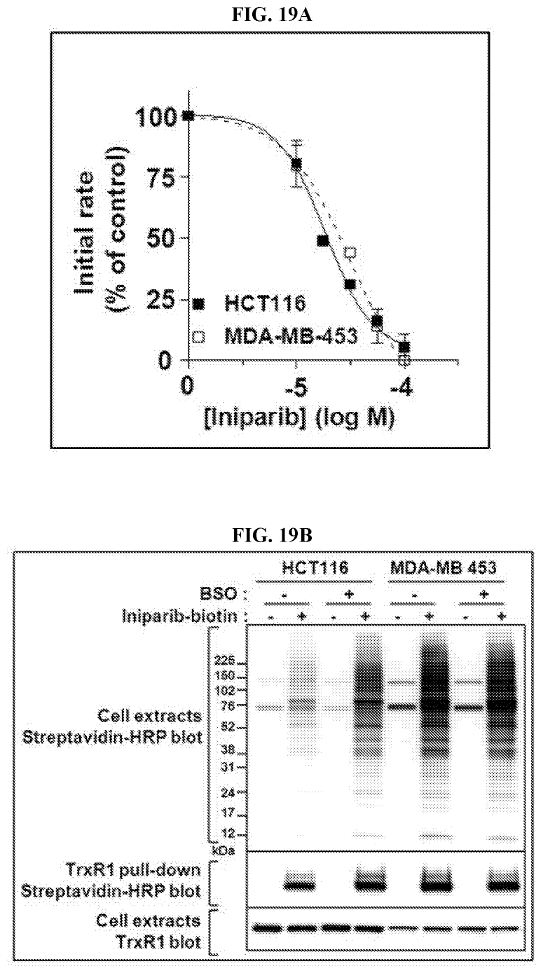

Next, HCT116 cell extracts analyzed in FIG. 13C were processed for either TrxR1 or TrxR2 pull-down. As revealed in FIG. 13D, both TrxR1 and TrxR2 were targeted by Iniparib-biotin and the modification of the two enzymes was time dependent. Interestingly, the results point to a much earlier labeling of cytosolic TrxR1 than mitochondrial TrxR2 by bioactivated Iniparib (FIG. 13E). Also it should be noted that the amount of TrxR1 was at least ten time higher than that of TrxR2 in these cells (FIG. 14A, in-gel protein staining) as described for other cell lines (27,28). TrxRs' modification by Iniparib resulting in reductase activity inhibition was found in all the cell lines studied and in particular in MDA-MB-453 cells without GSH depletion (FIG. 19).

Mass Spectrometry Shows that TrxR Selenocysteine is the Main Residue Targeted by Iniparib

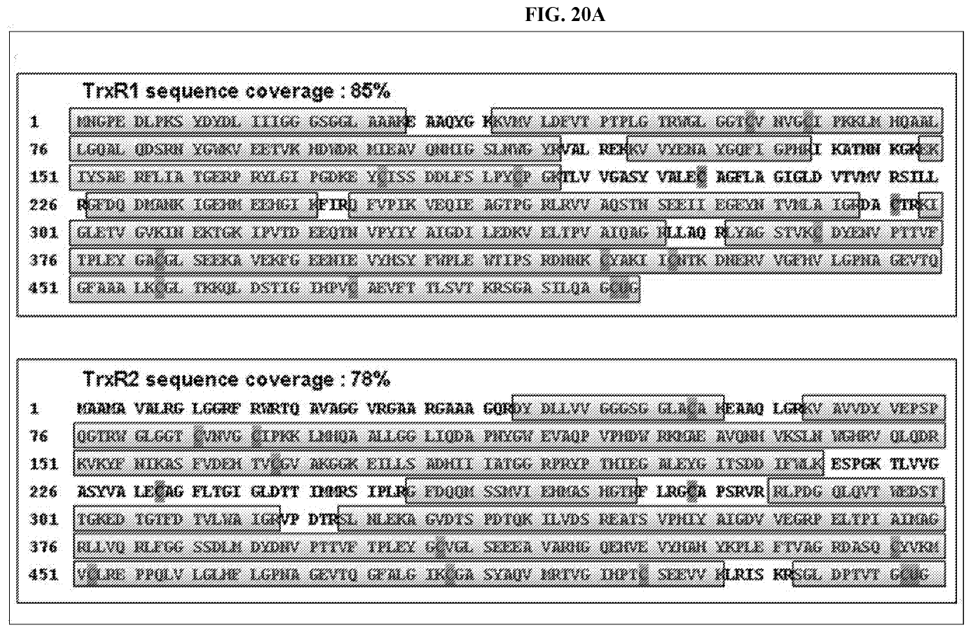

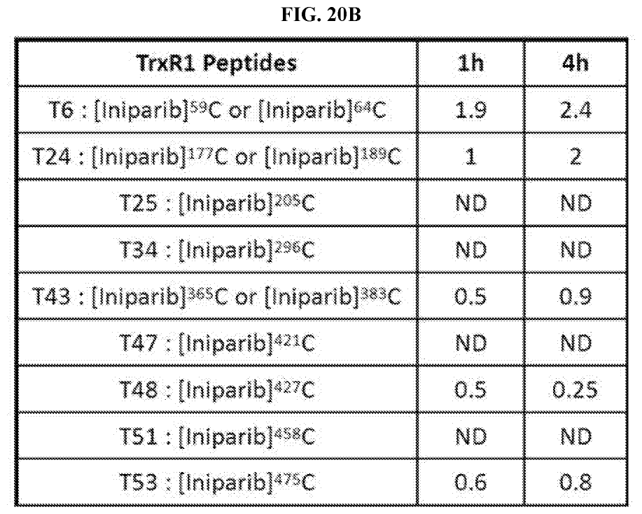

To investigate the nature of the adducts resulting from the interaction of the drug and the proteins, immunopurified TrxR1 and TrxR2 from GSH-depleted HCT116 cells treated or not with Iniparib for either 1 h or 4 h were separated by SDS-PAGE, and the ˜55 kDa bands (FIG. 14A) were cut, reduced, carbamidomethylated, and digested with trypsin as described in Example 1. The resulting tryptic peptides were analyzed by LC-MS/MS. The peptides analysis of the band corresponding to the control-cells pull-down performed with the anti-TrxR1 antibody allowed the clear identification of TrxR1 with a coverage of more than 85% of the sequence (FIG. 20). Similarly, the analysis of the band from the pull-down with the anti-TrxR2 antibody allowed the identification of TrxR2 with similar sequence coverage (FIG. 20). Quantification of peptides' intensity by mass spectrometry confirmed that TrxR1 expression level was at least ten-fold higher than that of TrxR2.

Analysis of the TrxR1 pull-down following 1 h and 4 h exposition of the cells to Iniparib resulted in very similar peptide identification as the control experiments. However a qualitative and quantitative comparison showed that several peptides displayed a shift in mass of +164 Da as expected from an adduct resulting from a nucleophilic aromatic substitution by bio activated Iniparib, where a thiol group substitutes the iodine in Iniparib. The modified peptide corresponded to the C-terminal tryptic peptide ⁴⁸⁸SGASLQAGCUG⁴⁹⁹ (FIG. 14C). MS/MS spectrum of this modified peptide allowed determining that the modification by Iniparib occurred selectively on the Sec residue as, from y₂, all y-ion series was shifted by +107 Da as compared with carbamidomethylated peptide in control sample (FIG. 14C). 50% of the total TrxR1 was modified on this residue after 1 h of incubation of the cells with Iniparib, and Iniparib-Sec modification affected 67% of the enzyme after 4 h. A low amount of TrxR1 was modified on both the two adjacent Cys and Sec residues (0.8 and 4.7% after 1 h and 4 h, respectively), relative to the Sec residue of the peptide ⁴⁸⁸SGASILQAGCUG⁴⁹⁹ (FIG. 14B). Further analysis showed that 5 other peptides were also modified on Cys residues which altogether represented after 4 h of incubation with Iniparib less than 7% of the total protein (FIG. 20).

Analysis of the proteins from the pull-down with anti-TrxR2 antibody led to qualitative similar results. The main modified peptide on TrxR2 was also the C-terminal one (⁵¹³SGLDPTVTGCUG⁵²⁴) with the main form of Iniparib adduct on the Sec residue (FIG. 14D). However, the quantification of modified peptides (FIG. 14B) indicated that TrxR2 was modified at a slower pace with respect to TrxR1. 5% of total TrxR2 showed one adduct of Iniparib on the Sec residue following 1 h of incubation of the cells with the prodrug, and 12% after 4 h. We were unable to detect the corresponding peptide with both Cys and Sec residues modified by Iniparib. Moreover, one additional peptide of TrxR2 was identified which was modified by Iniparib on a cysteine residue (modification amount inferior to 1% after 4 h of treatment). These results are in line with the slower labeling of TrxR2 when compared with TrxR1 shown in FIG. 13D.

Iniparib is Reduced and Activated by TrxR in a NADPH Dependent Manner and the Activated Form Inhibits the Enzyme's Reductase Activity

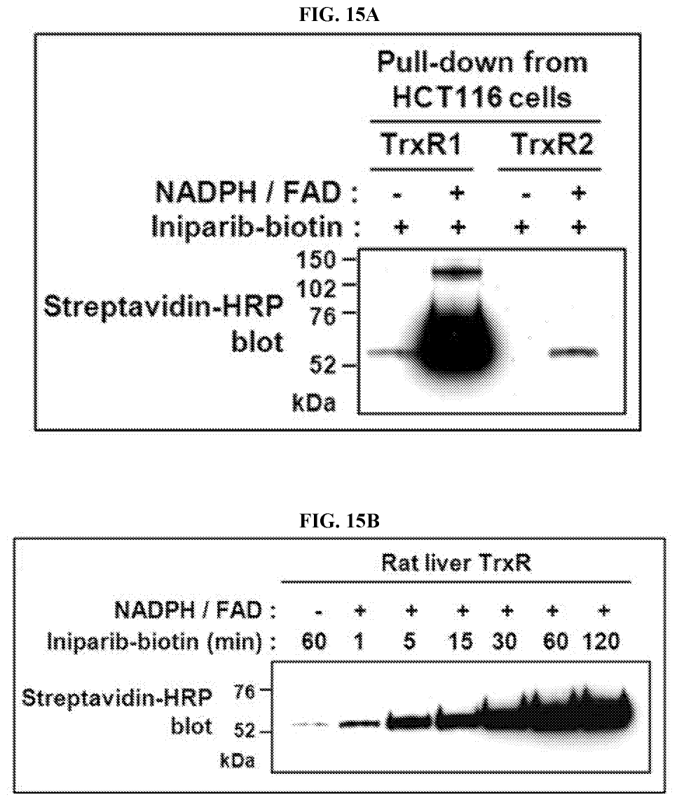

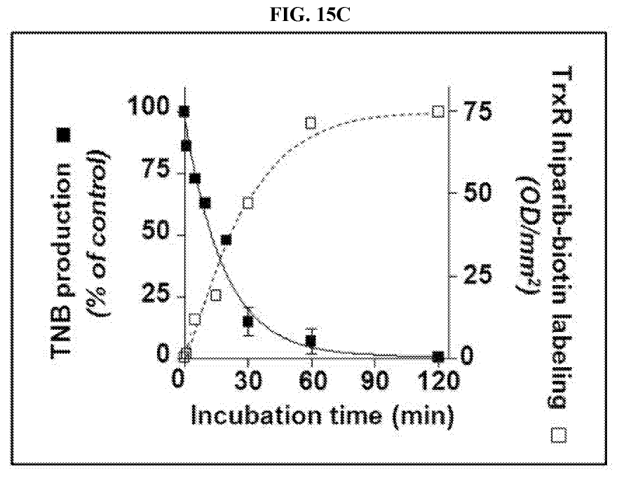

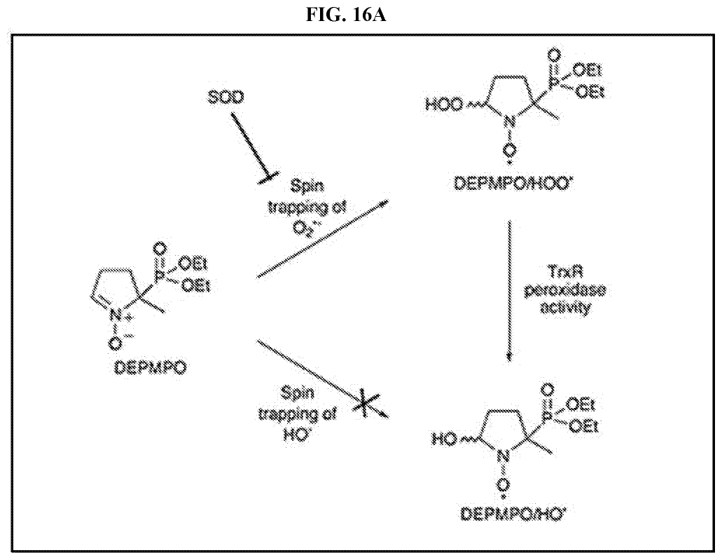

Because TrxR had been described as a reductase capable of either one- or two-electron reduction, it was investigated if TrxR could not only be a target for Iniparib, but also if it could reduce and activate the prodrug. In a first set of experiments, TrxR1 and TrxR2 from HCT 116 cells were immunoprecipitated and then the immunoisolated proteins were incubated with Iniparib-biotin in the presence or absence of NADPH. As illustrated in FIG. 15A, in the absence of NADPH almost no covalent labeling of TrxR1 was detected indicating that Iniparib as a prodrug is ineffective in forming adducts with free thiol groups of proteins. By contrast, when NADPH was provided as a source of electrons, Iniparib was able to form adducts with TrxR1 to a large extent, strongly suggesting that TrxR could bioactivate the prodrug. Similar results were observed with TrxR2.

This result was extended and confirmed using purified rat liver TrxR. As illustrated in FIG. 15B, as with TrxRs immuno-purified from the human HCT116 cells, the purified rat TrxR was able to bioactivate and to be covalently modified by Iniparib-biotin. And, when measured in parallel, the adduct formation was associated with the inhibition of the reductase activity of the enzyme. As illustrated in FIG. 15C, purified rat TrxR1 incubated with Iniparib in the presence of NADPH showed a time-dependent inhibition of its reductase activity, that followed closely the level of adduct formation. The IC₅₀-value for the purified rat TrxR was calculated to be 25 μM, in close agreement with that estimated for the human enzyme cells.

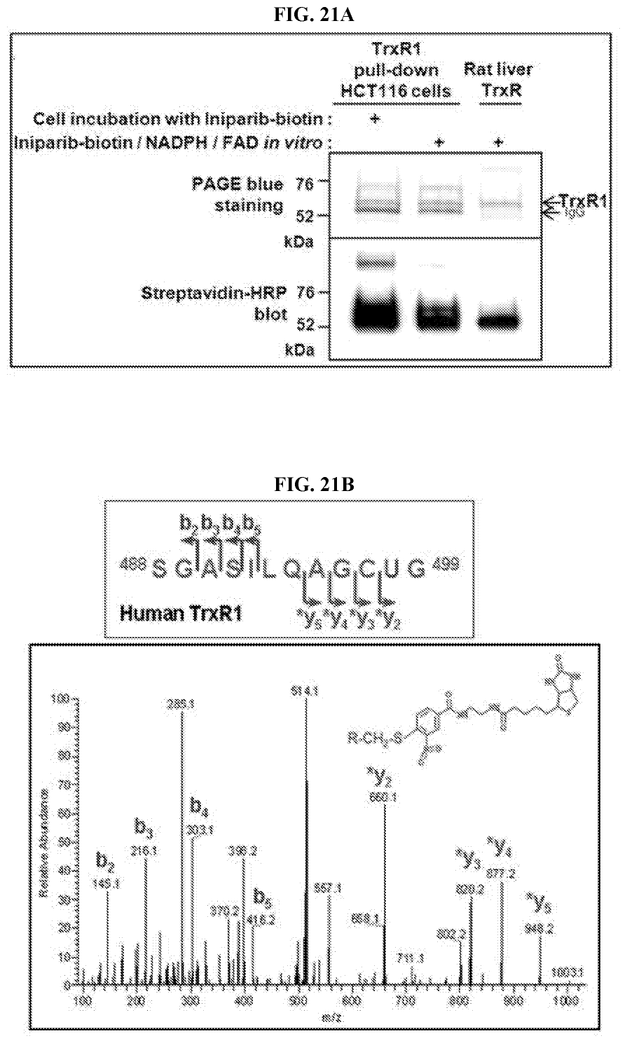

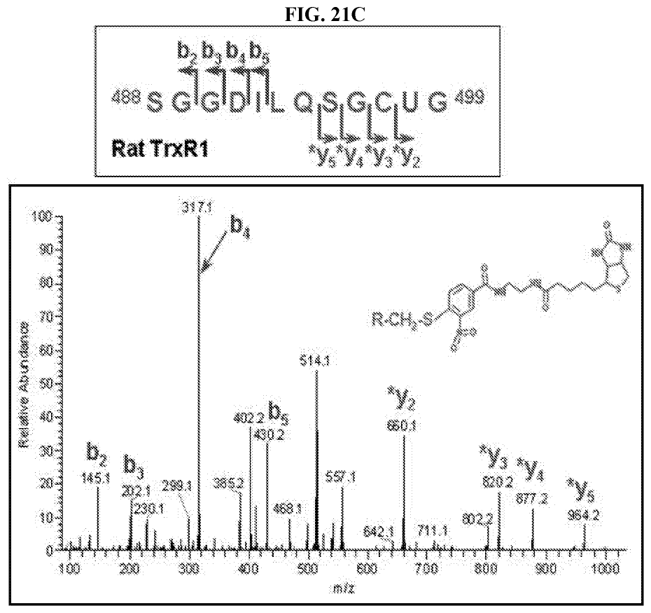

Mass spectrometry analysis of isolated TrxR modified in vitro with Iniparib-biotin in the presence of NADPH (FIG. 21) showed that the C-terminal selenocysteine is the main modified residue as demonstrated with bioactivated Iniparib in cells.

EPR Experiments Demonstrate that the Pro-Oxidant NADPH Oxidase Activity of TrxR is not Inhibited

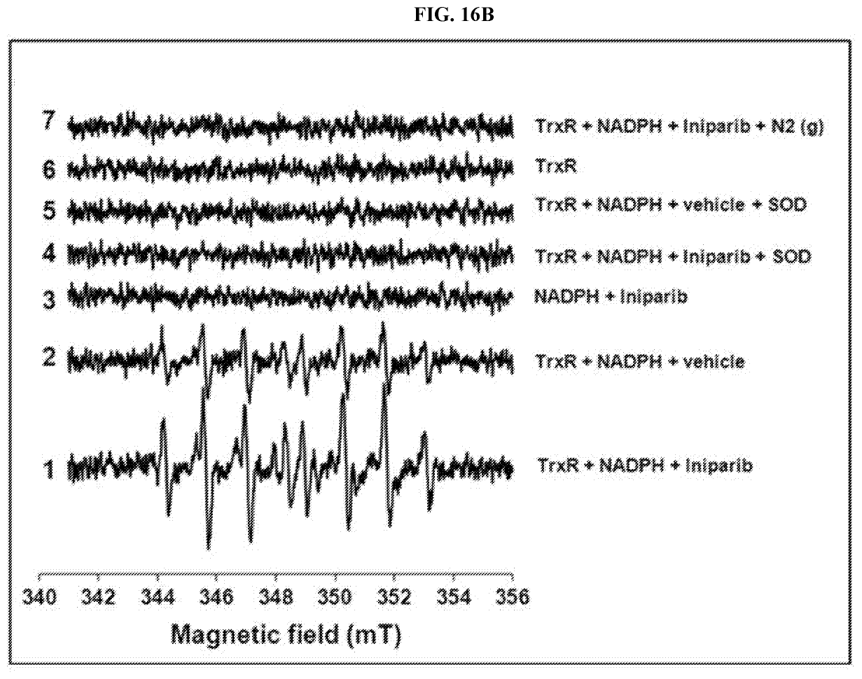

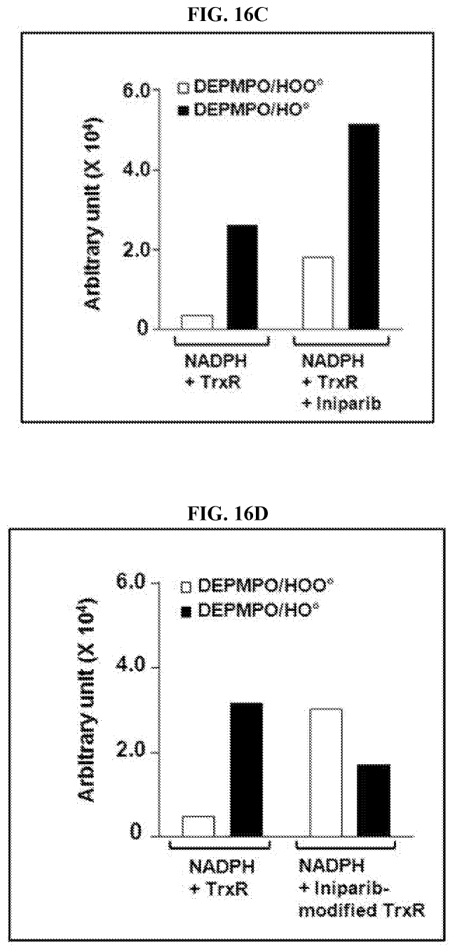

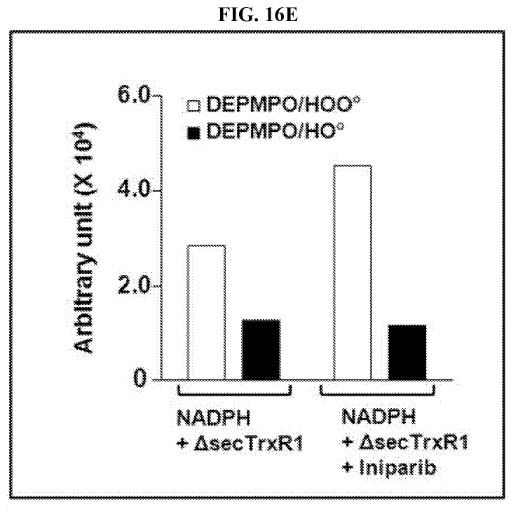

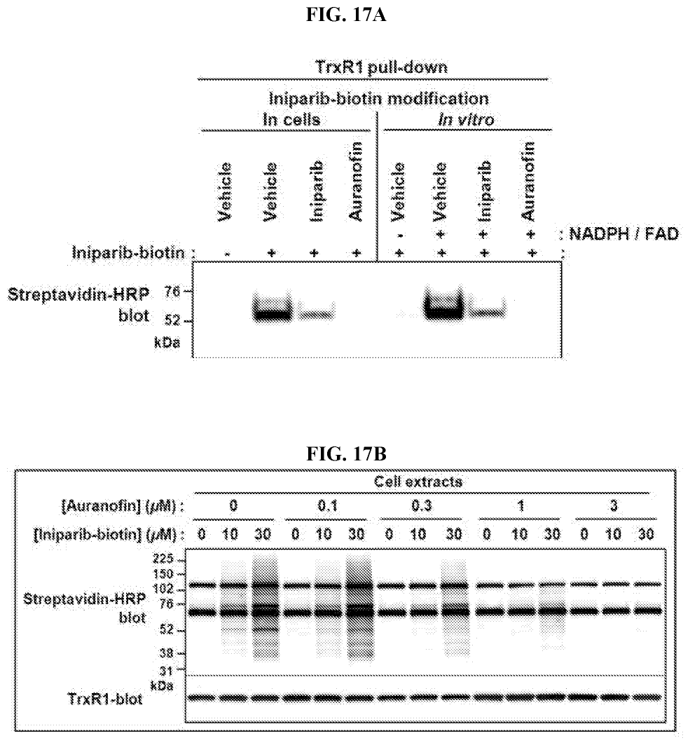

It was previously described that in addition to its reductase activity, TrxR also has pro-oxidant NADPH oxidase activity independent of the cysteine and selenocysteine C-terminal redox site. A Sec-dependent peroxidase activity was described by the same authors in their EPR experiments with DEPMPO, a spin trap for oxygen-centered radicals. This activity was evidenced by the ability of TrxR to reduce the peroxide function of the superoxide adduct DEPMPO/HO^{•}, to an alcohol function, yielding a structure equivalent to that formed by trapping of hydroxyl radical, DEPMPO/HO^{•} (see FIG. 16A). As a consequence, the sum of both DEPMPO adducts reflects the level of pro-oxidant NADPH oxidase activity of TrxR, while the ratio of DEPMPO/HO^{•} versus DEPMPO/HOO^{•} reflects the level of peroxidase activity of TrxR. To evaluate the possible effects of Iniparib on these activities, we performed EPR experiments with the spin trap DEPMPO to follow the generation of superoxide radical by TrxR in the absence of a substrate for the reductase activity and under aerobic conditions. As illustrated in FIG. 16B, the DEPMPO/HO• adduct was predominant with respect to DEPMPO/HOO•, and superoxide dismutase (SOD) eliminated both signals. They show that only spin trapping of superoxide occurs and that DEPMPO/HO^{•} results from the reduction of DEPMPO/HOO^{•} by TrxR peroxidase activity. Also both signals were O₂ dependent since under anaerobic conditions were not detected. Addition of Iniparib increased both signals, it increased five-fold the DEPMPO/HOO^{•} and doubled the DEPMPO/HO• signal suggesting an induction of the NADPH oxidase activity and a partial inhibition of the peroxidase activity (FIG. 16C). In another set of experiments, it was evaluated whether the Iniparib-induced variations in DEPMPO adduct signals resulted from the increased NADPH oxidase activity and the partially inhibited peroxidase activity. An Iniparib-modified form of TrxR was used, which is devoid of any reductase activity. EPR analysis show that the DEPMPO/HOO• signal was still increased with this Iniparib-modified form of TrxR as compared with the control TrxR, but the DEPMPO/HO• signal was reduced with respect to the non-modified enzyme (FIG. 16D). The EPR experiments was also performed using a TrxR1 devoid of the Sec residue, □Sec-TrxR1. In control experiments □Sec-TrxR1 showed a clear DEPMPO/HOO• signal, more intense than the one for DEPMPO/HO•. Addition of Iniparib in the absence of auranofin resulted in an increase in the DEPMPO/HOO• signal and a modest increase of the signal for DEPMPO/HO^{∘} (FIG. 16E).

Auranofin Inhibits Iniparib Bioactivation and Modification of TrxR1 and Other Protein Targets in HCT116 Cells

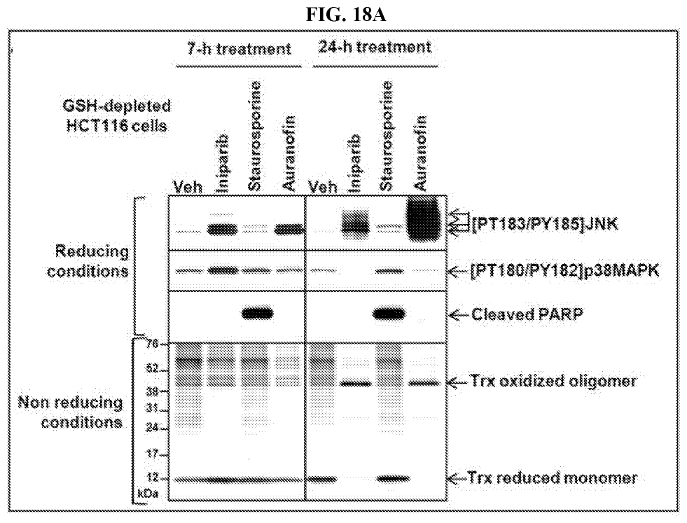

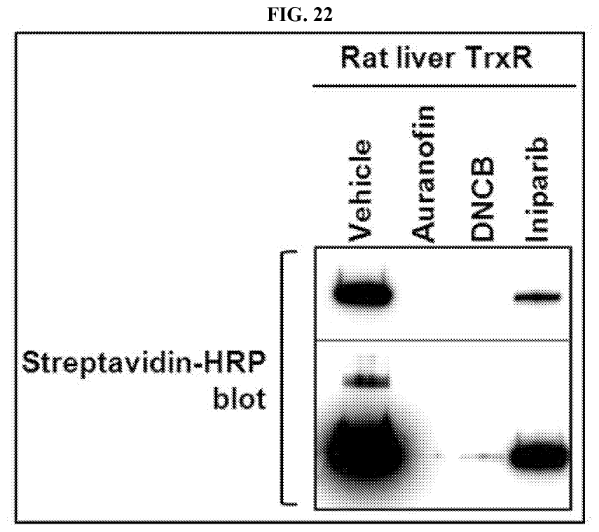

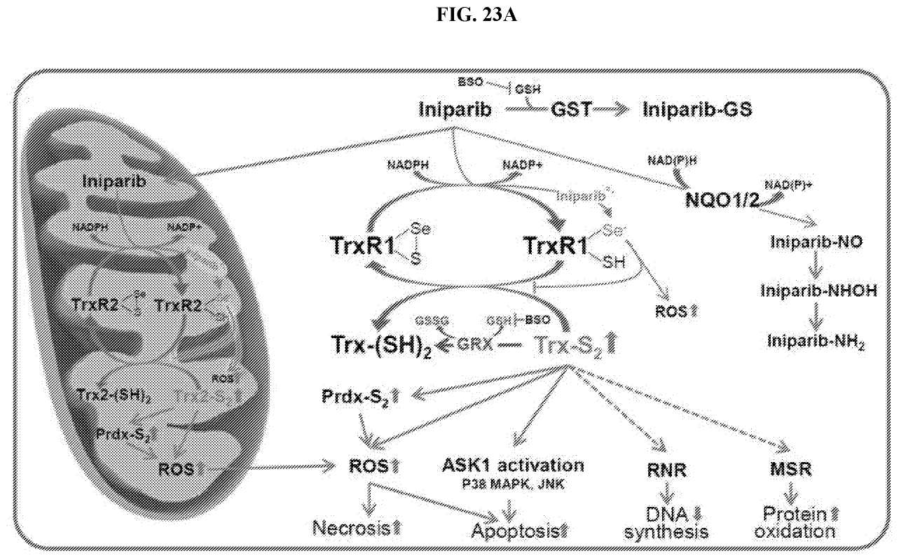

The effect of auranofin, a well characterized TrxR inhibitor, was investigated for the Iniparib/protein adduct formation. FIG. 17A shows that a 90-min transitory preincubation of GSH-depleted HCT116 cells with auranofin (at 10 μM) abolished TrxR1 labeling with Iniparib-biotin. In a complementary experiment, TrxR1 immunoprecipitated from cells treated for 90 min with auranofin was found unable to form adducts in vitro with Iniparib-biotin in the presence of NADPH. Same experiments were performed replacing auranofin by Iniparib during the 90 min preincubation of the cells prior to the addition of Iniparib-biotin. In this case only a partial inhibition of the labeling of TrxR1 was observed. These results are in line with the slower kinetics of inhibition displayed by Iniparib with respect to auranofin as inhibitors of TrxR1 described above. Similar observations were made when auranofin and Iniparib preincubations were performed in vitro with purified rat TrxR prior to addition of Iniparib-biotin (FIG. 22). It was then investigated whether or not auranofin could inhibit the whole process of protein modification by Iniparib-biotin in GSH-depleted HCT116 cells. As illustrated in FIG. 17B, auranofin blocked in a dose dependent manner cell proteins' labeling with Iniparib-biotin. At 0.1-0.3 μM the labeling of a 55-kDa protein, attributed to TrxR, was prevented and at higher concentrations, i.e. 1-3 μM, all the adduct formation with Iniparib-biotin in HCT116 cells were inhibited.

Trx is Oxidized, and JNK and p38MAPK Pathways are Activated Following Cell Treatment with Iniparib

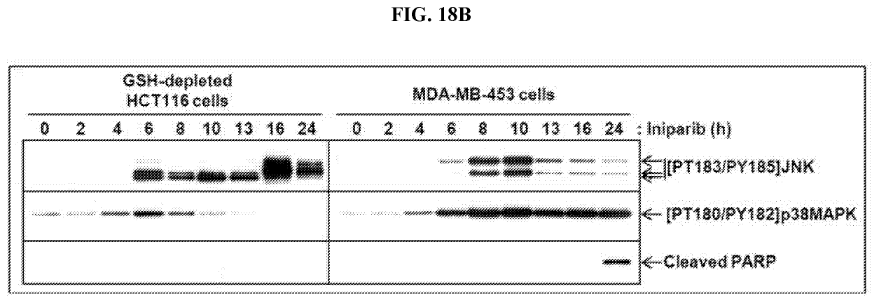

One of the consequences of inhibiting TrxR1 in cells is the accumulation of oxidized Trx, a protein substrate of TrxR1. Since accumulation of oxidized Txr results in activation of ASK1, through dissociation of the complex Trx/ASK1 and induction apoptosis, it was investigated, in cells treated with Iniparib, the redox state of Trx as well as the downstream pathway components of ASK1, JNK and p38MAPK. First, it was analyzed, in GSH-depleted HCT116 cells treated with Iniparib for 7 and 24 h, the oxidation of Trx and the phosphorylation of JNK and p38MAPK. Trx oxidation was studied here by the detection under nonreducing conditions of a disulfide-linked oligomeric form of Trx which likely corresponds to an ultimate oxidized state of the protein. As illustrated in FIG. 18A, this oligomeric/oxidized form of Trx accumulated after 24 h of Iniparib treatment with a concomitant disappearance of the monomeric/reduced form of Trx. Iniparib-induced Trx oxidation was accompanied by an activation of both p38MAPK and JNK phosphorylation. Auranofin treated cells showed similar Trx oligomerization/oxydation and JNK and p38MAPK activation as cells treated with Iniparib. Trx oxidation by Iniparib and Auranofin in GSH-depleted HCT116 cells was further confirmed by other method using iodoacetic acid alkylation of free cysteine and urea-PAGE. The TrxR independent inducer of apoptosis, staurosporine, was unable to induce oxidation of Trx, yet as expected staurosporine triggered PARP1 cleavage.

Similar results were observed when MDA-MB-453 cells were treated with Iniparib, however different levels of JNK and p38MAPK activation were observed. Thus, we decided to compare the time-course of Iniparib-activation of p38MAPK and JNK in HCT116 and MDA-M-453 cells. As shown in FIG. 18B, in both cells p38MAPK and JNK were activated as expected from TrxR inhibition. However, the kinetics of activation were different when comparing the two cell lines. JNK activation in GSH-depleted HCT116 cells occurred after 6 h of cell exposure to Iniparib and persisted until 24 h of treatment. In MDA-MB-453 cells the activation of JNK was apparent also at 6 h-8 h, and it peaked at 10 h and then faded. In contrast, p38MAPK activation was transient in GSH-depleted HCT116 cells (only observed between 4 and 8 h of Iniparib exposure) while it was persisting from 4 h to 24 h of treatment in MDA-MB-453 cells.

### Example 3—Iniparib is a Cytotoxic Anti-Tumor Prodrug Bioactivated by TrxR1/2 with a Sensitive Patient Stratum for PFS in Metastatic Triple Negative Breast Cancer

Materials

Cell line was purchased from the ATCC cell biology collection. Cell culture reagents were purchased from LifeTechnologies. All regular chemicals or reagents were obtained from Sigma-Aldrich Chemicals, unless otherwise specified.

Cell Culture

MDA-MB-231 (breast metastatic carcinoma) were cultured in DMEM medium supplemented either with 10% fetal bovine serum, 2 mM glutamine, 1 mM sodium pyruvate and 10 μg/ml ciprofloxacine (Euromedex) in a humidified 5% C02 atmosphere at 37° C.

Fluorescence Microscopy.

MDA-MB-231 cells were platted on polylysine D coated thin glass bottom microscope chambers (Ibidi). After 24 h of culture cells were first pre-incubated with BSO at 1 mM for 18 h and then treated with 100 μM iniparib-Biotin or its vehicle (DMSO 1%) for 30 min. For subcellular localization experiments mitochondria were stained with 100 nM Mitotracker Red CMX (Molecular probes) added for 10 min. This stain was performed before fixation (Paraformaldehyde 3.7% in PBS pH 7.4). Biotin was developed, after Triton X100 (0.3% in PBS, 15 min) permeabilization and saturation (1% BSA+1% gelatin in PBS: saturation buffer), with Alexa-488 streptavidin (Molecular Probes) conjugate (1 μg/ml in saturation buffer). Nuclei were stained with Hoechst (Molecular Probes) and samples mounted in antifading solution (Ibidi).

Cells were imaged with a PLAN NeoFluar 40× (NA 1.3) or 100×, (NA 1.46) oil objectives on a LSM510 (Zeiss) confocal microscope. Laser lines, filters and dichroic mirrors were selected for maximal separation of the green (Ex./Em. 488/530 nm) and the red fluorescence (Ex./Em. 543/LP 585 nm). Nuclei were observed (Ex./Em. 405/460 nm). For co-localization stacks of images separated by 400 nm along z-axis were acquired. Post-capture processing was done using LSM510 software, stacks of confocal images were deconvoluted using the ImageJ software.

TrxR Activity

TrxR activity was measured by the reduction of 5, 5′-dithiobis-2-nitrobenzoic acid (DTNB) according to the manufacturer's instructions (TrxR assay kit, Sigma). Briefly, all incubations were performed at 37° C. in 96-well microplates in 0.1 M potassium phosphate (pH 7.4), 10 mM EDTA and 240 μM NADPH. TrxR activity was measured by recording the initial increase in A412 during the first 10 min upon addition of 3 mM DTNB with a scanning multiwell spectrophotometer (Molecular devices). Endogenous TrxR activity was determined using clarified octyl-glucoside cell lysates (50 μg proteins). Initial velocities were derived from linear regression analyses and then plotted in double reciprocal plots to obtain the half time of TrxR inhibition using an one phase exponential decay analysis (Prism, GraphPad software).

Trx Western Blot Analysis

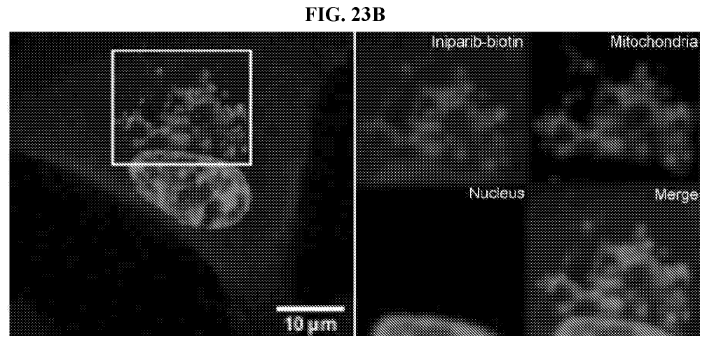

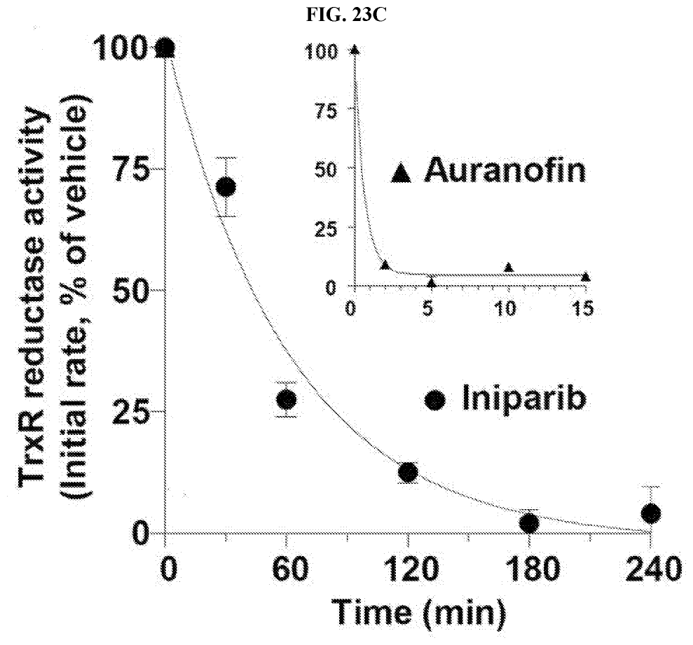

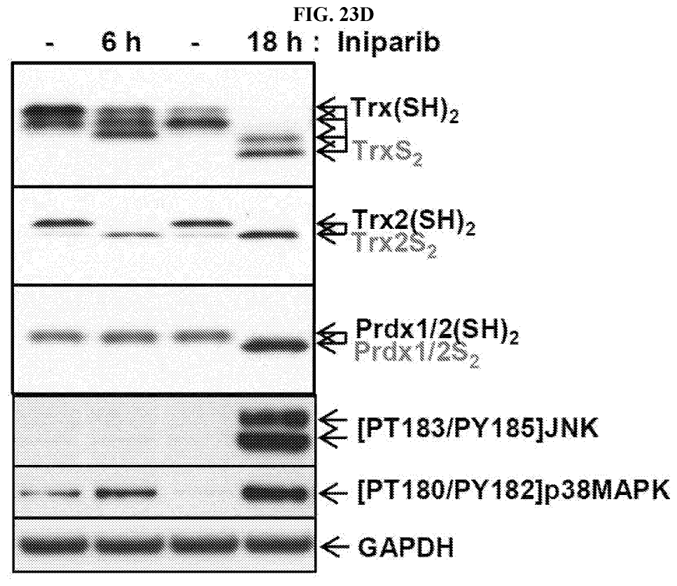

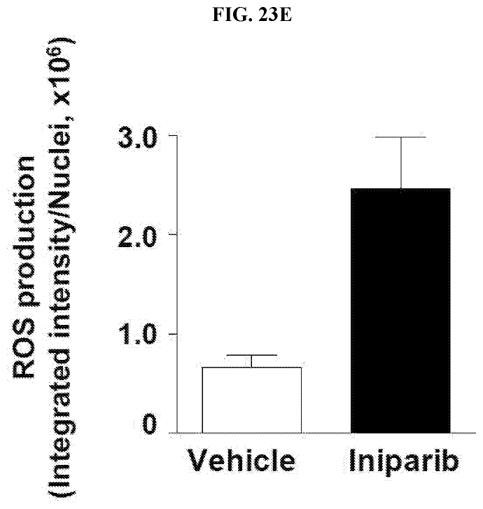

A modification of a standard Western blot allows quantification of the redox state of specific proteins by separation of reduced and oxidized forms by gel electrophoresis and detection of both forms with an antibody to an epitope that does not undergo oxidation-reduction (FIG. 23). Quantification is obtained directly from the relative intensities of the different bands. Two forms of the redox Western blot have been developed, separating on the basis of differing charge (thiols are modified with a charged alkylating reagent) or mass (thiols are modified with a high-mass alkylating reagent). Redox analysis of the mitochondrial compartment is performed using a redox Western blot analysis of thioredoxin-2 (Trx2). Trx2 is exclusively found in mitochondria, so an analysis of a cell or tissue extract provides specific information on redox in the mitochondria without fractionation. Analysis is performed following derivatization with AMS. Trx2 contains two cysteine residues, and the addition of two molecules of AMS increases the mass by approximately 1000 Da. Oxidized and AMS derivatized forms are separated by non-reducing SDS polyacrylamide gel electrophoresis and detected by immunoblotting.

ROS Production and Video-Microscopy.

MDA-MB-231 cells were platted in Ibidi® treated chambers. After 24 h of culture cells were first pre-incubated with BSO at 1 mM for 18 h and then treated with 100 μM iniparib or its vehicle (DMSO 1%) for 4 h. For ROS and nuclei detection, cells were respectively loaded with 5 μM CellROXOrange® (Molecular Probes) and 5 μM of DRAQ5® (Cell Signaling technology) in fresh media for 30 min.

Image acquisition was performed after washes with an Axiovert 200 Zeiss (Carl Zeiss Jena Germany) microscope equipped with a 40× C-Apochromat objective (N.A.=0.95). CellROXOrange® and DRAQ5® fluorescences were respectively excited with a LED light source (595 and 646 nm) and emitted light were collected at 565 and 681 nm. For quantification ImageJ software was used. Data is presented as Integrated Intensities/nuclei.

Clinical Trial Panel.

Phase 2 and Phase 3 clinical trials were conducted to test the efficacy of iniparib in combination with standard of care chemotherapy (gemcitabine and carboplatin) for patients with metastatic recurrence of triple negative (hormone receptor status) breast cancer. Formalin-fixed, paraffin-embedded (FFPE) archival samples from biopsy or surgery at original diagnosis of breast cancer were profiled on the exon-based Affymetrix Hugene1.0ST microarrays using an RNA extraction protocol adapted to the short fragment lengths resulting from RNA degradation in FFPE samples. It was considered that a corpus of n=210 patients for whom clinical outcome data was available in the form of PFS time and associated censoring statuses.

Processing of Gene Expression Data.

The raw gene expression data from profiling of an initial n=240 FFPE preserved samples, in the form of individual Affymetrix CEL files, was processed using MASS estimation. Quality control based on average array brightness excluded 30 outlier scans, and the n=210 remaining profiles were then normalized to each other using quantile normalization. Affymetrix probesets were mapped to genes, in a manner where multiple probesets mapping into the same gene were resolved by assigning the highest intensity to the corresponding gene, this procedure reducing the set of 33,297 probe sets to 20,756 mapped genes. The data was then log 2-transformed, and standardized by mean-subtraction and division by the standard deviation across all 210 samples, for each gene separately. Finally, the data matrix was subsetted to the intersection of the oxidative response set of 102 genes with the set of genes represented on the Hugene1.0ST arrays, resulting in profiles for p=82 genes across all n=210 samples.

Multivariate Cox Modeling, Based on Gene Expression Data and on an a Priori Oxidative Stress Gene Set.

A multivariate Cox model using supervised principal components was used to model progression free survival times regressed on gene expression data. The model was of the form

$$ξ = log ( \frac{λ ( t \vert x , z )}{{λ}_{0} ( t )} ) = {β}_{0} z + {\sum }_{i = 1}^{K} {\tilde{β}}_{i} {\tilde{x}}_{i} + z \cdot {\sum }_{i = 1}^{K} {\tilde{γ}}_{i} {\tilde{x}}_{i} \qquad Eq . ( 1 )$$

where by definition 5 is the “log-hazard-ratio” for a given individual, λ(t|z, x) the hazard function (or risk per unit time) for that individual, with covariate vector (z, x), and PFS time t, λ0(t) the baseline hazard function (the hazard which applies to an individual with all covariates exactly equal to 0), and where z is a binary indicator of treatment arm, with z=0 for the control and z=1 for the iniparib treatment arm. The symbol x refers to the gene expression vector with p=82 components (the subset of the oxidative stress gene set represented on the Affymetrix microarrays). Note that this model contains both direct and interaction terms, the coefficients \tilde{β}₁ accounting for the direct effects of gene expression (which might be called “prognostic” effects) and the coefficients \tilde{γ}₁ accounting for gene expression×treatment-arm effects (“predictive” effects). The coefficient β0 accounts for overall, gene-expression independent, treatment-arm effects.

In Eq.(1) the variables: \tilde{x}₁, l=1, . . . , K, denote projections onto the first K principal components of the data matrix of gene expression vectors, after preliminary reduction to a subset of mtop genes out of the original set of 82, where mtop is a variable parameter, 1≤mtop≤82. For a given mtop, the subset is determined by univariate feature selection (generating a Cox model for each gene independently and selecting the mtop genes with the smallest interaction-term P-values). Using 5-fold cross-validation, the values mtop=15 and K=1 were found to maximize signature selectivity, resulting in a maximal separation in differential survival times between groups over a broad range of thresholds (methods paper in preparation).

Note that with the principal component vectors determined, Eq.(1) can be written as

$$ξ = log ( \frac{λ ( t \vert x , z )}{{λ}_{0} ( t )} ) = {β}_{0} z + {\sum }_{i = 1}^{15} {β}_{i} {x}_{i} + z \cdot {\sum }_{i = 1}^{15} {γ}_{i} {x}_{i} \qquad Eq . ( 2 )$$

so that in this form the model predictions are formulated directly in terms of gene expression values, through the coefficients βᵢ and γᵢ (although model building and optimization were not done in terms of these coefficients). The 15 genes selected are given in Table 4.

The differential log-hazard ratio Δξ is defined as the log-ratio of hazards for individuals with the same gene expression profile, but in different treatment arms,

$$Δ ξ = log ( \frac{λ ( t \vert x , z = 1 )}{λ ( t \vert x , z = 0 )} ) = {β}_{0} z + {\sum }_{i = 1}^{15} {γ}_{i} {x}_{i} .$$

Objective Response Rate (ORR) Categories:

Were considered as responders patients experiencing either complete or partial response (CR, PR), while non-responders exhibited either stable or progressive disease (SD, PD).

Gene Expression Profiles and Mode of Action in Triple Negative Breast Cancer

The mode of action in a triple negative breast cancer-like cell line and the analysis of gene expression profiles of tissue samples collected in the clinical studies of iniparib in mTNBC were studied, including biomarker analysis using a set of genes related to the mechanism of action of iniparib.

Iniparib was shown as a prodrug that follows three main metabolic pathways, a) conjugation with glutathione (GSH) catalyzed by Glutathione-S-Transferases (GST) leading to drug inactivation b) 2-electron reduction by NAD(P)H:quinone oxidoreductase 1 and 2 (NQO1/2) leading to inactivation (however, the nitroso metabolite formed in this reaction has cytotoxic activity, its reactivity causes its rapid transformation in inactive metabolites), and c) one-electron reduction of the nitro group and production of a highly reactive nitrosyl radical in both the cytosol and in mitochondria catalyzed by thioredoxin reductase 1 and 2 (TrxR 1/2) respectively (see FIG. 23A).

In MDA-MB-231 cells treated with biotinylated-iniparib, covalently modified cytosolic and mitochondrial proteins were detected as early as 30 min following compound addition (FIG. 23B). Among the target enzymes, TrxR1 and TrxR2 modified at the highly reactive selenocysteine (Sec) residue Sec residue located in the C-terminal redox site were identified. As illustrated in FIG. 23C, treatment of MDA-MB-231 cells with iniparib results in an inhibition of the TrxR reductase activity, in line with results obtained in other cancer cell models. Auranofin, an inhibitor of TrxR, also results in inhibition of the reductase activity. The kinetics of inhibition of TrxR activity by auranofin is, however, faster than that of iniparib which requires enzymatic activation. The main substrate of TrxR is oxidized thioredoxin (Trx), and as illustrated in FIG. 23D, accumulation of oxidized Trx is visible as early as 6h post treatment, and increases further at 18h. As a further consequence of Trx inhibition, peroxiredoxin (Prdx) accumulates in its oxidized state. In some instances, ribonucleotide reductase (RNR), and methionine sulfoxide reductase activities (MSR) are impaired, leading to DNA damage, and additional protein oxidation, respectively (see FIG. 23A). This in turn leads to activation of p38MAPK and JNK phosphorylation (FIG. 23D, lower panel) driving cells into apoptosis. Inhibition of TrxR results in an imbalance in the cellular redox control system, and as shown in FIG. 23E, cells treated with iniparib clearly show an accumulation of ROS. Dysregulation of redox and ROS compensatory mechanisms thus appears to be major factors promoting tumor cell death. Overall, the activity of iniparib in MDA-MB-231 cells is similar to that observed in the HCT116 colon cancer cell line, and the cytotoxicity is greatly enhanced in GSH-depleted cells by incubation with an inhibitor of GSH synthesis such as L-buthionine sulfoximine (BSO). Cell depletion of GSH not only prevents iniparib detoxification by GST but may also switch off an alternative pathway of Trx regeneration involving glutaredoxin (GRX) (FIG. 23A)

Taken together, the data points to a complex mechanism of action for iniparib involving multiple pathways that can be grouped into the three main channels as discussed above and outlined in FIG. 23A. Thus, it appears that iniparib cytotoxicity involves entering the prodrug into the TrxR channel to undermine the cells capability to control redox homeostasis, and ROS regulation. This can occur if the sum of the competing inactivation channels through GSH conjugation, and NQO1/NQO2 catabolism, respectively, is quantitatively less important.

The cohort of patients in Phase 2 and Phase 3 trials that were conducted to test the efficacy of iniparib in combination with gemcitabine and carboplatin in patients with mTNBC were further reviewed for potential gene expression signatures. The control arm received a gemcitabine+carboplatin regimen. Formalin-fixed, paraffinembedded archival samples of breast cancer tissue were profiled using mRNA microarrays.

A population of n=210 patients were considered for whom suitable molecular data and clinical outcome data was available in the form of progression-free survival (PFS) and overall survival (OS). Given the similarity in design of the Phase 2 and Phase 3 trials, specimens collected from both trials were combined and included in the biomarker analysis to improve statistical power.

A multivariate Cox model was built using the standardized expression values of 15 oxidative stress genes selected from an original list of 110 genes associated with the “canonical” ROS pathways derived from the emerging understanding of the mechanism of action of iniparib. The 15-gene model was developed by optimization across a series of cross-validated models with variable numbers of genes, and used to predict differential log-hazard ratios for PFS between an iniparib-containing treatment arm, and a control arm treated with standard chemotherapy (see Methods). The 15 genes used in the analysis are shown in Table 4, together with their loadings for direct effects due to gene expression (beta coefficients) and for gene expression×treatment-arm interaction effects (gamma coefficients), where gamma >0 indicates increased survival risk with increasing gene expression in the iniparib versus the control arm. Genes 1 to 10, notably containing NQO2 and several GST variants, predict increased risk of progression for iniparib-treated patients relative to control when highly expressed (gamma >0). This is consistent with the majority of these genes encoding proteins being hypothesized to participate in the inactivation of iniparib as described above through GSH conjugation (GST), or two-electron reduction (NQO2). On the other hand, genes 11-15 in Table 4, predict decreased risk of progression in iniparib-treated patients relative to control when highly expressed (gamma <0). The proteins encoded by these 5 genes include TrxR2 and, importantly, several enzymes whose redox state is controlled by TrxR family members including TXN2, MSRB3, and MSRA. It is interesting that the mitochondrial TrxR2 is higher ranked than the cytosolic isoform. Its location in mitochondria and its imperative role in controlling mitochondrial redox status make the inactivation of this enzyme particularly.

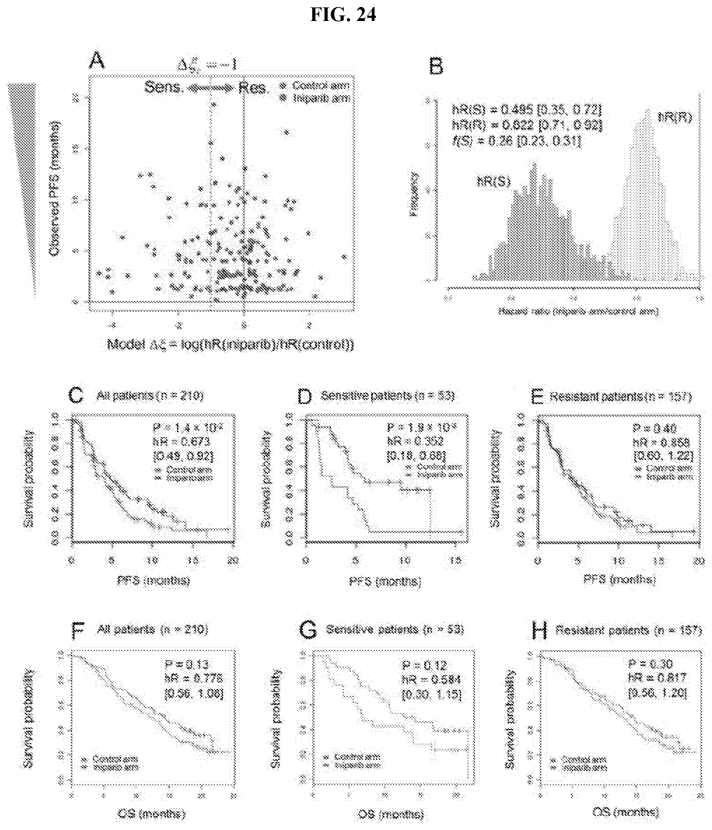

FIG. 24A shows a plot of PFS versus predicted differential log-hazard (Δξ, see Methods) for all n=210 patients by treatment arm. Patients with Δ<0 are predicted to have longer survival in the iniparib-containing treatment arm than in the control arm. This is qualitatively verified by the observation on the left-hand side of the diagram of a large number of blue dots (patients treated with iniparib) occurring well above most of the red dots (control patients). Conversely, heights of blue and red dots are largely intermingled on the right-hand side of the diagram, Δξ>0, indicating similar survival across treatment arms for this category of patients. FIG. 24B shows the results of 1000 independently and randomly sampled 5-fold cross-validations to establish the robustness of the signature's segregation of patients into putative response groups for a fixed threshold Δξc=−1, chosen to generate a reasonably strong predicted differential effect in the sensitive population. The resulting distributions of hazard ratios as shown are robustly separated, with 95% confidence intervals indicated in the inset. For the threshold Δξc=−1, the fraction f(S) of patients selected as sensitive by the signature represent approximately 25% of the total population.

FIG. 24C shows survival curves for the two treatment arms for all patients (n=210), displaying a moderate improvement in PFS with the addition of iniparib to chemotherapy (hR=0.673 [0.49, 0.92]95%) between the treatment arms. FIG. 24D displays a comparison for the sensitive patients only, after selection by the threshold Δξc=−1 (n=53), showing a considerably smaller hazard ratio between treatment arms for PFS for iniparib-treated patients than in the overall population (hR=0.352[0.18, 0.68]95%). FIG. 24E shows a comparison for the “resistant” patients (n=157), showing a larger hazard ratio for progression compared to either of the other populations (hR=0.858[0.60, 1.22]95%). In the biomarker-selected patient population of n=53 the median progression-free survival is 5.9 months for the iniparibtreated cohort versus only 2.60 months for the control arm. Panels 2F-H show the corresponding results for overall survival (OS). In particular, the sensitive subgroup analysis of the OS data is consistent with the PFS analysis (FIG. 24G versus FIG. 24D), although the magnitude of the biomarker effect on overall survival is numerically smaller.

A similar analysis using the sensitive and resistant categories defined above, but applied to the objective response rate (ORR, see Methods) instead of to survival times suggests that sensitive patients showed a greater ORR under iniparib than under control arm treatment, while the resistant group did not. Thus for the sensitive group of n=53 patients, 48% of responded to iniparib versus 26% to the control treatment (odds ratio 2.57[0.66, 11.48]_{95%}). For the resistant group, the corresponding response rates were 34% and 40% respectively (odds ratio 1.29[0.63, 2.65]_{95%}).

Stratifying the patients by number of lines of treatment, it was found that even among the 2nd/3rd line patients, who already exhibited a stronger differential response to iniparib treatment than 1st line patients, selection based on the signature resulted in yet greater PFS and OS benefits. More specifically, among 2nd/3rd line patients the signature-defined sensitive subgroup (n=24) had stronger OS benefit (HR=0.28[0.10, 0.77]_{95%}) than the corresponding resistant subgroup (n=82, HR=0.49[0.30, 0.81]_{95%}).

Table 4 illustrates a 15-gene expression signature for prediction of PFS in the two-arm clinical trials for triple negative breast cancer (TNBC) patients. The 15 genes resulting from optimal feature selection supported by cross-validation of the multivariate Cox model are shown together with their individual loadings for direct effects due to gene expression (beta coefficients), and for gene expression×treatment-arm interaction effects (gamma coefficients), where gamma >0 indicates increased survival risk with increasing gene expression, in the iniparib versus the control arm, and gamma <0 decreased risk. The 15-gene Cox model can be summarized by the equation

$$log ( λ ( x , z ) \text{/} {λ}_{0} ) = {β}_{0} z + {\sum }_{i = 1}^{15} {β}_{i} {x}_{i} + z \cdot {\sum }_{i = 1}^{15} {γ}_{i} {x}_{i} ,$$

where λ(x, z) is the hazard function, λ₀ the baseline hazard, xᵢ standardized gene expression for the i-th gene, and where z is a binary indicator of treatment arm (z=1 for iniparib arm, 0 for control arm).

TABLE 4

| Number | Gene | description | beta | gamma |
|---|---|---|---|---|
| 1 | NQO2 | NAD(P)H dehydrogenase | −0.098 | 0.318 |
| | | quinone 2 | | |
| 2 | GSTT2 | glutathione S-transferase theta 2 | −0.231 | 0.295 |
| 3 | GSTM3 | glutathione S-transferase | −0.249 | 0.293 |
| | | M3 (brain) | | |
| 4 | GLRX | glutaredoxin (thioltransferase) | −0.153 | 0.263 |
| 5 | SELO | selenoprotein O | −0.120 | 0.257 |
| 6 | PON1 | paraoxonase 1 | −0.027 | 0.246 |
| 7 | GSTO1 | glutathione S-transferase | −0.014 | 0.232 |
| | | omega 1 | | |
| 8 | GLRX3 | glutaredoxin 3 | 0.030 | 0.147 |
| 9 | SEPX1 | selenoprotein X 1 | 0.097 | 0.110 |
| 10 | TXNRD1 | thioredoxin reductase 1 | −0.113 | 0.067 |
| 11 | TXNRD2 | thioredoxin reductase 2 | −0.128 | −0.019 |
| 12 | TXN2 | thioredoxin 2 | 0.204 | −0.111 |
| 13 | MSRB3 | methionine sulfoxide | −0.003 | −0.211 |
| | | reductase B3 | | |
| 14 | MSRA | methionine sulfoxide | 0.149 | −0.331 |
| | | reductase A | | |
| 15 | GSTZ1 | glutathione transferase zeta 1 | 0.372 | −0.426 |

### Example 4—Phase I Clinical Trial—Glioblastoma Multiforme (GBM)

The Iniparib phase 1 GBM study was a multicenter study. It was a single arm, multi dose, dose escalating trial in newly diagnosed GBM patients. The total number of patients was 43, with about 5 patients per cohort. Patients who tolerated radiation (XRT) and temozolomide (TMZ) were recruited. Inclusion criteria included completion of XRT and TMZ without grade 3 or 4 toxicity and labs within acceptable range within 6 weeks of completing XRT. The end points included safety, maximum tolerated dose (MTD), and signal of activity.

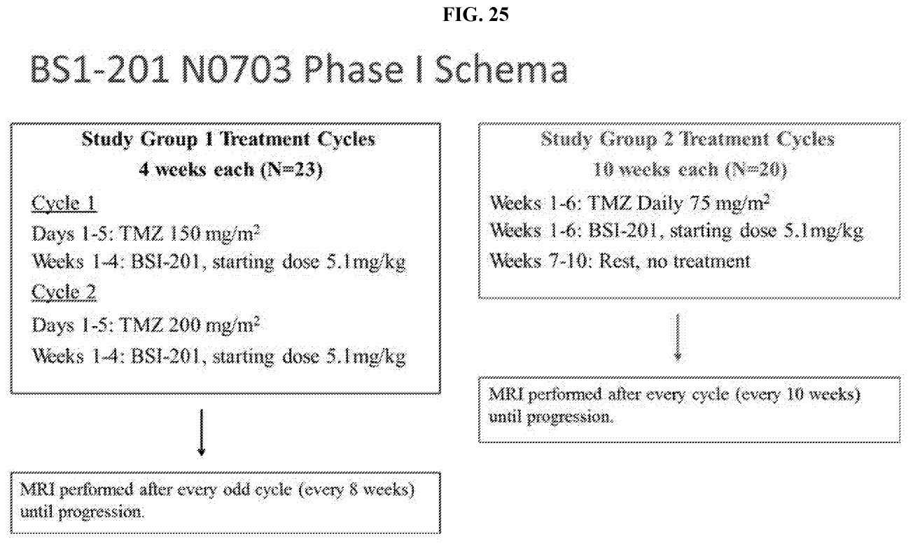

The patients were separated into two study groups with the following treatment schema (also see FIG. 25).

Study Group 1 Treatment Cycles 4 Weeks Each (N=23)

Cycle 1

Days 1-5: TMZ 150 mg/m²

Weeks 1-4: BSI-201, starting dose 5.1 mg/kg

Cycle 2

Days 1-5: TMZ 200 mg/m²

Weeks 1-4: BSI-201, starting dose 5.1 mg/kg

MRI performed after every odd cycle (every 8 weeks) until progression.

Study Group 2 Treatment Cycles 10 Weeks Each (N=20)

Weeks 1-6: TMZ Daily 75 mg/m²

Weeks 1-6: BSI-201, starting dose 5.1 mg/kg Weeks 7-10: Rest, no treatment

MRI performed after every cycle (every 10 weeks) until progression.

Continuous Reassessment Method (CRM) was used to determine dose escalation. PK was drawn at cycle 1, 2, 3 and off treatment. PD via PBMCs was drawn at cycle 1, 2, 3 and off treatment. No cytochrome P450-inducing anticonvulsants. However, Gliadel was permitted.

In Adjuvant Phase:

Group 1: TMZ (150-200 mg/m² given 5 days/month x 6 cycles)—standard dose with BSI-201 starting at 5.1 mg/kg.

Group 2: TMZ (75 mg/m² daily 42 days on 30 days off x 3 cycles)—metronomic dose with BSI-201 starting at 5.1 mg/kg.

Using modified continual reassessment method, MTD was defined for metronomic and standard dose TMZ. 6 dose levels were tested (lowest 5.1 mg/kg-highest 9.5 mg/kg IV 2×/wk). At 8.6 mg/kg (17.2 mg/kg/week), 1/9 patients had a DLT. The DLTs across both groups were: rash (1), hypersensitivity reaction (1), fatigue (1) and a thromboembolic event (1). Additional grade 3 toxicities were neutropenia, lymphopenia, nausea, and elevated AST. Phase 2 dose defined as 8 mg/kg IV 2×/wk with standard TMZ and 8.6 mg/kg IV 2×/wk with metronomic TMZ.

Table 5 illustrates the pharmacokinetics of iniparib. Data are presented as the geometric mean±SD for peak plasma concentrations (Cₘₐₓ) and the arithmetic average±SD for the metabolite/iniparib concentration ratio as expressed as a percentage. Dosing is IV 2×/week continuous. IABM and IABA are the two major metabolites of iniparib in plasma.

TABLE 5

| | | | | | | Metabolite/iniparib | |
|---|---|---|---|---|---|---|---|
| Dose | No. of | No. of | Cₘₐₓ (ng/mL) | | | (%) | |
| (mg/kg) | Patients | samples | Iniparib | IABM | IABA | IABM | IABA |
| 5.1 | 8 | 20 | 642 ± 315 | 5.7 ± 4.1 | 16.6 ± 5.2 | 1.1 ± 0.6 | 3.0 ± 2.0 |
| 6.1 | 5 | 11 | 928 ± 397 | 7.2 ± 1.8 | 14.9 ± 8.3 | 0.9 ± 0.6 | 2.0 ± 1.5 |
| 6.8 | 7 | 18 | 1,019 ± 463 | 8.4 ± 3.7 | 19.9 ± 6.6 | 1.1 ± 0.9 | 2.4 ± 1.4 |
| 8.0 | 4 | 5 | 3,687 ± 1,464 | 10.3 ± 2.9 | 14.5 ± 15.0 | 0.3 ± 0.1 | 0.7 ± 0.7 |
| 8.6 | 5 | 18 | 1,517 ± 987 | 9.7 ± 2.4 | 31.5 ± 10.4 | 0.8 ± 0.6 | 2.5 ± 1.4 |
| 9.5 | 4 | 7 | 2,139 ± 2,133 | 13.6 ± 5.3 | 19.8 ± 14.0 | 0.8 ± 0.6 | 2.0 ± 2.0 |

Table 6 illustrates the toxicity of iniparib. Grade 3-4 adverse events are shown with relationship of possible, or probable, or definite to iniparib.

TABLE 6

| | Group I N = 23 | Group II N = 20 | Total N = 43 |
|---|---|---|---|
| Adverse Event | No. (% of pts) | No. (% of pts) | No. (% of pts) |
| Allergic reaction | 1 (4) | | 1 (2) |
| Alanine | | 6 (30) | 6 (14) |
| Anemia | 13 (57) | 11 (55) | 24 (56) |
| Constipation | 9 (39) | 7 (35) | 16 (37) |
| Dizziness | 5 (22) | | 5 (12) |
| Fatigue | 15 (65) | 13 (65) | 28 (65) |
| Nausea | 9 (39) | 9 (45) | 18 (42) |
| Rash | 5 (22) | | 5 (12) |
| maculo-papular | | | |
| Thromboembolic | | 1 (5) | 1 (2) |
| event | | | |
| Lymphocyte count | | 6 (30) | 6 (14) |
| decreased | | | |
| Platelet decreased | 15 (65) | 6 (30) | 21 (49) |
| White counts | 11 (48) | 11 (55) | 22 51) |
| decreased | | | |

### Example 5—Phase II Clinical Trial—Glioblastoma Multiforme (GBM)

The primary objective of the Phase II study was to estimate the overall survival for adult patients with newly diagnosed glioblastoma multiforme (GBM) treated with BSI-201 (iniparib) at the MTDs during RT with concurrent and adjuvant TMZ. The secondary objective was to estimate the frequency of toxicity associated with this treatment regimen. 76 patients were recruited for this study. Corollary studies included PARP-1 expression in resected GBM and MGMT status in resected GBM.

Safety Run-In:

BSI-201 at one dose less than the MTD from Group 2 with TMX 75 mg/m²+XRT (3 patients), then

BSI-201 at Group 2 MTD with TMX 75 mg/m²+XRT (3 patients) to ensure safety of triple therapy.

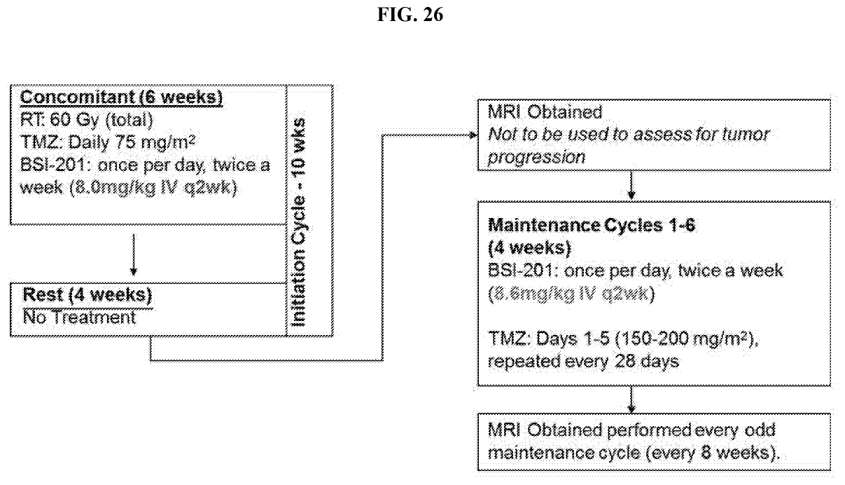

The following treatment schema (also see FIG. 26).

Concomitant (6 Weeks)

RT: 60 Gy (total) TMZ: Daily 75 mg/m²

BSI-201: once per day, twice a week (8.0 mg/kg IV q2 wk)

Rest (4 Weeks) with No Treatment.

Maintenance Cycles 1-6 (4 weeks)

BSI-201: once per day, twice a week (8.6 mg/kg IV q2 wk)

TMZ: Days 1-5 (150-200 mg/m²), repeated every 28 days

For assessing the efficacy of the treatment in terms of overall survival, the overall failure rate were estimated and compared to the failure rate of 0.6 per-person year of follow-up regarding the Phase III trial done by Stupp et al. in the same patient population treated with RT plus concomitant and adjuvant temozolomide.

The primary endpoint was death due to all causes. The survival time is defined from time of histological diagnosis to death occurrence. The overall failure rate was expressed as hazard of failure per person-year of follow-up. The total patient population for this part of the study was defined as all patients who have met the eligibility criteria, not met ineligibility criteria, and signed patient informed consent.

It was assumed that the patients in the study had an overall failure rate of 0.45 per person-year of planned follow-up. It is approximately 25% reduction in hazard rate compared to a hazard rate of 0.6 in the Phase III trial done by Stupp et al. With a total of 55 events among 76 patients, the study yield 80% power to detect an observed hazard ratio of 0.75 (0.45 vs. 0.6) at an alpha level of 0.1(one-sided) to be statistically significant. It yield above 90% power to detect a 30% reduction in hazard rate with observed hazard ratio of 0.7 (0.42 vs. 0.6) at an alpha level of 0.1 to be significant. The overall failure rate was estimated by dividing the number of events (deaths) by the total exposure time in the study cohort along with 95% confidence intervals. Survival probability and median time of survival was calculated using Kaplan-Meier method.

Table 7 illustrates the demographics of the patients.

TABLE 7

| | All Patients (N = 81) |
|---|---|
| Age: Median (Range) | 58 (27-80.9) |
| Gender: No. Male (%) | 51 (63) |
| RACE: | |
| White: No. (%) | 77 (95) |
| Ethnic Group: | |
| Hispanic or Latino: No. (%) | 1 (1) |
| Not Hispanic or Latino: No. (%) | 74 (91) |
| Unknown: No. (%) | 6 (7) |
| Anticonvulsant: | |
| Yes: No. (%) | 64 (79) |
| No: No. (%) | 17 (21) |
| KPS: | |
| 100: No. (%) | 9 (11) |
| 90: No. (%) | 40 (49) |
| 80: No. (%) | 22 (27) |
| 70: No. (%) | 8 (10) |
| 60: No (%) | 2 (2) |
| Mini Mental Score: Median (Range) | 29 (22-30) |
| Diagnosis: | |
| Glioblastoma Multiforme: No. (%) | 80 (99) |
| Gliosarcoma: No. (%) | 1 (1) |
| Surgical Procedure | |
| Craniotomy: No. (%) | 77 (95) |
| Biopsy: No. (%) | 4 (5) |

Table 8 illustrates toxicity and tolerability.

TABLE 8

| | | | | Stupp et al, |
|---|---|---|---|---|
| Adverse Events: N (%) | Grade 3 | Grade 4 | Total | 2005 |
| Acute kidney injury | 1 (1) | | 1 (1) | |
| Alanine aminotransferase | | 1 (1) | 1 (1) | |
| increased | | | | |
| Anemia | 2 (2) | | 2 (2) | |
| Aspartate aminotransferase | | 1 (1) | 1 (1) | |
| increased | | | | |
| Atrial fibrillation | 1 (1) | | 1 (1) | |
| Bronchial infection | 1 (1) | | 1 (1) | |
| Cognitive disturbance | 1 (1) | | 1 (1) | |
| Confusion | 1 (1) | | 1 (1) | |
| Dehydration | 1 (1) | | 1 (1) | |
| Dizziness | 1 (1) | | 1 (1) | |
| Dysphasia | 1 (1) | | 1 (1) | |
| Fatigue | 4 (5) | | 4 (5) | |
| Flushing | | 1 (1) | 1 (1) | |
| Generalized muscle weakness | 2 (2) | | 2 (2) | |
| Headache | 1 (1) | | 1 (1) | |
| Hyperkalemia | 1 (1) | | 1 (1) | |
| Hypertension | | 1 (1) | 1 (1) | |
| Hypokalemia | 1 (1) | | 1 (1) | |
| Hypotension | 1 (1) | | 1 (1) | |
| Hypoxia | 1 (1) | | 1 (1) | |
| Lymphocyte count decreased | 4 (5) | | 4 (5) | |
| Nausea | 2 (2) | | 2 (2) | |
| Neutrophil Count Decreased | 3 (4) | 5 (6) | 8 (10) | 7% |
| Platelet Count Decreased | 4 (5) | 11 (13) | 15 (18) | 12% |
| Rash maculo-papular | 3 (4) | | 3 (4) | |
| Skin and subcutaneous tissue | 1 (1) | | 1 (1) | |
| disorders | | | | |
| Vomiting | 1 (1) | | 1 (1) | |
| White Blood Cell Decreased | 5 (6) | 3 (4) | 8 (10) | 7% |

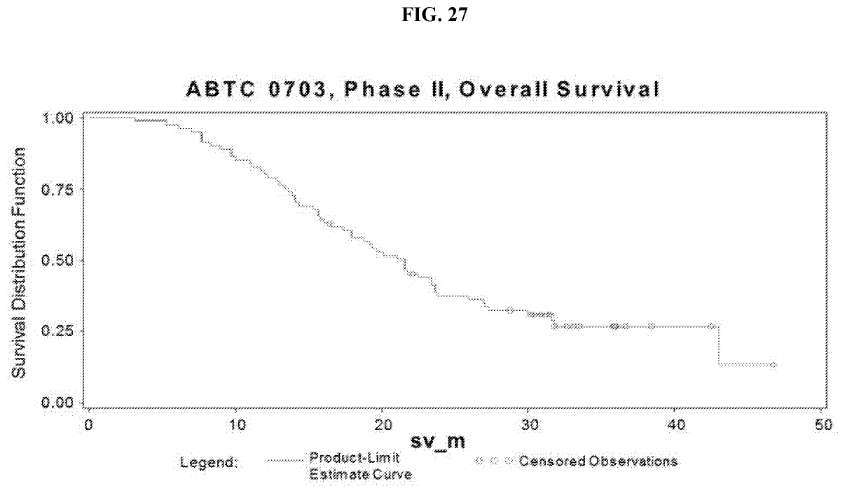

FIG. 27 and Table 9 show the overall survival analysis. Hazard rate of death at 0.6 was the null hypothesis to against an alternative hypothesis of 0.42 by the trial design. The treatment has achieved the target therapeutic effect which yielded a hazard rate of 0.403 per person year of follow-up.

TABLE 9

| | Median | Hazard Rate |
|---|---|---|
| | Overall Survival | (95% CI) (per person |
| Trial | Months, (95% CI) | year of follow-up) |
| ABTC0703 n = 76 | 21.6 (16.1-23.7) | 0.403 (0.308-0.526) |
| EORTC Stupp, 2005* | 14.6 | 0.6 (*conversion) |
| n = 287 | | |
| RTOG 0525, 2013 | 16.6 | 0.501 (*conversion) |
| STD arm, n = 411* | | |
| RTOG 0525, 2013 | 14.9 | 0.558 (*conversion) |
| DD arm, n = 422* | | |

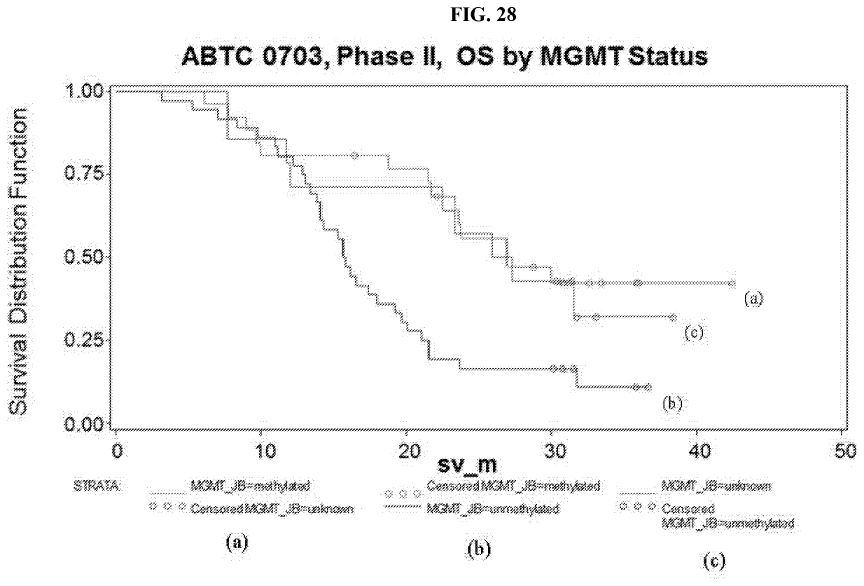

FIG. 28 and Table 10 illustrate overall survival by MGMT status.

TABLE 10

| | Median | Median Overall |
|---|---|---|
| | Overall Survival | Survival Months, |
| Trial | Months, (95% CI) | (95% CI) |
| MGMT | mOS, MGMT | mOS, MGMT |
| | methylated | unmethylated |
| ABTC0703 | 27 (n = 29) | 15.8 (n = 37) |
| EORTC Stupp, 2005 | 21.7 (n = 46) | 12.7 (n = 60) |
| RTOG 0525 | 21.4 (n = 122) sTMZ | 14.6 (n = 254) sTMZ |
| | 20.2 (n = 123) ddTMZ | 13.3 (n = 263) ddTMZ |

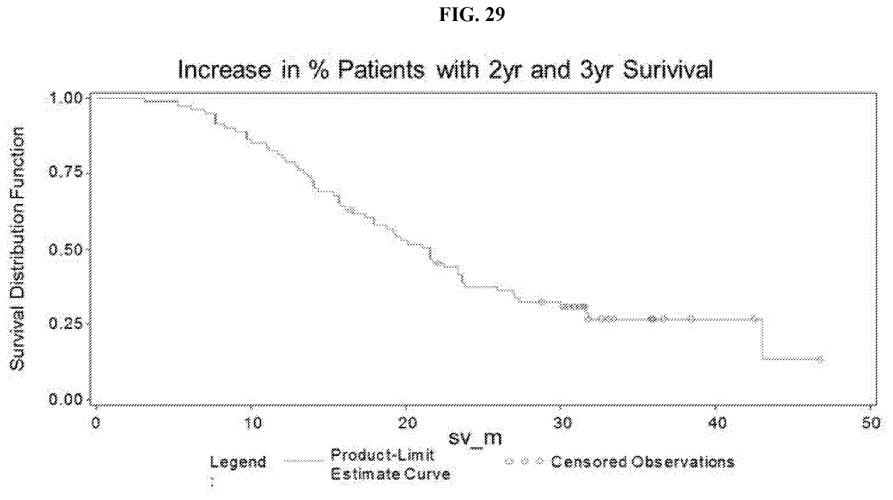

FIG. 29 and Table 11 illustrate an increase in the percentage of patients with 2 year survival and 3 year survival.

TABLE 11

| | 2 Year Survival | 3 Year Survival |
|---|---|---|
| Trial | (% patients) | (% patients) |
| Iniparib Phase 2 | 42.1 | 23.7 |
| N = 76 | | |
| EORTC Stupp, 2005 n = 287 | 27.2 | 16.0 |

Iniparib well tolerated at doses of 16 mg/kg weekly with radiation and TMZ and 17.2 mg/kg weekly with adjuvant TMZ

Single arm phase 2 met efficacy endpoint with at least a 25% reduced HR versus Stupp et al 2005

Also improved over RTOG 0525 (2013), but extrapolated and not pre-planned analysis.

### Example 6—A Phase 3, Multi-Center, Open-Label, Randomized Study of Gemcitabine/Carboplatin, With or Without BSI-201, in Patients With ER-, PR-, and Her2-Negative Metastatic Breast Cancer

The goal of this study was to determine the effect on overall survival and progression free survival by adding iniparib (BSI-201/SAR240550) to the combination of gemcitabine/carboplatin in adult patients with triple negative breast cancer (estrogen receptor (ER)-negative, progesterone receptor (PR)-negative, and human epidermal growth factor receptor 2 (HER2)-negative).

Study Type: Interventional

Study Design: Allocation: Randomized

Intervention Model: Parallel Assignment

Masking: Open Label

Primary Purpose: Treatment

Primary Outcome Measures:
progression free survival [Time Frame: until cut-off date established from deaths rate]
Progression free survival was defined as the time interval from the date of randomization to the date of first disease progression (as assessed by Independent Radiologic Review (IRR) based on Response Evaluation Criteria in Solid Tumor (RECIST) criteria), or the date of death due to any cause, whichever occurred first. In the absence disease progression or death, the participant was censored at the date of the last valid tumor assessment performed before the cut-off date.
Overall survival [Time Frame: until cut-off date established from deaths rate]
Overall survival was defined as the time interval from the date of randomization to the date of death due to any cause. In the absence of confirmation of death, participant was censored at the last date he/she was known to be alive, or at the cut-off date, whichever was earlier.

Secondary Outcome Measures:
Best overall response [Time Frame: until treatment discontinuation (assessment at the end of cycle 2 then every other cycle)]
Best overall response was defined as the best evaluation observed through the entire treatment period as assessed by Independent Radiologic Review [IRR] based on Response Evaluation Criteria in Solid Tumor (RECIST) criteria.
Objective response rate [Time Frame: until treatment discontinuation (assessment at the end of cycle 2 then every other cycle)]
Objective response rate was defined as the percentage of patients with IRR confirmed partial response or complete response prior to disease progression or treatment discontinuation.

519 participants were enrolled in this study. Participants were treated for 21-day cycles until disease progression, unacceptable toxicity, or consent withdrawal. After treatment discontinuation, participants were followed until end of study or death or receipt of new anticancer therapy, whichever was first.

In the Active Comparator Arm, gemcitabine/carboplatin was administered on Days 1 and 8 of 21-day cycle(s). The doses were:

Gemcitabine: 1000 mg/m² intravenous infusion (30±10 minutes).

Carboplatin: AUC 2 intravenous infusion (30±10 minutes or 60±10 minutes).

In the Experimental Arm, gemcitabine/carboplatin was administered on Days 1 and 8, and iniparib was administered on Days 1, 4, 8, and 11 of 21-day cycle(s). The doses were:

Gemcitabine: 1000 mg/m2 intravenous infusion (30±10 minutes).

Carboplatin: AUC 2 intravenous infusion (30±10 minutes or 60±10 minutes).

Iniparib: body weight adjusted dose, intravenous infusion (60±10 minutes).

Eligibility

Ages Eligible for Study: 18 Years and older (Adult, Senior)

Sexes Eligible for Study: Female

Accepts Healthy Volunteers: No

Inclusion Criteria:
Histologically documented breast cancer (either primary or metastatic site) that is ER-negative, PR-negative, and HER2 non-overexpressing by immunohistochemistry (0, 1) or fluorescence in situ hybridization (FISH).

Triple-negative tumors were defined by the following criteria:
HER2-non-overexpressing: FISH-negative (defined by ratio <2.2) or, immunohistochemical (IHC) 0, IHC 1+ or, IHC 2+ or IHC 3+ and FISH-negative.
ER- and PR-negative: <10% tumor staining by immunohistochemistry (IHC).
Never having received chemotherapy for metastatic disease or, having received 1 or 2 prior chemotherapy regimens in the metastatic setting (Prior adjuvant/neoadjuvant therapy was allowed);
Metastatic breast cancer (Stage IV) with measurable disease by RECIST 1.1 criteria;
Female, ≥18 years of age;
Eastern Cooperative Oncology Group (ECOG) performance status of 0-1;

Organ and marrow function as follows: absolute neutrophil count (ANC)≥1500/mm3, platelets ≥100,000/dL, hemoglobin ≥9 g/dL, bilirubin ≤1.5 mg/dL, serum creatinine ≤1.5 mg/dL or creatinine clearance ≥60 mL/min, alanine aminotransferase (ALT) and aspartate aminotransferase (AST)≤2.5 times the upper limit of normal if no liver involvement or <5 times the upper limit of normal with liver involvement;
Radiation therapy completed at least 14 days before study dosing on day 1; radiated lesions may not have served as measurable disease;
Central nervous system metastases allowed if subject did not require steroids, whole brain radiation therapy (XRT), gamma/cyber knife, and brain metastases were clinically stable without symptomatic progression;
For women of child bearing potential, documented negative pregnancy test within two weeks of study entry and agreement to acceptable birth control during the duration of the study therapy;
Tissue block (primary or metastatic) or readily available fresh frozen tumor tissue for PARP expression and other pharmacogenomic studies recommended (although its absence will not exclude subjects from participating);
No other diagnosis of malignancy (with exception of non melanoma skin cancer or a malignancy diagnosed ≥5 years ago);
Obtained informed consent;
Capability to understand and comply with the protocol and signed informed consent document.

Exclusion Criteria:
Systemic anticancer therapy within 14 days of the first dose of study drug;
Prior treatment with gemcitabine, carboplatin, cisplatin or iniparib
Had not recovered to grade <1 from adverse events (AEs) per National Cancer Institute Common Terminology Criteria for Adverse Events (NCI-CTCAE) v3.0 or to within 10% of baseline values due to investigational drugs or other medications administered more than 30 days prior to study enrollment;
Major medical conditions that might have affected study participation (e.g. uncontrolled pulmonary, renal, or hepatic dysfunction, uncontrolled infection, cardiac disease);
Concurrent radiation therapy intended to treat primary tumor not permitted throughout the course of the study; palliative radiation was acceptable;
Leptomeningeal disease or brain metastases requiring steroids or other therapeutic intervention;
Pregnancy or breastfeeding;
Inability or unwillingness to abide by the study protocol or cooperate fully with the investigator or designee.

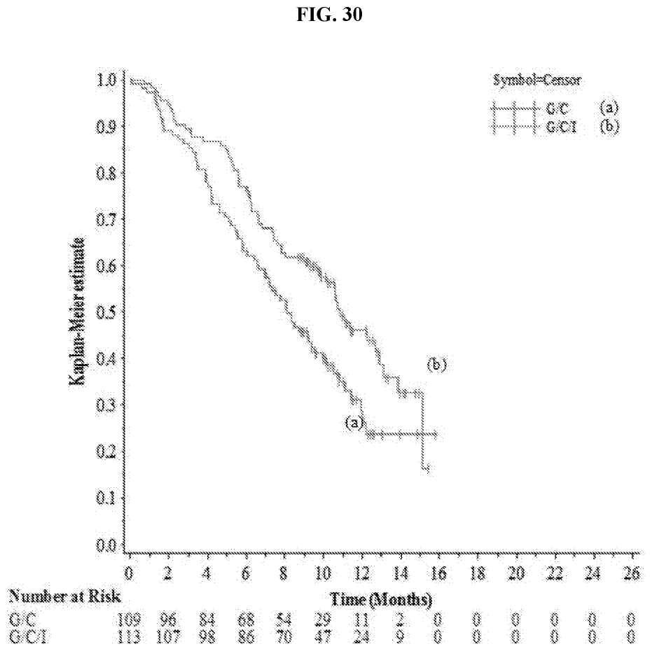

FIG. 30 and Table 12 show overall survival (OS) benefit achieved in 2^{nd}/3^{rd} line metastatic triple negative breast cancer (TNBC) at primary analysis. Median OS increased from 8.1 month to 10.8 month. The hazard ratio (HR) is 0.645. A 50% drop/improvement in risk of death was observed. The P-value is 0.0119. The primary planned analysis is at total of 260 deaths.

TABLE 12

| | G/C | G/C/I |
|---|---|---|
| OS | (N = 109) | (N = 113) |
| Median OS, mos (95% CI) | 8.1 | 10.8 |
| | (6.6, 10.0) | (9.7, 13.1) |

| | |
|---|---|
| HR (95% CI) | 0.645 (0.457, 0.911) |
| p-value | 0.0119 |

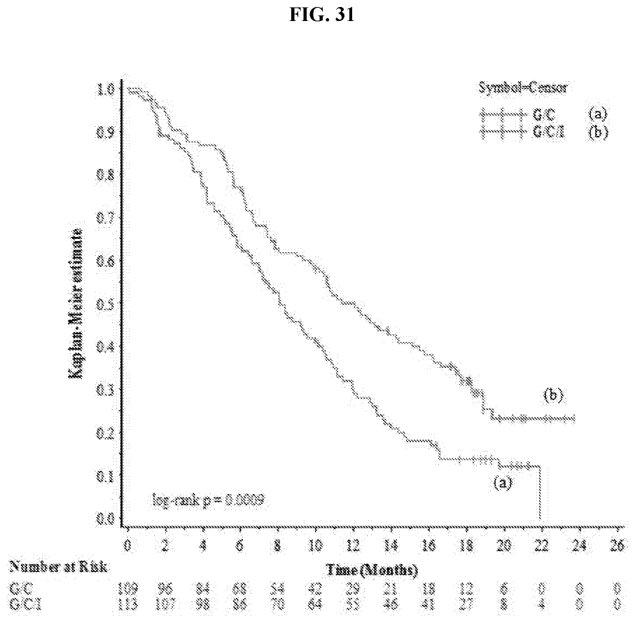

FIG. 31 and Table 13 show OA benefit achieved in 2^{nd}/3^{rd} line metastatic triple negative breast cancer (TNBC) at updated analysis. Median OA increased from 8.1 to 12.1 month. The hazard ratio (HR) is 0.604. A 50% drop improvement in risk of death was observed. The P-value is 0.0009. The updated analysis is with 100 additional deaths.

TABLE 13

| | G/C | G/C/I |
|---|---|---|
| OS | (N = 109) | (N = 113) |
| Median OS, mos (95% CI) | 8.1 | 12.1 |
| | (6.6, 10.2) | (9.9, 15.1) |

| | |
|---|---|
| HR (95% CI) | 0.604 (0.446, 0.817) |
| p-value | 0.0009 |

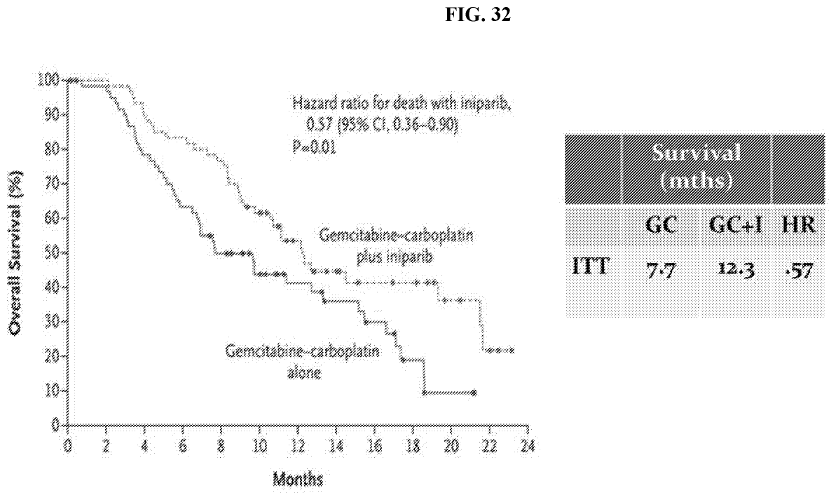

FIG. 32 illustrates overall survival (OS) benefit in all metastatic triple negative breast cancer (mTNBC) patients.

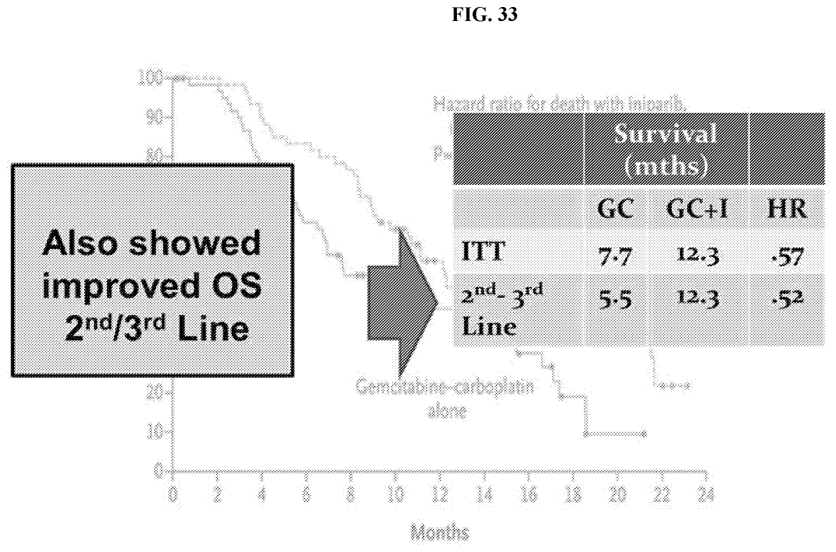

FIG. 33 illustrates overall survival (OS) benefit in 2^{nd}/3^{rd} line mTNBC patients.

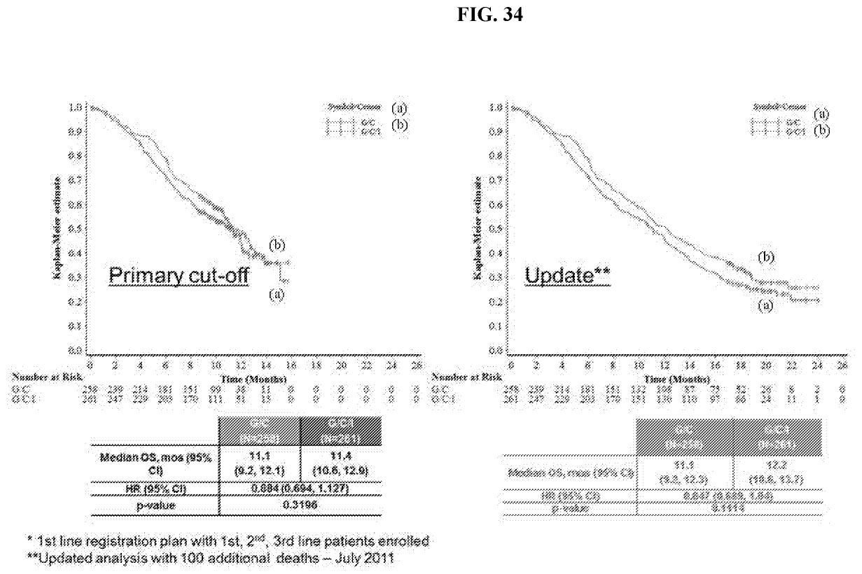

FIG. 34 illustrates Phase 3 clinical trial missed OS end point.

The length of Disease Free Interval (DFI) is an indicator of likely/potential survival. In some instances, DFI in TNBC is shorter than other breast cancer subtypes. No DFI eligibility restriction, or stratification, was included in the Phase 3 clinical trial. Manual compilation of DFI data is shown in Table 14.

TABLE 14

| DFI* by Patient Group | GC | GCI |
|---|---|---|
| DFI -- ITT | 15 months | 12 months |
| <12 mths | 44% | 51% |
| ≥12 mths | 56% | 49% |
| DFI - 1^{st} Line | (n = 149) | (n = 148) |
| Median | 15.9 months | 9.5 months |
| DFI - 2^{nd}/3^{rd} Line | (n = 109) | (n = 113) |
| Median | 13.8 months | 15.7 months |

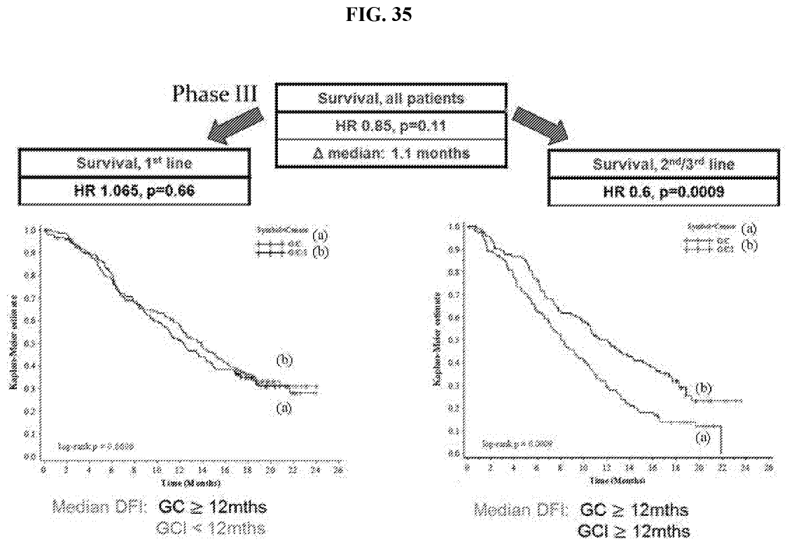

FIG. 35 illustrates impact of relapsed patient with short DFI on phase 3 OS.

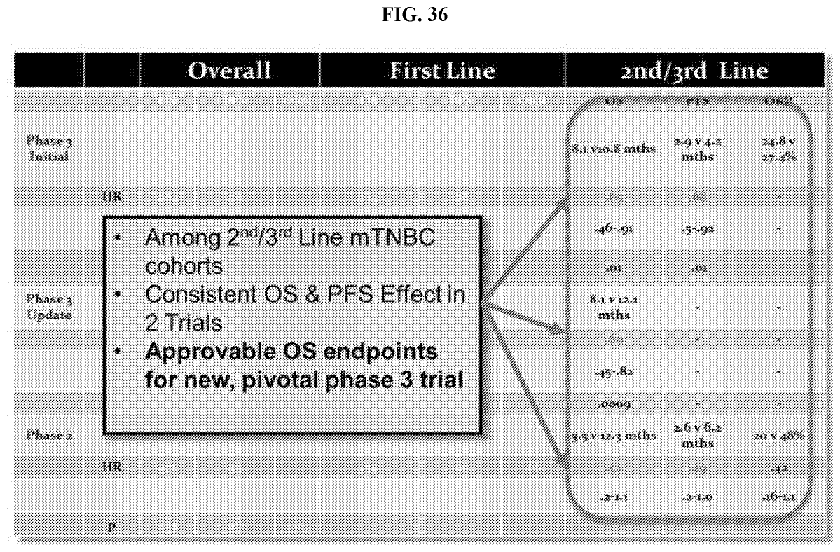

FIG. 36 illustrates 2^{nd}/3^{rd} line benefit across Phase 2 and Phase 3 trials and at endpoints.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method of treating a subject having a cancer, comprising:
a) determining whether the subject has an elevated expression of thioredoxin reductase (TrxR) or peroxiredoxin (PRDX) by i) measuring an expression level of TrxR or PRDX from a cancer sample obtained from the subject, and ii) determining whether the expression level of TrxR or PRDX from the cancer sample is elevated relative to a control sample; and
b) administering to the subject a therapeutically effective amount of 4-iodo-3-nitrobenzamide or a salt, metabolite or prodrug thereof, thereby treating the subject having the cancer characterized with the elevated expression of TrxR or PRDX.

2. A method of diagnosing and treating cancer in a subject, the method comprising:
a) obtaining a cancer sample from a human subject;
b) detecting whether an expression level of thioredoxin reductase (TrxR) or peroxiredoxin (PRDX) is elevated in the sample relative to an expression level of TrxR or PRDX in a control sample;
c) diagnosing the subject as having a cancer characterized with the elevated expression of TrxR or PRDX; and
d) administering an effective amount of 4-iodo-3-nitrobenzamide or a salt, metabolite or prodrug thereof to the diagnosed subject.

3. The method of claim 1 or 2, wherein the measuring comprises:
i) contacting a portion of the TrxR gene with a set of primers to produce amplified nucleic acids and determining the level of the amplified nucleic acids in the tumor sample;
ii) contacting a portion of the PRDX gene with a set of primers to produce amplified nucleic acids and determining the level of the amplified nucleic acids in the tumor sample; or
iii) a combination thereof.

4. The method of claim 1 or 2, wherein the detecting comprises:
i) contacting the sample with an anti-TrxR antibody and detecting binding between TrxR protein and the anti-TrxR antibody;
ii) contacting the sample with an anti-PRDX antibody and ii) detecting binding between PRDX protein and the anti-PRDX; or
iii) a combination thereof.

5. The method of claim 1 or 2, wherein TrxR is thioredoxin reductase 1 (TrxR-1).

6. The method of claim 1 or 2, wherein TrxR is thioredoxin reductase 2 (TrxR-2).

7. The method of claim 1 or 2, wherein the elevated expression level of TrxR:
is about 10%, 20%, 30, 40%, 50%, 60%, 70%, 80%, 90%, 95%, or 99% higher relative to the expression level of TrxR in a cell from the control sample; or
is about 1-fold, 2-fold, 3-fold, 4-fold, 5-fold, 6-fold, 7-fold, 8-fold, 9-fold, 10-fold, 15-fold, 20-fold or higher relative to the expression level of TrxR in a cell from the control sample.

8. The method of claim 1 or 2, wherein peroxiredoxin is peroxiredoxin-1 (PRDX-1).

9. The method of claim 8, wherein the elevated expression of peroxiredoxin-1 is determined by i) measuring an expression level of PRDX-1 from a tumor sample obtained from the subject, and ii) determining whether the expression level of PRDX-1 from the tumor sample is elevated relative to a control sample.

10. The method of claim 9, wherein the measuring comprises:
i) contacting a portion of the PRDX-1 gene with a set of primers to produce amplified nucleic acids and determining the level of the amplified nucleic acids in the tumor sample; or
ii) contacting the sample with an anti-PRDX-1 antibody and ii) detecting binding between PRDX-1 protein and the anti-PRDX-1 antibody.

11. The method of claim 1 or 2, wherein the elevated expression level of PRDX:
is about 10%, 20%, 30, 40%, 50%, 60%, 70%, 80%, 90%, 95%, or 99% higher relative to the expression level of PRDX in a cell from the control sample; or
is about 1-fold, 2-fold, 3-fold, 4-fold, 5-fold, 6-fold, 7-fold, 8-fold, 9-fold, 10-fold, 15-fold, 20-fold or higher relative to the expression level of PRDX in a cell from the control sample.

12. The method of claim 1 or 2, wherein the cancer is a solid tumor.

13. The method of claim 12, wherein the solid tumor comprises brain cancer, bladder cancer, breast cancer, colorectal cancer, lung cancer, or prostate cancer.

14. The method of claim 13, wherein the brain cancer comprises glioblastoma.

15. The method of claim 14, wherein the glioblastoma is primary glioblastoma.

16. The method of claim 14, wherein the glioblastoma is a secondary tumor.

17. The method of claim 1 or 2, wherein the subject has a grade III or grade IV glioblastoma.

18. The method of claim 12, wherein the cancer is breast cancer.

19. The method of claim 18, wherein the breast cancer is triple negative breast cancer.

20. The method of claim 1 or 2, wherein the cancer is a hematologic malignancy.

21. The method of claim 20, wherein the hematologic malignancy comprises T-cell leukemia.

22. The method of claim 21, wherein the T-cell leukemia comprises large granular lymphocytic leukemia, T-cell acute lymphoblastic leukemia (T-ALL) or T-cell prolymphocytic leukemia (T-PLL).

23. The method of claim 1 or 2, wherein the cancer is a melanoma.

24. The method of claim 1 or 2, wherein the cancer is a metastatic cancer, a relapsed cancer, or a refractory cancer.

25. The method of claim 1 or 2, wherein 4-iodo-3-nitrobenzamide or a salt, metabolite or prodrug thereof is administered to the subject:
a) at a range of about 5 mg/kg to about 40 mg/kg;
b) at a range of about 6 mg/kg to about 40 mg/kg, about 6 mg/kg to about 30 mg/kg, about 6 mg/kg to about 20 mg/kg, about 6 mg/kg to about 10 mg/kg, about 6 mg/kg to about 9 mg/kg, about 7 mg/kg to about 30 mg/kg, about 7 mg/kg to about 20 mg/kg, about 7 mg/kg to about 9 mg/kg, about 7 mg/kg to about 8 mg/kg, about 8 mg/kg to about 20 mg/kg, about 8 mg/kg to about 9 mg/kg, or about 8 mg/kg to about 8.6 mg/kg; or
c) at about 5 mg/kg, about 6 mg/kg, about 7 mg/kg, about 8 mg/kg, about 8.5 mg/kg, about 8.6 mg/kg, about 9 mg/kg, about 10 mg/kg, about 15 mg/kg, about 20 mg/kg, about 25 mg/kg, about 30 mg/kg, or about 40 mg/kg.

26. The method of claim 1 or 2, wherein the 4-iodo-3-nitrobenzamide or a salt, metabolite or prodrug thereof is administered to the subject once per day, for about twice a week, or for about four, five, or six weeks.

27. The method of claim 1 or 2, wherein the 4-iodo-3-nitrobenzamide or a salt, metabolite or prodrug thereof is administered to the subject continuously for about 1, 2, 3, 4 or more treatment cycles.

28. The method of claim 1 or 2, wherein the 4-iodo-3-nitrobenzamide or a salt, metabolite or prodrug thereof is administered to the subject intermittently for about 1, 2, 3, 4 or more treatment cycles.

29. The method of claim 27 or 28, wherein a treatment cycle is about 28 days.

30. The method of claim 1 or 2, further comprising administering to the subject an additional therapeutic agent.

31. The method of claim 30, wherein the additional therapeutic agent is an inhibitor of TrxR.

32. The method of claim 31, wherein the inhibitor of TrxR is:
a) epigallocatechin-3-O-gallate (EGCG), n-butyl 2-imidazolyl disulfide, 1-methylpropyl 2-imidazolyl disulfide, n-decyl 2-imidazolyl disulfide, an alkyl 2-imidazolyl disulfide analogue, auranofin, or a dinitrohalobenzene; or
b) phosphine gold(I), a gold(I) carbene complex, a gold(III)-dithiocarbamato complex, an arsenic derivative, or azelaic acid.

33. The method of claim 30, wherein the additional therapeutic agent is an inhibitor of PRDX.

34. The method of claim 33, wherein the inhibitor of PRDX is a pan-PRDX inhibitor.

35. The method of claim 34, wherein the inhibitor of PRDX is Conoidin A.

36. The method of claim 30, wherein the additional therapeutic agent is an inhibitor of glutathione (GSH).

37. The method of claim 36, wherein the inhibitor of GSH is L-buthionine sulfoximine (BSO).

38. The method of claim 30, wherein the additional therapeutic agent is temozolomide, radiation, or a standard-of-care chemotherapy.

39. The method of claim 30, wherein 4-iodo-3-nitrobenzamide or a salt, metabolite or prodrug thereof and the additional therapeutic agent are administered sequentially.

40. The method of claim 30, wherein 4-iodo-3-nitrobenzamide or a salt, metabolite or prodrug thereof and the additional therapeutic agent are administered concurrently.

41. The method of claim 1 or 2, wherein treatment of 4-iodo-3-nitrobenzamide or a salt, metabolite or prodrug thereof increases the length of disease free interval (DFI) relative to a subject not treated with 4-iodo-3-nitrobenzamide or a salt, metabolite or prodrug thereof.

42. The method of claim 1, wherein the control sample is a non-cancerous sample.

43. The method of claim 1 or 2, wherein the tumor sample is a tissue sample, a liquid sample, or a cell-free sample.

44. A method of treating a subject having a cancer characterized with an elevated expression of thioredoxin reductase (TrxR) or peroxiredoxin (PRDX), comprising:
administering to the subject a therapeutically effective amount of 4-iodo-3-nitrobenzamide or a salt, metabolite or prodrug thereof, thereby treating the subject having the cancer characterized with the elevated expression of TrxR or PRDX; wherein the subject is determined to have the elevated TrxR or PRDX by i) measuring an expression level of TrxR or PRDX from a cancer sample obtained from the subject, and ii) determining whether the expression level of TrxR or PRDX from the cancer sample is elevated relative to a control sample.

45. A method for treating a subject with 4-iodo-3-nitrobenzamide or a salt, metabolite or prodrug thereof, wherein the subject has a cancer, the method comprising:
determining whether the subject has an elevated expression of thioredoxin reductase (TrxR) or peroxiredoxin (PRDX) by:
i) measuring an expression level of TrxR or PRDX from a cancer sample obtained from the subject, and
ii) determining whether the expression level of TrxR or PRDX from the cancer sample is elevated relative to a control sample;
if the subject has an elevated expression of TrxR or PRDX, then administering 4-iodo-3-nitrobenzamide or a salt, metabolite or prodrug thereof to the subject, and
if the subject does not have an elevated expression of TrxR or PRDX, then administering a first-line treatment to the subject,
wherein a length of disease free interval (DFI) for the subject having an elevated expression of TrxR or PRDX is extended following administration of the treatment regimen comprising 4-iodo-3-nitrobenzamide or a salt, metabolite or prodrug thereof than it would be if the first-line treatment were administered.
